(19) United States
(12) Patent Application Publication
(10) Pub. No.: US 2026/0201474 A1
(43) Pub. Date: Jul. 16, 2026

(54) METHODS FOR PREDICTING CANCER-ASSOCIATED VENOUS THROMBOEMBOLISM USING CIRCULATING TUMOR DNA

(71) Applicants: MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE FOR CANCER RESEARCH, New York, NY (US)

(72) Inventors: Justin JEE, New York, NY (US); Simon MANTHA, New York, NY (US); Bob T. LI, New York, NY (US)

(21) Appl. No.: 19/128,981

(22) PCT Filed: Nov. 10, 2023
(86) PCT No.: PCT/US2023/079404

§ 371 Date: May 9, 2025

Related U.S. Application Data
(60) Provisional application No. 63/424,813, filed on Nov. 11, 2022.
(60) Provisional application No. 63/507,399, filed on Jun. 9, 2023.

(51) Int. Cl. C12Q 1/6886; A61K 31/235; A61K 31/366; A61K 31/37; A61K 31/40; A61K 31/405; A61K 31/44; A61K 31/4439; A61K 31/444; A61K 31/4545; A61K 31/47; A61K 31/505; A61K 31/5377; A61K 31/727; A61K 45/06; G16B 40/20; G16H 50/20

(52) U.S. Cl. CPC C12Q 1/6886; A61K 31/235; A61K 31/366; A61K 31/37; A61K 31/40; A61K 31/405; A61K 31/44; A61K 31/4439; A61K 31/444; A61K 31/4545; A61K 31/47; A61K 31/505; A61K 31/5377; A61K 31/727; A61K 45/06; G16B 40/20; G16H 50/20; C12Q 2600/156

(57) ABSTRACT

The present disclosure relates generally to methods for accurately predicting the risk of cancer-associated venous thromboembolism (CAT) and/or preventing CAT in cancer patients using ctDNA as a biomarker.

### CROSS-REFERENCE TO RELATED APPLICATIONS

This application claims the benefit of and priority to U.S. Provisional Patent Application No. 63/424,813, filed Nov. 11, 2022, and U.S. Provisional Patent Application No. 63/507,399, filed Jun. 9, 2023, the entire contents of which are incorporated herein by reference.

### TECHNICAL FIELD

The present technology relates generally to methods for accurately predicting the risk of cancer-associated venous thromboembolism (CAT) and/or preventing CAT in cancer patients using ctDNA as a biomarker.

### BACKGROUND

The following description of the background of the present technology is provided simply as an aid in understanding the present technology and is not admitted to describe or constitute prior art to the present technology.

Cancer associated thromboembolism (CAT) is a frequent complication of cancer with high morbidity. Biomarkers that effectively predict which patients are at highest risk of developing CAT are needed to assess which patients might benefit from prophylactic anticoagulation and further monitoring. The Khorana score, based on cancer type, pre-chemotherapy platelet and leukocyte count, hemoglobin, and body-mass index (BMI) is one such validated means of risk-stratifying patients for CAT (Khorana et al Blood 2008); it has been shown that patients with a high Khorana score are at high risk for CAT but that risk may be lowered by prophylactic anticoagulation (Khorana et al NEJM 2019, Carrier et al NEJM 2019). However, new molecular biomarkers may possess prognostic information not captured in laboratory, histopathologic, radiologic, or clinical variables (Jee et al ASCO 2021, ascopubs.org/doi/10.1200/JCO.2021.39.15_suppl.9009).

### SUMMARY OF THE PRESENT TECHNOLOGY

In one aspect, the present disclosure provides a method for preventing cancer associated thromboembolism (CAT) in a cancer patient in need thereof comprising (a) detecting ctDNA molecules in a biological sample obtained from the cancer patient, wherein the ctDNA molecules are detected at a variant allele fraction (VAF) detection limit of at least 0.1%-0.5% and (b) administering to the cancer patient an effective amount of anticoagulant therapy.

In another aspect, the present disclosure provides a method for preventing cancer associated thromboembolism (CAT) in a cancer patient in need thereof comprising administering to the cancer patient an effective amount of anticoagulant therapy, wherein a biological sample obtained from the cancer patient comprises detectable ctDNA molecules, wherein the ctDNA molecules are detected at a variant allele fraction (VAF) detection limit of at least 0.1%-0.5%.

Additionally or alternatively, in some embodiments of the methods disclosed herein, the ctDNA molecules are detected at a VAF detection limit of from about 0.1% to about 0.5%, from about 0.5% to about 2%, from about 2% to about 10% or from about 10% to about 99%. In certain embodiments, the ctDNA molecules are detected at a VAF detection limit of about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%.

In any of the preceding embodiments of the methods disclosed herein, the cancer patient is diagnosed with or suffers from a cancer selected from the group consisting of non-small cell lung cancer, breast cancer, pancreatic cancer, melanoma, retinoblastoma, prostate cancer, esophagogastric cancer, histiocytosis, germ cell tumor, endometrial cancer, small cell lung cancer, soft tissue sarcoma, Gastrointestinal Stromal Tumor, ovarian cancer, mature B-Cell neoplasms, small bowel cancer, renal cell carcinoma, thyroid cancer, ampullary cancer, appendiceal cancer, sellar tumor, uterine sarcoma, bone cancer, non-melanoma skin cancer, cervical cancer, mesothelioma, glioma, thymic tumor, gastrointestinal neuroendocrine tumor, salivary gland cancer, sex cord stromal tumor, anal cancer, mature T and NK neoplasms, peritoneal cancer, Head and neck cancer, choroid plexus tumor, leukemia, primary CNS melanocytic tumors, Myelodysplastic Syndromes, Peripheral Nervous System, mastocytosis, Wilms tumor, lymphatic cancer, vaginal cancer, Hodgkin lymphoma, adrenocortical carcinoma, brain tumors, embryonal tumors and Non-Hodgkin lymphoma. The cancer may be a Stage 1, Stage 2, Stage 3, or Stage 4 cancer. Additionally or alternatively, in some embodiments, the cancer patient has a Khorana Score ≥2 or <2 and/or has one or more organ sites of metastasis.

Additionally or alternatively, in some embodiments of the methods disclosed herein, the ctDNA molecules comprise one or more mutations (e.g., SNVs) in at least one cancer associated gene selected from the group consisting of AKT1, ALK, APC, AR, ARAF, ARIDIA, ARID2, ATM, B2M, BCL2, BCOR, BRAF, BRCA1, BRCA2, CARD11, CBFB, CCND1, CDH1, CDK4, CDKN2A, CIC, CREBBP, CTCF, CTNNB1, DICER1, DIS3, DNMT3A, EGFR, EIF1AX, EP300, ERBB2, ERBB3, ERCC2, ESR1, EZH2, FBXW7, FGFR1, FGFR2, FGFR3, FGFR4, FLT3, FOXA1, FOXL2, FOXO1, FUBP1, GATA3, GNA11, GNAQ, GNAS, H3F3A, HIST1H3B, HRAS, IDH1, IDH2, IKZF1, INPPL1, JAK1, KDM6A, KEAP1, KIT, KNSTRN, KRAS, MAP2K1, MAPK1, MAX, MED12, MET, MLH1, MSH2, MSH3, MSH6, MTOR, MYC, MYCN, MYD88, MYOD1, NF1, NFE2L2, NOTCH1, NRAS, NTRK1, NTRK2, NTRK3, NUP93, PAK7, PDGFRA, PIK3CA, PIK3CB, PIK3R1, PIK3R2, PMS2, POLE, PPP2RIA, PPP6C, PRKCI, PTCH1, PTEN, PTPN11, RAC1, RAF1, RB1, RET, RHOA, RIT1, ROS1, RRAS2, RXRA, SETD2, SF3B1, SMAD3, SMAD4, SMARCA4, SMARCB1, SOS1, SPOP, STAT3, STK11, STK19, TCF7L2, TGFBR1, TGFBR2, TP53, TP63, TSC1, TSC2, U2AF1, VHL, XPO1, and TERT. In certain embodiments, the ctDNA molecules comprise 2-20 mutations in the at the least one cancer associated gene.

In any and all embodiments of the methods disclosed herein, the ctDNA molecules comprise one or more rearrangements in at least one cancer associated gene selected from the group consisting of ALK, BRAF, EGFR, ETV6, FGFR2, FGFR3, MET, NTRK1, RET and ROS1. The one or more rearrangements may comprise indels, CNVs, and/or gene fusions. Additionally or alternatively, in some embodiments, the ctDNA molecules comprise 2-20 rearrangements in the at the least one cancer associated gene.

In any of the preceding embodiments of the methods disclosed herein, the biological sample is whole blood, serum or plasma. In some embodiments, the biological sample has a cfDNA concentration ranging from about 3 pg/μL to 5.5 ng/μL. In some embodiments, the biological sample has a cfDNA concentration of about 3 pg/μL, about 4 pg/μL, about 5 pg/μL, about 6 pg/μL, about 7 pg/μL, about 8 pg/μL, about 9 pg/μL, about 10 pg/μL, about 15 pg/μL, about 20 pg/μL, about 25 pg/μL, about 30 pg/μL, about 35 pg/μL, about 40 pg/μL, about 45 pg/μL, about 50 pg/μL, about 55 pg/μL, about 60 pg/μL, about 65 pg/μL, about 70 pg/μL, about 75 pg/μL, about 80 pg/μL, about 85 pg/μL, about 90 pg/μL, about 100 pg/μL, about 125 pg/μL, about 150 pg/μL, about 175 pg/μL, about 200 pg/μL, about 225 pg/μL, about 250 pg/μL, about 275 pg/μL, about 300 pg/μL, about 325 pg/μL, about 350 pg/μL, about 375 pg/μL, about 400 pg/μL, about 425 pg/μL, about 450 pg/μL, about 475 pg/μL, about 500 pg/μL, about 525 pg/μL, about 550 pg/μL, about 575 pg/μL, about 600 pg/μL, about 625 pg/μL, about 650 pg/μL, about 675 pg/μL, about 700 pg/μL, about 725 pg/μL, about 750 pg/μL, about 775 pg/μL, about 800 pg/μL, about 825 pg/μL, about 850 pg/μL, about 875 pg/μL, about 900 pg/μL, about 925 pg/μL, about 950 pg/μL, about 975 pg/μL, about 1 ng/μL, about 1.25 ng/μL, about 1.5 ng/μL, about 1.75 ng/μL, about 2 ng/μL, about 2.25 ng/μL, about 2.5 ng/μL, about 2.75 ng/μL, about 3 ng/μL, about 3.25 ng/μL, about 3.5 ng/μL, about 3.75 ng/μL, about 4 ng/μL, about 4.25 ng/μL, about 4.5 ng/μL, about 4.75 ng/μL, about 5 ng/μL, about 5.25 ng/μL, or about 5.5 ng/μL.

Additionally or alternatively, in some embodiments, the anticoagulant therapy comprises one or more of apixaban, betrixaban, dabigatran, edoxaban, fondaparinux, heparin, rivaroxaban, warfarin, Xa inhibitors, statins, or enoxaparin. Examples of statins include, but are not limited to atorvastatin, fluvastatin, lovastatin, pitavastatin, pravastatin, rosuvastatin, and simvastatin.

In any of the foregoing embodiments of the methods disclosed herein, the cancer patient is chemotherapy-naïve or has received/is receiving systemic chemotherapy. Systemic chemotherapy may comprise one or more of alkylating agents, antibiotics, antimetabolites, antimitotics, cyclin-dependent kinase inhibitors, epidermal growth factor receptor inhibitors, multikinase inhibitors, PARP inhibitors, platinum-based agents, selective estrogen receptor modulators (SERM), or VEGF inhibitors. Examples of chemotherapeutic agents include, but are not limited to, alkylating agents, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, bisphosphonate therapy agents and targeted biological therapy agents (e.g., therapeutic peptides described in U.S. Pat. No. 6,306,832, WO 2012007137, WO 2005000889, WO 2010096603 etc.). In some embodiments, the at least one additional therapeutic agent is a chemotherapeutic agent. Specific chemotherapeutic agents include, but are not limited to, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), bevacizumab, oxaliplatin, melphalan, etoposide, mechlorethamine, bleomycin, microtubule poisons, annonaceous acetogenins, or combinations thereof.

Additionally or alternatively, in some embodiments of the methods disclosed herein, the cancer patient is immunotherapy-naïve or has received/is receiving immunotherapy. Examples of immunotherapy include, but are not limited to, anti-PD-1 antibody, anti-PD-L1 antibody, anti-PD-L2 antibody, anti-CTLA-4 antibody, anti-TIM3 antibody, anti-4-1BB antibody, anti-CD73 antibody, anti-GITR antibody, and anti-LAG-3 antibody.

Additionally or alternatively, in certain embodiments of the methods disclosed herein, the cancer patient is radiotherapy-naïve or has received/is receiving radiotherapy. The radiotherapy may comprise external radiotherapy, radiotherapy implants (brachytherapy), pre-targeted radioimmunotherapy, radiotherapy injections, radioisotope therapy, or intrabeam radiotherapy.

In any and all embodiments of the methods disclosed herein, the CAT is pulmonary embolism or lower extremity deep vein thrombosis (DVT). In some embodiments, lower extremity DVT includes thrombi involving a common iliac vein, an external iliac vein, a common femoral vein, a superficial femoral vein, a deep femoral vein, a popliteal vein, a peroneal vein, an anterior tibial vein, a posterior tibial vein, or a deep calf vein.

In one aspect, the present disclosure provides a method for preventing cancer associated thromboembolism (CAT) in a lung cancer patient in need thereof comprising detecting ctDNA molecules in a biological sample obtained from the lung cancer patient, wherein the ctDNA molecules comprise at least one alteration in at least one cancer-associated gene selected from the group consisting of AKT1, ALK, B2M, BRAF, EGFR, ERBB2 (HER2), FGFR2, FGFR3, KEAP1, KRAS, MAP2K1 (MEK1), MET, NRAS, PIK3CA, RET, ROS1, STK11, TP53, NTRK1, FGFR1, MYC, PTEN, and RICTOR; and administering to the lung cancer patient an effective amount of anticoagulant therapy. The lung cancer may be non-small cell lung cancer (NSCLC) or small cell lung cancer (SCLC). In some embodiments, the lung cancer is Stage 1, Stage 2, Stage 3, or Stage 4.

In another aspect, the present disclosure provides a method for preventing cancer associated thromboembolism (CAT) in a lung cancer patient in need thereof comprising administering to the lung cancer patient an effective amount of anticoagulant therapy, wherein a biological sample obtained from the lung cancer patient comprises detectable ctDNA molecules comprising at least one alteration in at least one cancer-associated gene selected from the group consisting of AKT1, ALK, B2M, BRAF, EGFR, ERBB2 (HER2), FGFR2, FGFR3, KEAP1, KRAS, MAP2K1 (MEK1), MET, NRAS, PIK3CA, RET, ROS1, STK11, TP53, NTRK1, FGFR1, MYC, PTEN, and RICTOR. The lung cancer may be non-small cell lung cancer (NSCLC) or small cell lung cancer (SCLC). In certain embodiments, the lung cancer is Stage 1, Stage 2, Stage 3, or Stage 4.

Additionally or alternatively, in some embodiments, the anticoagulant therapy comprises one or more of apixaban, betrixaban, dabigatran, edoxaban, fondaparinux, heparin, rivaroxaban, warfarin, Xa inhibitors, statins, or enoxaparin. Examples of statins include, but are not limited to atorvastatin, fluvastatin, lovastatin, pitavastatin, pravastatin, rosuvastatin, and simvastatin.

In any of the preceding embodiments of the methods disclosed herein, the lung cancer patient has a Khorana Score <2 or ≥2. Additionally or alternatively, in certain embodiments, the at least one alteration is a SNV, an indel, a CNV, or a gene fusion.

Additionally or alternatively, in some embodiments of the methods disclosed herein, the at least one alteration is detected at a variant allele fraction (VAF) detection limit of 0.1%-0.5%. In certain embodiments, the detected ctDNA molecules comprise one alteration in the at the least one cancer associated gene. In other embodiments, the detected ctDNA molecules comprise 2-20 alterations in the at the least one cancer associated gene. Additionally or alternatively, in some embodiments of the methods disclosed herein, the ctDNA molecules are detected via polymerase chain reaction (PCR), real-time quantitative PCR (qPCR), droplet digital PCR (ddPCR), Reverse transcriptase-PCR (RT-PCR), microarray, RNA-Seq, or next-generation sequencing. In any of the preceding embodiments of the methods disclosed herein, the biological sample is whole blood, serum or plasma.

In any of the foregoing embodiments of the methods disclosed herein, the lung cancer patient is chemotherapy-naïve or has received/is receiving systemic chemotherapy. Examples of systemic chemotherapy include, but are not limited to, alkylating agents, antibiotics, antimetabolites, antimitotics, cyclin-dependent kinase inhibitors, epidermal growth factor receptor inhibitors, multikinase inhibitors, PARP inhibitors, platinum-based agents, selective estrogen receptor modulators (SERM), or VEGF inhibitors.

Additionally or alternatively, in some embodiments of the methods disclosed herein, the lung cancer patient is immunotherapy-naïve or has received/is receiving immunotherapy. Examples of immunotherapy include, but are not limited to, anti-PD-1 antibody, anti-PD-L1 antibody, anti-PD-L2 antibody, anti-CTLA-4 antibody, anti-TIM3 antibody, anti-4-1BB antibody, anti-CD73 antibody, anti-GITR antibody, and anti-LAG-3 antibody.

Additionally or alternatively, in certain embodiments of the methods disclosed herein, the lung cancer patient is radiotherapy-naïve or has received/is receiving radiotherapy. The radiotherapy may comprise external radiotherapy, radiotherapy implants (brachytherapy), pre-targeted radioimmunotherapy, radiotherapy injections, radioisotope therapy, or intrabeam radiotherapy.

In any and all embodiments of the methods disclosed herein, the CAT is pulmonary embolism or lower extremity deep vein thrombosis (DVT). In some embodiments, lower extremity DVT includes thrombi involving a common iliac vein, an external iliac vein, a common femoral vein, a superficial femoral vein, a deep femoral vein, a popliteal vein, a peroneal vein, an anterior tibial vein, a posterior tibial vein, or a deep calf vein.

Additionally or alternatively, in certain embodiments of the methods disclosed herein, the at least one alteration comprises a SNV and/or an indel in one or more of AKT1, ALK, B2M, BRAF, EGFR, ERBB2 (HER2), FGFR2, FGFR3, KEAP1, KRAS, MAP2K1 (MEK1), MET, NRAS, PIK3CA, RET, ROS1, STK11 and TP53. In some embodiments of the methods disclosed herein, the at least one alteration comprises a gene fusion in one or more of ALK, EGFR, FGFR2, FGFR3, NTRK1, RET, and ROS1. Additionally or alternatively, in some embodiments, the at least one alteration comprises a CNV in one or more of B2M, EGFR, ERBB2 (HER2), FGFR1, KRAS, MET, MYC, NTRK1, PIK3CA, PTEN, RICTOR, STK11, and TP53.

In one aspect, the present disclosure provides a method of training a machine learning classifier for estimating risk of cancer-associated venous thromboembolism (VTE) in cancer patients comprising: (a) receiving data on a cohort of subjects, the subjects in the cohort having a plurality of cancer types; (b) generating a training dataset based on the received data, wherein the training dataset comprises a plurality of features for each subject in the cohort, wherein the plurality of features comprises (i) cell free DNA concentration, (ii) maximum ctDNA VAF, (iii) ctDNA alterations in at least one cancer associated gene, and (iv) cancer type; and (c) applying a machine learning method to the training dataset to develop the machine learning classifier for estimating risk of cancer-associated VTE in cancer patients, wherein applying the machine learning method comprises: applying a machine learning technique to the training dataset; performing hyperparameter optimization to identify one or more machine learning models with an accuracy that exceeds an accuracy threshold for the classifier; and determining an optimal operating-point threshold based on optimization of sensitivity and specificity of the receiver operating characteristic (ROC) curves for the training dataset; wherein the classifier is configured to receive the plurality of features for cancer patients and generate predictors for risk of cancer-associated VTE in cancer patients. The subjects in the cohort may be chemotherapy-naïve or may have received systemic chemotherapy. Additionally or alternatively, in certain embodiments, the plurality of cancer types are selected from the group consisting of non-small cell lung cancer, breast cancer, pancreatic cancer, melanoma, retinoblastoma, prostate cancer, esophagogastric cancer, histiocytosis, germ cell tumor, endometrial cancer, small cell lung cancer, soft tissue sarcoma, Gastrointestinal Stromal Tumor, ovarian cancer, mature B-Cell neoplasms, small bowel cancer, renal cell carcinoma, thyroid cancer, ampullary cancer, appendiceal cancer, sellar tumor, uterine sarcoma, bone cancer, non-melanoma skin cancer, cervical cancer, mesothelioma, glioma, thymic tumor, gastrointestinal neuroendocrine tumor, salivary gland cancer, sex cord stromal tumor, anal cancer, mature T and NK neoplasms, peritoneal cancer, Head and neck cancer, choroid plexus tumor, leukemia, primary CNS melanocytic tumors, Myelodysplastic Syndromes, Peripheral Nervous System, mastocytosis, Wilms tumor, lymphatic cancer, vaginal cancer, Hodgkin lymphoma, adrenocortical carcinoma, brain tumors, embryonal tumors and Non-Hodgkin lymphoma.

The machine learning technique may model survival outcomes with competing risks. In some embodiments, the machine learning technique is a random forest technique, and the one or more machine learning models are random forest models. Additionally or alternatively, in certain embodiments, the machine learning classifier is an ensemble learning random forest classifier. Additionally or alternatively, in some embodiments, performing the hyperparameter optimization comprises performing an exhaustive grid search technique.

Additionally or alternatively, in some embodiments of the methods disclosed herein, the at least one cancer associated gene is selected from the group consisting of AKT1, ALK, APC, AR, ARAF, ARIDIA, ARID2, ATM, B2M, BCL2, BCOR, BRAF, BRCA1, BRCA2, CARD11, CBFB, CCND1, CDH1, CDK4, CDKN2A, CIC, CREBBP, CTCF, CTNNB1, DICER1, DIS3, DNMT3A, EGFR, EIF1AX, EP300, ERBB2, ERBB3, ERCC2, ESR1, EZH2, FBXW7, FGFR1, FGFR2, FGFR3, FGFR4, FLT3, FOXA1, FOXL2, FOXO1, FUBP1, GATA3, GNA11, GNAQ, GNAS, H3F3A, HIST1H3B, HRAS, IDH1, IDH2, IKZF1, INPPL1, JAK1, KDM6A, KEAP1, KIT, KNSTRN, KRAS, MAP2K1, MAPK1, MAX, MED12, MET, MLH1, MSH2, MSH3, MSH6, MTOR, MYC, MYCN, MYD88, MYOD1, NF1, NFE2L2, NOTCH1, NRAS, NTRK1, NTRK2, NTRK3, NUP93, PAK7, PDGFRA, PIK3CA, PIK3CB, PIK3R1, PIK3R2, PMS2, POLE, PPP2RIA, PPP6C, PRKCI, PTCH1, PTEN, PTPN11, RAC1, RAF1, RB1, RET, RHOA, RIT1, ROS1, RRAS2, RXRA, SETD2, SF3B1, SMAD3, SMAD4, SMARCA4, SMARCB1, SOS1, SPOP, STAT3, STK11, STK19, TCF7L2, TGFBR1, TGFBR2, TP53, TP63, TSC1, TSC2, U2AF1, VHL, XPO1, and TERT.

Additionally or alternatively, in some embodiments of the methods disclosed herein, the plurality of features further comprises platelet count, hemoglobin levels, leukocyte counts, body mass index (BMI), administration of chemotherapy, age, time from cancer diagnosis, race, and metastatic sites of disease. In certain embodiments, the metastatic sites of disease comprise one or more of adrenal gland, bone, brain, liver, lung, lymph, and pleura.

In any of the preceding embodiments, the method further comprises applying the classifier to data on a cancer patient to generate a predictor, and determining whether the cancer patient is at risk for cancer-associated VTE based on the predictor and the operating-point threshold. In some embodiments, the predictor comprises a cumulative incidence function (CIF) for cancer-associated VTE.

In any of the foregoing embodiments, the method further comprises administering an effective amount of anticoagulant therapy to the cancer patient predicted to be at risk for cancer-associated VTE based on the predictor and the operating-point threshold. Examples of anticoagulant therapy include, but are not limited to, apixaban, betrixaban, dabigatran, edoxaban, fondaparinux, heparin, rivaroxaban, warfarin, Xa inhibitors, statins, and enoxaparin. Examples of statins include, but are not limited to atorvastatin, fluvastatin, lovastatin, pitavastatin, pravastatin, rosuvastatin, and simvastatin.

In some embodiments, the cancer patient is chemotherapy-naïve or has received/is receiving systemic chemotherapy.

In one aspect, the present disclosure provides a method of estimating risk of cancer-associated venous thromboembolism (VTE) in a cancer patient using a machine learning classifier, the method comprising: receiving patient data corresponding to a plurality of features for the cancer patient; applying the machine learning classifier to the patient data to generate a predictor; and determining whether the cancer patient is at risk for cancer-associated VTE based on the predictor and an operating-point threshold, wherein the machine learning classifier is trained by: (a) receiving cohort data on a cohort of subjects, the subjects in the cohort having a plurality of cancer types; (b) generating a training dataset based on the received cohort data, wherein the training dataset comprises the plurality of features for each subject in the cohort, wherein the plurality of features comprises (i) cell free DNA concentration, (ii) maximum ctDNA VAF, (iii) ctDNA alterations in at least one cancer associated gene, and (iv) cancer type; and (c) applying a machine learning method to the training dataset to develop the machine learning classifier for estimating risk of cancer-associated VTE, wherein applying the machine learning method comprises: applying a machine learning technique to the training dataset; performing hyperparameter optimization to identify one or more machine learning models with an accuracy that exceeds an accuracy threshold for the machine learning classifier; and determining the optimal operating-point threshold based on optimization of sensitivity and specificity of the receiver operating characteristic (ROC) curves for the training dataset; wherein the machine learning classifier is configured to receive the plurality of features for cancer patients and generate predictors for risk of cancer-associated VTE in cancer patients. In some embodiments, the method further comprises administering an effective amount of anticoagulant therapy to the cancer patient predicted to be at risk for cancer-associated VTE based on the predictor and the operating-point threshold. Examples of anticoagulant therapy include, but are not limited to, apixaban, betrixaban, dabigatran, edoxaban, fondaparinux, heparin, rivaroxaban, warfarin, Xa inhibitors, statins, and enoxaparin. Examples of statins include, but are not limited to atorvastatin, fluvastatin, lovastatin, pitavastatin, pravastatin, rosuvastatin, and simvastatin. Additionally or alternatively, in some embodiments, the predictor comprises a cumulative incidence function (CIF) for cancer-associated VTE. The subjects in the cohort may be chemotherapy-naïve or may have received systemic chemotherapy. In any of the preceding embodiments of the methods disclosed herein, one or more of the plurality of features for the cancer patient are determined by assaying blood and/or sequencing tumor DNA.

Additionally or alternatively, in certain embodiments, the plurality of cancer types are selected from the group consisting of non-small cell lung cancer, breast cancer, pancreatic cancer, melanoma, retinoblastoma, prostate cancer, esophagogastric cancer, histiocytosis, germ cell tumor, endometrial cancer, small cell lung cancer, soft tissue sarcoma, Gastrointestinal Stromal Tumor, ovarian cancer, mature B-Cell neoplasms, small bowel cancer, renal cell carcinoma, thyroid cancer, ampullary cancer, appendiceal cancer, sellar tumor, uterine sarcoma, bone cancer, non-melanoma skin cancer, cervical cancer, mesothelioma, glioma, thymic tumor, gastrointestinal neuroendocrine tumor, salivary gland cancer, sex cord stromal tumor, anal cancer, mature T and NK neoplasms, peritoneal cancer, Head and neck cancer, choroid plexus tumor, leukemia, primary CNS melanocytic tumors, Myelodysplastic Syndromes, Peripheral Nervous System, mastocytosis, Wilms tumor, lymphatic cancer, vaginal cancer, Hodgkin lymphoma, adrenocortical carcinoma, brain tumors, embryonal tumors and Non-Hodgkin lymphoma.

The machine learning technique may model survival outcomes with competing risks. In some embodiments, the machine learning technique is a random forest technique, and the one or more machine learning models are random forest models. Additionally or alternatively, in certain embodiments, the machine learning classifier is an ensemble learning random forest classifier. Additionally or alternatively, in some embodiments, performing the hyperparameter optimization comprises performing an exhaustive grid search technique.

Additionally or alternatively, in some embodiments of the methods disclosed herein, the plurality of features further comprises platelet count, hemoglobin levels, leukocyte counts, body mass index (BMI), administration of chemotherapy, age, time from cancer diagnosis, race, and metastatic sites of disease.

Additionally or alternatively, in some embodiments of the methods disclosed herein, the at least one cancer associated gene is selected from the group consisting of AKT1, ALK, APC, AR, ARAF, ARIDIA, ARID2, ATM, B2M, BCL2, BCOR, BRAF, BRCA1, BRCA2, CARD11, CBFB, CCND1, CDH1, CDK4, CDKN2A, CIC, CREBBP, CTCF, CTNNB1, DICER1, DIS3, DNMT3A, EGFR, EIF1AX, EP300, ERBB2, ERBB3, ERCC2, ESR1, EZH2, FBXW7, FGFR1, FGFR2, FGFR3, FGFR4, FLT3, FOXA1, FOXL2, FOXO1, FUBP1, GATA3, GNA11, GNAQ, GNAS, H3F3A, HIST1H3B, HRAS, IDH1, IDH2, IKZF1, INPPL1, JAK1, KDM6A, KEAP1, KIT, KNSTRN, KRAS, MAP2K1, MAPK1, MAX, MED12, MET, MLH1, MSH2, MSH3, MSH6, MTOR, MYC, MYCN, MYD88, MYOD1, NF1, NFE2L2, NOTCH1, NRAS, NTRK1, NTRK2, NTRK3, NUP93, PAK7, PDGFRA, PIK3CA, PIK3CB, PIK3R1, PIK3R2, PMS2, POLE, PPP2RIA, PPP6C, PRKCI, PTCH1, PTEN, PTPN11, RAC1, RAF1, RB1, RET, RHOA, RIT1, ROS1, RRAS2, RXRA, SETD2, SF3B1, SMAD3, SMAD4, SMARCA4, SMARCB1, SOS1, SPOP, STAT3, STK11, STK19, TCF7L2, TGFBR1, TGFBR2, TP53, TP63, TSC1, TSC2, U2AF1, VHL, XPO1, and TERT.

In some embodiments, the cancer patient is chemotherapy-naïve or has received/is receiving systemic chemotherapy.

In any and all embodiments of the methods disclosed herein, one or more of the plurality of features for each subject in the cohort are determined by assaying blood and/or sequencing tumor DNA.

In any and all embodiments of the methods disclosed herein, the cancer-associated VTE is pulmonary embolism or lower extremity deep vein thrombosis (DVT), optionally wherein lower extremity DVT includes thrombi involving a common iliac vein, an external iliac vein, a common femoral vein, a superficial femoral vein, a deep femoral vein, a popliteal vein, a peroneal vein, an anterior tibial vein, a posterior tibial vein, or a deep calf vein.

In another aspect, the present disclosure provides a machine learning system for training a machine learning classifier for estimating risk of cancer-associated venous thromboembolism (VTE) in cancer patients, the system comprising a processor and a memory with instructions which, when executed by the processor, cause the processor to: (a) receive data on a cohort of subjects, the subjects in the cohort having a plurality of cancer types; (b) generate a training dataset based on the received data, wherein the training dataset comprises a plurality of features for each subject in the cohort, wherein the plurality of features comprises (i) cell free DNA concentration, (ii) maximum ctDNA VAF, (iii) ctDNA alterations in at least one cancer associated gene, and (iv) cancer type; and (c) apply a machine learning method to the training dataset to develop the machine learning classifier for estimating risk of cancer-associated VTE in cancer patients; wherein applying the machine learning method comprises: applying a machine learning technique to the training dataset; performing hyperparameter optimization to identify one or more machine learning models with an accuracy that exceeds an accuracy threshold for the machine learning classifier; and determining an optimal operating-point threshold based on optimization of sensitivity and specificity of the receiver operating characteristic (ROC) curves for the training dataset; wherein the machine learning classifier is configured to receive the plurality of features for cancer patients and generate predictors for risk of cancer-associated VTE in cancer patients. The subjects in the cohort may be chemotherapy-naïve or may have received systemic chemotherapy.

The machine learning technique may model survival outcomes with competing risks. In some embodiments, the machine learning technique is a random forest technique, and the one or more machine learning models are random forest models. Additionally or alternatively, in certain embodiments, the machine learning classifier is an ensemble learning random forest classifier.

Additionally or alternatively, in some embodiments, performing the hyperparameter optimization comprises performing an exhaustive grid search technique.

Additionally or alternatively, in some embodiments of the systems disclosed herein, the at least one cancer associated gene is selected from the group consisting of AKT1, ALK, APC, AR, ARAF, ARIDIA, ARID2, ATM, B2M, BCL2, BCOR, BRAF, BRCA1, BRCA2, CARD11, CBFB, CCND1, CDH1, CDK4, CDKN2A, CIC, CREBBP, CTCF, CTNNB1, DICER1, DIS3, DNMT3A, EGFR, EIF1AX, EP300, ERBB2, ERBB3, ERCC2, ESR1, EZH2, FBXW7, FGFR1, FGFR2, FGFR3, FGFR4, FLT3, FOXA1, FOXL2, FOXO1, FUBP1, GATA3, GNA11, GNAQ, GNAS, H3F3A, HIST1H3B, HRAS, IDH1, IDH2, IKZF1, INPPL1, JAK1, KDM6A, KEAP1, KIT, KNSTRN, KRAS, MAP2K1, MAPK1, MAX, MED12, MET, MLH1, MSH2, MSH3, MSH6, MTOR, MYC, MYCN, MYD88, MYOD1, NF1, NFE2L2, NOTCH1, NRAS, NTRK1, NTRK2, NTRK3, NUP93, PAK7, PDGFRA, PIK3CA, PIK3CB, PIK3R1, PIK3R2, PMS2, POLE, PPP2RIA, PPP6C, PRKCI, PTCH1, PTEN, PTPN11, RAC1, RAF1, RB1, RET, RHOA, RIT1, ROS1, RRAS2, RXRA, SETD2, SF3B1, SMAD3, SMAD4, SMARCA4, SMARCB1, SOS1, SPOP, STAT3, STK11, STK19, TCF7L2, TGFBR1, TGFBR2, TP53, TP63, TSC1, TSC2, U2AF1, VHL, XPO1, and TERT.

Additionally or alternatively, in some embodiments of the systems disclosed herein, the plurality of features further comprises platelet count, hemoglobin levels, leukocyte counts, body mass index (BMI), administration of chemotherapy, age, time from cancer diagnosis, race, and metastatic sites of disease. Metastatic sites of disease may comprise one or more of adrenal gland, bone, brain, liver, lung, lymph, and pleura.

Additionally or alternatively, in certain embodiments of the systems disclosed herein, the plurality of cancer types are selected from the group consisting of non-small cell lung cancer, breast cancer, pancreatic cancer, melanoma, retinoblastoma, prostate cancer, esophagogastric cancer, histiocytosis, germ cell tumor, endometrial cancer, small cell lung cancer, soft tissue sarcoma, Gastrointestinal Stromal Tumor, ovarian cancer, mature B-Cell neoplasms, small bowel cancer, renal cell carcinoma, thyroid cancer, ampullary cancer, appendiceal cancer, sellar tumor, uterine sarcoma, bone cancer, non-melanoma skin cancer, cervical cancer, mesothelioma, glioma, thymic tumor, gastrointestinal neuroendocrine tumor, salivary gland cancer, sex cord stromal tumor, anal cancer, mature T and NK neoplasms, peritoneal cancer, Head and neck cancer, choroid plexus tumor, leukemia, primary CNS melanocytic tumors, Myelodysplastic Syndromes, Peripheral Nervous System, mastocytosis, Wilms tumor, lymphatic cancer, vaginal cancer, Hodgkin lymphoma, adrenocortical carcinoma, brain tumors, embryonal tumors and Non-Hodgkin lymphoma.

In any of the preceding embodiments of the systems described herein, the instructions further cause the processor to apply the machine learning classifier to data on a cancer patient to generate a predictor, and determine whether the cancer patient is at risk for cancer-associated VTE based on the predictor and the operating-point threshold. In some embodiments, the predictor comprises a cumulative incidence function (CIF) for cancer-associated VTE.

In any of the foregoing embodiments of the systems described herein, the instructions further cause the processor to recommend an anticoagulant therapy to the cancer patient predicted to be at risk for cancer-associated VTE based on the predictor and the operating-point threshold. Examples of anticoagulant therapy include, but are not limited to, apixaban, betrixaban, dabigatran, edoxaban, fondaparinux, heparin, rivaroxaban, warfarin, Xa inhibitors, statins, and enoxaparin. Examples of statins include, but are not limited to atorvastatin, fluvastatin, lovastatin, pitavastatin, pravastatin, rosuvastatin, and simvastatin.

In some embodiments, the cancer patient is chemotherapy-naïve or has received/is receiving systemic chemotherapy.

In yet another aspect, the present disclosure provides a computing system for estimating risk of cancer-associated venous thromboembolism (VTE) in a cancer patient, the computing system comprising a processor and a memory with instructions which, when executed by the processor, cause the processor to: receive patient data corresponding to a plurality of features for the cancer patient; apply a machine learning classifier to the patient data to generate a predictor; and determine whether the cancer patient is at risk for cancer-associated VTE based on the predictor and an operating-point threshold, wherein the classifier is trained by: (a) receiving cohort data on a cohort of subjects, the subjects in the cohort having a plurality of cancer types; (b) generating a training dataset based on the received cohort data, wherein the training dataset comprises the plurality of features for each subject in the cohort, wherein the plurality of features comprises (i) cell free DNA concentration, (ii) maximum ctDNA VAF, (iii) ctDNA alterations in at least one cancer associated gene, and (iv) cancer type; and (c) applying a machine learning method to the training dataset to develop the machine learning classifier for estimating risk of cancer-associated VTE, wherein applying the machine learning method comprises: applying a machine learning technique to the training dataset; performing hyperparameter optimization to identify one or more machine learning models with an accuracy that exceeds an accuracy threshold for the machine learning classifier; and determining the optimal operating-point threshold based on optimization of sensitivity and specificity of the receiver operating characteristic (ROC) curves for the training dataset; wherein the machine learning classifier is configured to receive the plurality of features for cancer patients and generate predictors for risk of cancer-associated VTE in cancer patients.

The machine learning technique may model survival outcomes with competing risks. In some embodiments, the machine learning technique is a random forest technique, and the one or more machine learning models are random forest models. Additionally or alternatively, in certain embodiments, the machine learning classifier is an ensemble learning random forest classifier.

Additionally or alternatively, in some embodiments, performing the hyperparameter optimization comprises performing an exhaustive grid search technique.

Additionally or alternatively, in some embodiments of the systems disclosed herein, the plurality of features further comprises platelet count, hemoglobin levels, leukocyte counts, body mass index (BMI), administration of chemotherapy, age, time from cancer diagnosis, race, and metastatic sites of disease.

In certain embodiments, the at least one cancer associated gene is selected from the group consisting of AKT1, ALK, APC, AR, ARAF, ARIDIA, ARID2, ATM, B2M, BCL2, BCOR, BRAF, BRCA1, BRCA2, CARD11, CBFB, CCND1, CDH1, CDK4, CDKN2A, CIC, CREBBP, CTCF, CTNNB1, DICER1, DIS3, DNMT3A, EGFR, EIF1AX, EP300, ERBB2, ERBB3, ERCC2, ESR1, EZH2, FBXW7, FGFR1, FGFR2, FGFR3, FGFR4, FLT3, FOXA1, FOXL2, FOXO1, FUBP1, GATA3, GNA11, GNAQ, GNAS, H3F3A, HIST1H3B, HRAS, IDH1, IDH2, IKZF1, INPPL1, JAK1, KDM6A, KEAP1, KIT, KNSTRN, KRAS, MAP2K1, MAPK1, MAX, MED12, MET, MLH1, MSH2, MSH3, MSH6, MTOR, MYC, MYCN, MYD88, MYOD1, NF1, NFE2L2, NOTCH1, NRAS, NTRK1, NTRK2, NTRK3, NUP93, PAK7, PDGFRA, PIK3CA, PIK3CB, PIK3R1, PIK3R2, PMS2, POLE, PPP2RIA, PPP6C, PRKCI, PTCH1, PTEN, PTPN11, RAC1, RAF1, RB1, RET, RHOA, RIT1, ROS1, RRAS2, RXRA, SETD2, SF3B1, SMAD3, SMAD4, SMARCA4, SMARCB1, SOS1, SPOP, STAT3, STK11, STK19, TCF7L2, TGFBR1, TGFBR2, TP53, TP63, TSC1, TSC2, U2AF1, VHL, XPO1, and TERT

In any of the preceding embodiments of the systems described herein, the instructions further cause the processor to recommend an anticoagulant therapy to the cancer patient predicted to be at risk for cancer-associated VTE based on the predictor and the operating-point threshold. In some embodiments, the predictor comprises a cumulative incidence function (CIF) for cancer-associated VTE. Examples of anticoagulant therapy include, but are not limited to, apixaban, betrixaban, dabigatran, edoxaban, fondaparinux, heparin, rivaroxaban, warfarin, Xa inhibitors, statins, and enoxaparin. Examples of statins include, but are not limited to atorvastatin, fluvastatin, lovastatin, pitavastatin, pravastatin, rosuvastatin, and simvastatin.

Additionally or alternatively, in certain embodiments of the systems disclosed herein, the plurality of cancer types are selected from the group consisting of non-small cell lung cancer, breast cancer, pancreatic cancer, melanoma, retinoblastoma, prostate cancer, esophagogastric cancer, histiocytosis, germ cell tumor, endometrial cancer, small cell lung cancer, soft tissue sarcoma, Gastrointestinal Stromal Tumor, ovarian cancer, mature B-Cell neoplasms, small bowel cancer, renal cell carcinoma, thyroid cancer, ampullary cancer, cervical cancer, mesothelioma, glioma, thymic tumor, gastrointestinal neuroendocrine tumor, salivary gland cancer, sex cord stromal tumor, anal cancer, mature T and NK neoplasms, peritoneal cancer, Head and neck cancer, choroid plexus tumor, leukemia, primary CNS melanocytic tumors, Myelodysplastic Syndromes, Peripheral Nervous System, mastocytosis, Wilms tumor, lymphatic cancer, vaginal cancer, Hodgkin lymphoma, adrenocortical carcinoma, brain tumors, embryonal tumors and Non-Hodgkin lymphoma.

In some embodiments, the cancer patient is chemotherapy-naïve or has received/is receiving systemic chemotherapy.

In any and all embodiments of the systems disclosed herein, one or more of the plurality of features for each subject in the cohort are determined by assaying blood and/or sequencing tumor DNA.

In one aspect, the present disclosure provides a non-transitory computer-readable storage medium comprising instructions which, when executed by a processor of a machine learning system, configure the machine learning system to train a machine learning classifier to estimate risk of cancer-associated venous thromboembolism (VTE) in cancer patients, wherein the instructions are configured to cause the processor to: (a) receive data on a cohort of subjects, the subjects in the cohort having a plurality of cancer types; (b) generate a training dataset based on the received data, wherein the training dataset comprises a plurality of features for each subject in the cohort, the plurality of features comprising (i) cell free DNA concentration, (ii) maximum ctDNA VAF, (iii) ctDNA alterations in at least one cancer associated gene, and (iv) cancer type; and (c) apply a machine learning method to the training dataset to develop the machine learning classifier for estimating risk of cancer-associated VTE in cancer patients; wherein applying the machine learning method comprises: applying a machine learning technique to the training dataset; performing hyperparameter optimization to identify one or more machine learning models with an accuracy that exceeds an accuracy threshold for the machine learning classifier; and determining an optimal operating-point threshold based on optimization of sensitivity and specificity of the receiver operating characteristic (ROC) curves for the training dataset; wherein the machine learning classifier is configured to receive the plurality of features for cancer patients and generate predictors for risk of cancer-associated VTE in cancer patients. The subjects in the cohort may be chemotherapy-naïve or may have received systemic chemotherapy.

The machine learning technique may model survival outcomes with competing risks. In some embodiments, the machine learning technique is a random forest technique, and the one or more machine learning models are random forest models. Additionally or alternatively, in certain embodiments, the machine learning classifier is an ensemble learning random forest classifier.

Additionally or alternatively, in some embodiments, performing the hyperparameter optimization comprises performing an exhaustive grid search technique.

Additionally or alternatively, in some embodiments of the computer-readable storage medium disclosed herein, the at least one cancer associated gene is selected from the group consisting of AKT1, ALK, APC, AR, ARAF, ARIDIA, ARID2, ATM, B2M, BCL2, BCOR, BRAF, BRCA1, BRCA2, CARD11, CBFB, CCND1, CDH1, CDK4, CDKN2A, CIC, CREBBP, CTCF, CTNNB1, DICER1, DIS3, DNMT3A, EGFR, EIF1AX, EP300, ERBB2, ERBB3, ERCC2, ESR1, EZH2, FBXW7, FGFR1, FGFR2, FGFR3, FGFR4, FLT3, FOXA1, FOXL2, FOXO1, FUBP1, GATA3, GNA11, GNAQ, GNAS, H3F3A, HIST1H3B, HRAS, IDH1, IDH2, IKZF1, INPPL1, JAK1, KDM6A, KEAP1, KIT, KNSTRN, KRAS, MAP2K1, MAPK1, MAX, MED12, MET, MLH1, MSH2, MSH3, MSH6, MTOR, MYC, MYCN, MYD88, MYOD1, NF1, NFE2L2, NOTCH1, NRAS, NTRK1, NTRK2, NTRK3, NUP93, PAK7, PDGFRA, PIK3CA, PIK3CB, PIK3R1, PIK3R2, PMS2, POLE, PPP2RIA, PPP6C, PRKCI, PTCH1, PTEN, PTPN11, RAC1, RAF1, RB1, RET, RHOA, RIT1, ROS1, RRAS2, RXRA, SETD2, SF3B1, SMAD3, SMAD4, SMARCA4, SMARCB1, SOS1, SPOP, STAT3, STK11, STK19, TCF7L2, TGFBR1, TGFBR2, TP53, TP63, TSC1, TSC2, U2AF1, VHL, XPO1, and TERT.

Additionally or alternatively, in some embodiments of the computer-readable storage medium disclosed herein, the plurality of features further comprises platelet count, hemoglobin levels, leukocyte counts, body mass index (BMI), administration of chemotherapy, age, time from cancer diagnosis, race, and metastatic sites of disease. Metastatic sites of disease may comprise one or more of adrenal gland, bone, brain, liver, lung, lymph, and pleura.

In any of the preceding embodiments of the computer-readable storage medium described herein, the instructions further cause the processor to apply the machine learning classifier to data on a cancer patient to generate a predictor, and determine whether the cancer patient is at risk for cancer-associated VTE based on the predictor and the operating-point threshold. In some embodiments, the predictor comprises a cumulative incidence function (CIF) for cancer-associated VTE.

Additionally or alternatively, in certain embodiments of the computer-readable storage medium disclosed herein, the plurality of cancer types are selected from the group consisting of non-small cell lung cancer, breast cancer, pancreatic cancer, melanoma, retinoblastoma, prostate cancer, esophagogastric cancer, histiocytosis, germ cell tumor, endometrial cancer, small cell lung cancer, soft tissue sarcoma, Gastrointestinal Stromal Tumor, ovarian cancer, mature B-Cell neoplasms, small bowel cancer, renal cell carcinoma, thyroid cancer, ampullary cancer, appendiceal cancer, sellar tumor, uterine sarcoma, bone cancer, non-melanoma skin cancer, cervical cancer, mesothelioma, glioma, thymic tumor, gastrointestinal neuroendocrine tumor, salivary gland cancer, sex cord stromal tumor, anal cancer, mature T and NK neoplasms, peritoneal cancer, Head and neck cancer, choroid plexus tumor, leukemia, primary CNS melanocytic tumors, Myelodysplastic Syndromes, Peripheral Nervous System, mastocytosis, Wilms tumor, lymphatic cancer, vaginal cancer, Hodgkin lymphoma, adrenocortical carcinoma, brain tumors, embryonal tumors and Non-Hodgkin lymphoma.

In any of the preceding embodiments of the computer-readable storage medium described herein, the instructions further cause the processor to recommend an anticoagulant therapy to the cancer patient predicted to be at risk for cancer-associated VTE based on the predictor and the operating-point threshold. Examples of anticoagulant therapy include, but are not limited to, apixaban, betrixaban, dabigatran, edoxaban, fondaparinux, heparin, rivaroxaban, warfarin, Xa inhibitors, statins, and enoxaparin. Examples of statins include, but are not limited to atorvastatin, fluvastatin, lovastatin, pitavastatin, pravastatin, rosuvastatin, and simvastatin.

In some embodiments, the cancer patient is chemotherapy-naïve or has received/is receiving systemic chemotherapy.

In another aspect, the present disclosure provides a non-transitory computer-readable storage medium comprising instructions which, when executed by a processor of a computing system, configure the computing system to estimate risk of cancer-associated venous thromboembolism (VTE) in a cancer patient, wherein the instructions are configured to cause the processor to: receive patient data corresponding to a plurality of features for the cancer patient; apply a machine learning classifier to the patient data to generate a predictor; and determine whether the cancer patient is at risk for cancer-associated VTE based on the predictor and an operating-point threshold, wherein the classifier is trained by: (a) receiving cohort data on a cohort of subjects, the subjects in the cohort having a plurality of cancer types; (b) generating a training dataset based on the received cohort data, wherein the training dataset comprises the plurality of features for each subject in the cohort, wherein the plurality of features comprises (i) cell free DNA concentration, (ii) maximum ctDNA VAF, (iii) ctDNA alterations in at least one cancer associated gene, and (iv) cancer type; and (c) applying a machine learning method to the training dataset to develop the machine learning classifier for estimating risk of cancer-associated VTE, wherein applying the machine learning method comprises: applying a machine learning technique to the training dataset; performing hyperparameter optimization to identify one or more machine learning models with an accuracy that exceeds an accuracy threshold for the machine learning classifier; and determining the optimal operating-point threshold based on optimization of sensitivity and specificity of the receiver operating characteristic (ROC) curves for the training dataset; wherein the machine learning classifier is configured to receive the plurality of features for cancer patients and generate predictors for risk of cancer-associated VTE in cancer patients.

The machine learning technique may model survival outcomes with competing risks. In some embodiments, the machine learning technique is a random forest technique, and the one or more machine learning models are random forest models. Additionally or alternatively, in certain embodiments, the machine learning classifier is an ensemble learning random forest classifier.

Additionally or alternatively, in some embodiments, performing the hyperparameter optimization comprises performing an exhaustive grid search technique.

Additionally or alternatively, in some embodiments of the computer-readable storage medium disclosed herein, the at least one cancer associated gene is selected from the group consisting of AKT1, ALK, APC, AR, ARAF, ARIDIA, ARID2, ATM, B2M, BCL2, BCOR, BRAF, BRCA1, BRCA2, CARD11, CBFB, CCND1, CDH1, CDK4, FGFR4, FLT3, FOXA1, FOXL2, FOXO1, FUBP1, GATA3, GNA11, GNAQ, GNAS, H3F3A, HIST1H3B, HRAS, IDH1, IDH2, IKZF1, INPPL1, JAK1, KDM6A, KEAP1, KIT, KNSTRN, KRAS, MAP2K1, MAPK1, MAX, MED12, MET, MLH1, MSH2, MSH3, MSH6, MTOR, MYC, MYCN, MYD88, MYOD1, NF1, NFE2L2, NOTCH1, NRAS, NTRK1, NTRK2, NTRK3, NUP93, PAK7, PDGFRA, PIK3CA, PIK3CB, PIK3R1, PIK3R2, PMS2, POLE, PPP2RIA, PPP6C, PRKCI, PTCH1, PTEN, PTPN11, RAC1, RAF1, RB1, RET, RHOA, RIT1, ROS1, RRAS2, RXRA, SETD2, SF3B1, SMAD3, SMAD4, SMARCA4, SMARCB1, SOS1, SPOP, STAT3, STK11, STK19, TCF7L2, TGFBR1, TGFBR2, TP53, TP63, TSC1, TSC2, U2AF1, VHL, XPO1, and TERT.

Additionally or alternatively, in some embodiments of the computer-readable storage medium disclosed herein, the plurality of features further comprises platelet count, hemoglobin levels, leukocyte counts, body mass index (BMI), administration of chemotherapy, age, time from cancer diagnosis, race, and metastatic sites of disease.

In any of the preceding embodiments of the computer-readable storage medium described herein, the instructions further cause the processor to recommend an anticoagulant therapy to the cancer patient predicted to be at risk for cancer-associated VTE based on the predictor and the operating-point threshold. In some embodiments, the predictor comprises a cumulative incidence function (CIF) for cancer-associated VTE. Examples of anticoagulant therapy include, but are not limited to, apixaban, betrixaban, dabigatran, edoxaban, fondaparinux, heparin, rivaroxaban, warfarin, Xa inhibitors, statins, and enoxaparin. Examples of statins include, but are not limited to atorvastatin, fluvastatin, lovastatin, pitavastatin, pravastatin, rosuvastatin, and simvastatin.

Additionally or alternatively, in certain embodiments of the computer-readable storage medium disclosed herein, the plurality of cancer types are selected from the group consisting of non-small cell lung cancer, breast cancer, pancreatic cancer, melanoma, retinoblastoma, prostate cancer, esophagogastric cancer, histiocytosis, germ cell tumor, endometrial cancer, small cell lung cancer, soft tissue sarcoma, Gastrointestinal Stromal Tumor, ovarian cancer, mature B-Cell neoplasms, small bowel cancer, renal cell carcinoma, thyroid cancer, ampullary cancer, appendiceal cancer, sellar tumor, uterine sarcoma, bone cancer, non-melanoma skin cancer, cervical cancer, mesothelioma, glioma, thymic tumor, gastrointestinal neuroendocrine tumor, salivary gland cancer, sex cord stromal tumor, anal plexus tumor, leukemia, primary CNS melanocytic tumors, Myelodysplastic Syndromes, Peripheral Nervous System, mastocytosis, Wilms tumor, lymphatic cancer, vaginal cancer, Hodgkin lymphoma, adrenocortical carcinoma, brain tumors, embryonal tumors and Non-Hodgkin lymphoma.

In some embodiments, the cancer patient is chemotherapy-naïve or has received/is receiving systemic chemotherapy.

In any of the preceding embodiments of the computer-readable storage medium disclosed herein, one or more of the plurality of features for the cancer patient are determined by assaying blood and/or sequencing tumor DNA.

### BRIEF DESCRIPTION OF THE DRAWINGS

FIG. 1 shows the number of ctDNA alterations within the patient cohort (n=480).

FIG. 2 shows the correlation between patients with ctDNA alteration and risk for CAT.

FIG. 3 shows the relationship between alterations in specific individual cancer genes and risk for CAT.

FIG. 4 shows the correlation between CAT risk and ctDNA variant allele fraction (VAF).

FIG. 5 demonstrates that ctDNA levels are not correlated with Khorana Score or its individual components.

FIG. 6 demonstrates that ctDNA predicts CAT risk in a manner that is orthogonal to the Khorana Score.

FIGS. 7A-7D demonstrate that ctDNA is associated with CAT risk. FIG. 7A: Aalen-Johansen survival curves for CAT from time of plasma draw with death as a competing risk in the MSK-ACCESS cohort. FIG. 7B: Survival curves with ctDNA+ cohort stratified by VAF quartile. FIG. 7C: Cox proportional hazard for CAT if ctDNA+ by cancer type. Number of patients per cancer type shown in FIG. 11. FIG. 7D: Cox proportional hazard for CAT if ctDNA+ for the listed genes adjusted (in a multivariate Cox proportional hazards model) for the cancer types in FIG. 7C.

FIG. 8A: Multivariate Cox proportional hazards model with the listed variables. +ctDNA=any ctDNA mutation or copy number change. FIG. 8B: Random survival forest trained on only listed subset of variables (KS=Khorana Score. MSK-ACCESS-circulating tumor (ct) DNA variant allele fraction, cell-free (cf) DNA concentration, detection of gene-level alterations, Demographics+=Sex, self-reported race (White, Asian, Black or Other), and closest albumin level to ctDNA draw, All=all variables in separate categories combined. C-index reported to time of CAT from time of ctDNA draw in 5-fold cross-validation experiments. Error bars are 95% confidence intervals. FIG. 8C: Permutation variable importances (for all variables with >0.001 importance) in the “All” RSF in FIG. 8B. FIG. 8D: Aalen-Johansen survival curves for CAT from time of plasma draw with death as a competing risk stratified by the risk decile from the “All” RSF in FIG. 8B.

FIGS. 9A-9B: Assessing the potential benefit of previous anticoagulation therapy for preventing CAT stratified by ctDNA presence in a real-world dataset. Aalen-Johansen survival curves for CAT from time of plasma draw with death as a competing risk with or without previous Xa inhibition in ctDNA+ (FIG. 9A) and ctDNA- (FIG. 9B) patients.

FIGS. 10A-10B: Assessing the potential benefit of previous statin use for preventing CAT stratified by ctDNA presence in a real-world dataset. Aalen-Johansen survival curves for CAT from time of plasma draw with death as a competing risk with or without previous statin use in ctDNA+ (FIG. 10A) and ctDNA- (FIG. 10B) patients.

FIG. 11 shows the number of patients with each cancer type included in the pan-cancer study described herein.

FIG. 12A is a block diagram depicting an embodiment of a network environment comprising a client device in communication with server device.

FIG. 12B is a block diagram depicting a cloud computing environment comprising client device in communication with cloud service providers.

FIGS. 12C and 12D are block diagrams depicting embodiments of computing devices useful in connection with the methods and systems described herein.

FIG. 13 depicts a system that includes a computing device and a sample processing system according to various potential embodiments.

FIG. 14 shows the AUC metrics for the Khorana Score, Liquid biopsy and combined models.

### DETAILED DESCRIPTION

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

CAT is an important complication of cancer for which effective pharmacological prophylaxis methods exist. However, currently available prediction rules have limited accuracy in stratifying patients for CAT risk. Accordingly, approaches to enhance the overall benefit of CAT prophylaxis in cancer patients will be contingent on improved methods for predicting risk. The present disclosure demonstrates that ctDNA is a useful biomarker for accurately predicting the risk of cancer-associated thromboembolism in lung cancer patients. These results were unexpected because the methods of the present technology do not correlate with/are not dependent on conventional clinical prediction scores (e.g., Khorana Score) for predicting CAT risk. Indeed, ctDNA predicts CAT risk in a way that is orthogonal/statistically independent to the Khorana Score.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

The term “adapter” refers to a short, chemically synthesized, nucleic acid sequence which can be used to ligate to the end of a nucleic acid sequence in order to facilitate attachment to another molecule. The adapter can be single-stranded or double-stranded. An adapter can incorporate a short (typically less than 50 base pairs) sequence useful for PCR amplification or sequencing.

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, an “alteration” of a gene or gene product (e.g., a marker gene or gene product) refers to the presence of a mutation or mutations within the gene or gene product, e.g., a mutation, which affects the quantity or activity of the gene or gene product, as compared to the normal or wild-type gene. The genetic alteration can result in changes in the quantity, structure, and/or activity of the gene or gene product in a cancer tissue or cancer cell, as compared to its quantity, structure, and/or activity, in a normal or healthy tissue or cell (e.g., a control). For example, an alteration which is predictive of CAT can have an altered nucleotide sequence (e.g., a mutation), amino acid sequence, chromosomal translocation, intra-chromosomal inversion, copy number, expression level, protein level, protein activity, in a cancer tissue or cancer cell, as compared to a normal, healthy tissue or cell. Exemplary mutations include, but are not limited to, point mutations (e.g., silent, missense, or nonsense), deletions, insertions, inversions, linking mutations, duplications, translocations, inter- and intra-chromosomal rearrangements. Mutations can be present in the coding or non-coding region of the gene.

As used herein, “C-index” refers to the proportion of all pairs of patients with usable data in whom the predicted and observed outcomes are ranked appropriately. A higher c-index indicates a better-performing model in that it more correctly ranks relative patient risk (in this case for CAT). See, e.g., Harrell et al JAMA 247 (18): 2543-2546 (1982).

The terms “cancer” or “tumor” are used interchangeably and refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell. As used herein, the term “cancer” includes premalignant, as well as malignant cancers. In some embodiments, the cancer is bladder cancer, breast cancer, colorectal cancer, esophagogastric cancer, gynecological cancer (e.g., uterine cancer, cervical cancer, ovarian cancer), head and neck cancer, hepatobiliary cancer, high-grade glioma, low-grade glioma, lung cancer, melanoma, pancreatic cancer, prostate cancer, renal cancer, or soft tissue sarcoma.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, a “deletion” refers to a mutation (or a genetic alteration) in which part of a DNA sequence at a chromosome location is absent or lost compared to that observed in a reference genome. A deletion may occur within a gene or may encompass one or more genes. A “homozygous deletion” refers to the loss of both alleles of a gene within a genome. A homozygous deletion may comprise a partial or complete loss of each copy (maternal and paternal) of the gene sequence.

“Detecting” as used herein refers to determining the presence of a mutation or alteration in a nucleic acid of interest in a sample. Detection does not require the method to provide 100% sensitivity. Analysis of nucleic acid markers can be performed using techniques known in the art including, but not limited to, sequence analysis, and electrophoretic analysis. Non-limiting examples of sequence analysis include Maxam-Gilbert sequencing, Sanger sequencing, capillary array DNA sequencing, thermal cycle sequencing (Sears et al., Biotechniques, 13:626-633 (1992)), solid-phase sequencing (Zimmerman et al., Methods Mol. Cell Biol, 3:39-42 (1992)), sequencing with mass spectrometry such as matrix-assisted laser desorption/ionization time-of-flight mass spectrometry (MALDI-TOF/MS; Fu et al., Nat. Biotechnol, 16:381-384 (1998)), and sequencing by hybridization. Chee et al., Science, 274:610-614 (1996); Drmanac et al., Science, 260:1649-1652 (1993); Drmanac et al., Nat. Biotechnol, 16:54-58 (1998). Non-limiting examples of electrophoretic analysis include slab gel electrophoresis such as agarose or polyacrylamide gel electrophoresis, capillary electrophoresis, and denaturing gradient gel electrophoresis. Additionally, next generation sequencing methods can be performed using commercially available kits and instruments from companies such as the Life Technologies/Ion Torrent PGM or Proton, the Illumina HiSEQ or MiSEQ, and the Roche/454 next generation sequencing system.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

“Gene” as used herein refers to a DNA sequence that comprises regulatory and coding sequences necessary for the production of an RNA, which may have a non-coding function (e.g., a ribosomal or transfer RNA) or which may include a polypeptide or a polypeptide precursor. The RNA or polypeptide may be encoded by a full length coding sequence or by any portion of the coding sequence so long as the desired activity or function is retained. Although a sequence of the nucleic acids may be shown in the form of DNA, a person of ordinary skill in the art recognizes that the corresponding RNA sequence will have a similar sequence with the thymine being replaced by uracil, i.e., “T” is replaced with “U.”

“Next-generation sequencing or NGS” as used herein, refers to any sequencing method that determines the nucleotide sequence of either individual nucleic acid molecules (e.g., in single molecule sequencing) or clonally expanded proxies for individual nucleic acid molecules in a high throughput parallel fashion (e.g., greater than 103, 104, 105 or more molecules are sequenced simultaneously). In one embodiment, the relative abundance of the nucleic acid species in the library can be estimated by counting the relative number of occurrences of their cognate sequences in the data generated by the sequencing experiment. Next generation sequencing methods are known in the art, and are described, e.g., in Metzker, M. Nature Biotechnology Reviews 11:31-46 (2010).

As used herein, a “sample” refers to a substance that is being assayed for the presence of a mutation in a nucleic acid of interest. Processing methods to release or otherwise make available a nucleic acid for detection are well known in the art and may include steps of nucleic acid manipulation. A biological sample may be a body fluid or a tissue sample. In some cases, a biological sample may consist of or comprise blood, plasma, sera, urine, feces, epidermal sample, vaginal sample, skin sample, cheek swab, sperm, amniotic fluid, cultured cells, bone marrow sample, tumor biopsies, aspirate and/or chorionic villi, cultured cells, and the like. Fresh, fixed or frozen tissues may also be used. In one embodiment, the sample is preserved as a frozen sample or as formaldehyde- or paraformaldehyde-fixed paraffin-embedded (FFPE) tissue preparation. For example, the sample can be embedded in a matrix, e.g., an FFPE block or a frozen sample. Whole blood samples of about 0.5 to 5 ml collected with EDTA, ACD or heparin as anti-coagulant are suitable.

As used herein, the terms “subject”, “patient”, or “individual” can be an individual organism, a vertebrate, a mammal, or a human. In some embodiments, the subject, patient or individual is a human.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

The terms “variant allele fraction,” “VAF,” “mutant allele fraction” or “MAF” refer to fractions of a mutant allele over the total number of mutant (alternate allele) plus wild-type alleles (reference allele). ctDNA VAF represents % ctDNA alteration reported as percentage and computed as the number of mutated DNA molecules divided by the total number (mutated plus wild-type) of DNA fragments at that allele. Most of the cell-free DNA is wild-type (germline); therefore, the median VAF of somatic alterations is <0.5%.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

Methods for Detecting Polynucleotides Associated with Elevated VTE Risk

Polynucleotides associated with elevated VTE risk may be detected by a variety of methods known in the art. Non-limiting examples of detection methods are described below. The detection assays in the methods of the present technology may include purified or isolated DNA (genomic or cDNA), RNA or protein or the detection step may be performed directly from a biological sample without the need for further DNA, RNA or protein purification/isolation.

Nucleic Acid Amplification and/or Detection

Polynucleotides associated with elevated VTE risk can be detected by the use of nucleic acid amplification techniques that are well known in the art. The starting material may be genomic DNA, cDNA, RNA, ctDNA, cfDNA, or mRNA. Nucleic acid amplification can be linear or exponential. Specific variants or mutations may be detected by the use of amplification methods with the aid of oligonucleotide primers or probes designed to interact with or hybridize to a particular target sequence in a specific manner, thus amplifying only the target variant.

Non-limiting examples of nucleic acid amplification techniques include polymerase chain reaction (PCR), real-time quantitative PCR (qPCR), digital PCR (dPCR), reverse transcriptase polymerase chain reaction (RT-PCR), nested PCR, ligase chain reaction (see Abravaya, K. et al., Nucleic Acids Res. (1995), 23:675-682), branched DNA signal amplification (see Urdea, M. S. et al., AIDS (1993), 7 (suppl 2): S11-S14), amplifiable RNA reporters, Q-beta replication, transcription-based amplification, boomerang DNA amplification, strand displacement activation, cycling probe technology, isothermal nucleic acid sequence based amplification (NASBA) (see Kievits, T. et al., J Virological Methods (1991), 35:273-286), Invader Technology, next-generation sequencing technology or other sequence replication assays or signal amplification assays.

Primers: Oligonucleotide primers for use in amplification methods can be designed according to general guidance well known in the art as described herein, as well as with specific requirements as described herein for each step of the particular methods described. In some embodiments, oligonucleotide primers for cDNA synthesis and PCR are 10 to 100 nucleotides in length, preferably between about 15 and about 60 nucleotides in length, more preferably 25 and about 50 nucleotides in length, and most preferably between about 25 and about 40 nucleotides in length.

Tₘ of a polynucleotide affects its hybridization to another polynucleotide (e.g., the annealing of an oligonucleotide primer to a template polynucleotide). In certain embodiments of the disclosed methods, the oligonucleotide primer used in various steps selectively hybridizes to a target template or polynucleotides derived from the target template (i.e., first and second strand cDNAs and amplified products). Typically, selective hybridization occurs when two polynucleotide sequences are substantially complementary (at least about 65% complementary over a stretch of at least 14 to 25 nucleotides, preferably at least about 75%, more preferably at least about 90% complementary). See Kanehisa, M., Polynucleotides Res. (1984), 12:203, incorporated herein by reference. As a result, it is expected that a certain degree of mismatch at the priming site is tolerated. Such mismatch may be small, such as a mono-, di- or tri-nucleotide. In certain embodiments, 100% complementarity exists.

Probes: Probes are capable of hybridizing to at least a portion of the nucleic acid of interest or a reference nucleic acid (i.e., wild-type sequence). Probes may be an oligonucleotide, artificial chromosome, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may be used for detecting and/or capturing/purifying a nucleic acid of interest.

Typically, probes can be about 10 nucleotides, about 20 nucleotides, about 25 nucleotides, about 30 nucleotides, about 35 nucleotides, about 40 nucleotides, about 50 nucleotides, about 60 nucleotides, about 75 nucleotides, or about 100 nucleotides long. However, longer probes are possible. Longer probes can be about 200 nucleotides, about 300 nucleotides, about 400 nucleotides, about 500 nucleotides, about 750 nucleotides, about 1,000 nucleotides, about 1,500 nucleotides, about 2,000 nucleotides, about 2,500 nucleotides, about 3,000 nucleotides, about 3,500 nucleotides, about 4,000 nucleotides, about 5,000 nucleotides, about 7,500 nucleotides, or about 10,000 nucleotides long.

Probes may also include a detectable label or a plurality of detectable labels. The detectable label associated with the probe can generate a detectable signal directly. Additionally, the detectable label associated with the probe can be detected indirectly using a reagent, wherein the reagent includes a detectable label, and binds to the label associated with the probe.

In some embodiments, detectably labeled probes can be used in hybridization assays including, but not limited to Northern blots, Southern blots, microarray, dot or slot blots, and in situ hybridization assays such as fluorescent in situ hybridization (FISH) to detect a target nucleic acid sequence within a biological sample. Certain embodiments may employ hybridization methods for measuring expression of a polynucleotide gene product, such as mRNA. Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif, 1987); Young and Davis, PNAS. 80:1194 (1983).

Detectably labeled probes can also be used to monitor the amplification of a target nucleic acid sequence. In some embodiments, detectably labeled probes present in an amplification reaction are suitable for monitoring the amount of amplicon(s) produced as a function of time. Examples of such probes include, but are not limited to, the 5′-exonuclease assay (TAQMAN® probes described herein (see also U.S. Pat. No. 5,538,848) various stem-loop molecular beacons (see for example, U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, for example, Kubista et al., 2001, SPIE 4264:53-58), non-FRET probes (see, for example, U.S. Pat. No. 6,150,097), Sunrise®/Amplifluor™ probes (U.S. Pat. No. 6,548,250), stem-loop and duplex Scorpion probes (Solinas et al., 2001, Nucleic Acids Research 29: E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem. Res. Toxicol. 15:118-126; and Yu et al., 2001, J. Am. Chem. Soc 14:11155-11161.

In some embodiments, the detectable label is a fluorophore. Suitable fluorescent moieties include but are not limited to the following fluorophores working individually or in combination: 4-acetamido-4′-isothiocyanatostilbene-2,2′disulfonic acid; acridine and derivatives: acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2-aminoethyl)aminonaphthalene-1-sulfonic acid (EDANS); 4-amino-N-[3-vinylsulfonyl)phenyl] naphthalimide-3,5 disulfonate (Lucifer Yellow VS); N-(4-anilino-1-naphthyl) maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120), 7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)amino fluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescem (TET); fiuorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron®Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; tetramethyl rhodamine isothiocyanate (TRITC); and VIC®. Detector probes can also comprise sulfonate derivatives of fluorescenin dyes with S03 instead of the carboxylate group, phosphoramidite forms of fluorescein, phosphoramidite forms of CY 5 (commercially available for example from Amersham).

Detectably labeled probes can also include quenchers, including without limitation black hole quenchers (Biosearch), Iowa Black (IDT), QSY quencher (Molecular Probes), and Dabsyl and Dabcel sulfonate/carboxylate Quenchers (Epoch).

Detectably labeled probes can also include two probes, wherein for example a fluorophore is on one probe, and a quencher is on the other probe, wherein hybridization of the two probes together on a target quenches the signal, or wherein hybridization on the target alters the signal signature via a change in fluorescence.

In some embodiments, interchelating labels such as ethidium bromide, SYBR® Green I (Molecular Probes), and PicoGreen® (Molecular Probes) are used, thereby allowing visualization in real-time, or at the end point, of an amplification product in the absence of a detector probe. In some embodiments, real-time visualization may involve the use of both an intercalating detector probe and a sequence-based detector probe. In some embodiments, the detector probe is at least partially quenched when not hybridized to a complementary sequence in the amplification reaction, and is at least partially unquenched when hybridized to a complementary sequence in the amplification reaction.

In some embodiments, the amount of probe that gives a fluorescent signal in response to an excited light typically relates to the amount of nucleic acid produced in the amplification reaction. Thus, in some embodiments, the amount of fluorescent signal is related to the amount of product created in the amplification reaction. In such embodiments, one can therefore measure the amount of amplification product by measuring the intensity of the fluorescent signal from the fluorescent indicator.

Primers or probes may be designed to selectively hybridize to any portion of a nucleic acid sequence encoding a polypeptide selected from among AKT1, ALK, B2M, BRAF, EGFR, ERBB2 (HER2), FGFR2, FGFR3, KEAP1, KRAS, MAP2K1 (MEK1), MET, NRAS, PIK3CA, RET, ROS1, STK11, TP53, NTRK1, FGFR1, MYC, PTEN, and RICTOR. Exemplary nucleic acid sequences of the human orthologs of these genes are provided below:

NM_005163.2 Homo sapiens AKT serine/threonine kinase 1 (AKT1), transcript
variant 1, mRNA
(SEQ ID NO: 1)
TAATTATGGGTCTGTAACCACCCTGGACTGGGTGCTCCTCACTGACGGACTTGTCTGAACCTCTCTTTGT
CTCCAGCGCCCAGCACTGGGCCTGGCAAAACCTGAGACGCCCGGTACATGTTGGCCAAATGAATGAACCA
GATTCAGACCGGCAGGGGCGCTGTGGTTTAGGAGGGGCCTGGGGTTTCTCCCAGGAGGTTTTTGGGCTTG
CGCTGGAGGGCTCTGGACTCCCGTTTGCGCCAGTGGCCTGCATCCTGGTCCTGTCTTCCTCATGTTTGAA
TTTCTTTGCTTTCCTAGTCTGGGGAGCAGGGAGGAGCCCTGTGCCCTGTCCCAGGATCCATGGGTAGGAA
CACCATGGACAGGGAGAGCAAACGGGGCCATCTGTCACCAGGGGCTTAGGGAAGGCCGAGCCAGCCTGGG
TCAAAGAAGTCAAAGGGGCTGCCTGGAGGAGGCAGCCTGTCAGCTGGTGCATCAGAGGCTGTGGCCAGGC
CAGCTGGGCTCGGGGAGCGCCAGCCTGAGAGGAGCGCGTGAGCGTCGCGGGAGCCTCGGGCACCATGAGC
GACGTGGCTATTGTGAAGGAGGGTTGGCTGCACAAACGAGGGGAGTACATCAAGACCTGGCGGCCACGCT
ACTTCCTCCTCAAGAATGATGGCACCTTCATTGGCTACAAGGAGCGGCCGCAGGATGTGGACCAACGTGA
GGCTCCCCTCAACAACTTCTCTGTGGCGCAGTGCCAGCTGATGAAGACGGAGCGGCCCCGGCCCAACACC
TTCATCATCCGCTGCCTGCAGTGGACCACTGTCATCGAACGCACCTTCCATGTGGAGACTCCTGAGGAGC
GGGAGGAGTGGACAACCGCCATCCAGACTGTGGCTGACGGCCTCAAGAAGCAGGAGGAGGAGGAGATGGA
CTTCCGGTCGGGCTCACCCAGTGACAACTCAGGGGCTGAAGAGATGGAGGTGTCCCTGGCCAAGCCCAAG
CACCGCGTGACCATGAACGAGTTTGAGTACCTGAAGCTGCTGGGCAAGGGCACTTTCGGCAAGGTGATCC
TGGTGAAGGAGAAGGCCACAGGCCGCTACTACGCCATGAAGATCCTCAAGAAGGAAGTCATCGTGGCCAA
GGACGAGGTGGCCCACACACTCACCGAGAACCGCGTCCTGCAGAACTCCAGGCACCCCTTCCTCACAGCC
CTGAAGTACTCTTTCCAGACCCACGACCGCCTCTGCTTTGTCATGGAGTACGCCAACGGGGGCGAGCTGT
TCTTCCACCTGTCCCGGGAGCGTGTGTTCTCCGAGGACCGGGCCCGCTTCTATGGCGCTGAGATTGTGTC
AGCCCTGGACTACCTGCACTCGGAGAAGAACGTGGTGTACCGGGACCTCAAGCTGGAGAACCTCATGCTG
GACAAGGACGGGCACATTAAGATCACAGACTTCGGGCTGTGCAAGGAGGGGATCAAGGACGGTGCCACCA
TGAAGACCTTTTGCGGCACACCTGAGTACCTGGCCCCCGAGGTGCTGGAGGACAATGACTACGGCCGTGC
AGTGGACTGGTGGGGGCTGGGCGTGGTCATGTACGAGATGATGTGCGGTCGCCTGCCCTTCTACAACCAG
GACCATGAGAAGCTTTTTGAGCTCATCCTCATGGAGGAGATCCGCTTCCCGCGCACGCTTGGTCCCGAGG
CCAAGTCCTTGCTTTCAGGGCTGCTCAAGAAGGACCCCAAGCAGAGGCTTGGCGGGGGCTCCGAGGACGC
CAAGGAGATCATGCAGCATCGCTTCTTTGCCGGTATCGTGTGGCAGCACGTGTACGAGAAGAAGCTCAGC
CCACCCTTCAAGCCCCAGGTCACGTCGGAGACTGACACCAGGTATTTTGATGAGGAGTTCACGGCCCAGA
TGATCACCATCACACCACCTGACCAAGATGACAGCATGGAGTGTGTGGACAGCGAGCGCAGGCCCCACTT
CCCCCAGTTCTCCTACTCGGCCAGCGGCACGGCCTGAGGCGGCGGTGGACTGCGCTGGACGATAGCTTGG
AGGGATGGAGAGGCGGCCTCGTGCCATGATCTGTATTTAATGGTTTTTATTTCTCGGGTGCATTTGAGAG
AAGCCACGCTGTCCTCTCGAGCCCAGATGGAAAGACGTTTTTGTGCTGTGGGCAGCACCCTCCCCCGCAG
CGGGGTAGGGAAGAAAACTATCCTGCGGGTTTTAATTTATTTCATCCAGTTTGTTCTCCGGGTGTGGCCT
CAGCCCTCAGAACAATCCGATTCACGTAGGGAAATGTTAAGGACTTCTGCAGCTATGCGCAATGTGGCAT
TGGGGGGCCGGGCAGGTCCTGCCCATGTGTCCCCTCACTCTGTCAGCCAGCCGCCCTGGGCTGTCTGTCA
CCAGCTATCTGTCATCTCTCTGGGGCCCTGGGCCTCAGTTCAACCTGGTGGCACCAGATGCAACCTCACT
ATGGTATGCTGGCCAGCACCCTCTCCTGGGGGTGGCAGGCACACAGCAGCCCCCCAGCACTAAGGCCGTG
TCTCTGAGGACGTCATCGGAGGCTGGGCCCCTGGGATGGGACCAGGGATGGGGGATGGGCCAGGGTTTAC
CCAGTGGGACAGAGGAGCAAGGTTTAAATTTGTTATTGTGTATTATGTTGTTCAAATGCATTTTGGGGGT
TTTTAATCTTTGTGACAGGAAAGCCCTCCCCCTTCCCCTTCTGTGTCACAGTTCTTGGTGACTGTCCCAC
CGGGAGCCTCCCCCTCAGATGATCTCTCCACGGTAGCACTTGACCTTTTCGACGCTTAACCTTTCCGCTG
TCGCCCCAGGCCCTCCCTGACTCCCTGTGGGGGTGGCCATCCCTGGGCCCCTCCACGCCTCCTGGCCAGA
CGCTGCCGCTGCCGCTGCACCACGGCGTTTTTTTACAACATTCAACTTTAGTATTTTTACTATTATAATA
TAATATGGAACCTTCCCTCCAAATTCTTCAATAAAAGTTGCTTTTCAAAAAAAAAAAAAAAAAAAAAA
NM 004304.5 Homo sapiens ALK receptor tyrosine kinase (ALK), transcript
variant 1, mRNA
(SEQ ID NO: 2)
AGATGCGATCCAGCGGCTCTGGGGGCGGCAGCGGTGGTAGCAGCTGGTACCTCCCGCCGCCTCTGTTCGG
AGGGTCGCGGGGCACCGAGGTGCTTTCCGGCCGCCCTCTGGTCGGCCACCCAAAGCCGCGGGCGCTGATG
ATGGGTGAGGAGGGGGCGGCAAGATTTCGGGCGCCCCTGCCCTGAACGCCCTCAGCTGCTGCCGCCGGGG
CCGCTCCAGTGCCTGCGAACTCTGAGGAGCCGAGGCGCCGGTGAGAGCAAGGACGCTGCAAACTTGCGCA
GCGCGGGGGCTGGGATTCACGCCCAGAAGTTCAGCAGGCAGACAGTCCGAAGCCTTCCCGCAGCGGAGAG
ATAGCTTGAGGGTGCGCAAGACGGCAGCCTCCGCCCTCGGTTCCCGCCCAGACCGGGCAGAAGAGCTTGG
AGGAGCCAAAAGGAACGCAAAAGGCGGCCAGGACAGCGTGCAGCAGCTGGGAGCCGCCGTTCTCAGCCTT
AAAAGTTGCAGAGATTGGAGGCTGCCCCGAGAGGGGACAGACCCCAGCTCCGACTGCGGGGGGCAGGAGA
GGACGGTACCCAACTGCCACCTCCCTTCAACCATAGTAGTTCCTCTGTACCGAGCGCAGCGAGCTACAGA
CGGGGGCGCGGCACTCGGCGCGGAGAGCGGGAGGCTCAAGGTCCCAGCCAGTGAGCCCAGTGTGCTTGAG
TGTCTCTGGACTCGCCCCTGAGCTTCCAGGTCTGTTTCATTTAGACTCCTGCTCGCCTCCGTGCAGTTGG
GGGAAAGCAAGAGACTTGCGCGCACGCACAGTCCTCTGGAGATCAGGTGGAAGGAGCCGCTGGGTACCAA
GGACTGTTCAGAGCCTCTTCCCATCTCGGGGAGAGCGAAGGGTGAGGCTGGGCCCGGAGAGCAGTGTAAA
CGGCCTCCTCCGGCGGGATGGGAGCCATCGGGCTCCTGTGGCTCCTGCCGCTGCTGCTTTCCACGGCAGC
TGTGGGCTCCGGGATGGGGACCGGCCAGCGCGCGGGCTCCCCAGCTGCGGGGCCGCCGCTGCAGCCCCGG
GAGCCACTCAGCTACTCGCGCCTGCAGAGGAAGAGTCTGGCAGTTGACTTCGTGGTGCCCTCGCTCTTCC
GTGTCTACGCCCGGGACCTACTGCTGCCACCATCCTCCTCGGAGCTGAAGGCTGGCAGGCCCGAGGCCCG
CGGCTCGCTAGCTCTGGACTGCGCCCCGCTGCTCAGGTTGCTGGGGCCGGCGCCGGGGGTCTCCTGGACC
GCCGGTTCACCAGCCCCGGCAGAGGCCCGGACGCTGTCCAGGGTGCTGAAGGGCGGCTCCGTGCGCAAGC
TCCGGCGTGCCAAGCAGTTGGTGCTGGAGCTGGGCGAGGAGGCGATCTTGGAGGGTTGCGTCGGGCCCCC
CGGGGAGGCGGCTGTGGGGCTGCTCCAGTTCAATCTCAGCGAGCTGTTCAGTTGGTGGATTCGCCAAGGC
GAAGGGCGACTGAGGATCCGCCTGATGCCCGAGAAGAAGGCGTCGGAAGTGGGCAGAGAGGGAAGGCTGT
CCGCGGCAATTCGCGCCTCCCAGCCCCGCCTTCTCTTCCAGATCTTCGGGACTGGTCATAGCTCCTTGGA
ATCACCAACAAACATGCCTTCTCCTTCTCCTGATTATTTTACATGGAATCTCACCTGGATAATGAAAGAC
TCCTTCCCTTTCCTGTCTCATCGCAGCCGATATGGTCTGGAGTGCAGCTTTGACTTCCCCTGTGAGCTGG
AGTATTCCCCTCCACTGCATGACCTCAGGAACCAGAGCTGGTCCTGGCGCCGCATCCCCTCCGAGGAGGC
CTCCCAGATGGACTTGCTGGATGGGCCTGGGGCAGAGCGTTCTAAGGAGATGCCCAGAGGCTCCTTTCTC
CTTCTCAACACCTCAGCTGACTCCAAGCACACCATCCTGAGTCCGTGGATGAGGAGCAGCAGTGAGCACT
GCACACTGGCCGTCTCGGTGCACAGGCACCTGCAGCCCTCTGGAAGGTACATTGCCCAGCTGCTGCCCCA
CAACGAGGCTGCAAGAGAGATCCTCCTGATGCCCACTCCAGGGAAGCATGGTTGGACAGTGCTCCAGGGA
AGAATCGGGCGTCCAGACAACCCATTTCGAGTGGCCCTGGAATACATCTCCAGTGGAAACCGCAGCTTGT
CTGCAGTGGACTTCTTTGCCCTGAAGAACTGCAGTGAAGGAACATCCCCAGGCTCCAAGATGGCCCTGCA
GAGCTCCTTCACTTGTTGGAATGGGACAGTCCTCCAGCTTGGGCAGGCCTGTGACTTCCACCAGGACTGT
GCCCAGGGAGAAGATGAGAGCCAGATGTGCCGGAAACTGCCTGTGGGTTTTTACTGCAACTTTGAAGATG
GCTTCTGTGGCTGGACCCAAGGCACACTGTCACCCCACACTCCTCAATGGCAGGTCAGGACCCTAAAGGA
TGCCCGGTTCCAGGACCACCAAGACCATGCTCTATTGCTCAGTACCACTGATGTCCCCGCTTCTGAAAGT
GCTACAGTGACCAGTGCTACGTTTCCTGCACCGATCAAGAGCTCTCCATGTGAGCTCCGAATGTCCTGGC
TCATTCGTGGAGTCTTGAGGGGAAACGTGTCCTTGGTGCTAGTGGAGAACAAAACCGGGAAGGAGCAAGG
CAGGATGGTCTGGCATGTCGCCGCCTATGAAGGCTTGAGCCTGTGGCAGTGGATGGTGTTGCCTCTCCTC
GATGTGTCTGACAGGTTCTGGCTGCAGATGGTCGCATGGTGGGGACAAGGATCCAGAGCCATCGTGGCTT
TTGACAATATCTCCATCAGCCTGGACTGCTACCTCACCATTAGCGGAGAGGACAAGATCCTGCAGAATAC
AGCACCCAAATCAAGAAACCTGTTTGAGAGAAACCCAAACAAGGAGCTGAAACCCGGGGAAAATTCACCA
AGACAGACCCCCATCTTTGACCCTACAGTTCATTGGCTGTTCACCACATGTGGGGCCAGCGGGCCCCATG
GCCCCACCCAGGCACAGTGCAACAACGCCTACCAGAACTCCAACCTGAGCGTGGAGGTGGGGAGCGAGGG
CCCCCTGAAAGGCATCCAGATCTGGAAGGTGCCAGCCACCGACACCTACAGCATCTCGGGCTACGGAGCT
GCTGGCGGGAAAGGCGGGAAGAACACCATGATGCGGTCCCACGGCGTGTCTGTGCTGGGCATCTTCAACC
TGGAGAAGGATGACATGCTGTACATCCTGGTTGGGCAGCAGGGAGAGGACGCCTGCCCCAGTACAAACCA
GTTAATCCAGAAAGTCTGCATTGGAGAGAACAATGTGATAGAAGAAGAAATCCGTGTGAACAGAAGCGTG
CATGAGTGGGCAGGAGGCGGAGGAGGAGGGGGTGGAGCCACCTACGTATTTAAGATGAAGGATGGAGTGC
CGGTGCCCCTGATCATTGCAGCCGGAGGTGGTGGCAGGGCCTACGGGGCCAAGACAGACACGTTCCACCC
AGAGAGACTGGAGAATAACTCCTCGGTTCTAGGGCTAAACGGCAATTCCGGAGCCGCAGGTGGTGGAGGT
GGCTGGAATGATAACACTTCCTTGCTCTGGGCCGGAAAATCTTTGCAGGAGGGTGCCACCGGAGGACATT
CCTGCCCCCAGGCCATGAAGAAGTGGGGGTGGGAGACAAGAGGGGGTTTCGGAGGGGGTGGAGGGGGGTG
CTCCTCAGGTGGAGGAGGCGGAGGATATATAGGCGGCAATGCAGCCTCAAACAATGACCCCGAAATGGAT
GGGGAAGATGGGGTTTCCTTCATCAGTCCACTGGGCATCCTGTACACCCCAGCTTTAAAAGTGATGGAAG
GCCACGGGGAAGTGAATATTAAGCATTATCTAAACTGCAGTCACTGTGAGGTAGACGAATGTCACATGGA
CCCTGAAAGCCACAAGGTCATCTGCTTCTGTGACCACGGGACGGTGCTGGCTGAGGATGGCGTCTCCTGC
ATTGTGTCACCCACCCCGGAGCCACACCTGCCACTCTCGCTGATCCTCTCTGTGGTGACCTCTGCCCTCG
TGGCCGCCCTGGTCCTGGCTTTCTCCGGCATCATGATTGTGTACCGCCGGAAGCACCAGGAGCTGCAAGC
CATGCAGATGGAGCTGCAGAGCCCTGAGTACAAGCTGAGCAAGCTCCGCACCTCGACCATCATGACCGAC
TACAACCCCAACTACTGCTTTGCTGGCAAGACCTCCTCCATCAGTGACCTGAAGGAGGTGCCGCGGAAAA
ACATCACCCTCATTCGGGGTCTGGGCCATGGCGCCTTTGGGGAGGTGTATGAAGGCCAGGTGTCCGGAAT
GCCCAACGACCCAAGCCCCCTGCAAGTGGCTGTGAAGACGCTGCCTGAAGTGTGCTCTGAACAGGACGAA
CTGGATTTCCTCATGGAAGCCCTGATCATCAGCAAATTCAACCACCAGAACATTGTTCGCTGCATTGGGG
TGAGCCTGCAATCCCTGCCCCGGTTCATCCTGCTGGAGCTCATGGGGGGGGAGACCTCAAGTCCTTCCT
CCGAGAGACCCGCCCTCGCCCGAGCCAGCCCTCCTCCCTGGCCATGCTGGACCTTCTGCACGTGGCTCGG
GACATTGCCTGTGGCTGTCAGTATTTGGAGGAAAACCACTTCATCCACCGAGACATTGCTGCCAGAAACT
GCCTCTTGACCTGTCCAGGCCCTGGAAGAGTGGCCAAGATTGGAGACTTCGGGATGGCCCGAGACATCTA
CAGGGCGAGCTACTATAGAAAGGGAGGCTGTGCCATGCTGCCAGTTAAGTGGATGCCCCCAGAGGCCTTC
ATGGAAGGAATATTCACTTCTAAAACAGACACATGGTCCTTTGGAGTGCTGCTATGGGAAATCTTTTCTC
TTGGATATATGCCATACCCCAGCAAAAGCAACCAGGAAGTTCTGGAGTTTGTCACCAGTGGAGGCCGGAT
GGACCCACCCAAGAACTGCCCTGGGCCTGTATACCGGATAATGACTCAGTGCTGGCAACATCAGCCTGAA
GACAGGCCCAACTTTGCCATCATTTTGGAGAGGATTGAATACTGCACCCAGGACCCGGATGTAATCAACA
CCGCTTTGCCGATAGAATATGGTCCACTTGTGGAAGAGGAAGAGAAAGTGCCTGTGAGGCCCAAGGACCC
TGAGGGGGTTCCTCCTCTCCTGGTCTCTCAACAGGCAAAACGGGAGGAGGAGCGCAGCCCAGCTGCCCCA
CCACCTCTGCCTACCACCTCCTCTGGCAAGGCTGCAAAGAAACCCACAGCTGCAGAGATCTCTGTTCGAG
TCCCTAGAGGGCCGGCCGTGGAAGGGGGACACGTGAATATGGCATTCTCTCAGTCCAACCCTCCTTCGGA
GTTGCACAAGGTCCACGGATCCAGAAACAAGCCCACCAGCTTGTGGAACCCAACGTACGGCTCCTGGTTT
ACAGAGAAACCCACCAAAAAGAATAATCCTATAGCAAAGAAGGAGCCACACGACAGGGGTAACCTGGGGC
TGGAGGGAAGCTGTACTGTCCCACCTAACGTTGCAACTGGGAGACTTCCGGGGGCCTCACTGCTCCTAGA
GCCCTCTTCGCTGACTGCCAATATGAAGGAGGTACCTCTGTTCAGGCTACGTCACTTCCCTTGTGGGAAT
GTCAATTACGGCTACCAGCAACAGGGCTTGCCCTTAGAAGCCGCTACTGCCCCTGGAGCTGGTCATTACG
AGGATACCATTCTGAAAAGCAAGAATAGCATGAACCAGCCTGGGCCCTGAGCTCGGTCGCACACTCACTT
CTCTTCCTTGGGATCCCTAAGACCGTGGAGGAGAGAGAGGCAATGGCTCCTTCACAAACCAGAGACCAAA
TGTCACGTTTTGTTTTGTGCCAACCTATTTTGAAGTACCACCAAAAAAGCTGTATTTTGAAAATGCTTTA
GAAAGGTTTTGAGCATGGGTTCATCCTATTCTTTCGAAAGAAGAAAATATCATAAAAATGAGTGATAAAT
ACAAGGCCCAGATGTGGTTGCATAAGGTTTTTATGCATGTTTGTTGTATACTTCCTTATGCTTCTTTCAA
ATTGTGTGTGCTCTGCTTCAATGTAGTCAGAATTAGCTGCTTCTATGTTTCATAGTTGGGGTCATAGATG
TTTCCTTGCCTTGTTGATGTGGACATGAGCCATTTGAGGGGAGAGGGAACGGAAATAAAGGAGTTATTTG
TAATGACTAA
XM_005254549.4: Homo sapiens beta-2-microglobulin (B2M), transcript
variant X1, mRNA
(SEQ ID NO: 3)
ATTCCTGAAGCTGACAGCATTCGGGCCGAGATGTCTCGCTCCGTGGCCTTAGCTGTGCTCGCGCTACTCT
CTCTTTCTGGCCTGGAGGCTATCCAGCGTACTCCAAAGATTCAGGTTTACTCACGTCATCCAGCAGAGAA
TGGAAAGTCAAATTTCCTGAATTGCTATGTGTCTGGGTTTCATCCATCCGACATTGAAGTTGACTTACTG
AAGAATGGAGAGAGAATTGAAAAAGTGGAGCATTCAGACTTGTCTTTCAGCAAGGACTGGTCTTTCTATC
TCTTGTACTACACTGAATTCACCCCCACTGAAAAAGATGAGTATGCCTGCCGTGTGAACCATGTGACTTT
GTCACAGCCCAAGATAGTTAAGTGGGGTAAGTCTTACATTCTTTTGTAAGCTGCTGAAAGTTGTGTATGA
GTAGTCATATCATAAAGCTGCTTTGATATAAAAAAGGTCTATGGCCATACTACCCTGAATGAGTCCCATC
CCATCTGATATAAACAATCTGCATATTGGGATTGTCAGGGAATGTTCTTAAAGATCAGATTAGTGGCACC
TGCTGAGATACTGATGCACAGCATGGTTTCTGAACCAGTAGTTTCCCTGCAGTTGAGCAGGGAGCAGCAG
CAGCACTTGCACAAATACATATACACTCTTAACACTTCTTACCTACTGGCTTCCTCTAGCTTTTGTGGCA
GCTTCAGGTATATTTAGCACTGAACGAACATCTCAAGAAGGTATAGGCCTTTGTTTGTAAGTCCTGCTGT
CCTAGCATCCTATAATCCTGGACTTCTCCAGTACTTTCTGGCTGGATTGGTATCTGAGGCTAGTAGGAAG
GGCTTGTTCCTGCTGGGTAGCTCTAAACAATGTATTCATGGGTAGGAACAGCAGCCTATTCTGCCAGCCT
TATTTCTAACCATTTTAGACATTTGTTAGTACATGGTATTTTAAAAGTAAAACTTAATGTCTTCCTT
NM_001354609.2 Homo sapiens B-Raf proto-oncogene, serine/threonine kinase
(BRAF), transcript variant 2, mRNA
(SEQ ID NO: 4)
CTTCCCCCAATCCCCTCAGGCTCGGCTGCGCCCGGGGCCGCGGGCCGGTACCTGAGGTGGCCCAGGCGCC
CTCCGCCCGCGGCGCCGCCCGGGCCGCTCCTCCCCGCGCCCCCCGCGCCCCCCGCTCCTCCGCCTCCGCC
TCCGCCTCCGCCTCCCCCAGCTCTCCGCCTCCCTTCCCCCTCCCCGCCCGACAGCGGCCGCTCGGGCCCC
GGCTCTCGGTTATAAGATGGCGGCGCTGAGCGGTGGCGGTGGTGGCGGCGCGGAGCCGGGCCAGGCTCTG
TTCAACGGGGACATGGAGCCCGAGGCCGGCGCCGGCGCCGGCGCCGCGGCCTCTTCGGCTGCGGACCCTG
CCATTCCGGAGGAGGTGTGGAATATCAAACAAATGATTAAGTTGACACAGGAACATATAGAGGCCCTATT
GGACAAATTTGGTGGGGAGCATAATCCACCATCAATATATCTGGAGGCCTATGAAGAATACACCAGCAAG
CTAGATGCACTCCAACAAAGAGAACAACAGTTATTGGAATCTCTGGGGAACGGAACTGATTTTTCTGTTT
CTAGCTCTGCATCAATGGATACCGTTACATCTTCTTCCTCTTCTAGCCTTTCAGTGCTACCTTCATCTCT
TTCAGTTTTTCAAAATCCCACAGATGTGGCACGGAGCAACCCCAAGTCACCACAAAAACCTATCGTTAGA
GTCTTCCTGCCCAACAAACAGAGGACAGTGGTACCTGCAAGGTGTGGAGTTACAGTCCGAGACAGTCTAA
AGAAAGCACTGATGATGAGAGGTCTAATCCCAGAGTGCTGTGCTGTTTACAGAATTCAGGATGGAGAGAA
GAAACCAATTGGTTGGGACACTGATATTTCCTGGCTTACTGGAGAAGAATTGCATGTGGAAGTGTTGGAG
AATGTTCCACTTACAACACACAACTTTGTACGAAAAACGTTTTTCACCTTAGCATTTTGTGACTTTTGTC
GAAAGCTGCTTTTCCAGGGTTTCCGCTGTCAAACATGTGGTTATAAATTTCACCAGCGTTGTAGTACAGA
AGTTCCACTGATGTGTGTTAATTATGACCAACTTGATTTGCTGTTTGTCTCCAAGTTCTTTGAACACCAC
CCAATACCACAGGAAGAGGCGTCCTTAGCAGAGACTGCCCTAACATCTGGATCATCCCCTTCCGCACCCG
CCTCGGACTCTATTGGGCCCCAAATTCTCACCAGTCCGTCTCCTTCAAAATCCATTCCAATTCCACAGCC
CTTCCGACCAGCAGATGAAGATCATCGAAATCAATTTGGGCAACGAGACCGATCCTCATCAGCTCCCAAT
GTGCATATAAACACAATAGAACCTGTCAATATTGATGACTTGATTAGAGACCAAGGATTTCGTGGTGATG
GAGGATCAACCACAGGTTTGTCTGCTACCCCCCCTGCCTCATTACCTGGCTCACTAACTAACGTGAAAGC
CTTACAGAAATCTCCAGGACCTCAGCGAGAAAGGAAGTCATCTTCATCCTCAGAAGACAGGAATCGAATG
AAAACACTTGGTAGACGGGACTCGAGTGATGATTGGGAGATTCCTGATGGGCAGATTACAGTGGGACAAA
GAATTGGATCTGGATCATTTGGAACAGTCTACAAGGGAAAGTGGCATGGTGATGTGGCAGTGAAAATGTT
GAATGTGACAGCACCTACACCTCAGCAGTTACAAGCCTTCAAAAATGAAGTAGGAGTACTCAGGAAAACA
CGACATGTGAATATCCTACTCTTCATGGGCTATTCCACAAAGCCACAACTGGCTATTGTTACCCAGTGGT
GTGAGGGCTCCAGCTTGTATCACCATCTCCATATCATTGAGACCAAATTTGAGATGATCAAACTTATAGA
TATTGCACGACAGACTGCACAGGGCATGGATTACTTACACGCCAAGTCAATCATCCACAGAGACCTCAAG
AGTAATAATATATTTCTTCATGAAGACCTCACAGTAAAAATAGGTGATTTTGGTCTAGCTACAGTGAAAT
CTCGATGGAGTGGGTCCCATCAGTTTGAACAGTTGTCTGGATCCATTTTGTGGATGGCACCAGAAGTCAT
CAGAATGCAAGATAAAAATCCATACAGCTTTCAGTCAGATGTATATGCATTTGGAATTGTTCTGTATGAA
TTGATGACTGGACAGTTACCTTATTCAAACATCAACAACAGGGACCAGATAATTTTTATGGTGGGACGAG
GATACCTGTCTCCAGATCTCAGTAAGGTACGGAGTAACTGTCCAAAAGCCATGAAGAGATTAATGGCAGA
GTGCCTCAAAAAGAAAAGAGATGAGAGACCACTCTTTCCCCAAATTCTCGCCTCTATTGAGCTGCTGGCC
CGCTCATTGCCAAAAATTCACCGCAGTGCATCAGAACCCTCCTTGAATCGGGCTGGTTTCCAAACAGAGG
ATTTTAGTCTATATGCTTGTGCTTCTCCAAAAACACCCATCCAGGCAGGGGGATATGGAGAATTTGCAGC
CTTCAAGTAGCCACCATCATGGCAGCATCTGCTCTTATTTCTTAAGTCTTGTGTTCGTACAATTTGTTAA
CATCAAAACACAGTTCTGTTCCTCAAATCTTTTTTTAAAGATACAAAATTTCCAATGCATAAGCTGATGT
GGAACAGAATGGAATTTCCCATCCAACAAAAGAGGAAAGAATGTTTTAGGAACCAGAATTCTCTGCTGCC
AGTGTTTCTTCAACAAAAATACCACGAGCATACAAGTCTGCCCAGTCCCAGGAAGAAAGAGGAGAGACCC
TGAATTCTGACCTTTTGATGGTCAGGCATGATGGAAAGAAACTGCTGCTACAGCTTGGGAGATTTGCTAT
GGAAAGTCTGCCAGTCAACTTTGCCCTTCTAACCACCAGATCAATTTGTGGCTGATCATCTGATGGGGCA
GTTTCAATCACCAAGCATCGTTCTCTTTCCTGTTCTGGAATTTTGTTTTGGAGCTCTTTCCCCTAGTGAC
CACCAGTTAGTTTCTGAGGGATGGAACAAAAATGCAGCTTGCCCTTTCTATGTGGTGCGTGTTCAGGCCT
TGACAGATTTTATCAAAAGGAAACTATTTTATTTAAATGGAGGCTGAGTGGTGAGTAGATGTGTCTTGGT
ATGGAGGAAAAGGGCATGCTGCATCTTCTTCCTGACCTCCGGGGTCTCTGGCCTTTTGTTTCCTTGCTCA
CTGAGGGGTCTGTCTAACCAAGCAGGCTAGATAGTGCTGGCACACATTGCCTTCTTTCTCATTGGGTCCA
GCAATGAAGATAAGTGTTTGGGTTTTTTTTTTTTCCTCCACAATGTAGCAAATTCTCAGGAAATACAGTT
TATATCTTCCTCCTATGCTCTTCCAGTCACCAACTACTTATGCGGCTACTTTGTCCAGGGCACAAAATGC
CGTGGCAGTATCTAACTAAACCCCCACAAAACTGCTTAATAACAGTTTTGAATGTGAGAAATTTAGATAA
TTTAAATATAAGGTACAGGTTTTAATTTCTGAGTTTCTTCTTTTCTATTTTTATTAAAAAGAAAATAATT
TTCAGATTTAATTGAATTGGAAAAAAACAATACTTCCCACCAGAATTATATATCCTGAAAATTGTATTTT
TGTTATATAAACAACTTTTAAGAAAGATCATTATCCTTTTCTCTACCTAAATATGAGGAGTCTTAGCATA
ATGACAAATATTTATAATTTTTCAATTAATGGTACTTGCTGGATCCACACTAACATCTTTGCTAATAATC
TCATTGTTTCTTCCAACTGATTCCTAACACTATATCCCACATCTTCTTTCTAGTCTTTTATCTAGAATAT
GCAACCTAAAATAAAAATGGTGGCGTCTCCATTCATTCTCCTTCTTCCTTTTTTCCCAAGCCTGGTCTTC
AAAAGGTTGGGCAATTTGGCAGCTGAATTCCCAGACAGAGAATAGAGCAATTTTAGGGATATTAGGACTG
AGGGAGGGTGTGGGAAAGCTGTCATCAGTTGTTTTTATAGAAAGAACTGGCATTCATTAAGAACCTAAAT
CTTATCTTTGCACAAATGGAAAATATAACCTAGTTATAGCTTCCTTTGGCCTTTATTAAAGGGTAATATC
AATCACAGTCATAGCAAAGAAAGCGGATGTATTAATGGCAAATTAATGGAAAACCTCCCTTATCAGGAAT
CTAGACTCAGAATTTAGGAACACAAATCAAATCAGACCAACCAAGCTATAGCCAAGGACTTGAAAGAAAT
TAAACAAGACCCAGAATAAATCAAGGAATTAGAAATTGTTATTTAAAAATTTCAGATTGTAACTCCAGGC
CCTGCTGTCTATATTGCAGCCACTAAAAGCTCACTACCATTAGATTTTTGCTAACATACATGTATTCAGA
AGAAAGCCTATTGAAATTTTCATTGTCTTGTAAAAGGTTGTCCTAGTAAAATGGAAAAGATCCTTAAGTT
ATTAATCAGTTTGAAAAGCAAATTTGTTTTTAAGTTTTACATCAGCAGGGCAGTGTCTTACAAAATTCAG
AAATTGCAAAGGTGGAAATAATTCACGCTGATTTGAAGAACATCTTCTGTGCAATAATACTGCCTCTCTT
GAAAAGCATTGGCTGTTTTTTCTTTTTAAATATATCTCTAGATGCTTTTAAATGTGGCTGTGTTCCCTTT
ACCAAGATTGGCTTCAAGTTTCCGCAGGTAGAGAGACCTGGGCTTGAACAAGAGGATGTGTTTCATGTCC
TGCTGAGGAGGTAGAACATGTGCAGCCTGGGTCCGGGACTGCCTCCGTGGGGCAGGGGCAGGGGCGGTAC
CATTAGGGAGGAAGCTTAGCATTTCAGTTTCTTAAACAATATTCAGGGTGATACACTTTTTCTTCCCTTG
CATTTTAGAATAGGCTGGTATCTCATTTGAACGGGGGAGCAGACTTGATCTCAAATGAAGCTGTGCCCAG
GAGCCAGGCTTAGCATATTGAGATTTTTATAGATACCTTAAAAAATAAAATATTTAAACCTCTCTTTTCT
TCCTTTTTCTATGAAATAGGTTTTTTCTCTAGTTTACAAATGACATGAAAATAGGTTTTATTTGTGTTTT
ATCTGCTTTATTTTTTGATGCTTAGACAACAGTTAGACTTACTGAGCTCCTAAAAAAACGAGGAAGAAGT
CCTTATTTGTGAAAAGCACTTTATGAGTAATTGTATAGACAGTATGTGGCTGCGTCACTGATCATCTTGT
AAGGGTGTAACAGTCTTGTCTGTAAAGTGGCTGCAGTGCCTTCTGTAGTGTGTTTTATTTTTGGTAGGGA
GAGGTGAAGCCTTCTGAAAAATTTGAGAGCAACTACAGAGGATTGTTTGTAACTGTGTAGTATTCCTGAT
GGACTTTTTTCATCGTTAGAGTCAAGGACCTAGACTTTTGCCACTGAAATAATATTGACCAAAAAAATAG
TTTATAAAAGGGATTTGTGAATAGAAAATTCAGTGTGATCATTTGTTGTTAATGTGCACCTTAAAAGAAG
ATTCTGTCTAGCTGTCAAATTCTGGTTCCCGAATATCTCACCCCTGATTGTATTTGAGATCTAGTAGGGC
ATACTGGGGCATTTTAGAAGATAAAATCCCATACAAATGATATATGCTATATTTATGTTGGTGTTGGAGA
AGAAAGAGCAGTATATAAAGAAATAATTCAAGACTGCAGCACTGTCAACCTGAAACTTTGTAAATATTTC
CTAGCTTCTGGTTTGGTGCGGTGACAGCACTTTCATCACAGGATGTTACCTTGTATTCACCAGGCGGAGT
GCGAGCTGCTGCACATCCTCCTCAGATCTCACCTGTCCCCACTGTACATCCACCCGCCAGCTGCTTGCAA
ACCTCATCTCTAGCTTTAGTTCGAAACCACATTGCAGGGTTCAGGTGACCTCTACAAAAAACTACCTCTT
CAGAATGAGGTAATGAATAGTTATTTATTTTAAAATATGAAAAGTCAGGAGCTCTAGAACATGACGATGA
TTTAAGATTTTAACTTTTTTGTGTACTTGTATTTGAGCACTCTCATTTTGTCCTAAAGGGCATTATACAT
TTAAGCAGTAATACTGTAAAAAAATGTGTTGCTCGGAATATCTGAATGTTGTTGAAAGTGGTGCCAGAAC
CGGTTTAGGGGTACGTTTCAGAATCTTAACCTTGAGTCAATTGCATGAAATTAAATAGCTGTGGTATCAC
TTCACTAACAGTGATGTAATTTTAATTTTCAGTAGGCTTGGCATGACAGTACATCCTCATAATGAGTTTG
CTGCAGCTTTGTCACATGCACAGGCATTCATAGAAAGACCACCCAGCTAAGAGGGTAGAATGATTACTCT
TTTTGCAAGATTCTCTTCTTTGTCCAAGTTGGCATTGTTAGTGCTAGGAATACCAGCACCTTGAGACGAG
CAGATTCCAACCATTAGGCTATAAACACCATAGCCAGAGATGGAAGGTTTACTGTGAGTATGAACAGCAA
ATAGCTTACAGGTCATGAGTTGAAATGGTGTAGGTGAGGCTCTAGAAAAATACCTTGACAATTTGCCAAA
TGATCTTACTGTGCCTTCATGATGCAATAAAAAAGCTAACATTTTAGCAGAAATCAGTGATTTGTGAAGA
GAGCAGCCACTCTGGTTTAACTCAGCTGTGTTAATAATTTTTAGAGTGCAATTTAGACTGCATAGGTAAA
TGCACTAAAGAGTTTATAGCCAAAATCACATTTAACAATGAGAAAACACACAGGTAAATTTTCAGTGAAC
AAAATTATTTTTTTAAAGCACATAATCCCTAGTATAGTCAGATATATTTATCACATAGAGCAACTAGGTT
GCAAATATAGTTCAGTGACATTTCTAGAGAAACTTTTTCTACTCCCATAGGCTCTTCAAAGCATGGAACT
TTTATACAACAGAAATGTTGACAGAAATTGCTGTAGTTTAGGGTTGAAGTACTGTATGATGGGCAGCAAT
CATGTATTAACTTAGAAGGGGAAATTGAAATATAGGACCGAATTTGGTTTTATCAGTTTCCAGAGTACTG
CTGCCAACCTAGACACTGATTTTTCAGAGTTTGAAATGTAAATTTCTTCCCGGGACTTGATTGCACATGA
AGCTGGACTGCGTTAGTCATCCTGTCCCAAAGCGCTGTGGGGGCCAGGGTGGAGGTCTCAAGGCATCCTT
TATGACCTGGCCATTGGATGTAAAAGAAAACATATTCCATGCTGTGGTTCTTGTATCTTGTTTCATTCCT
CACCATTGAAAGAGAAAGTCCATGTATTGTCTCCAGCACATCCTTGAAATGTTATACTGGGATGGATTAC
TGATGCCCATCGGTAGTTGAGCCCCAGAAGAGGGTAGTAGCATCTCTGCCTCAGGTGATGATTTGTAGCT
TGGCCAGAGGAGAGCGGAGTCACCAGTATATCTGTGGTCCATGTTGCTAGCTCTGGTAAAATTAAAAATA
CTGGTAAGATGTTTGTTTTATTAGTACACTAGACAGTAAGCTCTGTTTTGTTGTTTTCAAATAACCTATT
TTCACTTTTGTTTGGGCAAAGACATTTAAATTGAAATTCAATTCTAATTTTTGTTAATTGTGGAAAGGGT
AATTAACAGTTCCTATCAGGTATTTTTAATGTGGAAAAGGACAGAAACCCAACTCCTAAAATCTTAAATT
AAGGTAACAGTGCTTTAAAAAAAAAAAATGCATGGGGCAATTAGTCGGCAACTCAATGAGTGACTAAAGT
ACTTTTATTTAACATCCACAACTTCAACTGTTAAGTTTTATTAATTACTAAATCAGCTTTATTAAAATGT
TGACATTTATTTAGCTATTTTGAATAATTATAGTGACTTGACGAGTGTGTATGAGGACACAGCCAATGTA
AGCCAGTGTATCCATTTTTTAGAGGTGCATTTTTTTTTAAAGAATTCTGTAGATAGAAGTGCTCTGAAAA
CAACTAAAATATGTTTATTCATGGTAGTATCAAAAAATGTTTGTACAAACCATCTGCTTCTCCCGGCCAG
CCGAGTTCATTCTCCAGCACCGTGACCGCTGGTTCTCATGTACAGCACATATGCGGGAGAGTTGGCAGAA
AATTTGTGAAGAGATGCCGCAAAGGAAGGGTCTGTTGACGGGTGGGATTGGGGGTTTTGATGAAGTTGCT
TAGTCCTGGTTTTGTTTTGAAAATTACTGCGTTGCATTTTTGTGTTAAGTTTTTGAACCCACGTGTGTTT
TGGTGGAGTATGAGTTGGAAGTCACTGCAAACTAGCATAAACAACAAAGCTCACAGAGTAGGCACAGATG
TAGAGAACAGAGACCAAAATGGGGTGAGGTGGCAGTAAATCTAGGATAGGGAAAAATTAATGTGAGGGTG
GGAAATAAACTGTAATTACCTGAAATCAAATGTAAGAGTGCAATAAGTATGCTTTTTATTCTAAGCTGTG
AACGGTTTTTTTAAGAATCATTCCTTCCTAATACATTTGTGTATGTTCCATAGCTGATTAAAACCAGCTA
TATCAACATATAATGCCTTTTTATTCATGTTAATGACCAACGTAAGTGGCTAGCCTTTATGTCTTATTTA
TCTTCATGTTATGTTAGTTTACATACAGGGGTGTATGTCTCTGTGCTGTCCCCTTCTCCTGCCTTCATTT
TAAAATGCATCCATGGGTCCTCCGTGTTTCCTTTGGCCATGCCACATATATAGACTCAGTTTGGCCTTCA
TGATATCGCCTGATTTTTGAGGACTGTATCACAGTGATATGTATTTGTGGTAATCTCATTTGTTGGTTGT
ACATCTGATCCTTTCCTCAACATGGCAATTGCTGCCTTTCCTAAGATAGGATCATACAACTGATCAGGGG
ATTGAATTTGATCATTCATCAACATGTGTCTCTGAATTTTATTCAGTAGTTGTCATTGCTCTTTGGTTTA
GACCAAGAAAAAGGAAATCCCCCCTTTTCATGTATTCCTTGGTTTGAGGACATGACTCCTGTAAGGGAGA
GGAAAGGGAGATGCTTCCTGTTTGAACTGCAGTGAATTCACGGTTCCTGTTTCACCACTCCAAACCTTAT
GGCGACTCACACACACATTCCTCTTTTCTGTTACTGCCAAAGGTTCGGGTTTAGTACACTTCAGTTCCAC
TCAAGCATTGAAAAGGTTCTCGTGGAGTCTGGGGCGTGCCCAGTGAAAAGATGGGGACTTTTTAATTGTC
CACAGACCTCTCTATACCTGCTTTGCAAAAATTACAATGGAGTAACTATTTTTAAAGCTTATTTTTCAAT
TCATAAAAAAGACATTTATTTTCAGTCAAATGGATGATGTCTCCCTCTTTTCCCCTATTCTCAATGTTTG
CTTGAATCTTTTATTATTTTTTTTAATTCTCCCCCATACCCACTTCCTGATACTTTGGTTCTCTTTCCTG
CTCAGGTCCCTTCATTTGTACTTTGGAGTTTTTCTCATGTAAATTTGTATAACAGAAAATATTGTTCAGT
TTGGATAGAAAGCATGGAGAATAAAAAAAGATAGCTGAAATTCAGATTGAAGAAATTTATTTCTGTGTAA
AGTTATTTAAAAACTGTATTATATAAAAGGCAAAAAAAGTTCTATGTACTTGATGTGAATATGCGAATAC
TGCTATAATAAAGATTGACTGCATGGA
XM_047419953.1: Homo sapiens epidermal growth factor receptor (EGFR),
transcript variant X2, mRNA
(SEQ ID NO: 5)
ACTCCTTCATGGAATCTAAAAAATTGTATTCAGAGAAGCAGAGAGTGGAATGGTGGTTACCAGGGGCTGG
GAAGGTGTGAGCTTGGGGAGATTTGGTGAAAGGACATAGAATCTCAGTTAGACAGGAGGAATAAGTTAAA
GAGATCTATTGCACATCATGGTAACTGTAGTTAGTGACAATGTATTGTATACATGAAAATTGCTAAGAGA
GTAGATTTTAAGTGTTCTCACCACACCAAAAAAAGGTATGTGCAGTAATACAGTCATTAATTAGCTTGAT
GTAGCCATTCCACAATGGATACATATATCAAAACATCATGTTGTATACCATAAATATATACTGTCTCTTT
ATGTAAATTTAAAAATAAGATAAAATAAATGTTATTCACTTGTCGTGGATGTGGTGGGGACAGGTGTGGG
ATAGCCCTCCCTGTACAACTAGGACCCAGGGGTGATCTAGTGACACTAGCCATTTATCAGGACGTATGGG
TGCCAGTCAGGATGATAAAGCTTCCTTTTGGCCACTATACTACTTAGAAATGCCCTGCAAAAGGTGCACA
TCAAAGATTGAAAGCTCAATCCTGGATTTTAAGTGCTTCAAAAGTGCACTTAATTGCCACATTTTTGTCA
AACATTTTCCCAGGTAGTATTTTTCCTCATGTAAAACAACAGCAATTTAATTTGAACAGAAAGCATTTTG
AAACATACTTTTGGCAGGGTTCCTTGCAGATCAGAATGGAAATGATTAACAGGGCAATTATCAATCATGG
ACTTTTGGCGGCAGAAGGAACTGTATTGTTTGGTACAGTCTGGGCCAGGGCCACACACCGTAACGGAGAT
ACTCTATTCTGTGGACGGTTGGAGGGGGCTGTGCTGAGCAGGGTAACTGCATCTTTTCCTAGACTGTTCA
CACTGCTGCCACGAAGGAGTCTTGTTTAGACTGGACCTGGCTTTCTTCTTCGCAATGAGTGTTGCAGACT
CCCGACAAAGGCCAGGTGGTAAAGTGTGGTGTCTGTGAGCGAGAGCCTGAGATGCCTGAGCTGACCTGTC
CTCAGCCACCTGCCATCGTGCAGAGTTTGCCAAGGCACGAGTAACAAGCTCACGCAGTTGGGCACTTTTG
AAGATCATTTTCTCAGCCTCCAGAGGATGTTCAATAACTGTGAGGTGGTCCTTGGGAATTTGGAAATTAC
CTATGTGCAGAGGAATTATGATCTTTCCTTCTTAAAGACCATCCAGGAGGTGGCTGGTTATGTCCTCATT
GCCCTCAACACAGTGGAGCGAATTCCTTTGGAAAACCTGCAGATCATCAGAGGAAATATGTACTACGAAA
ATTCCTATGCCTTAGCAGTCTTATCTAACTATGATGCAAATAAAACCGGACTGAAGGAGCTGCCCATGAG
AAATTTACAGGAAATCCTGCATGGCGCCGTGCGGTTCAGCAACAACCCTGCCCTGTGCAACGTGGAGAGC
ATCCAGTGGCGGGACATAGTCAGCAGTGACTTTCTCAGCAACATGTCGATGGACTTCCAGAACCACCTGG
GCAGCTGCCAAAAGTGTGATCCAAGCTGTCCCAATGGGAGCTGCTGGGGTGCAGGAGAGGAGAACTGCCA
GAAACTGACCAAAATCATCTGTGCCCAGCAGTGCTCCGGGCGCTGCCGTGGCAAGTCCCCCAGTGACTGC
TGCCACAACCAGTGTGCTGCAGGCTGCACAGGCCCCCGGGAGAGCGACTGCCTGGTCTGCCGCAAATTCC
GAGACGAAGCCACGTGCAAGGACACCTGCCCCCCACTCATGCTCTACAACCCCACCACGTACCAGATGGA
TGTGAACCCCGAGGGCAAATACAGCTTTGGTGCCACCTGCGTGAAGAAGTGTCCCCGTAATTATGTGGTG
ACAGATCACGGCTCGTGCGTCCGAGCCTGTGGGGCCGACAGCTATGAGATGGAGGAAGACGGCGTCCGCA
AGTGTAAGAAGTGCGAAGGGCCTTGCCGCAAAGTGTGTAACGGAATAGGTATTGGTGAATTTAAAGACTC
ACTCTCCATAAATGCTACGAATATTAAACACTTCAAAAACTGCACCTCCATCAGTGGCGATCTCCACATC
CTGCCGGTGGCATTTAGGGGTGACTCCTTCACACATACTCCTCCTCTGGATCCACAGGAACTGGATATTC
TGAAAACCGTAAAGGAAATCACAGGGTTTTTGCTGATTCAGGCTTGGCCTGAAAACAGGACGGACCTCCA
TGCCTTTGAGAACCTAGAAATCATACGCGGCAGGACCAAGCAACATGGTCAGTTTTCTCTTGCAGTCGTC
AGCCTGAACATAACATCCTTGGGATTACGCTCCCTCAAGGAGATAAGTGATGGAGATGTGATAATTTCAG
GAAACAAAAATTTGTGCTATGCAAATACAATAAACTGGAAAAAACTGTTTGGGACCTCCGGTCAGAAAAC
CAAAATTATAAGCAACAGAGGTGAAAACAGCTGCAAGGCCACAGGCCAGGTCTGCCATGCCTTGTGCTCC
CCCGAGGGCTGCTGGGGCCCGGAGCCCAGGGACTGCGTCTCTTGCCGGAATGTCAGCCGAGGCAGGGAAT
GCGTGGACAAGTGCAACCTTCTGGAGGGTGAGCCAAGGGAGTTTGTGGAGAACTCTGAGTGCATACAGTG
CCACCCAGAGTGCCTGCCTCAGGCCATGAACATCACCTGCACAGGACGGGGACCAGACAACTGTATCCAG
TGTGCCCACTACATTGACGGCCCCCACTGCGTCAAGACCTGCCCGGCAGGAGTCATGGGAGAAAACAACA
CCCTGGTCTGGAAGTACGCAGACGCCGGCCATGTGTGCCACCTGTGCCATCCAAACTGCACCTACGGATG
CACTGGGCCAGGTCTTGAAGGCTGTCCAACGAATGGGCCTAAGATCCCGTCCATCGCCACTGGGATGGTG
GGGGCCCTCCTCTTGCTGCTGGTGGTGGCCCTGGGGATCGGCCTCTTCATGCGAAGGCGCCACATCGTTC
GGAAGCGCACGCTGCGGAGGCTGCTGCAGGAGAGGGAGCTTGTGGAGCCTCTTACACCCAGTGGAGAAGC
TCCCAACCAAGCTCTCTTGAGGATCTTGAAGGAAACTGAATTCAAAAAGATCAAAGTGCTGGGCTCCGGT
GCGTTCGGCACGGTGTATAAGGGACTCTGGATCCCAGAAGGTGAGAAAGTTAAAATTCCCGTCGCTATCA
AGGAATTAAGAGAAGCAACATCTCCGAAAGCCAACAAGGAAATCCTCGATGAAGCCTACGTGATGGCCAG
CGTGGACAACCCCCACGTGTGCCGCCTGCTGGGCATCTGCCTCACCTCCACCGTGCAGCTCATCACGCAG
CTCATGCCCTTCGGCTGCCTCCTGGACTATGTCCGGGAACACAAAGACAATATTGGCTCCCAGTACCTGC
TCAACTGGTGTGTGCAGATCGCAAAGGGCATGAACTACTTGGAGGACCGTCGCTTGGTGCACCGCGACCT
GGCAGCCAGGAACGTACTGGTGAAAACACCGCAGCATGTCAAGATCACAGATTTTGGGCTGGCCAAACTG
CTGGGTGCGGAAGAGAAAGAATACCATGCAGAAGGAGGCAAAGTGCCTATCAAGTGGATGGCATTGGAAT
CAATTTTACACAGAATCTATACCCACCAGAGTGATGTCTGGAGCTACGGGGTGACTGTTTGGGAGTTGAT
GACCTTTGGATCCAAGCCATATGACGGAATCCCTGCCAGCGAGATCTCCTCCATCCTGGAGAAAGGAGAA
CGCCTCCCTCAGCCACCCATATGTACCATCGATGTCTACATGATCATGGTCAAGTGCTGGATGATAGACG
CAGATAGTCGCCCAAAGTTCCGTGAGTTGATCATCGAATTCTCCAAAATGGCCCGAGACCCCCAGCGCTA
CCTTGTCATTCAGGGGGATGAAAGAATGCATTTGCCAAGTCCTACAGACTCCAACTTCTACCGTGCCCTG
ATGGATGAAGAAGACATGGACGACGTGGTGGATGCCGACGAGTACCTCATCCCACAGCAGGGCTTCTTCA
GCAGCCCCTCCACGTCACGGACTCCCCTCCTGAGCTCTCTGAGTGCAACCAGCAACAATTCCACCGTGGC
TTGCATTGATAGAAATGGGCTGCAAAGCTGTCCCATCAAGGAAGACAGCTTCTTGCAGCGATACAGCTCA
GACCCCACAGGCGCCTTGACTGAGGACAGCATAGACGACACCTTCCTCCCAGTGCCTGAATACATAAACC
AGTCCGTTCCCAAAAGGCCCGCTGGCTCTGTGCAGAATCCTGTCTATCACAATCAGCCTCTGAACCCCGC
GCCCAGCAGAGACCCACACTACCAGGACCCCCACAGCACTGCAGTGGGCAACCCCGAGTATCTCAACACT
GTCCAGCCCACCTGTGTCAACAGCACATTCGACAGCCCTGCCCACTGGGCCCAGAAAGGCAGCCACCAAA
TTAGCCTGGACAACCCTGACTACCAGCAGGACTTCTTTCCCAAGGAAGCCAAGCCAAATGGCATCTTTAA
GGGCTCCACAGCTGAAAATGCAGAATACCTAAGGGTCGCGCCACAAAGCAGTGAATTTATTGGAGCATGA
CCACGGAGGATAGTATGAGCCCTAAAAATCCAGACTCTTTCGATACCCAGGACCAAGCCACAGCAGGTCC
TCCATCCCAACAGCCATGCCCGCATTAGCTCTTAGACCCACAGACTGGTTTTGCAACGTTTACACCGACT
AGCCAGGAAGTACTTCCACCTCGGGCACATTTTGGGAAGTTGCATTCCTTTGTCTTCAAACTGTGAAGCA
TTTACAGAAACGCATCCAGCAAGAATATTGTCCCTTTGAGCAGAAATTTATCTTTCAAAGAGGTATATTT
GAAAAAAAAAAAAAGTATATGTGAGGATTTTTATTGATTGGGGATCTTGGAGTTTTTCATTGTCGCTATT
GATTTTTACTTCAATGGGCTCTTCCAACAAGGAAGAAGCTTGCTGGTAGCACTTGCTACCCTGAGTTCAT
CCAGGCCCAACTGTGAGCAAGGAGCACAAGCCACAAGTCTTCCAGAGGATGCTTGATTCCAGTGGTTCTG
CTTCAAGGCTTCCACTGCAAAACACTAAAGATCCAAGAAGGCCTTCATGGCCCCAGCAGGCCGGATCGGT
ACTGTATCAAGTCATGGCAGGTACAGTAGGATAAGCCACTCTGTCCCTTCCTGGGCAAAGAAGAAACGGA
GGGGATGGAATTCTTCCTTAGACTTACTTTTGTAAAAATGTCCCCACGGTACTTACTCCCCACTGATGGA
CCAGTGGTTTCCAGTCATGAGCGTTAGACTGACTTGTTTGTCTTCCATTCCATTGTTTTGAAACTCAGTA
TGCTGCCCCTGTCTTGCTGTCATGAAATCAGCAAGAGAGGATGACACATCAAATAATAACTCGGATTCCA
GCCCACATTGGATTCATCAGCATTTGGACCAATAGCCCACAGCTGAGAATGTGGAATACCTAAGGATAGC
ACCGCTTTTGTTCTCGCAAAAACGTATCTCCTAATTTGAGGCTCAGATGAAATGCATCAGGTCCTTTGGG
GCATAGATCAGAAGACTACAAAAATGAAGCTGCTCTGAAATCTCCTTTAGCCATCACCCCAACCCCCCAA
AATTAGTTTGTGTTACTTATGGAAGATAGTTTTCTCCTTTTACTTCACTTCAAAAGCTTTTTACTCAAAG
AGTATATGTTCCCTCCAGGTCAGCTGCCCCCAAACCCCCTCCTTACGCTTTGTCACACAAAAAGTGTCTC
TGCCTTGAGTCATCTATTCAAGCACTTACAGCTCTGGCCACAACAGGGCATTTTACAGGTGCGAATGACA
GTAGCATTATGAGTAGTGTGGAATTCAGGTAGTAAATATGAAACTAGGGTTTGAAATTGATAATGCTTTC
ACAACATTTGCAGATGTTTTAGAAGGAAAAAAGTTCCTTCCTAAAATAATTTCTCTACAATTGGAAGATT
GGAAGATTCAGCTAGTTAGGAGCCCACCTTTTTTCCTAATCTGTGTGTGCCCTGTAACCTGACTGGTTAA
CAGCAGTCCTTTGTAAACAGTGTTTTAAACTCTCCTAGTCAATATCCACCCCATCCAATTTATCAAGGAA
GAAATGGTTCAGAAAATATTTTCAGCCTACAGTTATGTTCAGTCACACACACATACAAAATGTTCCTTTT
GCTTTTAAAGTAATTTTTGACTCCCAGATCAGTCAGAGCCCCTACAGCATTGTTAAGAAAGTATTTGATT
TTTGTCTCAATGAAAATAAAACTATATTCATTTCCACTCTATTATGCTCTCAAATACCCCTAAGCATCTA
TACTAGCCTGGTATGGGTATGAAAGATACAAAGATAAATAAAACATAGTCCCTGATTCTAAGAAATTCAC
AATTTAGCAAAGGAAATGGACTCATAGATGCTAACCTTAAAACAACGTGACAAATGCCAGACAGGACCCA
TCAGCCAGGCACTGTGAGAGCACAGAGCAGGGAGGTTGGGTCCTGCCTGAGGAGACCTGGAAGGGAGGCC
TCACAGGAGGATGACCAGGTCTCAGTCAGCGGGGAGGTGGAAAGTGCAGGTGCATCAGGGGCACCCTGAC
CGAGGAAACAGCTGCCAGAGGCCTCCACTGCTAAAGTCCACATAAGGCTGAGGTCAGTCACCCTAAACAA
CCTGCTCCCTCTAAGCCAGGGGATGAGCTTGGAGCATCCCACAAGTTCCCTAAAAGTTGCAGCCCCCAGG
GGGATTTTGAGCTATCATCTCTGCACATGCTTAGTGAGAAGACTACACAACATTTCTAAGAATCTGAGAT
TTTATATTGTCAGTTAACCACTTTCATTATTCATTCACCTCAGGACATGCAGAAATATTTCAGTCAGAAC
TGGGAAACAGAAGGACCTACATTCTGCTGTCACTTATGTGTCAAGAAGCAGATGATCGATGAGGCAGGTC
AGTTGTAAGTGAGTCACATTGTAGCATTAAATTCTAGTATTTTTGTAGTTTGAAACAGTAACTTAATAAA
AGAGCAAAAGCTATTCTAGCTTTCTTCTTCATATTTTAATTTTCCACCATAAAGTTTAGTTGCTAAATTC
TATTAATTTTAAGATTGTGCTTCCCAAAATAGTTCTCACTTCATCTGTCCAGGGAGGCACAGTTCTGTCT
GGTAGAAGCCGCAAAGCCCTTAGCCTCTTCACGGATCTGGCGACTGTGATGGGCAGGTCAGGAGAGGAGC
TGCCCAAAGTCCCATGATTTTCACCTAACAGCCCTGATCAGTCAGTACTCAAAGCTTGGACTCCATCCCT
GAAGGTCTTCCTGATTGATAGCCTGGCCTTAATACCCTACAGAAAGCCTGTCCATTGGCTGTTTCTTCCT
CAGTCAGTTCCTGGAAGACCTTACCCCATGACCCCAGCTTCAGATGTGGTCTTTGGAAACAGAGGTCGAA
GGAAAGTAAGGAGCTGAGAGCTCACATTCATAGGTGCCGCCAGCCTTCGTGCATCTTCTTGCATCATCTC
TAAGGAGCTCCTCTAATTACACCATGCCCGTCACCCCATGAGGGATCAGAGAAGGGATGAGTCTTCTAAA
CTCTATATTCGCTGTGAGTCCAGGTTGTAAGGGGGAGCACTGTGGATGCATCCTATTGCACTCCAGCTGA
TGACACCAAAGCTTAGGTGTTTGCTGAAAGTTCTTGATGTTGTGACTTACCACCCCTGCCTCACAACTGC
AGACATAAGGGGACTATGGATTGCTTAGCAGGAAAGGCACTGGTTCTCAAGGGCGGCTGCCCTTGGGAAT
CTTCTGGTCCCAACCAGAAAGACTGTGGCTTGATTTTCTCAGGTGCAGCCCAGCCGTAGGGCCTTTTCAG
AGCACCCCCTGGTTATTGCAACATTCATCAAAGTTTCTAGAACCTCTGGCCTAAAGGAAGGGCCTGGTGG
GATCTACTTGGCACTCGCTGGGGGGCCACCCCCCAGTGCCACTCTCACTAGGCCTCTGATTGCACTTGTG
TAGGATGAAGCTGGTGGGTGATGGGAACTCAGCACCTCCCCTCAGGCAGAAAAGAATCATCTGTGGAGCT
TCAAAAGAAGGGGCCTGGAGTCTCTGCAGACCAATTCAACCCAAATCTCGGGGGCTCTTTCATGATTCTA
ATGGGCAACCAGGGTTGAAACCCTTATTTCTAGGGTCTTCAGTTGTACAAGACTGTGGGTCTGTACCAGA
GCCCCCGTCAGAGTAGAATAAAAGGCTGGGTAGGGTAGAGATTCCCATGTGCAGTGGAGAGAACAATCTG
CAGTCACTGATAAGCCTGAGACTTGGCTCATTTCAAAAGCGTTCAATTCATCCTCACCAGCAGTTCAGCT
GGAAAGGGGCAAATACCCCCACCTGAGCTTTGAAAACGCCCTGGGACCCTCTGCATTCTCTAAGTAAGTT
ATAGAAACCAGTCTCTTCCCTCCTTTGTGAGTGAGCTGCTATTCCACGTAGGCAACACCTGTTGAAATTG
CCCTCAATGTCTACTCTGCATTTCTTTCTTGTGATAAGCACACACTTTTATTGCAACATAATGATCTGCT
CACATTTCCTTGCCTGGGGGCTGTAAAACCTTACAGAACAGAAATCCTTGCCTCTTTCACCAGCCACACC
TGCCATACCAGGGGTACAGCTTTGTACTATTGAAGACACAGACAGGATTTTTAAATGTAAATCTATTTTT
GTAACTTTGTTGCGGGATATAGTTCTCTTTATGTAGCACTGAACTTTGTACAATATATTTTTAGAAACTC
ATTTTTCTACTAAAACAAACACAGTTTACTTTAGAGAGACTGCAATAGAATCAAAATTTGAAACTGAAAT
CTTTGTTTAAAAGGGTTAAGTTGAGGCAAGAGGAAAGCCCTTTCTCTCTCTTATAAAAAGGCACAACCTC
ATTGGGGAGCTAAGCTAGGTCATTGTCATGGTGAAGAAGAGAAGCATCGTTTTTATATTTAGGAAATTTT
AAAAGATGATGGAAAGCACATTTAGCTTGGTCTGAGGCAGGTTCTGTTGGGGCAGTGTTAATGGAAAGGG
CTCACTGTTGTTACTACTAGAAAAATCCAGTTGCATGCCATACTCTCATCATCTGCCAGTGTAACCCTGT
ACATGTAAGAAAAGCAATAACATAGCACTTTGTTGGTTTATATATATAATGTGACTTCAATGCAAATTTT
ATTTTTATATTTACAATTGATATGCATTTACCAGTATAAACTAGACATGTCTGGAGAGCCTAATAATGTT
CAGCACACTTTGGTTAGTTCACCAACAGTCTTACCAAGCCTGGGCCCAGCCACCCTAGAGAAGTTATTCA
GCCCTGGCTGCAGTGACATCACCTGAGGAGCTTTTAAAAGCTTGAAGCCCAGCTACACCTCAGACCGATT
AAACGCAAATCTCTGGGGCTGAAACCCAAGCATTCGTAGTTTTTAAAGCTCCTGAGGTCATTCCAATGTG
CGGCCAAAGTTGAGAACTACTGGCCTAGGGATTAGCCACAAGGACATGGACTTGGAGGCAAATTCTGCAG
GTGTATGTGATTCTCAGGCCTAGAGAGCTAAGACACAAAGACCTCCACATCTGTCGCTGAGAGTCAAGAA
CCTGAACAGAGTTTCCATGAAGGTTCTCCAAGCACTAGAAGGGAGAGTGTCTAAACAATGGTTGAAAAGC
AAAGGAAATATAAAACAGACACCTCTTTCCATTTCCTAAGGTTTCTCTCTTTATTAAGGGTGGACTAGTA
ATAAAATATAATATTCTTGCTGCTTATGCAGCTGACATTGTTGCCCTCCCTAAAGCAACCAAGTAGCCTT
TATTTCCCACAGTGAAAGAAAACGCTGGCCTATCAGTTACATTACAAAAGGCAGATTTCAAGAGGATTGA
GTAAGTAGTTGGATGGCTTTCATAAAAACAAGAATTCAAGAAGAGGATTCATGCTTTAAGAAACATTTGT
TATACATTCCTCACAAATTATACCTGGGATAAAAACTATGTAGCAGGCAGTGTGTTTTCCTTCCATGTCT
CTCTGCACTACCTGCAGTGTGTCCTCTGAGGCTGCAAGTCTGTCCTATCTGAATTCCCAGCAGAAGCACT
AAGAAGCTCCACCCTATCACCTAGCAGATAAAACTATGGGGAAAACTTAAATCTGTGCATACATTTCTGG
ATGCATTTACTTATCTTTAAAAAAAAAGGAATCCTATGACCTGATTTGGCCACAAAAATAATCTTGCTGT
ACAATACAATCTCTTGGAAATTAAGAGATCCTATGGATTTGATGACTGGTATTAGAGGTGACAATGTAAC
CGATTAACAACAGACAGCAATAACTTCGTTTTAGAAACATTCAAGCAATAGCTTTATAGCTTCAACATAT
GGTACGTTTTAACCTTGAAAGTTTTGCAATGATGAAAGCAGTATTTGTACAAATGAAAAGCAGAATTCTC
TTTTATATGGTTTATACTGTTGATCAGAAATGTTGATTGTGCATTGAGTATTAAAAAATTAGATGTATAT
TATTCATTGTTCTTTACTCCTGAGTACCTTATAATAATAATAATGTATTCTTTGTTAACAA
NM_001005862.3 Homo sapiens erb-b2 receptor tyrosine kinase 2 (ERBB2),
transcript variant 2, mRNA
(SEQ ID NO: 6)
GTTCTTTATTCTACTCTCCGCTGAAGTCCACACAGTTTAAATTAAAGTTCCCGGATTTTTGTGGGCGCCT
GCCCCGCCCCTCGTCCCCCTGCTGTGTCCATATATCGAGGCGATAGGGTTAAGGGAAGGCGGACGCCTGA
TGGGTTAATGAGCAAACTGAAGTGTTTTCCATGATCTTTTTTGAGTCGCAATTGAAGTACCACCTCCCGA
GGGTGATTGCTTCCCCATGCGGGGTAGAACCTTTGCTGTCCTGTTCACCACTCTACCTCCAGCACAGAAT
TTGGCTTATGCCTACTCAATGTGAAGATGATGAGGATGAAAACCTTTGTGATGATCCACTTCCACTTAAT
GAATGGTGGCAAAGCAAAGCTATATTCAAGACCACATGCAAAGCTACTCCCTGAGCAAAGAGTCACAGAT
AAAACGGGGGCACCAGTAGAATGGCCAGGACAAACGCAGTGCAGCACAGAGACTCAGACCCTGGCAGCCA
TGCCTGCGCAGGCAGTGATGAGAGTGACATGTACTGTTGTGGACATGCACAAAAGTGAGTGTGCACCGGC
ACAGACATGAAGCTGCGGCTCCCTGCCAGTCCCGAGACCCACCTGGACATGCTCCGCCACCTCTACCAGG
GCTGCCAGGTGGTGCAGGGAAACCTGGAACTCACCTACCTGCCCACCAATGCCAGCCTGTCCTTCCTGCA
GGATATCCAGGAGGTGCAGGGCTACGTGCTCATCGCTCACAACCAAGTGAGGCAGGTCCCACTGCAGAGG
CTGCGGATTGTGCGAGGCACCCAGCTCTTTGAGGACAACTATGCCCTGGCCGTGCTAGACAATGGAGACC
CGCTGAACAATACCACCCCTGTCACAGGGGCCTCCCCAGGAGGCCTGCGGGAGCTGCAGCTTCGAAGCCT
CACAGAGATCTTGAAAGGAGGGGTCTTGATCCAGCGGAACCCCCAGCTCTGCTACCAGGACACGATTTTG
TGGAAGGACATCTTCCACAAGAACAACCAGCTGGCTCTCACACTGATAGACACCAACCGCTCTCGGGCCT
GCCACCCCTGTTCTCCGATGTGTAAGGGCTCCCGCTGCTGGGGAGAGAGTTCTGAGGATTGTCAGAGCCT
GACGCGCACTGTCTGTGCCGGTGGCTGTGCCCGCTGCAAGGGGCCACTGCCCACTGACTGCTGCCATGAG
CAGTGTGCTGCCGGCTGCACGGGCCCCAAGCACTCTGACTGCCTGGCCTGCCTCCACTTCAACCACAGTG
GCATCTGTGAGCTGCACTGCCCAGCCCTGGTCACCTACAACACAGACACGTTTGAGTCCATGCCCAATCC
CGAGGGCCGGTATACATTCGGCGCCAGCTGTGTGACTGCCTGTCCCTACAACTACCTTTCTACGGACGTG
GGATCCTGCACCCTCGTCTGCCCCCTGCACAACCAAGAGGTGACAGCAGAGGATGGAACACAGCGGTGTG
AGAAGTGCAGCAAGCCCTGTGCCCGAGTGTGCTATGGTCTGGGCATGGAGCACTTGCGAGAGGTGAGGGC
AGTTACCAGTGCCAATATCCAGGAGTTTGCTGGCTGCAAGAAGATCTTTGGGAGCCTGGCATTTCTGCCG
GAGAGCTTTGATGGGGACCCAGCCTCCAACACTGCCCCGCTCCAGCCAGAGCAGCTCCAAGTGTTTGAGA
CTCTGGAAGAGATCACAGGTTACCTATACATCTCAGCATGGCCGGACAGCCTGCCTGACCTCAGCGTCTT
CCAGAACCTGCAAGTAATCCGGGGACGAATTCTGCACAATGGCGCCTACTCGCTGACCCTGCAAGGGCTG
GGCATCAGCTGGCTGGGGCTGCGCTCACTGAGGGAACTGGGCAGTGGACTGGCCCTCATCCACCATAACA
CCCACCTCTGCTTCGTGCACACGGTGCCCTGGGACCAGCTCTTTCGGAACCCGCACCAAGCTCTGCTCCA
CACTGCCAACCGGCCAGAGGACGAGTGTGTGGGCGAGGGCCTGGCCTGCCACCAGCTGTGCGCCCGAGGG
CACTGCTGGGGTCCAGGGCCCACCCAGTGTGTCAACTGCAGCCAGTTCCTTCGGGGCCAGGAGTGCGTGG
AGGAATGCCGAGTACTGCAGGGGCTCCCCAGGGAGTATGTGAATGCCAGGCACTGTTTGCCGTGCCACCC
TGAGTGTCAGCCCCAGAATGGCTCAGTGACCTGTTTTGGACCGGAGGCTGACCAGTGTGTGGCCTGTGCC
CACTATAAGGACCCTCCCTTCTGCGTGGCCCGCTGCCCCAGCGGTGTGAAACCTGACCTCTCCTACATGC
CCATCTGGAAGTTTCCAGATGAGGAGGGCGCATGCCAGCCTTGCCCCATCAACTGCACCCACTCCTGTGT
GGACCTGGATGACAAGGGCTGCCCCGCCGAGCAGAGAGCCAGCCCTCTGACGTCCATCATCTCTGCGGTG
GTTGGCATTCTGCTGGTCGTGGTCTTGGGGGTGGTCTTTGGGATCCTCATCAAGCGACGGCAGCAGAAGA
TCCGGAAGTACACGATGCGGAGACTGCTGCAGGAAACGGAGCTGGTGGAGCCGCTGACACCTAGCGGAGC
GATGCCCAACCAGGCGCAGATGCGGATCCTGAAAGAGACGGAGCTGAGGAAGGTGAAGGTGCTTGGATCT
GGCGCTTTTGGCACAGTCTACAAGGGCATCTGGATCCCTGATGGGGAGAATGTGAAAATTCCAGTGGCCA
TCAAAGTGTTGAGGGAAAACACATCCCCCAAAGCCAACAAAGAAATCTTAGACGAAGCATACGTGATGGC
TGGTGTGGGCTCCCCATATGTCTCCCGCCTTCTGGGCATCTGCCTGACATCCACGGTGCAGCTGGTGACA
CAGCTTATGCCCTATGGCTGCCTCTTAGACCATGTCCGGGAAAACCGCGGACGCCTGGGCTCCCAGGACC
TGCTGAACTGGTGTATGCAGATTGCCAAGGGGATGAGCTACCTGGAGGATGTGCGGCTCGTACACAGGGA
CTTGGCCGCTCGGAACGTGCTGGTCAAGAGTCCCAACCATGTCAAAATTACAGACTTCGGGCTGGCTCGG
CTGCTGGACATTGACGAGACAGAGTACCATGCAGATGGGGGCAAGGTGCCCATCAAGTGGATGGCGCTGG
AGTCCATTCTCCGCCGGCGGTTCACCCACCAGAGTGATGTGTGGAGTTATGGTGTGACTGTGTGGGAGCT
GATGACTTTTGGGGCCAAACCTTACGATGGGATCCCAGCCCGGGAGATCCCTGACCTGCTGGAAAAGGGG
GAGCGGCTGCCCCAGCCCCCCATCTGCACCATTGATGTCTACATGATCATGGTCAAATGTTGGATGATTG
ACTCTGAATGTCGGCCAAGATTCCGGGAGTTGGTGTCTGAATTCTCCCGCATGGCCAGGGACCCCCAGCG
CTTTGTGGTCATCCAGAATGAGGACTTGGGCCCAGCCAGTCCCTTGGACAGCACCTTCTACCGCTCACTG
CTGGAGGACGATGACATGGGGGACCTGGTGGATGCTGAGGAGTATCTGGTACCCCAGCAGGGCTTCTTCT
GTCCAGACCCTGCCCCGGGCGCTGGGGGCATGGTCCACCACAGGCACCGCAGCTCATCTACCAGGAGTGG
CGGTGGGGACCTGACACTAGGGCTGGAGCCCTCTGAAGAGGAGGCCCCCAGGTCTCCACTGGCACCCTCC
GAAGGGGCTGGCTCCGATGTATTTGATGGTGACCTGGGAATGGGGGCAGCCAAGGGGCTGCAAAGCCTCC
CCACACATGACCCCAGCCCTCTACAGCGGTACAGTGAGGACCCCACAGTACCCCTGCCCTCTGAGACTGA
TGGCTACGTTGCCCCCCTGACCTGCAGCCCCCAGCCTGAATATGTGAACCAGCCAGATGTTCGGCCCCAG
CCCCCTTCGCCCCGAGAGGGCCCTCTGCCTGCTGCCCGACCTGCTGGTGCCACTCTGGAAAGGCCCAAGA
CTCTCTCCCCAGGGAAGAATGGGGTCGTCAAAGACGTTTTTGCCTTTGGGGGTGCCGTGGAGAACCCCGA
GTACTTGACACCCCAGGGAGGAGCTGCCCCTCAGCCCCACCCTCCTCCTGCCTTCAGCCCAGCCTTCGAC
AACCTCTATTACTGGGACCAGGACCCACCAGAGCGGGGGGCTCCACCCAGCACCTTCAAAGGGACACCTA
CGGCAGAGAACCCAGAGTACCTGGGTCTGGACGTGCCAGTGTGAACCAGAAGGCCAAGTCCGCAGAAGCC
CTGATGTGTCCTCAGGGAGCAGGGAAGGCCTGACTTCTGCTGGCATCAAGAGGTGGGAGGGCCCTCCGAC
CACTTCCAGGGGAACCTGCCATGCCAGGAACCTGTCCTAAGGAACCTTCCTTCCTGCTTGAGTTCCCAGA
TGGCTGGAAGGGGTCCAGCCTCGTTGGAAGAGGAACAGCACTGGGGAGTCTTTGTGGATTCTGAGGCCCT
GCCCAATGAGACTCTAGGGTCCAGTGGATGCCACAGCCCAGCTTGGCCCTTTCCTTCCAGATCCTGGGTA
CTGAAAGCCTTAGGGAAGCTGGCCTGAGAGGGGAAGCGGCCCTAAGGGAGTGTCTAAGAACAAAAGCGAC
CCATTCAGAGACTGTCCCTGAAACCTAGTACTGCCCCCCATGAGGAAGGAACAGCAATGGTGTCAGTATC
CAGGCTTTGTACAGAGTGCTTTTCTGTTTAGTTTTTACTTTTTTTGTTTTGTTTTTTTAAAGATGAAATA
AAGACCCAGGGGGAGAATGGGTGTTGTATGGGGAGGCAAGTGTGGGGGGTCCTTCTCCACACCCACTTTG
TCCATTTGCAAATATATTTTGGAAAACA
NM_000141.5 Homo sapiens fibroblast growth factor receptor 2 (FGFR2),
transcript variant 1, mRNA
(SEQ ID NO: 7)
GAGAGCGCGGTGGAGAGCCGAGCGGGCGGGCGGCGGGTGCGGAGCGGGCGAGGGAGCGCGCGCGGCCGCC
ACAAAGCTCGGGCGCCGCGGGGCTGCATGCGGCGTACCTGGCCCGGCGCGGCGACTGCTCTCCGGGCTGG
CGGGGGCCGGCCGCGAGCCCCGGGGGCCCCGAGGCCGCAGCTTGCCTGCGCGCTCTGAGCCTTCGCAACT
CGCGAGCAAAGTTTGGTGGAGGCAACGCCAAGCCTGAGTCCTTTCTTCCTCTCGTTCCCCAAATCCGAGG
GCAGCCCGCGGGCGTCATGCCCGCGCTCCTCCGCAGCCTGGGGTACGCGTGAAGCCCGGGAGGCTTGGCG
CCGGCGAAGACCCAAGGACCACTCTTCTGCGTTTGGAGTTGCTCCCCGCAACCCCGGGCTCGTCGCTTTC
TCCATCCCGACCCACGCGGGGCGCGGGGACAACACAGGTCGCGGAGGAGCGTTGCCATTCAAGTGACTGC
AGCAGCAGCGGCAGCGCCTCGGTTCCTGAGCCCACCGCAGGCTGAAGGCATTGCGCGTAGTCCATGCCCG
TAGAGGAAGTGTGCAGATGGGATTAACGTCCACATGGAGATATGGAAGAGGACCGGGGATTGGTACCGTA
ACCATGGTCAGCTGGGGTCGTTTCATCTGCCTGGTCGTGGTCACCATGGCAACCTTGTCCCTGGCCCGGC
CCTCCTTCAGTTTAGTTGAGGATACCACATTAGAGCCAGAAGAGCCACCAACCAAATACCAAATCTCTCA
ACCAGAAGTGTACGTGGCTGCGCCAGGGGAGTCGCTAGAGGTGCGCTGCCTGTTGAAAGATGCCGCCGTG
ATCAGTTGGACTAAGGATGGGGTGCACTTGGGGCCCAACAATAGGACAGTGCTTATTGGGGAGTACTTGC
AGATAAAGGGCGCCACGCCTAGAGACTCCGGCCTCTATGCTTGTACTGCCAGTAGGACTGTAGACAGTGA
AACTTGGTACTTCATGGTGAATGTCACAGATGCCATCTCATCCGGAGATGATGAGGATGACACCGATGGT
GCGGAAGATTTTGTCAGTGAGAACAGTAACAACAAGAGAGCACCATACTGGACCAACACAGAAAAGATGG
AAAAGCGGCTCCATGCTGTGCCTGCGGCCAACACTGTCAAGTTTCGCTGCCCAGCCGGGGGGAACCCAAT
GCCAACCATGCGGTGGCTGAAAAACGGGAAGGAGTTTAAGCAGGAGCATCGCATTGGAGGCTACAAGGTA
CGAAACCAGCACTGGAGCCTCATTATGGAAAGTGTGGTCCCATCTGACAAGGGAAATTATACCTGTGTAG
TGGAGAATGAATACGGGTCCATCAATCACACGTACCACCTGGATGTTGTGGAGCGATCGCCTCACCGGCC
CATCCTCCAAGCCGGACTGCCGGCAAATGCCTCCACAGTGGTCGGAGGAGACGTAGAGTTTGTCTGCAAG
GTTTACAGTGATGCCCAGCCCCACATCCAGTGGATCAAGCACGTGGAAAAGAACGGCAGTAAATACGGGC
CCGACGGGCTGCCCTACCTCAAGGTTCTCAAGGCCGCCGGTGTTAACACCACGGACAAAGAGATTGAGGT
TCTCTATATTCGGAATGTAACTTTTGAGGACGCTGGGGAATATACGTGCTTGGCGGGTAATTCTATTGGG
ATATCCTTTCACTCTGCATGGTTGACAGTTCTGCCAGCGCCTGGAAGAGAAAAGGAGATTACAGCTTCCC
CAGACTACCTGGAGATAGCCATTTACTGCATAGGGGTCTTCTTAATCGCCTGTATGGTGGTAACAGTCAT
CCTGTGCCGAATGAAGAACACGACCAAGAAGCCAGACTTCAGCAGCCAGCCGGCTGTGCACAAGCTGACC
AAACGTATCCCCCTGCGGAGACAGGTAACAGTTTCGGCTGAGTCCAGCTCCTCCATGAACTCCAACACCC
CGCTGGTGAGGATAACAACACGCCTCTCTTCAACGGCAGACACCCCCATGCTGGCAGGGGTCTCCGAGTA
TGAACTTCCAGAGGACCCAAAATGGGAGTTTCCAAGAGATAAGCTGACACTGGGCAAGCCCCTGGGAGAA
GGTTGCTTTGGGCAAGTGGTCATGGCGGAAGCAGTGGGAATTGACAAAGACAAGCCCAAGGAGGCGGTCA
CCGTGGCCGTGAAGATGTTGAAAGATGATGCCACAGAGAAAGACCTTTCTGATCTGGTGTCAGAGATGGA
GATGATGAAGATGATTGGGAAACACAAGAATATCATAAATCTTCTTGGAGCCTGCACACAGGATGGGCCT
CTCTATGTCATAGTTGAGTATGCCTCTAAAGGCAACCTCCGAGAATACCTCCGAGCCCGGAGGCCACCCG
GGATGGAGTACTCCTATGACATTAACCGTGTTCCTGAGGAGCAGATGACCTTCAAGGACTTGGTGTCATG
CACCTACCAGCTGGCCAGAGGCATGGAGTACTTGGCTTCCCAAAAATGTATTCATCGAGATTTAGCAGCC
AGAAATGTTTTGGTAACAGAAAACAATGTGATGAAAATAGCAGACTTTGGACTCGCCAGAGATATCAACA
ATATAGACTATTACAAAAAGACCACCAATGGGCGGCTTCCAGTCAAGTGGATGGCTCCAGAAGCCCTGTT
TGATAGAGTATACACTCATCAGAGTGATGTCTGGTCCTTCGGGGTGTTAATGTGGGAGATCTTCACTTTA
GGGGGCTCGCCCTACCCAGGGATTCCCGTGGAGGAACTTTTTAAGCTGCTGAAGGAAGGACACAGAATGG
ATAAGCCAGCCAACTGCACCAACGAACTGTACATGATGATGAGGGACTGTTGGCATGCAGTGCCCTCCCA
GAGACCAACGTTCAAGCAGTTGGTAGAAGACTTGGATCGAATTCTCACTCTCACAACCAATGAGGAATAC
TTGGACCTCAGCCAACCTCTCGAACAGTATTCACCTAGTTACCCTGACACAAGAAGTTCTTGTTCTTCAG
GAGATGATTCTGTTTTTTCTCCAGACCCCATGCCTTACGAACCATGCCTTCCTCAGTATCCACACATAAA
CGGCAGTGTTAAAACATGAATGACTGTGTCTGCCTGTCCCCAAACAGGACAGCACTGGGAACCTAGCTAC
ACTGAGCAGGGAGACCATGCCTCCCAGAGCTTGTTGTCTCCACTTGTATATATGGATCAGAGGAGTAAAT
AATTGGAAAAGTAATCAGCATATGTGTAAAGATTTATACAGTTGAAAACTTGTAATCTTCCCCAGGAGGA
GAAGAAGGTTTCTGGAGCAGTGGACTGCCACAAGCCACCATGTAACCCCTCTCACCTGCCGTGCGTACTG
GCTGTGGACCAGTAGGACTCAAGGTGGACGTGCGTTCTGCCTTCCTTGTTAATTTTGTAATAATTGGAGA
AGATTTATGTCAGCACACACTTACAGAGCACAAATGCAGTATATAGGTGCTGGATGTATGTAAATATATT
CAAATTATGTATAAATATATATTATATATTTACAAGGAGTTATTTTTTGTATTGATTTTAAATGGATGTC
CCAATGCACCTAGAAAATTGGTCTCTCTTTTTTTAATAGCTATTTGCTAAATGCTGTTCTTACACATAAT
TTCTTAATTTTCACCGAGCAGAGGTGGAAAAATACTTTTGCTTTCAGGGAAAATGGTATAACGTTAATTT
ATTAATAAATTGGTAATATACAAAACAATTAATCATTTATAGTTTTTTTTGTAATTTAAGTGGCATTTCT
ATGCAGGCAGCACAGCAGACTAGTTAATCTATTGCTTGGACTTAACTAGTTATCAGATCCTTTGAAAAGA
GAATATTTACAATATATGACTAATTTGGGGAAAATGAAGTTTTGATTTATTTGTGTTTAAATGCTGCTGT
CAGACGATTGTTCTTAGACCTCCTAAATGCCCCATATTAAAAGAACTCATTCATAGGAAGGTGTTTCATT
TTGGTGTGCAACCCTGTCATTACGTCAACGCAACGTCTAACTGGACTTCCCAAGATAAATGGTACCAGCG
TCCTCTTAAAAGATGCCTTAATCCATTCCTTGAGGACAGACCTTAGTTGAAATGATAGCAGAATGTGCTT
CTCTCTGGCAGCTGGCCTTCTGCTTCTGAGTTGCACATTAATCAGATTAGCCTGTATTCTCTTCAGTGAA
TTTTGATAATGGCTTCCAGACTCTTTGGCGTTGGAGACGCCTGTTAGGATCTTCAAGTCCCATCATAGAA
AATTGAAACACAGAGTTGTTCTGCTGATAGTTTTGGGGATACGTCCATCTTTTTAAGGGATTGCTTTCAT
CTAATTCTGGCAGGACCTCACCAAAAGATCCAGCCTCATACCTACATCAGACAAAATATCGCCGTTGTTC
CTTCTGTACTAAAGTATTGTGTTTTGCTTTGGAAACACCCACTCACTTTGCAATAGCCGTGCAAGATGAA
TGCAGATTACACTGATCTTATGTGTTACAAAATTGGAGAAAGTATTTAATAAAACCTGTTAATTTTTATA
CTGACAATAAAAATGTTTCTACAGATATTAATGTTAACAAGACAAAATAAATGTCACGCAACTTATTTTT
TTAA
NM_001163213.2 Homo sapiens fibroblast growth factor receptor 3 (FGFR3),
transcript variant 3, mRNA
(SEQ ID NO: 8)
AGTGCGCGGTGGCGGCGGCGTCGCGGGCAGCTGGCGCCGCGCGGTCCTGCTCTGCCGGTCGCACGGACGC
ACCGGCGGGCCGCCGGCCGGAGGGACGGGGGGGGAGCTGGGCCCGCGGACAGCGAGCCGGAGCGGGAGCC
GCGCGTAGCGAGCCGGGCTCCGGCGCTCGCCAGTCTCCCGAGCGGCGCCCGCCTCCCGCCGGTGCCCGCG
CCGGGCCGTGGGGGGCAGCATGCCCGCGCGCGCTGCCTGAGGACGCCGCGGCCCCCGCCCCCGCCATGGG
CGCCCCTGCCTGCGCCCTCGCGCTCTGCGTGGCCGTGGCCATCGTGGCCGGCGCCTCCTCGGAGTCCTTG
GGGACGGAGCAGCGCGTCGTGGGGCGAGCGGCAGAAGTCCCGGGCCCAGAGCCCGGCCAGCAGGAGCAGT
TGGTCTTCGGCAGCGGGGATGCTGTGGAGCTGAGCTGTCCCCCGCCCGGGGGTGGTCCCATGGGGCCCAC
TGTCTGGGTCAAGGATGGCACAGGGCTGGTGCCCTCGGAGCGTGTCCTGGTGGGGCCCCAGCGGCTGCAG
GTGCTGAATGCCTCCCACGAGGACTCCGGGGCCTACAGCTGCCGGCAGCGGCTCACGCAGCGCGTACTGT
GCCACTTCAGTGTGCGGGTGACAGACGCTCCATCCTCGGGAGATGACGAAGACGGGGAGGACGAGGCTGA
GGACACAGGTGTGGACACAGGGGCCCCTTACTGGACACGGCCCGAGCGGATGGACAAGAAGCTGCTGGCC
GTGCCGGCCGCCAACACCGTCCGCTTCCGCTGCCCAGCCGCTGGCAACCCCACTCCCTCCATCTCCTGGC
TGAAGAACGGCAGGGAGTTCCGCGGCGAGCACCGCATTGGAGGCATCAAGCTGCGGCATCAGCAGTGGAG
CCTGGTCATGGAAAGCGTGGTGCCCTCGGACCGCGGCAACTACACCTGCGTCGTGGAGAACAAGTTTGGC
AGCATCCGGCAGACGTACACGCTGGACGTGCTGGAGCGCTCCCCGCACCGGCCCATCCTGCAGGCGGGGC
TGCCGGCCAACCAGACGGCGGTGCTGGGCAGCGACGTGGAGTTCCACTGCAAGGTGTACAGTGACGCACA
GCCCCACATCCAGTGGCTCAAGCACGTGGAGGTGAATGGCAGCAAGGTGGGCCCGGACGGCACACCCTAC
GTTACCGTGCTCAAGTCCTGGATCAGTGAGAGTGTGGAGGCCGACGTGCGCCTCCGCCTGGCCAATGTGT
CGGAGCGGGACGGGGGCGAGTACCTCTGTCGAGCCACCAATTTCATAGGCGTGGCCGAGAAGGCCTTTTG
GCTGAGCGTTCACGGGCCCCGAGCAGCCGAGGAGGAGCTGGTGGAGGCTGACGAGGCGGGCAGTGTGTAT
GCAGGCATCCTCAGCTACGGGGTGGGCTTCTTCCTGTTCATCCTGGTGGTGGCGGCTGTGACGCTCTGCC
GCCTGCGCAGCCCCCCCAAGAAAGGCCTGGGCTCCCCCACCGTGCACAAGATCTCCCGCTTCCCGCTCAA
GCGACAGGTGTCCCTGGAGTCCAACGCGTCCATGAGCTCCAACACACCACTGGTGCGCATCGCAAGGCTG
TCCTCAGGGGAGGGCCCCACGCTGGCCAATGTCTCCGAGCTCGAGCTGCCTGCCGACCCCAAATGGGAGC
TGTCTCGGGCCCGGCTGACCCTGGGCAAGCCCCTTGGGGAGGGCTGCTTCGGCCAGGTGGTCATGGCGGA
GGCCATCGGCATTGACAAGGACCGGGCCGCCAAGCCTGTCACCGTAGCCGTGAAGATGCTGAAAGACGAT
GCCACTGACAAGGACCTGTCGGACCTGGTGTCTGAGATGGAGATGATGAAGATGATCGGGAAACACAAAA
ACATCATCAACCTGCTGGGCGCCTGCACGCAGGGCGGGCCCCTGTACGTGCTGGTGGAGTACGCGGCCAA
GGGTAACCTGCGGGAGTTTCTGCGGGCGCGGCGGCCCCCGGGCCTGGACTACTCCTTCGACACCTGCAAG
CCGCCCGAGGAGCAGCTCACCTTCAAGGACCTGGTGTCCTGTGCCTACCAGGTGGCCCGGGGCATGGAGT
ACTTGGCCTCCCAGAAGTGCATCCACAGGGACCTGGCTGCCCGCAATGTGCTGGTGACCGAGGACAACGT
GATGAAGATCGCAGACTTCGGGCTGGCCCGGGACGTGCACAACCTCGACTACTACAAGAAGACGACCAAC
GGCCGGCTGCCCGTGAAGTGGATGGCGCCTGAGGCCTTGTTTGACCGAGTCTACACTCACCAGAGTGACG
TCTGGTCCTTTGGGGTCCTGCTCTGGGAGATCTTCACGCTGGGGGGCTCCCCGTACCCCGGCATCCCTGT
GGAGGAGCTCTTCAAGCTGCTGAAGGAGGGCCACCGCATGGACAAGCCCGCCAACTGCACACACGACCTG
TACATGATCATGCGGGAGTGCTGGCATGCCGCGCCCTCCCAGAGGCCCACCTTCAAGCAGCTGGTGGAGG
ACCTGGACCGTGTCCTTACCGTGACGTCCACCGACGAGTACCTGGACCTGTCGGCGCCTTTCGAGCAGTA
CTCCCCGGGTGGCCAGGACACCCCCAGCTCCAGCTCCTCAGGGGACGACTCCGTGTTTGCCCACGACCTG
CTGCCCCCGGCCCCACCCAGCAGTGGGGGCTCGCGGACGTGAAGGGCCACTGGTCCCCAACAATGTGAGG
GGTCCCTAGCAGCCCACCCTGCTGCTGGTGCACAGCCACTCCCCGGCATGAGACTCAGTGCAGATGGAGA
GACAGCTACACAGAGCTTTGGTCTGTGTGTGTGTGTGTGCGTGTGTGTGTGTGTGTGTGCACATCCGCGT
GTGCCTGTGTGCGTGCGCATCTTGCCTCCAGGTGCAGAGGTACCCTGGGTGTCCCCGCTGCTGTGCAACG
GTCTCCTGACTGGTGCTGCAGCACCGAGGGGCCTTTGTTCTGGGGGGACCCAGTGCAGAATGTAAGTGGG
CCCACCCGGTGGGACCCCCGTGGGGCAGGGAGCTGGGCCCGACATGGCTCCGGCCTCTGCCTTTGCACCA
CGGGACATCACAGGGTGGGCCTCGGCCCCTCCCACACCCAAAGCTGAGCCTGCAGGGAAGCCCCACATGT
CCAGCACCTTGTGCCTGGGGTGTTAGTGGCACCGCCTCCCCACCTCCAGGCTTTCCCACTTCCCACCCTG
CCCCTCAGAGACTGAAATTACGGGTACCTGAAGATGGGAGCCTTTACCTTTTATGCAAAAGGTTTATTCC
GGAAACTAGTGTACATTTCTATAAATAGATGCTGTGTATATGGTATATATACATATATATATATAACATA
TATGGAAGAGGAAAAGGCTGGTACAACGGAGGCCTGCGACCCTGGGGGCACAGGAGGCAGGCATGGCCCT
GGGCGGGGCGTGGGGGGGCGTGGAGGGAGGCCCCAGGGGGTCTCACCCATGCAAGCAGAGGACCAGGGCC
TTTTCTGGCACCGCAGTTTTGTTTTAAAACTGGACCTGTATATTTGTAAAGCTATTTATGGGCCCCTGGC
ACTCTTGTTCCCACACCCCAACACTTCCAGCATTTAGCTGGCCACATGGCGGAGAGTTTTAATTTTTAAC
TTATTGACAACCGAGAAGGTTTATCCCGCCGATAGAGGGACGGCCAAGAATGTACGTCCAGCCTGCCCCG
GAGCTGGAGGATCCCCTCCAAGCCTAAAAGGTTGTTAATAGTTGGAGGTGATTCCAGTGAAGATATTTTA
TTTCCTTTGTCCTTTTTCAGGAGAATTAGATTTCTATAGGATTTTTCTTTAGGAGATTTATTTTTTGGAC
TTCAAAGCAAGCTGGTATTTTCATACAAATTCTTCTAATTGCTGTGTGTCCCAGGCAGGGAGACGGTTTC
CAGGGAGGGGCCGGCCCTGTGTGCAGGTTCCGATGTTATTAGATGTTACAAGTTTATATATATCTATATA
TATAATTTATTGAGTTTTTACAAGATGTATTTGTTGTAGACTTAACACTTCTTACGCAATGCTTCTAGAG
TTTTATAGCCTGGACTGCTACCTTTCAAAGCTTGGAGGGAAGCCGTGAATTCAGTTGGTTCGTTCTGTAC
TGTTACTGGGCCCTGAGTCTGGGCAGCTGTCCCTTGCTTGCCTGCAGGGCCATGGCTCAGGGTGGTCTCT
TCTTGGGGCCCAGTGCATGGTGGCCAGAGGTGTCACCCAAACCGGCAGGTGCGATTTTGTTAACCCAGCG
ACGAACTTTCCGAAAAATAAAGACACCTGGTTGCTAA
NM_203500.2 Homo sapiens kelch like ECH associated protein 1 (KEAP1),
transcript variant 1, mRNA
(SEQ ID NO: 9)
CTTTTCGGGCGTCCCGAGGCCGCTCCCCAACCGACAACCAAGACCCCGCAGGCCACGCAGCCCTGGAGCC
GAGGCCCCCCGACGGCGGAGGCGCCCGCGGGTCCCCTACAGCCAAGGTCCCTGAGTGCCAGAGGTGGTGG
TGTTGCTTATCTTCTGGAACCCCATGCAGCCAGATCCCAGGCCTAGCGGGGCTGGGGCCTGCTGCCGATT
CCTGCCCCTGCAGTCACAGTGCCCTGAGGGGGCAGGGGACGCGGTGATGTACGCCTCCACTGAGTGCAAG
GCGGAGGTGACGCCCTCCCAGCATGGCAACCGCACCTTCAGCTACACCCTGGAGGATCATACCAAGCAGG
CCTTTGGCATCATGAACGAGCTGCGGCTCAGCCAGCAGCTGTGTGACGTCACACTGCAGGTCAAGTACCA
GGATGCACCGGCCGCCCAGTTCATGGCCCACAAGGTGGTGCTGGCCTCATCCAGCCCTGTCTTCAAGGCC
ATGTTCACCAACGGGCTGCGGGAGCAGGGCATGGAGGTGGTGTCCATTGAGGGTATCCACCCCAAGGTCA
TGGAGCGCCTCATTGAATTCGCCTACACGGCCTCCATCTCCATGGGCGAGAAGTGTGTCCTCCACGTCAT
GAACGGTGCTGTCATGTACCAGATCGACAGCGTTGTCCGTGCCTGCAGTGACTTCCTGGTGCAGCAGCTG
GACCCCAGCAATGCCATCGGCATCGCCAACTTCGCTGAGCAGATTGGCTGTGTGGAGTTGCACCAGCGTG
CCCGGGAGTACATCTACATGCATTTTGGGGAGGTGGCCAAGCAAGAGGAGTTCTTCAACCTGTCCCACTG
CCAACTGGTGACCCTCATCAGCCGGGACGACCTGAACGTGCGCTGCGAGTCCGAGGTCTTCCACGCCTGC
ATCAACTGGGTCAAGTACGACTGCGAACAGCGACGGTTCTACGTCCAGGCGCTGCTGCGGGCCGTGCGCT
GCCACTCGTTGACGCCGAACTTCCTGCAGATGCAGCTGCAGAAGTGCGAGATCCTGCAGTCCGACTCCCG
CTGCAAGGACTACCTGGTCAAGATCTTCGAGGAGCTCACCCTGCACAAGCCCACGCAGGTGATGCCCTGC
CGGGCGCCCAAGGTGGGCCGCCTGATCTACACCGCGGGCGGCTACTTCCGACAGTCGCTCAGCTACCTGG
AGGCTTACAACCCCAGTGACGGCACCTGGCTCCGGTTGGCGGACCTGCAGGTGCCGCGGAGCGGCCTGGC
CGGCTGCGTGGTGGGCGGGCTGTTGTACGCCGTGGGCGGCAGGAACAACTCGCCCGACGGCAACACCGAC
TCCAGCGCCCTGGACTGTTACAACCCCATGACCAATCAGTGGTCGCCCTGCGCCCCCATGAGCGTGCCCC
GTAACCGCATCGGGGTGGGGGTCATCGATGGCCACATCTATGCCGTCGGCGGCTCCCACGGCTGCATCCA
CCACAACAGTGTGGAGAGGTATGAGCCAGAGCGGGATGAGTGGCACTTGGTGGCCCCAATGCTGACACGA
AGGATCGGGGTGGGCGTGGCTGTCCTCAATCGTCTCCTTTATGCCGTGGGGGGCTTTGACGGGACAAACC
GCCTTAATTCAGCTGAGTGTTACTACCCAGAGAGGAACGAGTGGCGAATGATCACAGCAATGAACACCAT
CCGAAGCGGGGCAGGCGTCTGCGTCCTGCACAACTGTATCTATGCTGCTGGGGGCTATGATGGTCAGGAC
CAGCTGAACAGCGTGGAGCGCTACGATGTGGAAACAGAGACGTGGACTTTCGTAGCCCCCATGAAGCACC
GGCGAAGTGCCCTGGGGATCACTGTCCACCAGGGGAGAATCTACGTCCTTGGAGGCTATGATGGTCACAC
GTTCCTGGACAGTGTGGAGTGTTACGACCCAGATACAGACACCTGGAGCGAGGTGACCCGAATGACATCG
GGCCGGAGTGGGGTGGGCGTGGCTGTCACCATGGAGCCCTGCCGGAAGCAGATTGACCAGCAGAACTGTA
CCTGTTGAGGCACTTTTGTTTCTTGGGCAAAAATACAGTCCAATGGGGAGTATCATTGTTTTTGTACAAA
AACCGGGACTAAAAGAAAAGACAGCACTGCAAATAACCCATCTTCCGGGAAGGGAGGCCAGGATGCCTCA
GTGTTAAAATGACATCTCAAAAGAAGTCCAAAGCGGGAATCATGTGCCCCTCAGCGGAGCCCCGGGAGTG
TCCAAGACAGCCTGGCTGGGAAAGGGGGTGTGGAAAGAGCAGGCTTCCAGGAGAGAGGCCCCCAAACCCT
CTGGCCGGGTAATAGGCCTGGGTCCCACTCACCCATGCCGGCAGCTGTCACCATGTGATTTATTCTTGGA
TACCTGGGAGGGGGCCAATGGGGGCCTCAGGGGGAGGCCCCCTCTGGAAATGTGGTTCCCAGGGATGGGC
CTGTACATAGAAGCCACCGGATGGCACTTCCCCACCGGATGGACAGTTATTTTGTTGATAAGTAACCCTG
TAATTTTCCAAGGAAAATAAAGAACAGACTAACTAGTGTCTTTCA
NM_033360.4 Homo sapiens KRAS proto-oncogene, GTPase (KRAS),
transcript variant a, mRNA
(SEQ ID NO: 10)
CTAGGCGGCGGCCGCGGCGGCGGAGGCAGCAGCGGCGGCGGCAGTGGCGGCGGCGAAGGTGGCGGCGGCT
CGGCCAGTACTCCCGGCCCCCGCCATTTCGGACTGGGAGCGAGCGCGGCGCAGGCACTGAAGGCGGCGGC
GGGGCCAGAGGCTCAGCGGCTCCCAGGTGCGGGAGAGAGGCCTGCTGAAAATGACTGAATATAAACTTGT
GGTAGTTGGAGCTGGTGGCGTAGGCAAGAGTGCCTTGACGATACAGCTAATTCAGAATCATTTTGTGGAC
GAATATGATCCAACAATAGAGGATTCCTACAGGAAGCAAGTAGTAATTGATGGAGAAACCTGTCTCTTGG
ATATTCTCGACACAGCAGGTCAAGAGGAGTACAGTGCAATGAGGGACCAGTACATGAGGACTGGGGAGGG
CTTTCTTTGTGTATTTGCCATAAATAATACTAAATCATTTGAAGATATTCACCATTATAGAGAACAAATT
AAAAGAGTTAAGGACTCTGAAGATGTACCTATGGTCCTAGTAGGAAATAAATGTGATTTGCCTTCTAGAA
CAGTAGACACAAAACAGGCTCAGGACTTAGCAAGAAGTTATGGAATTCCTTTTATTGAAACATCAGCAAA
GACAAGACAGAGAGTGGAGGATGCTTTTTATACATTGGTGAGAGAGATCCGACAATACAGATTGAAAAAA
ATCAGCAAAGAAGAAAAGACTCCTGGCTGTGTGAAAATTAAAAAATGCATTATAATGTAATCTGGGTGTT
GATGATGCCTTCTATACATTAGTTCGAGAAATTCGAAAACATAAAGAAAAGATGAGCAAAGATGGTAAAA
AGAAGAAAAAGAAGTCAAAGACAAAGTGTGTAATTATGTAAATACAATTTGTACTTTTTTCTTAAGGCAT
ACTAGTACAAGTGGTAATTTTTGTACATTACACTAAATTATTAGCATTTGTTTTAGCATTACCTAATTTT
TTTCCTGCTCCATGCAGACTGTTAGCTTTTACCTTAAATGCTTATTTTAAAATGACAGTGGAAGTTTTTT
TTTCCTCTAAGTGCCAGTATTCCCAGAGTTTTGGTTTTTGAACTAGCAATGCCTGTGAAAAAGAAACTGA
ATACCTAAGATTTCTGTCTTGGGGCTTTTGGTGCATGCAGTTGATTACTTCTTATTTTTCTTACCAATTG
TGAATGTTGGTGTGAAACAAATTAATGAAGCTTTTGAATCATCCCTATTCTGTGTTTTATCTAGTCACAT
AAATGGATTAATTACTAATTTCAGTTGAGACCTTCTAATTGGTTTTTACTGAAACATTGAGGGAACACAA
ATTTATGGGCTTCCTGATGATGATTCTTCTAGGCATCATGTCCTATAGTTTGTCATCCCTGATGAATGTA
AAGTTACACTGTTCACAAAGGTTTTGTCTCCTTTCCACTGCTATTAGTCATGGTCACTCTCCCCAAAATA
TTATATTTTTTCTATAAAAAGAAAAAAATGGAAAAAAATTACAAGGCAATGGAAACTATTATAAGGCCAT
TTCCTTTTCACATTAGATAAATTACTATAAAGACTCCTAATAGCTTTTCCTGTTAAGGCAGACCCAGTAT
GAAATGGGGATTATTATAGCAACCATTTTGGGGCTATATTTACATGCTACTAAATTTTTATAATAATTGA
AAAGATTTTAACAAGTATAAAAAATTCTCATAGGAATTAAATGTAGTCTCCCTGTGTCAGACTGCTCTTT
CATAGTATAACTTTAAATCTTTTCTTCAACTTGAGTCTTTGAAGATAGTTTTAATTCTGCTTGTGACATT
AAAAGATTATTTGGGCCAGTTATAGCTTATTAGGTGTTGAAGAGACCAAGGTTGCAAGGCCAGGCCCTGT
GTGAACCTTTGAGCTTTCATAGAGAGTTTCACAGCATGGACTGTGTCCCCACGGTCATCCAGTGTTGTCA
TGCATTGGTTAGTCAAAATGGGGAGGGACTAGGGCAGTTTGGATAGCTCAACAAGATACAATCTCACTCT
GTGGTGGTCCTGCTGACAAATCAAGAGCATTGCTTTTGTTTCTTAAGAAAACAAACTCTTTTTTAAAAAT
TACTTTTAAATATTAACTCAAAAGTTGAGATTTTGGGGTGGTGGTGTGCCAAGACATTAATTTTTTTTTT
AAACAATGAAGTGAAAAAGTTTTACAATCTCTAGGTTTGGCTAGTTCTCTTAACACTGGTTAAATTAACA
TTGCATAAACACTTTTCAAGTCTGATCCATATTTAATAATGCTTTAAAATAAAAATAAAAACAATCCTTT
TGATAAATTTAAAATGTTACTTATTTTAAAATAAATGAAGTGAGATGGCATGGTGAGGTGAAAGTATCAC
TGGACTAGGAAGAAGGTGACTTAGGTTCTAGATAGGTGTCTTTTAGGACTCTGATTTTGAGGACATCACT
TACTATCCATTTCTTCATGTTAAAAGAAGTCATCTCAAACTCTTAGTTTTTTTTTTTTACAACTATGTAA
TTTATATTCCATTTACATAAGGATACACTTATTTGTCAAGCTCAGCACAATCTGTAAATTTTTAACCTAT
GTTACACCATCTTCAGTGCCAGTCTTGGGCAAAATTGTGCAAGAGGTGAAGTTTATATTTGAATATCCAT
TCTCGTTTTAGGACTCTTCTTCCATATTAGTGTCATCTTGCCTCCCTACCTTCCACATGCCCCATGACTT
GATGCAGTTTTAATACTTGTAATTCCCCTAACCATAAGATTTACTGCTGCTGTGGATATCTCCATGAAGT
TTTCCCACTGAGTCACATCAGAAATGCCCTACATCTTATTTCCTCAGGGCTCAAGAGAATCTGACAGATA
CCATAAAGGGATTTGACCTAATCACTAATTTTCAGGTGGTGGCTGATGCTTTGAACATCTCTTTGCTGCC
CAATCCATTAGCGACAGTAGGATTTTTCAAACCTGGTATGAATAGACAGAACCCTATCCAGTGGAAGGAG
AATTTAATAAAGATAGTGCTGAAAGAATTCCTTAGGTAATCTATAACTAGGACTACTCCTGGTAACAGTA
ATACATTCCATTGTTTTAGTAACCAGAAATCTTCATGCAATGAAAAATACTTTAATTCATGAAGCTTACT
TTTTTTTTTTGGTGTCAGAGTCTCGCTCTTGTCACCCAGGCTGGAATGCAGTGGCGCCATCTCAGCTCAC
TGCAACCTCCATCTCCCAGGTTCAAGCGATTCTCGTGCCTCGGCCTCCTGAGTAGCTGGGATTACAGGCG
TGTGCCACTACACTCAACTAATTTTTGTATTTTTAGGAGAGACGGGGTTTCACCCTGTTGGCCAGGCTGG
TCTCGAACTCCTGACCTCAAGTGATTCACCCACCTTGGCCTCATAAACCTGTTTTGCAGAACTCATTTAT
TCAGCAAATATTTATTGAGTGCCTACCAGATGCCAGTCACCACACAAGGCACTGGGTATATGGTATCCCC
AAACAAGAGACATAATCCCGGTCCTTAGGTAGTGCTAGTGTGGTCTGTAATATCTTACTAAGGCCTTTGG
TATACGACCCAGAGATAACACGATGCGTATTTTAGTTTTGCAAAGAAGGGGTTTGGTCTCTGTGCCAGCT
CTATAATTGTTTTGCTACGATTCCACTGAAACTCTTCGATCAAGCTACTTTATGTAAATCACTTCATTGT
TTTAAAGGAATAAACTTGATTATATTGTTTTTTTATTTGGCATAACTGTGATTCTTTTAGGACAATTACT
GTACACATTAAGGTGTATGTCAGATATTCATATTGACCCAAATGTGTAATATTCCAGTTTTCTCTGCATA
AGTAATTAAAATATACTTAAAAATTAATAGTTTTATCTGGGTACAAATAAACAGGTGCCTGAACTAGTTC
ACAGACAAGGAAACTTCTATGTAAAAATCACTATGATTTCTGAATTGCTATGTGAAACTACAGATCTTTG
GAACACTGTTTAGGTAGGGTGTTAAGACTTACACAGTACCTCGTTTCTACACAGAGAAAGAAATGGCCAT
ACTTCAGGAACTGCAGTGCTTATGAGGGGATATTTAGGCCTCTTGAATTTTTGATGTAGATGGGCATTTT
TTTAAGGTAGTGGTTAATTACCTTTATGTGAACTTTGAATGGTTTAACAAAAGATTTGTTTTTGTAGAGA
TTTTAAAGGGGGAGAATTCTAGAAATAAATGTTACCTAATTATTACAGCCTTAAAGACAAAAATCCTTGT
TGAAGTTTTTTTAAAAAAAGCTAAATTACATAGACTTAGGCATTAACATGTTTGTGGAAGAATATAGCAG
ACGTATATTGTATCATTTGAGTGAATGTTCCCAAGTAGGCATTCTAGGCTCTATTTAACTGAGTCACACT
GCATAGGAATTTAGAACCTAACTTTTATAGGTTATCAAAACTGTTGTCACCATTGCACAATTTTGTCCTA
ATATATACATAGAAACTTTGTGGGGCATGTTAAGTTACAGTTTGCACAAGTTCATCTCATTTGTATTCCA
TTGATTTTTTTTTTCTTCTAAACATTTTTTCTTCAAACAGTATATAACTTTTTTTAGGGGATTTTTTTTT
AGACAGCAAAAACTATCTGAAGATTTCCATTTGTCAAAAAGTAATGATTTCTTGATAATTGTGTAGTAAT
GTTTTTTAGAACCCAGCAGTTACCTTAAAGCTGAATTTATATTTAGTAACTTCTGTGTTAATACTGGATA
GCATGAATTCTGCATTGAGAAACTGAATAGCTGTCATAAAATGAAACTTTCTTTCTAAAGAAAGATACTC
ACATGAGTTCTTGAAGAATAGTCATAACTAGATTAAGATCTGTGTTTTAGTTTAATAGTTTGAAGTGCCT
GTTTGGGATAATGATAGGTAATTTAGATGAATTTAGGGGAAAAAAAAGTTATCTGCAGATATGTTGAGGG
CCCATCTCTCCCCCCACACCCCCACAGAGCTAACTGGGTTACAGTGTTTTATCCGAAAGTTTCCAATTCC
ACTGTCTTGTGTTTTCATGTTGAAAATACTTTTGCATTTTTCCTTTGAGTGCCAATTTCTTACTAGTACT
ATTTCTTAATGTAACATGTTTACCTGGAATGTATTTTAACTATTTTTGTATAGTGTAAACTGAAACATGC
ACATTTTGTACATTGTGCTTTCTTTTGTGGGACATATGCAGTGTGATCCAGTTGTTTTCCATCATTTGGT
TGCGCTGACCTAGGAATGTTGGTCATATCAAACATTAAAAATGACCACTCTTTTAATTGAAATTAACTTT
TAAATGTTTATAGGAGTATGTGCTGTGAAGTGATCTAAAATTTGTAATATTTTTGTCATGAACTGTACTA
CTCCTAATTATTGTAATGTAATAAAAATAGTTACAGTGAC
NM_001411065.1 Homo sapiens mitogen-activated protein kinase kinase 1
(MAP2K1), transcript variant 2, mRNA
(SEQ ID NO: 11)
AGAGAAGCCAGCAAGTAGTTGAGTGTGACGGGTGCATCGGTTCGGGTCGAAGGAAATGAAGCTGGAGAGG
ACCAACTTGGAGGCCTTGCAGAAGAAGCTGGAGGAGCTAGAGCTTGATGAGCAGCAGCGAAAGCGCCTTG
AGGCCTTTCTTACCCAGAAGCAGAAGGTGGGAGAACTGAAGGATGACGACTTTGAGAAGATCAGTGAGCT
GGGGGCTGGCAATGGCGGTGTGGTGTTCAAGGTCTCCCACAAGCCTTCTGGCCTGGTCATGGCCAGAAAG
CTAATTCATCTGGAGATCAAACCCGCAATCCGGAACCAGATCATAAGGGAGCTGCAGGTTCTGCATGAGT
GCAACTCTCCGTACATCGTGGGCTTCTATGGTGCGTTCTACAGCGATGGCGAGATCAGTATCTGCATGGA
GCACATGGTAATAAAAGGCCTGACATATCTGAGGGAGAAGCACAAGATCATGCACAGAGATGTCAAGCCC
TCCAACATCCTAGTCAACTCCCGTGGGGAGATCAAGCTCTGTGACTTTGGGGTCAGCGGGCAGCTCATCG
ACTCCATGGCCAACTCCTTCGTGGGCACAAGGTCCTACATGTCGCCAGAAAGACTCCAGGGGACTCATTA
CTCTGTGCAGTCAGACATCTGGAGCATGGGACTGTCTCTGGTAGAGATGGCGGTTGGGAGGTATCCCATC
CCTCCTCCAGATGCCAAGGAGCTGGAGCTGATGTTTGGGTGCCAGGTGGAAGGAGATGCGGCTGAGACCC
CACCCAGGCCAAGGACCCCCGGGAGGCCCCTTAGCTCATACGGAATGGACAGCCGACCTCCCATGGCAAT
TTTTGAGTTGTTGGATTACATAGTCAACGAGCCTCCTCCAAAACTGCCCAGTGGAGTGTTCAGTCTGGAA
TTTCAAGATTTTGTGAATAAATGCTTAATAAAAAACCCCGCAGAGAGAGCAGATTTGAAGCAACTCATGG
TTCATGCTTTTATCAAGAGATCTGATGCTGAGGAAGTGGATTTTGCAGGTTGGCTCTGCTCCACCATCGG
CCTTAACCAGCCCAGCACACCAACCCATGCTGCTGGCGTCTAAGTGTTTGGGAAGCAACAAAGAGCGAGT
CCCCTGCCCGGTGGTTTGCCATGTCGCTTTTGGGCCTCCTTCCCATGCCTGTCTCTGTTCAGATGTGCAT
TTCACCTGTGACAAAGGATGAAGAACACAGCATGTGCCAAGATTCTACTCTTGTCATTTTTAATATTACT
GTCTTTATTCTTATTACTATTATTGTTCCCCTAAGTGGATTGGCTTTGTGCTTGGGGCTATTTGTGTGTA
TGCTGATGATCAAAACCTGTGCCAGGCTGAATTACAGTGAAATTTTGGTGAATGTGGGTAGTCATTCTTA
CAATTGCACTGCTGTTCCTGCTCCATGACTGGCTGTCTGCCTGTATTTTCGGGATTCTTTGACATTTGGT
GGTACTTTATTCTTGCTGGGCATACTTTCTCTCTAGGAGGGAGCCTTGTGAGATCCTTCACAGGCAGTGC
ATGTGAAGCATGCTTTGCTGCTATGAAAATGAGCATCAGAGAGTGTACATCATGTTATTTTATTATTATT
ATTTGCTTTTCATGTAGAACTCAGCAGTTGACATCCAAATCTAGCCAGAGCCCTTCACTGCCATGATAGC
TGGGGCTTCACCAGTCTGTCTACTGTGGTGATCTGTAGACTTCTGGTTGTATTTCTATATTTATTTTCAG
TATACTGTGTGGGATACTTAGTGGTATGTCTCTTTAAGTTTTGATTAATGTTTCTTAAATGGAATTATTT
TGAATGTCACAAATTGATCAAGATATTAAAATGTCGGATTTATCTTTCCCCATATCCAAGTACCAATGCT
GTTGTAAACAACGTGTATAGTGCCTAAAATTGTATGAAAATCCTTTTAACCATTTTAACCTAGATGTTTA
ACAAATCTAATCTCTTATTCTAATAAATATACTATGAAATAAAAAAAAAAGGATGAAAGCTA
NM_001127500.3 Homo sapiens MET proto-oncogene, receptor tyrosine kinase
(MET), transcript variant 1, mRNA
(SEQ ID NO: 12)
AGACACGTGCTGGGGGGGGCAGGCGAGCGCCTCAGTCTGGTCGCCTGGCGGTGCCTCCGGCCCCAACGCG
CCCGGGCCGCCGCGGGCCGCGCGCGCCGATGCCCGGCTGAGTCACTGGCAGGGCAGCGCGCGTGTGGGAA
GGGGCGGAGGGAGTGCGGCCGGCGGGCGGGCGGGGCGCTGGGCTCAGCCCGGCCGCAGGTGACCCGGAGG
CCCTCGCCGCCCGCGGCGCCCCGAGCGCTTTGTGAGCAGATGCGGAGCCGAGTGGAGGGCGCGAGCCAGA
TGCGGGGCGACAGCTGACTTGCTGAGAGGAGGCGGGGAGGCGCGGAGCGCGCGTGTGGTCCTTGCGCCGC
TGACTTCTCCACTGGTTCCTGGGCACCGAAAGATAAACCTCTCATAATGAAGGCCCCCGCTGTGCTTGCA
CCTGGCATCCTCGTGCTCCTGTTTACCTTGGTGCAGAGGAGCAATGGGGAGTGTAAAGAGGCACTAGCAA
AGTCCGAGATGAATGTGAATATGAAGTATCAGCTTCCCAACTTCACCGCGGAAACACCCATCCAGAATGT
CATTCTACATGAGCATCACATTTTCCTTGGTGCCACTAACTACATTTATGTTTTAAATGAGGAAGACCTT
CAGAAGGTTGCTGAGTACAAGACTGGGCCTGTGCTGGAACACCCAGATTGTTTCCCATGTCAGGACTGCA
GCAGCAAAGCCAATTTATCAGGAGGTGTTTGGAAAGATAACATCAACATGGCTCTAGTTGTCGACACCTA
CTATGATGATCAACTCATTAGCTGTGGCAGCGTCAACAGAGGGACCTGCCAGCGACATGTCTTTCCCCAC
AATCATACTGCTGACATACAGTCGGAGGTTCACTGCATATTCTCCCCACAGATAGAAGAGCCCAGCCAGT
GTCCTGACTGTGTGGTGAGCGCCCTGGGAGCCAAAGTCCTTTCATCTGTAAAGGACCGGTTCATCAACTT
CTTTGTAGGCAATACCATAAATTCTTCTTATTTCCCAGATCATCCATTGCATTCGATATCAGTGAGAAGG
CTAAAGGAAACGAAAGATGGTTTTATGTTTTTGACGGACCAGTCCTACATTGATGTTTTACCTGAGTTCA
GAGATTCTTACCCCATTAAGTATGTCCATGCCTTTGAAAGCAACAATTTTATTTACTTCTTGACGGTCCA
AAGGGAAACTCTAGATGCTCAGACTTTTCACACAAGAATAATCAGGTTCTGTTCCATAAACTCTGGATTG
CATTCCTACATGGAAATGCCTCTGGAGTGTATTCTCACAGAAAAGAGAAAAAAGAGATCCACAAAGAAGG
AAGTGTTTAATATACTTCAGGCTGCGTATGTCAGCAAGCCTGGGGCCCAGCTTGCTAGACAAATAGGAGC
CAGCCTGAATGATGACATTCTTTTCGGGGTGTTCGCACAAAGCAAGCCAGATTCTGCCGAACCAATGGAT
CGATCTGCCATGTGTGCATTCCCTATCAAATATGTCAACGACTTCTTCAACAAGATCGTCAACAAAAACA
ATGTGAGATGTCTCCAGCATTTTTACGGACCCAATCATGAGCACTGCTTTAATAGGACACTTCTGAGAAA
TTCATCAGGCTGTGAAGCGCGCCGTGATGAATATCGAACAGAGTTTACCACAGCTTTGCAGCGCGTTGAC
TTATTCATGGGTCAATTCAGCGAAGTCCTCTTAACATCTATATCCACCTTCATTAAAGGAGACCTCACCA
TAGCTAATCTTGGGACATCAGAGGGTCGCTTCATGCAGGTTGTGGTTTCTCGATCAGGACCATCAACCCC
TCATGTGAATTTTCTCCTGGACTCCCATCCAGTGTCTCCAGAAGTGATTGTGGAGCATACATTAAACCAA
AATGGCTACACACTGGTTATCACTGGGAAGAAGATCACGAAGATCCCATTGAATGGCTTGGGCTGCAGAC
ATTTCCAGTCCTGCAGTCAATGCCTCTCTGCCCCACCCTTTGTTCAGTGTGGCTGGTGCCACGACAAATG
TGTGCGATCGGAGGAATGCCTGAGCGGGACATGGACTCAACAGATCTGTCTGCCTGCAATCTACAAGGTT
TTCCCAAATAGTGCACCCCTTGAAGGAGGGACAAGGCTGACCATATGTGGCTGGGACTTTGGATTTCGGA
GGAATAATAAATTTGATTTAAAGAAAACTAGAGTTCTCCTTGGAAATGAGAGCTGCACCTTGACTTTAAG
TGAGAGCACGATGAATACATTGAAATGCACAGTTGGTCCTGCCATGAATAAGCATTTCAATATGTCCATA
ATTATTTCAAATGGCCACGGGACAACACAATACAGTACATTCTCCTATGTGGATCCTGTAATAACAAGTA
TTTCGCCGAAATACGGTCCTATGGCTGGTGGCACTTTACTTACTTTAACTGGAAATTACCTAAACAGTGG
GAATTCTAGACACATTTCAATTGGTGGAAAAACATGTACTTTAAAAAGTGTGTCAAACAGTATTCTTGAA
TGTTATACCCCAGCCCAAACCATTTCAACTGAGTTTGCTGTTAAATTGAAAATTGACTTAGCCAACCGAG
AGACAAGCATCTTCAGTTACCGTGAAGATCCCATTGTCTATGAAATTCATCCAACCAAATCTTTTATTAG
TACTTGGTGGAAAGAACCTCTCAACATTGTCAGTTTTCTATTTTGCTTTGCCAGTGGTGGGAGCACAATA
ACAGGTGTTGGGAAAAACCTGAATTCAGTTAGTGTCCCGAGAATGGTCATAAATGTGCATGAAGCAGGAA
GGAACTTTACAGTGGCATGTCAACATCGCTCTAATTCAGAGATAATCTGTTGTACCACTCCTTCCCTGCA
ACAGCTGAATCTGCAACTCCCCCTGAAAACCAAAGCCTTTTTCATGTTAGATGGGATCCTTTCCAAATAC
TTTGATCTCATTTATGTACATAATCCTGTGTTTAAGCCTTTTGAAAAGCCAGTGATGATCTCAATGGGCA
ATGAAAATGTACTGGAAATTAAGGGAAATGATATTGACCCTGAAGCAGTTAAAGGTGAAGTGTTAAAAGT
TGGAAATAAGAGCTGTGAGAATATACACTTACATTCTGAAGCCGTTTTATGCACGGTCCCCAATGACCTG
CTGAAATTGAACAGCGAGCTAAATATAGAGTGGAAGCAAGCAATTTCTTCAACCGTCCTTGGAAAAGTAA
TAGTTCAACCAGATCAGAATTTCACAGGATTGATTGCTGGTGTTGTCTCAATATCAACAGCACTGTTATT
ACTACTTGGGTTTTTCCTGTGGCTGAAAAAGAGAAAGCAAATTAAAGATCTGGGCAGTGAATTAGTTCGC
TACGATGCAAGAGTACACACTCCTCATTTGGATAGGCTTGTAAGTGCCCGAAGTGTAAGCCCAACTACAG
AAATGGTTTCAAATGAATCTGTAGACTACCGAGCTACTTTTCCAGAAGATCAGTTTCCTAATTCATCTCA
GAACGGTTCATGCCGACAAGTGCAGTATCCTCTGACAGACATGTCCCCCATCCTAACTAGTGGGGACTCT
GATATATCCAGTCCATTACTGCAAAATACTGTCCACATTGACCTCAGTGCTCTAAATCCAGAGCTGGTCC
AGGCAGTGCAGCATGTAGTGATTGGGCCCAGTAGCCTGATTGTGCATTTCAATGAAGTCATAGGAAGAGG
GCATTTTGGTTGTGTATATCATGGGACTTTGTTGGACAATGATGGCAAGAAAATTCACTGTGCTGTGAAA
TCCTTGAACAGAATCACTGACATAGGAGAAGTTTCCCAATTTCTGACCGAGGGAATCATCATGAAAGATT
TTAGTCATCCCAATGTCCTCTCGCTCCTGGGAATCTGCCTGCGAAGTGAAGGGTCTCCGCTGGTGGTCCT
ACCATACATGAAACATGGAGATCTTCGAAATTTCATTCGAAATGAGACTCATAATCCAACTGTAAAAGAT
CTTATTGGCTTTGGTCTTCAAGTAGCCAAAGGCATGAAATATCTTGCAAGCAAAAAGTTTGTCCACAGAG
ACTTGGCTGCAAGAAACTGTATGCTGGATGAAAAATTCACAGTCAAGGTTGCTGATTTTGGTCTTGCCAG
AGACATGTATGATAAAGAATACTATAGTGTACACAACAAAACAGGTGCAAAGCTGCCAGTGAAGTGGATG
GCTTTGGAAAGTCTGCAAACTCAAAAGTTTACCACCAAGTCAGATGTGTGGTCCTTTGGCGTGCTCCTCT
GGGAGCTGATGACAAGAGGAGCCCCACCTTATCCTGACGTAAACACCTTTGATATAACTGTTTACTTGTT
GCAAGGGAGAAGACTCCTACAACCCGAATACTGCCCAGACCCCTTATATGAAGTAATGCTAAAATGCTGG
CACCCTAAAGCCGAAATGCGCCCATCCTTTTCTGAACTGGTGTCCCGGATATCAGCGATCTTCTCTACTT
TCATTGGGGAGCACTATGTCCATGTGAACGCTACTTATGTGAACGTAAAATGTGTCGCTCCGTATCCTTC
TCTGTTGTCATCAGAAGATAACGCTGATGATGAGGTGGACACACGACCAGCCTCCTTCTGGGAGACATCA
TAGTGCTAGTACTATGTCAAAGCAACAGTCCACACTTTGTCCAATGGTTTTTTCACTGCCTGACCTTTAA
AAGGCCATCGATATTCTTTGCTCTTGCCAAAATTGCACTATTATAGGACTTGTATTGTTATTTAAATTAC
TGGATTCTAAGGAATTTCTTATCTGACAGAGCATCAGAACCAGAGGCTTGGTCCCACAGGCCACGGACCA
ATGGCCTGCAGCCGTGACAACACTCCTGTCATATTGGAGTCCAAAACTTGAATTCTGGGTTGAATTTTTT
AAAAATCAGGTACCACTTGATTTCATATGGGAAATTGAAGCAGGAAATATTGAGGGCTTCTTGATCACAG
AAAACTCAGAAGAGATAGTAATGCTCAGGACAGGAGCGGCAGCCCCAGAACAGGCCACTCATTTAGAATT
CTAGTGTTTCAAAACACTTTTGTGTGTTGTATGGTCAATAACATTTTTCATTACTGATGGTGTCATTCAC
CCATTAGGTAAACATTCCCTTTTAAATGTTTGTTTGTTTTTTGAGACAGGATCTCACTCTGTTGCCAGGG
CTGTAGTGCAGTGGTGTGATCATAGCTCACTGCAACCTCCACCTCCCAGGCTCAAGCCTCCCGAATAGCT
GGGACTACAGGCGCACACCACCATCCCCGGCTAATTTTTGTATTTTTTGTAGAGACGGGGTTTTGCCATG
TTGCCAAGGCTGGTTTCAAACTCCTGGACTCAAGAAATCCACCCACCTCAGCCTCCCAAAGTGCTAGGAT
TACAGGCATGAGCCACTGCGCCCAGCCCTTATAAATTTTTGTATAGACATTCCTTTGGTTGGAAGAATAT
TTATAGGCAATACAGTCAAAGTTTCAAAATAGCATCACACAAAACATGTTTATAAATGAACAGGATGTAA
TGTACATAGATGACATTAAGAAAATTTGTATGAAATAATTTAGTCATCATGAAATATTTAGTTGTCATAT
AAAAACCCACTGTTTGAGAATGATGCTACTCTGATCTAATGAATGTGAACATGTAGATGTTTTGTGTGTA
TTTTTTTAAATGAAAACTCAAAATAAGACAAGTAATTTGTTGATAAATATTTTTAAAGATAACTCAGCAT
GTTTGTAAAGCAGGATACATTTTACTAAAAGGTTCATTGGTTCCAATCACAGCTCATAGGTAGAGCAAAG
AAAGGGTGGATGGATTGAAAAGATTAGCCTCTGTCTCGGTGGCAGGTTCCCACCTCGCAAGCAATTGGAA
ACAAAACTTTTGGGGAGTTTTATTTTGCATTAGGGTGTGTTTTATGTTAAGCAAAACATACTTTAGAAAC
AAATGAAAAAGGCAATTGAAAATCCCAGCTATTTCACCTAGATGGAATAGCCACCCTGAGCAGAACTTTG
TGATGCTTCATTCTGTGGAATTTTGTGCTTGCTACTGTATAGTGCATGTGGTGTAGGTTACTCTAACTGG
TTTTGTCGACGTAAACATTTAAAGTGTTATATTTTTTATAAAAATGTTTATTTTTAATGATATGAGAAAA
ATTTTGTTAGGCCACAAAAACACTGCACTGTGAACATTTTAGAAAAGGTATGTCAGACTGGGATTAATGA
CAGCATGATTTTCAATGACTGTAAATTGCGATAAGGAAATGTACTGATTGCCAATACACCCCACCCTCAT
TACATCATCAGGACTTGAAGCCAAGGGTTAACCCAGCAAGCTACAAAGAGGGTGTGTCACACTGAAACTC
AATAGTTGAGTTTGGCTGTTGTTGCAGGAAAATGATTATAACTAAAAGCTCTCTGATAGTGCAGAGACTT
ACCAGAAGACACAAGGAATTGTACTGAAGAGCTATTACAATCCAAATATTGCCGTTTCATAAATGTAATA
AGTAATACTAATTCACAGAGTATTGTAAATGGTGGATGACAAAAGAAAATCTGCTCTGTGGAAAGAAAGA
ACTGTCTCTACCAGGGTCAAGAGCATGAACGCATCAATAGAAAGAACTCGGGGAAACATCCCATCAACAG
GACTACACACTTGTATATACATTCTTGAGAACACTGCAATGTGAAAATCACGTTTGCTATTTATAAACTT
GTCCTTAGATTAATGTGTCTGGACAGATTGTGGGAGTAAGTGATTCTTCTAAGAATTAGATACTTGTCAC
TGCCTATACCTGCAGCTGAACTGAATGGTACTTCGTATGTTAATAGTTGTTCTGATAAATCATGCAATTA
AAGTAAAGTGATGCAA
NM_002524.5 Homo sapiens NRAS proto-oncogene, GTPase (NRAS), mRNA
(SEQ ID NO: 13)
GGGGCCGGAAGTGCCGCTCCTTGGTGGGGGCTGTTCATGGCGGTTCCGGGGTCTCCAACATTTTTCCCGG
CTGTGGTCCTAAATCTGTCCAAAGCAGAGGCAGTGGAGCTTGAGGTTCTTGCTGGTGTGAAATGACTGAG
TACAAACTGGTGGTGGTTGGAGCAGGTGGTGTTGGGAAAAGCGCACTGACAATCCAGCTAATCCAGAACC
ACTTTGTAGATGAATATGATCCCACCATAGAGGATTCTTACAGAAAACAAGTGGTTATAGATGGTGAAAC
CTGTTTGTTGGACATACTGGATACAGCTGGACAAGAAGAGTACAGTGCCATGAGAGACCAATACATGAGG
ACAGGCGAAGGCTTCCTCTGTGTATTTGCCATCAATAATAGCAAGTCATTTGCGGATATTAACCTCTACA
GGGAGCAGATTAAGCGAGTAAAAGACTCGGATGATGTACCTATGGTGCTAGTGGGAAACAAGTGTGATTT
GCCAACAAGGACAGTTGATACAAAACAAGCCCACGAACTGGCCAAGAGTTACGGGATTCCATTCATTGAA
ACCTCAGCCAAGACCAGACAGGGTGTTGAAGATGCTTTTTACACACTGGTAAGAGAAATACGCCAGTACC
GAATGAAAAAACTCAACAGCAGTGATGATGGGACTCAGGGTTGTATGGGATTGCCATGTGTGGTGATGTA
ACAAGATACTTTTAAAGTTTTGTCAGAAAAGAGCCACTTTCAAGCTGCACTGACACCCTGGTCCTGACTT
CCCTGGAGGAGAAGTATTCCTGTTGCTGTCTTCAGTCTCACAGAGAAGCTCCTGCTACTTCCCCAGCTCT
CAGTAGTTTAGTACAATAATCTCTATTTGAGAAGTTCTCAGAATAACTACCTCCTCACTTGGCTGTCTGA
CCAGAGAATGCACCTCTTGTTACTCCCTGTTATTTTTCTGCCCTGGGTTCTTCCACAGCACAAACACACC
TCTGCCACCCCAGGTTTTTCATCTGAAAAGCAGTTCATGTCTGAAACAGAGAACCAAACCGCAAACGTGA
AATTCTATTGAAAACAGTGTCTTGAGCTCTAAAGTAGCAACTGCTGGTGATTTTTTTTTTCTTTTTACTG
TTGAACTTAGAACTATGCTAATTTTTGGAGAAATGTCATAAATTACTGTTTTGCCAAGAATATAGTTATT
ATTGCTGTTTGGTTTGTTTATAATGTTATCGGCTCTATTCTCTAAACTGGCATCTGCTCTAGATTCATAA
ATACAAAAATGAATACTGAATTTTGAGTCTATCCTAGTCTTCACAACTTTGACGTAATTAAATCCAACTT
TCACAGTGAAGTGCCTTTTTCCTAGAAGTGGTTTGTAGACTTCCTTTATAATATTTCAGTGGAATAGATG
TCTCAAAAATCCTTATGCATGAAATGAATGTCTGAGATACGTCTGTGACTTATCTACCATTGAAGGAAAG
CTATATCTATTTGAGAGCAGATGCCATTTTGTACATGTATGAAATTGGTTTTCCAGAGGCCTGTTTTGGG
GCTTTCCCAGGAGAAAGATGAAACTGAAAGCACATGAATAATTTCACTTAATAATTTTTACCTAATCTCC
ACTTTTTTCATAGGTTACTACCTATACAATGTATGTAATTTGTTTCCCCTAGCTTACTGATAAACCTAAT
ATTCAATGAACTTCCATTTGTATTCAAATTTGTGTCATACCAGAAAGCTCTACATTTGCAGATGTTCAAA
TATTGTAAAACTTTGGTGCATTGTTATTTAATAGCTGTGATCAGTGATTTTCAAACCTCAAATATAGTAT
ATTAACAAATTACATTTTCACTGTATATCATGGTATCTTAATGATGTATATAATTGCCTTCAATCCCCTT
CTCACCCCACCCTCTACAGCTTCCCCCACAGCAATAGGGGCTTGATTATTTCAGTTGAGTAAAGCATGGT
GCTAATGGACCAGGGTCACAGTTTCAAAACTTGAACAATCCAGTTAGCATCACAGAGAAAGAAATTCTTC
TGCATTTGCTCATTGCACCAGTAACTCCAGCTAGTAATTTTGCTAGGTAGCTGCAGTTAGCCCTGCAAGG
AAAGAAGAGGTCAGTTAGCACAAACCCTTTACCATGACTGGAAAACTCAGTATCACGTATTTAAACATTT
TTTTTTCTTTTAGCCATGTAGAAACTCTAAATTAAGCCAATATTCTCATTTGAGAATGAGGATGTCTCAG
CTGAGAAACGTTTTAAATTCTCTTTATTCATAATGTTCTTTGAAGGGTTTAAAACAAGATGTTGATAAAT
CTAAGCTGATGAGTTTGCTCAAAACAGGAAGTTGAAATTGTTGAGACAGGAATGGAAAATATAATTAATT
GATACCTATGAGGATTTGGAGGCTTGGCATTTTAATTTGCAGATAATACCCTGGTAATTCTCATGAAAAA
TAGACTTGGATAACTTTTGATAAAAGACTAATTCCAAAATGGCCACTTTGTTCCTGTCTTTAATATCTAA
ATACTTACTGAGGTCCTCCATCTTCTATATTATGAATTTTCATTTATTAAGCAAATGTCATATTACCTTG
AAATTCAGAAGAGAAGAAACATATACTGTGTCCAGAGTATAATGAACCTGCAGAGTTGTGCTTCTTACTG
CTAATTCTGGGAGCTTTCACAGTACTGTCATCATTTGTAAATGGAAATTCTGCTTTTCTGTTTCTGCTCC
TTCTGGAGCAGTGCTACTCTGTAATTTTCCTGAGGCTTATCACCTCAGTCATTTCTTTTTTAAATGTCTG
TGACTGGCAGTGATTCTTTTTCTTAAAAATCTATTAAATTTGATGTCAAATTAGGGAGAAAGATAGTTAC
TCATCTTGGGCTCTTGTGCCAATAGCCCTTGTATGTATGTACTTAGAGTTTTCCAAGTATGTTCTAAGCA
CAGAAGTTTCTAAATGGGGCCAAAATTCAGACTTGAGTATGTTCTTTGAATACCTTAAGAAGTTACAATT
AGCCGGGCATGGTGGCCCGTGCCTGTAGTCCCAGCTACTTGAGAGGCTGAGGCAGGAGAATCACTTCAAC
CCAGGAGGTGGAGGTTACAGTGAGCAGAGATCGTGCCACTGCACTCCAGCCTGGGTGACAAGAGAGACTT
GTCTCCAAAAAAAAAGTTACACCTAGGTGTGAATTTTGGCACAAAGGAGTGACAAACTTATAGTTAAAAG
CTGAATAACTTCAGTGTGGTATAAAACGTGGTTTTTAGGCTATGTTTGTGATTGCTGAAAAGAATTCTAG
TTTACCTCAAAATCCTTCTCTTTCCCCAAATTAAGTGCCTGGCCAGCTGTCATAAATTACATATTCCTTT
TGGTTTTTTTAAAGGTTACATGTTCAAGAGTGAAAATAAGATGTTCTGTCTGAAGGCTACCATGCCGGAT
CTGTAAATGAACCTGTTAAATGCTGTATTTGCTCCAACGGCTTACTATAGAATGTTACTTAATACAATAT
CATACTTATTACAATTTTTACTATAGGAGTGTAATAGGTAAAATTAATCTCTATTTTAGTGGGCCCATGT
TTAGTCTTTCACCATCCTTTAAACTGCTGTGAATTTTTTTGTCATGACTTGAAAGCAAGGATAGAGAAAC
ACTTTAGAGATATGTGGGGTTTTTTTACCATTCCAGAGCTTGTGAGCATAATCATATTTGCTTTATATTT
ATAGTCATGAACTCCTAAGTTGGCAGCTACAACCAAGAACCAAAAAATGGTGCGTTCTGCTTCTTGTAAT
TCATCTCTGCTAATAAATTATAAGAAGCAAGGAAAATTAGGGAAAATATTTTATTTGGATGGTTTCTATA
AACAAGGGACTATAATTCTTGTACATTATTTTTCATCTTTGCTGTTTCTTTGAGCAGTCTAATGTGCCAC
ACAATTATCTAAGGTATTTGTTTTCTATAAGAATTGTTTTAAAAGTATTCTTGTTACCAGAGTAGTTGTA
TTATATTTCAAAACGTAAGATGATTTTTAAAAGCCTGAGTACTGACCTAAGATGGAATTGTATGAACTCT
GCTCTGGAGGGAGGGGAGGATGTCCGTGGAAGTTGTAAGACTTTTATTTTTTTGTGCCATCAAATATAGG
TAAAAATAATTGTGCAATTCTGCTGTTTAAACAGGAACTATTGGCCTCCTTGGCCCTAAATGGAAGGGCC
GATATTTTAAGTTGATTATTTTATTGTAAATTAATCCAACCTAGTTCTTTTTAATTTGGTTGAATGTTTT
TTCTTGTTAAATGATGTTTAAAAAATAAAAACTGGAAGTTCTTGGCTTAGTCATAA
NM_006218.4 Homo sapiens phosphatidylinositol-4,5-bisphosphate 3-kinase
catalytic subunit alpha (PIK3CA), mRNA
(SEQ ID NO: 14)
AGTTCCGGTGCCGCCGCTGCGGCCGCTGAGGTGTCGGGCTGCTGCTGCCGCGGCCGCTGGGACTGGGGCT
GGGGCCGCCGGCGAGGCAGGGCTCGGGCCCGGCCGGGCAGCTCCGGAGCGGCGGGGGAGAGGGGCCGGGA
GGCGGGGGCCGTGCCGCCCGCTCTCCTCTCCCTCGGCGCCGCCGCCGCCGCCCGCGGGGCTGGGACCCGA
TGCGGTTAGAGCCGCGGAGCCTGGAAGAGCCCCGAGCGTTTCTGCTTTGGGACAACCATACATCTAATTC
CTTAAAGTAGTTTTATATGTAAAACTTGCAAAGAATCAGAACAATGCCTCCACGACCATCATCAGGTGAA
CTGTGGGGCATCCACTTGATGCCCCCAAGAATCCTAGTAGAATGTTTACTACCAAATGGAATGATAGTGA
CTTTAGAATGCCTCCGTGAGGCTACATTAATAACCATAAAGCATGAACTATTTAAAGAAGCAAGAAAATA
CCCCCTCCATCAACTTCTTCAAGATGAATCTTCTTACATTTTCGTAAGTGTTACTCAAGAAGCAGAAAGG
GAAGAATTTTTTGATGAAACAAGACGACTTTGTGACCTTCGGCTTTTTCAACCCTTTTTAAAAGTAATTG
AACCAGTAGGCAACCGTGAAGAAAAGATCCTCAATCGAGAAATTGGTTTTGCTATCGGCATGCCAGTGTG
TGAATTTGATATGGTTAAAGATCCAGAAGTACAGGACTTCCGAAGAAATATTCTGAACGTTTGTAAAGAA
GCTGTGGATCTTAGGGACCTCAATTCACCTCATAGTAGAGCAATGTATGTCTATCCTCCAAATGTAGAAT
CTTCACCAGAATTGCCAAAGCACATATATAATAAATTAGATAAAGGGCAAATAATAGTGGTGATCTGGGT
AATAGTTTCTCCAAATAATGACAAGCAGAAGTATACTCTGAAAATCAACCATGACTGTGTACCAGAACAA
GTAATTGCTGAAGCAATCAGGAAAAAAACTCGAAGTATGTTGCTATCCTCTGAACAACTAAAACTCTGTG
TTTTAGAATATCAGGGCAAGTATATTTTAAAAGTGTGTGGATGTGATGAATACTTCCTAGAAAAATATCC
TCTGAGTCAGTATAAGTATATAAGAAGCTGTATAATGCTTGGGAGGATGCCCAATTTGATGTTGATGGCT
AAAGAAAGCCTTTATTCTCAACTGCCAATGGACTGTTTTACAATGCCATCTTATTCCAGACGCATTTCCA
CAGCTACACCATATATGAATGGAGAAACATCTACAAAATCCCTTTGGGTTATAAATAGTGCACTCAGAAT
AAAAATTCTTTGTGCAACCTACGTGAATGTAAATATTCGAGACATTGATAAGATCTATGTTCGAACAGGT
ATCTACCATGGAGGAGAACCCTTATGTGACAATGTGAACACTCAAAGAGTACCTTGTTCCAATCCCAGGT
GGAATGAATGGCTGAATTATGATATATACATTCCTGATCTTCCTCGTGCTGCTCGACTTTGCCTTTCCAT
TTGCTCTGTTAAAGGCCGAAAGGGTGCTAAAGAGGAACACTGTCCATTGGCATGGGGAAATATAAACTTG
TTTGATTACACAGACACTCTAGTATCTGGAAAAATGGCTTTGAATCTTTGGCCAGTACCTCATGGATTAG
AAGATTTGCTGAACCCTATTGGTGTTACTGGATCAAATCCAAATAAAGAAACTCCATGCTTAGAGTTGGA
GTTTGACTGGTTCAGCAGTGTGGTAAAGTTCCCAGATATGTCAGTGATTGAAGAGCATGCCAATTGGTCT
GTATCCCGAGAAGCAGGATTTAGCTATTCCCACGCAGGACTGAGTAACAGACTAGCTAGAGACAATGAAT
TAAGGGAAAATGACAAAGAACAGCTCAAAGCAATTTCTACACGAGATCCTCTCTCTGAAATCACTGAGCA
GGAGAAAGATTTTCTATGGAGTCACAGACACTATTGTGTAACTATCCCCGAAATTCTACCCAAATTGCTT
CTGTCTGTTAAATGGAATTCTAGAGATGAAGTAGCCCAGATGTATTGCTTGGTAAAAGATTGGCCTCCAA
TCAAACCTGAACAGGCTATGGAACTTCTGGACTGTAATTACCCAGATCCTATGGTTCGAGGTTTTGCTGT
TCGGTGCTTGGAAAAATATTTAACAGATGACAAACTTTCTCAGTATTTAATTCAGCTAGTACAGGTCCTA
AAATATGAACAATATTTGGATAACTTGCTTGTGAGATTTTTACTGAAGAAAGCATTGACTAATCAAAGGA
TTGGGCACTTTTTCTTTTGGCATTTAAAATCTGAGATGCACAATAAAACAGTTAGCCAGAGGTTTGGCCT
GCTTTTGGAGTCCTATTGTCGTGCATGTGGGATGTATTTGAAGCACCTGAATAGGCAAGTCGAGGCAATG
GAAAAGCTCATTAACTTAACTGACATTCTCAAACAGGAGAAGAAGGATGAAACACAAAAGGTACAGATGA
AGTTTTTAGTTGAGCAAATGAGGCGACCAGATTTCATGGATGCTCTACAGGGCTTTCTGTCTCCTCTAAA
CCCTGCTCATCAACTAGGAAACCTCAGGCTTGAAGAGTGTCGAATTATGTCCTCTGCAAAAAGGCCACTG
TGGTTGAATTGGGAGAACCCAGACATCATGTCAGAGTTACTGTTTCAGAACAATGAGATCATCTTTAAAA
ATGGGGATGATTTACGGCAAGATATGCTAACACTTCAAATTATTCGTATTATGGAAAATATCTGGCAAAA
TCAAGGTCTTGATCTTCGAATGTTACCTTATGGTTGTCTGTCAATCGGTGACTGTGTGGGACTTATTGAG
GTGGTGCGAAATTCTCACACTATTATGCAAATTCAGTGCAAAGGCGGCTTGAAAGGTGCACTGCAGTTCA
ACAGCCACACACTACATCAGTGGCTCAAAGACAAGAACAAAGGAGAAATATATGATGCAGCCATTGACCT
GTTTACACGTTCATGTGCTGGATACTGTGTAGCTACCTTCATTTTGGGAATTGGAGATCGTCACAATAGT
AACATCATGGTGAAAGACGATGGACAACTGTTTCATATAGATTTTGGACACTTTTTGGATCACAAGAAGA
AAAAATTTGGTTATAAACGAGAACGTGTGCCATTTGTTTTGACACAGGATTTCTTAATAGTGATTAGTAA
AGGAGCCCAAGAATGCACAAAGACAAGAGAATTTGAGAGGTTTCAGGAGATGTGTTACAAGGCTTATCTA
GCTATTCGACAGCATGCCAATCTCTTCATAAATCTTTTCTCAATGATGCTTGGCTCTGGAATGCCAGAAC
TACAATCTTTTGATGACATTGCATACATTCGAAAGACCCTAGCCTTAGATAAAACTGAGCAAGAGGCTTT
GGAGTATTTCATGAAACAAATGAATGATGCACATCATGGTGGCTGGACAACAAAAATGGATTGGATCTTC
CACACAATTAAACAGCATGCATTGAACTGAAAAGATAACTGAGAAAATGAAAGCTCACTCTGGATTCCAC
ACTGCACTGTTAATAACTCTCAGCAGGCAAAGACCGATTGCATAGGAATTGCACAATCCATGAACAGCAT
TAGAATTTACAGCAAGAACAGAAATAAAATACTATATAATTTAAATAATGTAAACGCAAACAGGGTTTGA
TAGCACTTAAACTAGTTCATTTCAAAATTAAGCTTTAGAATAATGCGCAATTTCATGTTATGCCTTAAGT
CCAAAAAGGTAAACTTTGAAGATTGTTTGTATCTTTTTTTAAAAAACAAAACAAAACAAAAATCCCCAAA
ATATATAGAAATGATGGAGAAGGAAAAAGTGATGGTTTTTTTTGTCTTGCAAATGTTCTATGTTTTGAAA
TGTGGACACAACAAAGGCTGTTATTGCATTAGGTGTAAGTAAACTGGAGTTTATGTTAAATTACATTGAT
TGGAAAAGAATGAAAATTTCTTATTTTTCCATTGCTGTTCAATTTATAGTTTGAAGTGGGTTTTTGACTG
CTTGTTTAATGAAGAAAAATGCTTGGGGTGGAAGGGACTCTTGAGATTTCACCAGAGACTTTTTCTTTTT
AATAAATCAAACCTTTTGATGATTTGAGGTTTTATCTGCAGTTTTGGAAGCAGTCACAAATGAGACCTGT
TATAAGGTGGTATTTTTTTTTTTCTTCTGGACAGTATTTAAAGGATCTTATTCTTATTTCCCAGGGAAAT
TCTGGGCTCCCACAAAGTAAAAAAAAAAAAAAATCATAGAAAAAGAATGAGCAGGAATAGTTCTTATTCC
AGAATTGTACAGTATTCACCTTAAGTTGATTTTTTTTCTCCTTCTGCAATTGAACTGAATACATTTTTCA
TGCATGTTTTCCAGAAAATAGAAGTATTAATGTTATTAAAAAGATTATTTTTTTTATTAAAGGCTATTTA
TATTATAGAAACTATCATTAATATATATTCTTTATTTACATGATCTGTCCCATAGTCATGCATTGTTTTG
CACCCCAAATTTTTTATTGTTCATAGCAGCATGGTCAGCTTTCTTCTTGATCTATAGATGAGGCTCAGGC
ACTATCCCATTTATACCAATAACCAGTGTATAACTACTTAAGGAAAACATAAAAACTTCATCTTCTTTCC
TTTTATTTCTTATGTGAATCTCCCGTCTTCCATTCTCTTTTATAATTGAGAATGTCTCAATCATATGAAA
TTAGTTACCAGAATTAACACAATTTAGACTATCTTCCTGATTCCTTAAACCCCTTTACTGAAGTATACTC
ATGAATAATACTTTAAAATATGGGGGAATAGAAACCATGAACTTTTTACCTTTTTAAACTATTTATCCAT
ATCTCCAAAGTAGAACATTAAACCATTTTAAGATATGTCTCATTCCCAAGTAGTCAGAGCTCACTCTCCA
ACTTTATTAAATACTATTTGAGCACAGGACACATTCTTAAACATTTTGAAAAACATTAACCCAAGATGTA
GAGGCTACTGCTAGTCGTCATTCTAGAATCTGATATTTTACTCTGTATTTGAAATGAATGATTAATGTCC
TAGGAAATTAGCTTTAGCAGATGTCCAGGTGCCACATCAAAAAAGTGCAATAATTATTGACAGTTTTTTA
GATTAGGCATATTATTGGAAAACAACTTTATAAAGAGTGAACATTGTATACTCTAGTAAAACAGCATCAC
TTTAAAAATATTCATTTATGAAATCTGTTACCTATAGTTGAAGTCTTGAGTAGTGAACAAGGGACTCTAA
TACCAATACTCTTAATATCTGGCTATTTTAGATCCCTTAAAGGGCATAATTATTGGAAATTTAGGTATTT
CACTAAAGCATGTATATAATATTGCCAACAAGAAAAGTAAATTTGAAGATTAAGGGAACTTACTTCTGCA
AACTGTCTTGCGATAGTTAAGCAGAATTTAAACTCTGTTTTAAGCAGGAAACCAGAAAGATTATTTTGCA
GTTGTAGAAGATTTCATAACTTATTAAAACTTATTAACATTTTGTGTTGTTTAGATATAGGCAGTTGATA
CATACTAACATCCCAGCCTTTTCAATATCAGGGTTAAATTATAGGAAAACTCAGTAAAATGGTACAAATC
TGAAAGTTTGATGGTAGAAACTGAAGATTTAACAGAGAACTGTGTTTTACCCGAGTGCCAAAAATGCTGT
GAGCCTCCTTGCACAAAATTTATACCACTTTTGCATTTTTATCTATCAGTCCAGATAGTTGTCTCCCCTC
CTTCTCCCAGGACCTCTCCACCATTAAAATGCACAAACCACATGGCCGATTTCACCATTTACATTTATTT
TCAAAAGTTACTACAACCAAATTAATTCTATTAGAAGAAATGTAGACAAATTCTATAAAGACTATAGATT
GTGACCTAAGAAAGAAATGAGGCAAAGAACCAAACATTGAATTAAATGCTACATGGGTGACTAAGATCTG
TTTCAAGTCAGTGATAATATAGCCACTTCTGGGTACTTCAGTATCAGAGATCAGTTCTCGTGGTTTAGAC
AGTTCCTATCTATAGCTGACTATCCTTGTCCTTGAATATGGTGTAACTGACTATTGGCTCTACAGTTTTA
TTGGGCCACTTAAGAAATATTTCCTTGAATAATTATTTTGAGAAAAAGTCTAAAAGTAATAAAAATAATT
TTAAACACACTGTAGTAAGAAATGACTGTTGGAAAATTATGCTTTCACTTTCTACCATATTCTCAGCTAT
ACAAAACCATTTATTTTGAAGATTTTTAGACTACTGTTAATTTGAAATCTGTTACTCTTATTGTGGAATT
TGTTTTTTTAAAAAAGATGTTTCTAATTGGATTTTTAAAAGAAGAATGGAATTTGGTTGCTATTTTACAA
TAGAACCTAAGCTTTTTGTGGTTCTTAGTGTCCTATGTAAAACTTAGTGTCAAAGTAATCAACTTTGAGA
TTTTCCCTTCTATTCTGCTTTATATTAAAAGCCCATTAGAAAATGGGAACCTGGTGAATATATAATGAAT
TGTAAAATATTTTAATGTGTAACTTTTTCAACTGTGAAACTGACTTGATTTTTTGATGAAAACAGCTGCT
GATAAAGTATTTTGTGTAAAGTGTAGTTCTTATTAATCAGGAAAATGATGACTTGATTAGACTGTATATG
CCCTCTTGGATTTTATTTTAAATGGATTGGTGACTTTCACATAGGTAAAACACAGTCCATCTGTATTCTT
TTTTCCATCAAAAATCGAGTGATTTGGAATTATAAAAAAATTGTGAGCAGCCTATTTGAAAGGCATCATG
GAAATTTCACAGCACAATAACACGGATTTGTTTTTTCTTAATGATGTAAATCCGTTTAATTCATACTTTG
ATCAATAGCCCATGCTTGCCAACTCTGAAGAAATTTAATTTCCAGCAGTATTTTAAAGCTAGCCTGTTAA
CTTTTTCTGAATATTTAAAGTTCCTCTTTTTTCTATGTCTGCACAAACTGCAGACCTGGGCTGGACCCAC
ATACTCAAGAGTCCACCTTAAGAAATTATTTTGATGTCCAAGACATCACTAAAATATTTAAGTTTAAAGA
TAATATGTGGTGTTAATAGATTGTGGTGCTTTTACTATTTAAAGACAACTTTCATACTTCAGATGTTTTT
GAGAAGAGGGGAATGTGAGGGGAGGGGGCAGAACAGGGAGGAGTTGTTTGAATGAATTACATTCTTTATA
TCCATCCTGCTCATTTGGGGCATGTCTTTAAGAGAAGGCTGAAAGTTGTGAGAGTATATTGTATACCGTA
AGAGAATCAACTCTTCATCATGGATGGGATTGTGAAGGCTGAACTATAAAATTCAGCATTGACAGCATCC
TCAATTAATAATTCTTGGTGACAGAATAATACAGCTGGGCTGTTTTTTAAAATATAAACAATACCATTTT
TAATTATTACATTAAAAATTGTAAATATATCTATGTGCCATGGCCTGGGAAGCCTGCTTTCTTTTTTCAT
AAAAATTATTTTTACTGTATGAAAAGATCATGGGGTTTAGCTCAAAATATCTGTGGTCCTGATAAAATTG
GATTGGTAACTCTACCTCAGAAGGAAAATGGGAAAAAAAAATAGATGAGTCACAATTCAATACTTCAAGC
TCAGAAACTGTGCAGATCACTGAATTTTAGATTTATAAAGTCAGAGTTGGCATGCCTTGTTTTTAATGAT
ATGGAAGACCTTAAGAAAAAAACTTGGCTGAAGTTTAATCGTTGGTCCAGCCATTTGAAAAAGGCAATAG
TTTGAGGAGGTTCCCGAATTCGGCATTTGAAATTCATTTTGTTCTCTCTTCTTCATTATTAGTGCATTTG
GTGTGTGTATACTTGCACACAATTCTGTTTGTGTACACACTGCTTGCTTAGCCCTAGTCAAGAGGCATCT
TTTATAAAAGGTGTAAAGAAATATCAAGGTTCTAAAATTCGGAAGAGTTTAGAATTTATTAGGAGTTTCC
CAAGTTGGGATGTTAGTCTTTAAATAAACTTCATGCACCTATTCCACTTAAGGTTTTGCACCTCCTTTTT
ATTAGTGCAGTGCCATTTCTTCTGCTTGATTTTAGGTATGTTAATATTCCAGCCTTGCTAGTTAGCATAA
AGTGACAGGTGTGAGCCATGAGGAAATTTTCTGACTTAATTTGTACACAACTACATATAAGAGTTTTAGT
GGAGGAAAAAAATTAGTCCCTTGTGCGTATACAGTAGTTAGGTAAATGATTTTTCTACCAACAGTATACT
CCATTCCTCATGTAGGTAAGTACAGAAAAGGTTTTTAAATGTATTTTTTTAGCCAGTTAAAGTCTATGAA
TCTATCTGCAACCTTATTTAATCTGTCACTATAATAATTTTGTGGTTATGCTAAGAACCATGTATACTTT
TAGGTATTCTTATTTTTGTCAATTTTTCTAGGTTGGCAAGGAGGCAGAAAACCTTCATTGTTTCATATTA
AAATATAATTAGACTAAACTTAATTCTAGTATGAATTTCCAAAATCATTATCTATTTATTTCATTTTTAT
TTAATTTTGTTTTTATTTCATTTTTAAAAGTCCCTTGTTCAATTTAACTTATGTTCCTAAGAGAGGTTGG
AGAACTTGGCCTTCATCTGATTTCAAAAATGTTTTGAGTTTCAAATGAAGTTAATGGTTTCAGTGTGATT
CAGTCCTCAGACCTAATTGGGTTGAATAAAATCTAAAAGAATATACCCTTTTGGAGCATAACATTTTAAT
ACCTTGGGGAATGTGGCACTACCAAAAGAAGACTACTAACACGTCAGATGTTCACCTGGAAGCTTTATCA
AGAAATTCGAACCACCCTTTTGGCCCCATTAATTGTAGCAAGTTTATTTCTCTATATTTTGTCATTCAGT
GAATTGAAGTCCTGTGGTATACTGCATTCATTAGAAGAAAAACGTTTTTAATGTCCTTTTAATGATGGCC
CAGAAAGCATTTGACACAGCAAGATGCATGTGTTACTATATTGAGAATATAGAATAATAACAGTATCACT
AAATTTAAGACCTCTTCCCAGTCTTGCTGTTCCTAGCAAGAAGTTTGGCCTGTGACTGCACTTACTGTTT
ATGCTCATCAGAAACTGTCAATGTCTGCTTTTCTTTAACTCTGCAGTCTGTAACATCACGCTGTTTATTA
AAAAAAAAAAGAAAAATTA
NM_001406743.1 Homo sapiens ret proto-oncogene (RET), transcript variant 1,
mRNA
(SEQ ID NO: 15)
AGTCCCGCGACCGAAGCAGGGCGCGCAGCAGCGCTGAGTGCCCCGGAACGTGCGTCGCGCCCCCAGTGTC
CGTCGCGTCCGCCGCGCCCCGGGCGGGGATGGGGCGGCCAGACTGAGCGCCGCACCCGCCATCCAGACCC
GCCGGCCCTAGCCGCAGTCCCTCCAGCCGTGGCCCCAGCGCGCACGGGCGATGGCGAAGGCGACGTCCGG
TGCCGCGGGGCTGCGTCTGCTGTTGCTGCTGCTGCTGCCGCTGCTAGGCAAAGTGGCATTGGGCCTCTAC
TTCTCGAGGGATGCTTACTGGGAGAAGCTGTATGTGGACCAGGCAGCCGGCACGCCCTTGCTGTACGTCC
ATGCCCTGCGGGACGCCCCTGAGGAGGTGCCCAGCTTCCGCCTGGGCCAGCATCTCTACGGCACGTACCG
CACACGGCTGCATGAGAACAACTGGATCTGCATCCAGGAGGACACCGGCCTCCTCTACCTTAACCGGAGC
CTGGACCATAGCTCCTGGGAGAAGCTCAGTGTCCGCAACCGCGGCTTTCCCCTGCTCACCGTCTACCTCA
AGGTCTTCCTGTCACCCACATCCCTTCGTGAGGGCGAGTGCCAGTGGCCAGGCTGTGCCCGCGTATACTT
CTCCTTCTTCAACACCTCCTTTCCAGCCTGCAGCTCCCTCAAGCCCCGGGAGCTCTGCTTCCCAGAGACA
AGGCCCTCCTTCCGCATTCGGGAGAACCGACCCCCAGGCACCTTCCACCAGTTCCGCCTGCTGCCTGTGC
AGTTCTTGTGCCCCAACATCAGCGTGGCCTACAGGCTCCTGGAGGGTGAGGGTCTGCCCTTCCGCTGCGC
CCCGGACAGCCTGGAGGTGAGCACGCGCTGGGCCCTGGACCGCGAGCAGCGGGAGAAGTACGAGCTGGTG
GCCGTGTGCACCGTGCACGCCGGCGCGCGCGAGGAGGTGGTGATGGTGCCCTTCCCGGTGACCGTGTACG
ACGAGGACGACTCGGCGCCCACCTTCCCCGCGGGCGTCGACACCGCCAGCGCCGTGGTGGAGTTCAAGCG
GAAGGAGGACACCGTGGTGGCCACGCTGCGTGTCTTCGATGCAGACGTGGTACCTGCATCAGGGGAGCTG
GTGAGGCGGTACACAAGCACGCTGCTCCCCGGGGACACCTGGGCCCAGCAGACCTTCCGGGTGGAACACT
GGCCCAACGAGACCTCGGTCCAGGCCAACGGCAGCTTCGTGCGGGCGACCGTACATGACTATAGGCTGGT
TCTCAACCGGAACCTCTCCATCTCGGAGAACCGCACCATGCAGCTGGCGGTGCTGGTCAATGACTCAGAC
TTCCAGGGCCCAGGAGCGGGCGTCCTCTTGCTCCACTTCAACGTGTCGGTGCTGCCGGTCAGCCTGCACC
TGCCCAGTACCTACTCCCTCTCCGTGAGCAGGAGGGCTCGCCGATTTGCCCAGATCGGGAAAGTCTGTGT
GGAAAACTGCCAGGCATTCAGTGGCATCAACGTCCAGTACAAGCTGCATTCCTCTGGTGCCAACTGCAGC
ACGCTAGGGGTGGTCACCTCAGCCGAGGACACCTCGGGGATCCTGTTTGTGAATGACACCAAGGCCCTGC
GGCGGCCCAAGTGTGCCGAACTTCACTACATGGTGGTGGCCACCGACCAGCAGACCTCTAGGCAGGCCCA
GGCCCAGCTGCTTGTAACAGTGGAGGGGTCATATGTGGCCGAGGAGGCGGGCTGCCCCCTGTCCTGTGCA
GTCAGCAAGAGACGGCTGGAGTGTGAGGAGTGTGGCGGCCTGGGCTCCCCAACAGGCAGGTGTGAGTGGA
GGCAAGGAGATGGCAAAGGGATCACCAGGAACTTCTCCACCTGCTCTCCCAGCACCAAGACCTGCCCCGA
CGGCCACTGCGATGTTGTGGAGACCCAAGACATCAACATTTGCCCTCAGGACTGCCTCCGGGGCAGCATT
GTTGGGGGACACGAGCCTGGGGAGCCCCGGGGGATTAAAGCTGGCTATGGCACCTGCAACTGCTTCCCTG
AGGAGGAGAAGTGCTTCTGCGAGCCCGAAGACATCCAGGATCCACTGTGCGACGAGCTGTGCCGCACGGT
GATCGCAGCCGCTGTCCTCTTCTCCTTCATCGTCTCGGTGCTGCTGTCTGCCTTCTGCATCCACTGCTAC
CACAAGTTTGCCCACAAGCCACCCATCTCCTCAGCTGAGATGACCTTCCGGAGGCCCGCCCAGGCCTTCC
CGGTCAGCTACTCCTCTTCCGGTGCCCGCCGGCCCTCGCTGGACTCCATGGAGAACCAGGTCTCCGTGGA
TGCCTTCAAGATCCTGGAGGATCCAAAGTGGGAATTCCCTCGGAAGAACTTGGTTCTTGGAAAAACTCTA
GGAGAAGGCGAATTTGGAAAAGTGGTCAAGGCAACGGCCTTCCATCTGAAAGGCAGAGCAGGGTACACCA
CGGTGGCCGTGAAGATGCTGAAAGAGAACGCCTCCCCGAGTGAGCTGCGAGACCTGCTGTCAGAGTTCAA
CGTCCTGAAGCAGGTCAACCACCCACATGTCATCAAATTGTATGGGGCCTGCAGCCAGGATGGCCCGCTC
CTCCTCATCGTGGAGTACGCCAAATACGGCTCCCTGCGGGGCTTCCTCCGCGAGAGCCGCAAAGTGGGGC
CTGGCTACCTGGGCAGTGGAGGCAGCCGCAACTCCAGCTCCCTGGACCACCCGGATGAGCGGGCCCTCAC
CATGGGCGACCTCATCTCATTTGCCTGGCAGATCTCACAGGGGATGCAGTATCTGGCCGAGATGAAGCTC
GTTCATCGGGACTTGGCAGCCAGAAACATCCTGGTAGCTGAGGGGCGGAAGATGAAGATTTCGGATTTCG
GCTTGTCCCGAGATGTTTATGAAGAGGATTCCTACGTGAAGAGGAGCCAGGGTCGGATTCCAGTTAAATG
GATGGCAATTGAATCCCTTTTTGATCATATCTACACCACGCAAAGTGATGTATGGTCTTTTGGTGTCCTG
CTGTGGGAGATCGTGACCCTAGGGGGAAACCCCTATCCTGGGATTCCTCCTGAGCGGCTCTTCAACCTTC
TGAAGACCGGCCACCGGATGGAGAGGCCAGACAACTGCAGCGAGGAGATGTACCGCCTGATGCTGCAATG
CTGGAAGCAGGAGCCGGACAAAAGGCCGGTGTTTGCGGACATCAGCAAAGACCTGGAGAAGATGATGGTT
AAGAGGAGAGACTACTTGGACCTTGCGGCGTCCACTCCATCTGACTCCCTGATTTATGACGACGGCCTCT
CAGAGGAGGAGACACCGCTGGTGGACTGTAATAATGCCCCCCTCCCTCGAGCCCTCCCTTCCACATGGAT
TGAAAACAAACTCTATGGCATGTCAGACCCGAACTGGCCTGGAGAGAGTCCTGTACCACTCACGAGAGCT
GATGGCACTAACACTGGGTTTCCAAGATATCCAAATGATAGTGTATATGCTAACTGGATGCTTTCACCCT
CAGCGGCAAAATTAATGGACACGTTTGATAGTTAACATTTCTTTGTGAAAGATGCACAACACTCCTCCAG
TCTTGTGGGGGCAGCTTTTGGGAAGTCTCAGCAGCTCTTCTGGCTGTGTTGTCAGCACTGTAACTTCGCA
GAAAAGAGTCGGATTACCAAAACACTGCCTGCTCTTCAGACTTAAAGCACTGATAGGACTTAAAATAGTC
TCATTCAAATACTGTATTTTATATAGGCATTTCACAAAAACAGCAAAATTGTGGCATTTTGTGAGGCCAA
GGCTTGGATGCGTGTGTAATAGAGCCTTGTGGTGTGTGCGCACACACCCAGAGGGAGAGTTTGAAAAATG
CTTATTGGACACGTAACCTGGCTCTAATTTGGGCTGTTTTTCAGATACACTGTGATAAGTTCTTTTACAA
ATATCTATAGACATGGTAAACTTTTGGTTTTCAGATATGCTTAATGATAGTCTTACTAAATGCAGAAATA
AGAATAAACTTTCTCAAATTATTAAAAATGCCTACACAGTAAGTGTGAATTGCTGCAACAGGTTTGTTCT
CAGGAGGGTAAGAACTCCAGGTCTAAACAGCTGACCCAGTGATGGGGAATTTATCCTTGACCAATTTATC
CTTGACCAATAACCTAATTGTCTATTCCTGAGTTATAAAAGTCCCCATCCTTATTAGCTCTACTGGAATT
TTCATACACGTAAATGCAGAAGTTACTAAGTATTAAGTATTACTGAGTATTAAGTAGTAATCTGTCAGTT
ATTAAAATTTGTAAAATCTATTTATGAAAGGTCATTAAACCAGATCATGTTCCTTTTTTTGTAATCAAGG
TGACTAAGAAAATCAGTTGTGTAAATAAAATCATGTATCATAAAA
NM_002944.3 Homo sapiens ROS proto-oncogene 1, receptor tyrosine kinase
(ROS1), transcript variant 1, mRNA
(SEQ ID NO: 16)
GCACTTCTAAGAACTAACCTTTAGTCACTGGGTGACTTTATGGGAGTAAAAGGAAGCTGTTATGAAATAG
CTCTTATGGAACTGTTACAAGCTTTCAAGCATTCAAAGGTCTAAATGAAAAAGGCTAAGTATTATTTCAA
AAGGCAAGTATATCCTAATATAGCAAAACAAACAAAGCAAAATCCATCAGCTACTCCTCCAATTGAAGTG
ATGAAGCCCAAATAATTCATATAGCAAAATGGAGAAAATTAGACCGGCCATCTAAAAATCTGCCATTGGT
GAAGTGATGAAGAACATTTACTGTCTTATTCCGAAGCTTGTCAATTTTGCAACTCTTGGCTGCCTATGGA
TTTCTGTGGTGCAGTGTACAGTTTTAAATAGCTGCCTAAAGTCGTGTGTAACTAATCTGGGCCAGCAGCT
TGACCTTGGCACACCACATAATCTGAGTGAACCGTGTATCCAAGGATGTCACTTTTGGAACTCTGTAGAT
CAGAAAAACTGTGCTTTAAAGTGTCGGGAGTCGTGTGAGGTTGGCTGTAGCAGCGCGGAAGGTGCATATG
AAGAGGAAGTACTGGAAAATGCAGACCTACCAACTGCTCCCTTTGCTTCTTCCATTGGAAGCCACAATAT
GACATTACGATGGAAATCTGCAAACTTCTCTGGAGTAAAATACATCATTCAGTGGAAATATGCACAACTT
CTGGGAAGCTGGACTTATACTAAGACTGTGTCCAGACCGTCCTATGTGGTCAAGCCCCTGCACCCCTTCA
CTGAGTACATTTTCCGAGTGGTTTGGATCTTCACAGCGCAGCTGCAGCTCTACTCCCCTCCAAGTCCCAG
TTACAGGACTCATCCTCATGGAGTTCCTGAAACTGCACCTTTGATTAGGAATATTGAGAGCTCAAGTCCC
GACACTGTGGAAGTCAGCTGGGATCCACCTCAATTCCCAGGTGGACCTATTTTGGGTTATAACTTAAGGC
TGATCAGCAAAAATCAAAAATTAGATGCAGGGACACAGAGAACCAGTTTCCAGTTTTACTCCACTTTACC
AAATACTATCTACAGGTTTTCTATTGCAGCAGTAAATGAAGTTGGTGAGGGTCCAGAAGCAGAATCTAGT
ATTACCACTTCATCTTCAGCAGTTCAACAAGAGGAACAGTGGCTCTTTTTATCCAGAAAAACTTCTCTAA
GAAAGAGATCTTTAAAACATTTAGTAGATGAAGCACATTGCCTTCGGTTGGATGCTATATACCATAATAT
TACAGGAATATCTGTTGATGTCCACCAGCAAATTGTTTATTTCTCTGAAGGAACTCTCATATGGGCGAAG
AAGGCTGCCAACATGTCTGATGTATCTGACCTGAGAATTTTTTACAGAGGTTCAGGATTAATTTCTTCTA
TCTCCATAGATTGGCTTTATCAAAGAATGTATTTCATCATGGATGAACTGGTATGTGTCTGTGATTTAGA
GAACTGCTCAAACATCGAGGAAATTACTCCACCCTCTATTAGTGCACCTCAAAAAATTGTGGCTGATTCA
TACAATGGGTATGTCTTTTACCTCCTGAGAGATGGCATTTATAGAGCAGACCTTCCTGTACCATCTGGCC
GGTGTGCAGAAGCTGTGCGTATTGTGGAGAGTTGCACGTTAAAGGACTTTGCAATCAAGCCACAAGCCAA
GCGAATCATTTACTTCAATGACACTGCCCAAGTCTTCATGTCAACATTTCTGGATGGCTCTGCTTCCCAT
CTCATCCTACCTCGCATCCCCTTTGCTGATGTGAAAAGTTTTGCTTGTGAAAACAATGACTTTCTTGTCA
CAGATGGCAAGGTCATTTTCCAACAGGATGCTTTGTCTTTTAATGAATTCATCGTGGGATGTGACCTGAG
TCACATAGAAGAATTTGGGTTTGGTAACTTGGTCATCTTTGGCTCATCCTCCCAGCTGCACCCTCTGCCA
GGCCGCCCGCAGGAGCTTTCGGTGCTGTTTGGCTCTCACCAGGCTCTTGTTCAATGGAAGCCTCCTGCCC
TTGCCATAGGAGCCAATGTCATCCTGATCAGTGATATTATTGAACTCTTTGAATTAGGCCCTTCTGCCTG
GCAGAACTGGACCTATGAGGTGAAAGTATCCACCCAAGACCCTCCTGAAGTCACTCATATTTTCTTGAAC
ATAAGTGGAACCATGCTGAATGTACCTGAGCTGCAGAGTGCTATGAAATACAAGGTTTCTGTGAGAGCAA
GTTCTCCAAAGAGGCCAGGCCCCTGGTCAGAGCCCTCAGTGGGTACTACCCTGGTGCCAGCTAGTGAACC
ACCATTTATCATGGCTGTGAAAGAAGATGGGCTTTGGAGTAAACCATTAAATAGCTTTGGCCCAGGAGAG
TTCTTATCCTCTGATATAGGAAATGTGTCAGACATGGATTGGTATAACAACAGCCTCTACTACAGTGACA
CGAAAGGCGACGTTTTTGTGTGGCTGCTGAATGGGACGGATATCTCAGAGAATTATCACCTACCCAGCAT
TGCAGGAGCAGGGGCTTTAGCTTTTGAGTGGCTGGGTCACTTTCTCTACTGGGCTGGAAAGACATATGTG
ATACAAAGGCAGTCTGTGTTGACGGGACACACAGACATTGTTACCCACGTGAAGCTATTGGTGAATGACA
TGGTGGTGGATTCAGTTGGTGGATATCTCTACTGGACCACACTCTATTCAGTGGAAAGCACCAGACTAAA
TGGGGAAAGTTCCCTTGTACTACAGACACAGCCTTGGTTTTCTGGGAAAAAGGTAATTGCTCTAACTTTA
GACCTCAGTGATGGGCTCCTGTATTGGTTGGTTCAAGACAGTCAATGTATTCACCTGTACACAGCTGTTC
TTCGGGGACAGAGCACTGGGGATACCACCATCACAGAATTTGCAGCCTGGAGTACTTCTGAAATTTCCCA
GAATGCACTGATGTACTATAGTGGTCGGCTGTTCTGGATCAATGGCTTTAGGATTATCACAACTCAAGAA
ATAGGTCAGAAAACCAGTGTCTCTGTTTTGGAACCAGCCAGATTTAATCAGTTCACAATTATTCAGACAT
CCCTTAAGCCCCTGCCAGGGAACTTTTCCTTTACCCCTAAGGTTATTCCAGATTCTGTTCAAGAGTCTTC
ATTTAGGATTGAAGGAAATGCTTCAAGTTTTCAAATCCTGTGGAATGGTCCCCCTGCGGTAGACTGGGGT
GTAGTTTTCTACAGTGTAGAATTTAGTGCTCATTCTAAGTTCTTGGCTAGTGAACAACACTCTTTACCTG
TATTTACTGTGGAAGGACTGGAACCTTATGCCTTATTTAATCTTTCTGTCACTCCTTATACCTACTGGGG
AAAGGGCCCCAAAACATCTCTGTCACTTCGAGCACCTGAAACAGTTCCATCAGCACCAGAGAACCCCAGA
ATATTTATATTACCAAGTGGAAAATGCTGCAACAAGAATGAAGTTGTGGTGGAATTTAGGTGGAACAAAC
CTAAGCATGAAAATGGGGTGTTAACAAAATTTGAAATTTTCTACAATATATCCAATCAAAGTATTACAAA
CAAAACATGTGAAGACTGGATTGCTGTCAATGTCACTCCCTCAGTGATGTCTTTTCAACTTGAAGGCATG
AGTCCCAGATGCTTTATTGCCTTCCAGGTTAGGGCCTTTACATCTAAGGGGCCAGGACCATATGCTGACG
TTGTAAAGTCTACAACATCAGAAATCAACCCATTTCCTCACCTCATAACTCTTCTTGGTAACAAGATAGT
TTTTTTAGATATGGATCAAAATCAAGTTGTGTGGACGTTTTCAGCAGAAAGAGTTATCAGTGCCGTTTGC
TACACAGCTGATAATGAGATGGGATATTATGCTGAAGGGGACTCACTCTTTCTTCTGCACTTGCACAATC
GCTCTAGCTCTGAGCTTTTCCAAGATTCACTGGTTTTTGATATCACAGTTATTACAATTGACTGGATTTC
AAGGCACCTCTACTTTGCACTGAAAGAATCACAAAATGGAATGCAAGTATTTGATGTTGATCTTGAACAC
AAGGTGAAATATCCCAGAGAGGTGAAGATTCACAATAGGAATTCAACAATAATTTCTTTTTCTGTATATC
CTCTTTTAAGTCGCTTGTATTGGACAGAAGTTTCCAATTTTGGCTACCAGATGTTCTACTACAGTATTAT
CAGTCACACCTTGCACCGAATTCTGCAACCCACAGCTACAAACCAACAAAACAAAAGGAATCAATGTTCT
TGTAATGTGACTGAATTTGAGTTAAGTGGAGCAATGGCTATTGATACCTCTAACCTAGAGAAACCATTGA
TATACTTTGCCAAAGCACAAGAGATCTGGGCAATGGATCTGGAAGGCTGTCAGTGTTGGAGAGTTATCAC
AGTACCTGCTATGCTCGCAGGAAAAACCCTTGTTAGCTTAACTGTGGATGGAGATCTTATATACTGGATC
ATCACAGCAAAGGACAGCACACAGATTTATCAGGCAAAGAAAGGAAATGGGGCCATCGTTTCCCAGGTGA
AGGCCCTAAGGAGTAGGCATATCTTGGCTTACAGTTCAGTTATGCAGCCTTTTCCAGATAAAGCGTTTCT
GTCTCTAGCTTCAGACACTGTGGAACCAACTATACTTAATGCCACTAACACTAGCCTCACAATCAGATTA
CCTCTGGCCAAGACAAACCTCACATGGTATGGCATCACCAGCCCTACTCCAACATACCTGGTTTATTATG
CAGAAGTTAATGACAGGAAAAACAGCTCTGACTTGAAATATAGAATTCTGGAATTTCAGGACAGTATAGC
TCTTATTGAAGATTTACAACCATTTTCAACATACATGATACAGATAGCTGTAAAAAATTATTATTCAGAT
CCTTTGGAACATTTACCACCAGGAAAAGAGATTTGGGGAAAAACTAAAAATGGAGTACCAGAGGCAGTGC
AGCTCATTAATACAACTGTGCGGTCAGACACCAGCCTCATTATATCTTGGAGAGAATCTCACAAGCCAAA
TGGACCTAAAGAATCAGTCCGTTATCAGTTGGCAATCTCACACCTGGCCCTAATTCCTGAAACTCCTCTA
AGACAAAGTGAATTTCCAAATGGAAGGCTCACTCTCCTTGTTACTAGACTGTCTGGTGGAAATATTTATG
TGTTAAAGGTTCTTGCCTGCCACTCTGAGGAAATGTGGTGTACAGAGAGTCATCCTGTCACTGTGGAAAT
GTTTAACACACCAGAGAAACCTTATTCCTTGGTTCCAGAGAACACTAGTTTGCAATTTAATTGGAAGGCT
CCATTGAATGTTAACCTCATCAGATTTTGGGTTGAGCTACAGAAGTGGAAATACAATGAGTTTTACCATG
TTAAAACTTCATGCAGCCAAGGTCCTGCTTATGTCTGTAATATCACAAATCTACAACCTTATACTTCATA
TAATGTCAGAGTAGTGGTGGTTTATAAGACGGGAGAAAATAGCACCTCACTTCCAGAAAGCTTTAAGACA
AAAGCTGGAGTCCCAAATAAACCAGGCATTCCCAAATTACTAGAAGGGAGTAAAAATTCAATACAGTGGG
AGAAAGCTGAAGATAATGGATGTAGAATTACATACTATATCCTTGAGATAAGAAAGAGCACTTCAAATAA
TTTACAGAACCAGAATTTAAGGTGGAAGATGACATTTAATGGATCCTGCAGTAGTGTTTGCACATGGAAG
TCCAAAAACCTGAAAGGAATATTTCAGTTCAGAGTAGTAGCTGCAAATAATCTAGGGTTTGGTGAATATA
GTGGAATCAGTGAGAATATTATATTAGTTGGAGATGATTTTTGGATACCAGAAACAAGTTTCATACTTAC
TATTATAGTTGGAATATTTCTGGTTGTTACAATCCCACTGACCTTTGTCTGGCATAGAAGATTAAAGAAT
CAAAAAAGTGCCAAGGAAGGGGTGACAGTGCTTATAAACGAAGACAAAGAGTTGGCTGAGCTGCGAGGTC
TGGCAGCCGGAGTAGGCCTGGCTAATGCCTGCTATGCAATACATACTCTTCCAACCCAAGAGGAGATTGA
AAATCTTCCTGCCTTCCCTCGGGAAAAACTGACTCTGCGTCTCTTGCTGGGAAGTGGAGCCTTTGGAGAA
GTGTATGAAGGAACAGCAGTGGACATCTTAGGAGTTGGAAGTGGAGAAATCAAAGTAGCAGTGAAGACTT
TGAAGAAGGGTTCCACAGACCAGGAGAAGATTGAATTCCTGAAGGAGGCACATCTGATGAGCAAATTTAA
TCATCCCAACATTCTGAAGCAGCTTGGAGTTTGTCTGCTGAATGAACCCCAATACATTATCCTGGAACTG
ATGGAGGGAGGAGACCTTCTTACTTATTTGCGTAAAGCCCGGATGGCAACGTTTTATGGTCCTTTACTCA
CCTTGGTTGACCTTGTAGACCTGTGTGTAGATATTTCAAAAGGCTGTGTCTACTTGGAACGGATGCATTT
CATTCACAGGGATCTGGCAGCTAGAAATTGCCTTGTTTCCGTGAAAGACTATACCAGTCCACGGATAGTG
AAGATTGGAGACTTTGGACTCGCCAGAGACATCTATAAAAATGATTACTATAGAAAGAGAGGGGAAGGCC
TGCTCCCAGTTCGGTGGATGGCTCCAGAAAGTTTGATGGATGGAATCTTCACTACTCAATCTGATGTATG
GTCTTTTGGAATTCTGATTTGGGAGATTTTAACTCTTGGTCATCAGCCTTATCCAGCTCATTCCAACCTT
GATGTGTTAAACTATGTGCAAACAGGAGGGAGACTGGAGCCACCAAGAAATTGTCCTGATGATCTGTGGA
ATTTAATGACCCAGTGCTGGGCTCAAGAACCCGACCAAAGACCTACTTTTCATAGAATTCAGGACCAACT
TCAGTTATTCAGAAATTTTTTCTTAAATAGCATTTATAAGTCCAGAGATGAAGCAAACAACAGTGGAGTC
ATAAATGAAAGCTTTGAAGGTGAAGATGGCGATGTGATTTGTTTGAATTCAGATGACATTATGCCAGTTG
CTTTAATGGAAACGAAGAACCGAGAAGGGTTAAACTATATGGTACTTGCTACAGAATGTGGCCAAGGTGA
AGAAAAGTCTGAGGGTCCTCTAGGCTCCCAGGAATCTGAATCTTGTGGTCTGAGGAAAGAAGAGAAGGAA
CCACATGCAGACAAAGATTTCTGCCAAGAAAAACAAGTGGCTTACTGCCCTTCTGGCAAGCCTGAAGGCC
TGAACTATGCCTGTCTCACTCACAGTGGATATGGAGATGGGTCTGATTAATAGCGTTGTTTGGGAAATAG
AGAGTTGAGATAAACACTCTCATTCAGTAGTTACTGAAAGAAAACTCTGCTAGAATGATAAATGTCATGG
TGGTCTATAACTCCAAATAAACAATGCAACGTTCCTGATTTCTAATCTTGGTTCTGAGAGCCATTTGGTT
TCAGTTGTAGCAATCCCCATACCAGCTGCCTGACTTTCAGTAGAATTATGAGATGAACACTAAGCATGTG
GAAAGCTTAGGAAGACTCAGAAGTCTGGAAGGGAAACACTGCTCTCCCTTCTCCCTTGAGGTGCTTTAGG
CTCTTACCCACCTTTCAGTTTGGGCTGTAATAAAAATATCTTGGCCACATGTTTAGAGACAGAATAGGTG
TGTTCAGCGATATAAAGAAGAGGCTAAGGAGTAGGCTCAGGGGGGTCAACTGAACTACAGATAATCTCAA
ATGGGACCAAGGAAATGAGAAATAATTTCACACATACAGAAGAAACCAGCACCTGTGACTTGAGAAATCA
CTTGGAAAGCTGTTACTGCAATGATATATATATTATCTTTTTTTAATTTTTTTTTTTTTTTTTTGAGACG
AAGTCTTGCTCTGTTGCCCAGGCTGGAGTTCAATGGCACGATCTCGGCACTGCAAACTCCACCTCCTGGG
TTCAAGCAATTCTCGTGCCTCAGCCTCCTAAGTAGCTGGGATTACAGGCGTGTGCCACCACGCCCGGCTA
ATTTTTGTATTTTTAGTAGAGATGAGGTTTCACCATATTGGCCAGGCTGGTCTAGAACTCCTGACCTCGG
GATCCACCTGCCTTGGCCTCCCAAAGTGCTGGATTACAGGTGTGAGCCACCATGCCTAGCCGATATATAT
TGTCTTTAATCACTACTGTAAAATATTTTGTAGTTTTGAGGCTTACAACAGTAGATTCAGTCATGTTGAA
AATAAGACTGTGAAGATCTTTTAAGTCCTGAAGTTTTGCATTCTGTAATCTTCAGTTGTATAAAATCACT
CTGACTTGTGTGCTATTATGGAAATTAACTAGTATAAAGATTGCTATTTGCCATATCTATTTTATGTATA
AAATACTTAAGATTACATTTTGTATCAAATTATGCTTAAAATTAAATATAAATGATTATACAATGTTAA
NM_000455.5 Homo sapiens serine/threonine kinase 11 (STK11), transcript
variant 1, mRNA
(SEQ ID NO: 17)
GAGGTAAACAAGATGGCGGCGGCGTGTCGGGCGCGGAAGGGGGAGGCGGCCCGGGGCGCCCGCGAGTGAG
GCGCGGGGCGGCGAAGGGAGCGCGGGTGGCGGCACTTGCTGCCGCGGCCTTGGATGGGCTGGGCCCCCCT
CGCCGCTCCGCCTCCTCCACACGCGCGGCGGCCGCGGCGAGGGGGACGCGCCGCCCGGGGCCCGGCACCT
TCGGGAACCCCCCGGCCCGGAGCCTGCGGCCTGCGCCGCCTCGGCCGCCGGGAGCCCCGTGGAGCCCCCG
CCGCCGCGCCGCCCCGCGGACCGGACGCTGAGGGCACTCGGGGCGGGGCGCGCGCTCGGGCAGACGTTTG
CGGGGAGGGGGGCGCCTGCCGGGCCCCGGCGACCACCTTGGGGGTCGCGGGCCGGCTCGGGGGGCGCCCA
GTGCGGGCCCTCGCGGGCGCCGGGCAGCGACCAGCCCTGAGCGGAGCTGTTGGCCGCGGCGGGAGGCCTC
CCGGACGCCCCCAGCCCCCCGAACGCTCGCCCGGGCCGGCGGGAGTCGGCGCCCCCCGGGAGGTCCGCTC
GGTCGTCCGCGGCGGAGCGTTTGCTCCTGGGACAGGCGGTGGGACCGGGGCGTCGCCGGAGACGCCCCCA
GCGAAGTTGGGCTCTCCAGGTGTGGGGGTCCCGGGGGGTAGCGACGTCGCGGACCCGGCCTGTGGGATGG
GCGGCCCGGAGAAGACTGCGCTCGGCCGTGTTCATACTTGTCCGTGGGCCTGAGGTCCCCGGAGGATGAC
CTAGCACTGAAAAGCCCCGGCCGGCCTCCCCAGGGTCCCCGAGGACGAAGTTGACCCTGACCGGGCCGTC
TCCCAGTTCTGAGGCCCGGGTCCCACTGGAACTCGCGTCTGAGCCGCCGTCCCGGACCCCCGGTGCCCGC
CGGTCCGCAGACCCTGCACCGGGCTTGGACTCGCAGCCGGGACTGACGTGTAGAACAATCGTTTCTGTTG
GAAGAAGGGTTTTTCCCTTCCTTTTGGGGTTTTTGTTGCCTTTTTTTTTTCTTTTTTCTTTGTAAAATTT
TGGAGAAGGGAAGTCGGAACACAAGGAAGGACCGCTCACCCGCGGACTCAGGGCTGGCGGCGGGACTCCA
GGACCCTGGGTCCAGCATGGAGGTGGTGGACCCGCAGCAGCTGGGCATGTTCACGGAGGGCGAGCTGATG
TCGGTGGGTATGGACACGTTCATCCACCGCATCGACTCCACCGAGGTCATCTACCAGCCGCGCCGCAAGC
GGGCCAAGCTCATCGGCAAGTACCTGATGGGGGACCTGCTGGGGGAAGGCTCTTACGGCAAGGTGAAGGA
GGTGCTGGACTCGGAGACGCTGTGCAGGAGGGCCGTCAAGATCCTCAAGAAGAAGAAGTTGCGAAGGATC
CCCAACGGGGAGGCCAACGTGAAGAAGGAAATTCAACTACTGAGGAGGTTACGGCACAAAAATGTCATCC
AGCTGGTGGATGTGTTATACAACGAAGAGAAGCAGAAAATGTATATGGTGATGGAGTACTGCGTGTGTGG
CATGCAGGAAATGCTGGACAGCGTGCCGGAGAAGCGTTTCCCAGTGTGCCAGGCCCACGGGTACTTCTGT
CAGCTGATTGACGGCCTGGAGTACCTGCATAGCCAGGGCATTGTGCACAAGGACATCAAGCCGGGGAACC
TGCTGCTCACCACCGGTGGCACCCTCAAAATCTCCGACCTGGGCGTGGCCGAGGCACTGCACCCGTTCGC
GGCGGACGACACCTGCCGGACCAGCCAGGGCTCCCCGGCTTTCCAGCCGCCCGAGATTGCCAACGGCCTG
GACACCTTCTCCGGCTTCAAGGTGGACATCTGGTCGGCTGGGGTCACCCTCTACAACATCACCACGGGTC
TGTACCCCTTCGAAGGGGACAACATCTACAAGTTGTTTGAGAACATCGGGAAGGGGAGCTACGCCATCCC
GGGCGACTGTGGCCCCCCGCTCTCTGACCTGCTGAAAGGGATGCTTGAGTACGAACCGGCCAAGAGGTTC
TCCATCCGGCAGATCCGGCAGCACAGCTGGTTCCGGAAGAAACATCCTCCGGCTGAAGCACCAGTGCCCA
TCCCACCGAGCCCAGACACCAAGGACCGGTGGCGCAGCATGACTGTGGTGCCGTACTTGGAGGACCTGCA
CGGCGCGGACGAGGACGAGGACCTCTTCGACATCGAGGATGACATCATCTACACTCAGGACTTCACGGTG
CCCGGACAGGTCCCAGAAGAGGAGGCCAGTCACAATGGACAGCGCCGGGGCCTCCCCAAGGCCGTGTGTA
TGAACGGCACAGAGGCGGCGCAGCTGAGCACCAAATCCAGGGCGGAGGGCCGGGCCCCCAACCCTGCCCG
CAAGGCCTGCTCCGCCAGCAGCAAGATCCGCCGGCTGTCGGCCTGCAAGCAGCAGTGAGGCTGGCCGCCT
GCAGCCCGTGTCCAGGAGCCCCGCCAGGTGCCCGCGCCAGGCCCTCAGTCTTCCTGCCGGTTCCGCCCGC
CCTCCCGGAGAGGTGGCCGCCATGCTTCTGTGCCGACCACGCCCCAGGACCTCCGGAGCGCCCTGCAGGG
CCGGGCAGGGGGACAGCAGGGACCGGGCGCAGCCCTCCCCCCTCGGCCGCCCGGCAGTGCACGCGGCTTG
TTGACTTCGCAGCCCCGGGCGGAGCCTTCCCGGGCGGGCGTGGGAGGAGGGAGGCGGCCTCCATGCACTT
TATGTGGAGACTACTGGCCCCGCCCGTGGCCTCGTGCTCCGCAGGGCGCCCAGCGCCGTCCGGCGGCCCC
GCCGCAGACCAGCTGGCGGGTGTGGAGACCAGGCTCCTGACCCCGCCATGCATGCAGCGCCACCTGGAAG
CCGCGCGGCCGCTTTGGTTTTTTGTTTGGTTGGTTCCATTTTCTTTTTTTCTTTTTTTTTTTAAGAAAAA
ATAAAAGGTGGATTTGAGCTGTGGCTGTGAGGGGTGTTTGGGAGCTGCTGGGTGGCAGGGGGGCTGTGGG
GTCGGGCTCACGTCGCGGCCGCCTTTGCGCTCTCGGGTCACCCTGCTTTGGCGGCCCGGCCGGAGGGCAG
GACCCTCACCTCTCCCCCAAGGCCACTGCGCTCTTGGGACCCCAGAGAAAACCCGGAGCAAGCAGGAGTG
TGCGGTCAATATTTATATCATCCAGAAAAGAAAAACACGAGAAACGCCATCGCGGGATGGTGCAGACGCG
GCGGGGACTCGGAGGGTGCCGTGCGGGCGAGGCCGCCCAAATTTGGCAATAAATAAAGCTTGGGAAGCTT
GGA
NM_000546.6 Homo sapiens tumor protein p53 (TP53), transcript variant 1,
mRNA
(SEQ ID NO: 18)
CTCAAAAGTCTAGAGCCACCGTCCAGGGAGCAGGTAGCTGCTGGGCTCCGGGGACACTTTGCGTTCGGGC
TGGGAGCGTGCTTTCCACGACGGTGACACGCTTCCCTGGATTGGCAGCCAGACTGCCTTCCGGGTCACTG
CCATGGAGGAGCCGCAGTCAGATCCTAGCGTCGAGCCCCCTCTGAGTCAGGAAACATTTTCAGACCTATG
GAAACTACTTCCTGAAAACAACGTTCTGTCCCCCTTGCCGTCCCAAGCAATGGATGATTTGATGCTGTCC
CCGGACGATATTGAACAATGGTTCACTGAAGACCCAGGTCCAGATGAAGCTCCCAGAATGCCAGAGGCTG
CTCCCCCCGTGGCCCCTGCACCAGCAGCTCCTACACCGGCGGCCCCTGCACCAGCCCCCTCCTGGCCCCT
GTCATCTTCTGTCCCTTCCCAGAAAACCTACCAGGGCAGCTACGGTTTCCGTCTGGGCTTCTTGCATTCT
GGGACAGCCAAGTCTGTGACTTGCACGTACTCCCCTGCCCTCAACAAGATGTTTTGCCAACTGGCCAAGA
CCTGCCCTGTGCAGCTGTGGGTTGATTCCACACCCCCGCCCGGCACCCGCGTCCGCGCCATGGCCATCTA
CAAGCAGTCACAGCACATGACGGAGGTTGTGAGGCGCTGCCCCCACCATGAGCGCTGCTCAGATAGCGAT
GGTCTGGCCCCTCCTCAGCATCTTATCCGAGTGGAAGGAAATTTGCGTGTGGAGTATTTGGATGACAGAA
ACACTTTTCGACATAGTGTGGTGGTGCCCTATGAGCCGCCTGAGGTTGGCTCTGACTGTACCACCATCCA
CTACAACTACATGTGTAACAGTTCCTGCATGGGCGGCATGAACCGGAGGCCCATCCTCACCATCATCACA
CTGGAAGACTCCAGTGGTAATCTACTGGGACGGAACAGCTTTGAGGTGCGTGTTTGTGCCTGTCCTGGGA
GAGACCGGCGCACAGAGGAAGAGAATCTCCGCAAGAAAGGGGAGCCTCACCACGAGCTGCCCCCAGGGAG
CACTAAGCGAGCACTGCCCAACAACACCAGCTCCTCTCCCCAGCCAAAGAAGAAACCACTGGATGGAGAA
TATTTCACCCTTCAGATCCGTGGGCGTGAGCGCTTCGAGATGTTCCGAGAGCTGAATGAGGCCTTGGAAC
TCAAGGATGCCCAGGCTGGGAAGGAGCCAGGGGGGAGCAGGGCTCACTCCAGCCACCTGAAGTCCAAAAA
GGGTCAGTCTACCTCCCGCCATAAAAAACTCATGTTCAAGACAGAAGGGCCTGACTCAGACTGACATTCT
CCACTTCTTGTTCCCCACTGACAGCCTCCCACCCCCATCTCTCCCTCCCCTGCCATTTTGGGTTTTGGGT
CTTTGAACCCTTGCTTGCAATAGGTGTGCGTCAGAAGCACCCAGGACTTCCATTTGCTTTGTCCCGGGGC
TCCACTGAACAAGTTGGCCTGCACTGGTGTTTTGTTGTGGGGAGGAGGATGGGGAGTAGGACATACCAGC
TTAGATTTTAAGGTTTTTACTGTGAGGGATGTTTGGGAGATGTAAGAAATGTTCTTGCAGTTAAGGGTTA
GTTTACAATCAGCCACATTCTAGGTAGGGGCCCACTTCACCGTACTAACCAGGGAAGCTGTCCCTCACTG
TTGAATTTTCTCTAACTTCAAGGCCCATATCTGTGAAATGCTGGCATTTGCACCTACCTCACAGAGTGCA
TTGTGAGGGTTAATGAAATAATGTACATCTGGCCTTGAAACCACCTTTTATTACATGGGGTCTAGAACTT
GACCCCCTTGAGGGTGCTTGTTCCCTCTCCCTGTTGGTCGGTGGGTTGGTAGTTTCTACAGTTGGGCAGC
TGGTTAGGTAGAGGGAGTTGTCAAGTCTCTGCTGGCCCAGCCAAACCCTGTCTGACAACCTCTTGGTGAA
CCTTAGTACCTAAAAGGAAATCTCACCCCATCCCACACCCTGGAGGATTTCATCTCTTGTATATGATGAT
CTGGATCCACCAAGACTTGTTTTATGCTCAGGGTCAATTTCTTTTTTCTTTTTTTTTTTTTTTTTTCTTT
TTCTTTGAGACTGGGTCTCGCTTTGTTGCCCAGGCTGGAGTGGAGTGGCGTGATCTTGGCTTACTGCAGC
CTTTGCCTCCCCGGCTCGAGCAGTCCTGCCTCAGCCTCCGGAGTAGCTGGGACCACAGGTTCATGCCACC
ATGGCCAGCCAACTTTTGCATGTTTTGTAGAGATGGGGTCTCACAGTGTTGCCCAGGCTGGTCTCAAACT
CCTGGGCTCAGGCGATCCACCTGTCTCAGCCTCCCAGAGTGCTGGGATTACAATTGTGAGCCACCACGTC
CAGCTGGAAGGGTCAACATCTTTTACATTCTGCAAGCACATCTGCATTTTCACCCCACCCTTCCCCTCCT
TCTCCCTTTTTATATCCCATTTTTATATCGATCTCTTATTTTACAATAAAACTTTGCTGCCA
NM_002529.4 Homo sapiens neurotrophic receptor tyrosine kinase 1 (NTRK1),
transcript variant 2, mRNA
(SEQ ID NO: 19)
GGAGGCCTGGCAGCTGCAGCTGGGAGCGCACAGACGGCTGCCCCGCCTGAGCGAGGCGGGCGCCGCCGCG
ATGCTGCGAGGCGGACGGCGCGGGCAGCTTGGCTGGCACAGCTGGGCTGCGGGGCCGGGCAGCCTGCTGG
CTTGGCTGATACTGGCATCTGCGGGCGCCGCACCCTGCCCCGATGCCTGCTGCCCCCACGGCTCCTCGGG
ACTGCGATGCACCCGGGATGGGGCCCTGGATAGCCTCCACCACCTGCCCGGCGCAGAGAACCTGACTGAG
CTCTACATCGAGAACCAGCAGCATCTGCAGCATCTGGAGCTCCGTGATCTGAGGGGCCTGGGGGAGCTGA
GAAACCTCACCATCGTGAAGAGTGGTCTCCGTTTCGTGGCGCCAGATGCCTTCCATTTCACTCCTCGGCT
CAGTCGCCTGAATCTCTCCTTCAACGCTCTGGAGTCTCTCTCCTGGAAAACTGTGCAGGGCCTCTCCTTA
CAGGAACTGGTCCTGTCGGGGAACCCTCTGCACTGTTCTTGTGCCCTGCGCTGGCTACAGCGCTGGGAGG
AGGAGGGACTGGGCGGAGTGCCTGAACAGAAGCTGCAGTGTCATGGGCAAGGGCCCCTGGCCCACATGCC
CAATGCCAGCTGTGGTGTGCCCACGCTGAAGGTCCAGGTGCCCAATGCCTCGGTGGATGTGGGGGACGAC
GTGCTGCTGCGGTGCCAGGTGGAGGGGCGGGGCCTGGAGCAGGCCGGCTGGATCCTCACAGAGCTGGAGC
AGTCAGCCACGGTGATGAAATCTGGGGGTCTGCCATCCCTGGGGCTGACCCTGGCCAATGTCACCAGTGA
CCTCAACAGGAAGAACGTGACGTGCTGGGCAGAGAACGATGTGGGCCGGGCAGAGGTCTCTGTTCAGGTC
AACGTCTCCTTCCCGGCCAGTGTGCAGCTGCACACGGCGGTGGAGATGCACCACTGGTGCATCCCCTTCT
CTGTGGATGGGCAGCCGGCACCGTCTCTGCGCTGGCTCTTCAATGGCTCCGTGCTCAATGAGACCAGCTT
CATCTTCACTGAGTTCCTGGAGCCGGCAGCCAATGAGACCGTGCGGCACGGGTGTCTGCGCCTCAACCAG
CCCACCCACGTCAACAACGGCAACTACACGCTGCTGGCTGCCAACCCCTTCGGCCAGGCCTCCGCCTCCA
TCATGGCTGCCTTCATGGACAACCCTTTCGAGTTCAACCCCGAGGACCCCATCCCTGTCTCCTTCTCGCC
GGTGGACACTAACAGCACATCTGGAGACCCGGTGGAGAAGAAGGACGAAACACCTTTTGGGGTCTCGGTG
GCTGTGGGCCTGGCCGTCTTTGCCTGCCTCTTCCTTTCTACGCTGCTCCTTGTGCTCAACAAATGTGGAC
GGAGAAACAAGTTTGGGATCAACCGCCCGGCTGTGCTGGCTCCAGAGGATGGGCTGGCCATGTCCCTGCA
TTTCATGACATTGGGTGGCAGCTCCCTGTCCCCCACCGAGGGCAAAGGCTCTGGGCTCCAAGGCCACATC
ATCGAGAACCCACAATACTTCAGTGATGCCTGTGTTCACCACATCAAGCGCCGGGACATCGTGCTCAAGT
GGGAGCTGGGGGAGGGCGCCTTTGGGAAGGTCTTCCTTGCTGAGTGCCACAACCTCCTGCCTGAGCAGGA
CAAGATGCTGGTGGCTGTCAAGGCACTGAAGGAGGCGTCCGAGAGTGCTCGGCAGGACTTCCAGCGTGAG
GCTGAGCTGCTCACCATGCTGCAGCACCAGCACATCGTGCGCTTCTTCGGCGTCTGCACCGAGGGCCGCC
CCCTGCTCATGGTCTTTGAGTATATGCGGCACGGGGACCTCAACCGCTTCCTCCGATCCCATGGACCTGA
TGCCAAGCTGCTGGCTGGTGGGGAGGATGTGGCTCCAGGCCCCCTGGGTCTGGGGCAGCTGCTGGCCGTG
GCTAGCCAGGTCGCTGCGGGGATGGTGTACCTGGCGGGTCTGCATTTTGTGCACCGGGACCTGGCCACAC
GCAACTGTCTAGTGGGCCAGGGACTGGTGGTCAAGATTGGTGATTTTGGCATGAGCAGGGATATCTACAG
CACCGACTATTACCGTGTGGGAGGCCGCACCATGCTGCCCATTCGCTGGATGCCGCCCGAGAGCATCCTG
TACCGTAAGTTCACCACCGAGAGCGACGTGTGGAGCTTCGGCGTGGTGCTCTGGGAGATCTTCACCTACG
GCAAGCAGCCCTGGTACCAGCTCTCCAACACGGAGGCAATCGACTGCATCACGCAGGGACGTGAGTTGGA
GCGGCCACGTGCCTGCCCACCAGAGGTCTACGCCATCATGCGGGGCTGCTGGCAGCGGGAGCCCCAGCAA
CGCCACAGCATCAAGGATGTGCACGCCCGGCTGCAAGCCCTGGCCCAGGCACCTCCTGTCTACCTGGATG
TCCTGGGCTAGGGGGCCGGCCCAGGGGCTGGGAGTGGTTAGCCGGAATACTGGGGCCTGCCCTCAGCATC
CCCCATAGCTCCCAGCAGCCCCAGGGTGATCTCAAAGTATCTAATTCACCCTCAGCATGTGGGAAGGGAC
AGGTGGGGGCTGGGAGTAGAGGATGTTCCTGCTTCTCTAGGCAAGGTCCCGTCATAGCAATTATATTTAT
TATCCCTTG
NM_023110.3 Homo sapiens fibroblast growth factor receptor 1 (FGFR1),
transcript variant 1, mRNA
(SEQ ID NO: 20)
GCATAGCGCTCGGAGCGCTCTTGCGGCCACAGGCGCGGCGTCCTCGGCGGCGGGCGGCAGCTAGCGGGAG
CCGGGACGCCGGTGCAGCCGCAGCGCGCGGAGGAACCCGGGTGTGCCGGGAGCTGGGCGGCCACGTCCGG
ACGGGACCGAGACCCCTCGTAGCGCATTGCGGCGACCTCGCCTTCCCCGGCCGCGAGCGCGCCGCTGCTT
GAAAAGCCGCGGAACCCAAGGACTTTTCTCCGGTCCGAGCTCGGGGCGCCCCGCAGGGCGCACGGTACCC
GTGCTGCAGTCGGGCACGCCGCGGCGCCGGGGCCTCCGCAGGGCGATGGAGCCCGGTCTGCAAGGAAAGT
GAGGCGCCGCCGCTGCGTTCTGGAGGAGGGGGGCACAAGGTCTGGAGACCCCGGGTGGCGGACGGGAGCC
CTCCCCCCGCCCCGCCTCCGGGGCACCAGCTCCGGCTCCATTGTTCCCGCCCGGGCTGGAGGCGCCGAGC
ACCGAGCGCCGCCGGGAGTCGAGCGCCGGCCGCGGAGCTCTTGCGACCCCGCCAGGACCCGAACAGAGCC
CGGGGGCGGCGGGCCGGAGCCGGGGACGCGGGCACACGCCCGCTCGCACAAGCCACGGCGGACTCTCCCG
AGGCGGAACCTCCACGCCGAGCGAGGGTCAGTTTGAAAAGGAGGATCGAGCTCACTGTGGAGTATCCATG
GAGATGTGGAGCCTTGTCACCAACCTCTAACTGCAGAACTGGGATGTGGAGCTGGAAGTGCCTCCTCTTC
TGGGCTGTGCTGGTCACAGCCACACTCTGCACCGCTAGGCCGTCCCCGACCTTGCCTGAACAAGCCCAGC
CCTGGGGAGCCCCTGTGGAAGTGGAGTCCTTCCTGGTCCACCCCGGTGACCTGCTGCAGCTTCGCTGTCG
GCTGCGGGACGATGTGCAGAGCATCAACTGGCTGCGGGACGGGGTGCAGCTGGCGGAAAGCAACCGCACC
CGCATCACAGGGGAGGAGGTGGAGGTGCAGGACTCCGTGCCCGCAGACTCCGGCCTCTATGCTTGCGTAA
CCAGCAGCCCCTCGGGCAGTGACACCACCTACTTCTCCGTCAATGTTTCAGATGCTCTCCCCTCCTCGGA
GGATGATGATGATGATGATGACTCCTCTTCAGAGGAGAAAGAAACAGATAACACCAAACCAAACCGTATG
CCCGTAGCTCCATATTGGACATCCCCAGAAAAGATGGAAAAGAAATTGCATGCAGTGCCGGCTGCCAAGA
CAGTGAAGTTCAAATGCCCTTCCAGTGGGACCCCAAACCCCACACTGCGCTGGTTGAAAAATGGCAAAGA
ATTCAAACCTGACCACAGAATTGGAGGCTACAAGGTCCGTTATGCCACCTGGAGCATCATAATGGACTCT
GTGGTGCCCTCTGACAAGGGCAACTACACCTGCATTGTGGAGAATGAGTACGGCAGCATCAACCACACAT
ACCAGCTGGATGTCGTGGAGCGGTCCCCTCACCGGCCCATCCTGCAAGCAGGGTTGCCCGCCAACAAAAC
AGTGGCCCTGGGTAGCAACGTGGAGTTCATGTGTAAGGTGTACAGTGACCCGCAGCCGCACATCCAGTGG
CTAAAGCACATCGAGGTGAATGGGAGCAAGATTGGCCCAGACAACCTGCCTTATGTCCAGATCTTGAAGA
CTGCTGGAGTTAATACCACCGACAAAGAGATGGAGGTGCTTCACTTAAGAAATGTCTCCTTTGAGGACGC
AGGGGAGTATACGTGCTTGGCGGGTAACTCTATCGGACTCTCCCATCACTCTGCATGGTTGACCGTTCTG
GAAGCCCTGGAAGAGAGGCCGGCAGTGATGACCTCGCCCCTGTACCTGGAGATCATCATCTATTGCACAG
GGGCCTTCCTCATCTCCTGCATGGTGGGGTCGGTCATCGTCTACAAGATGAAGAGTGGTACCAAGAAGAG
TGACTTCCACAGCCAGATGGCTGTGCACAAGCTGGCCAAGAGCATCCCTCTGCGCAGACAGGTAACAGTG
TCTGCTGACTCCAGTGCATCCATGAACTCTGGGGTTCTTCTGGTTCGGCCATCACGGCTCTCCTCCAGTG
GGACTCCCATGCTAGCAGGGGTCTCTGAGTATGAGCTTCCCGAAGACCCTCGCTGGGAGCTGCCTCGGGA
CAGACTGGTCTTAGGCAAACCCCTGGGAGAGGGCTGCTTTGGGCAGGTGGTGTTGGCAGAGGCTATCGGG
CTGGACAAGGACAAACCCAACCGTGTGACCAAAGTGGCTGTGAAGATGTTGAAGTCGGACGCAACAGAGA
AAGACTTGTCAGACCTGATCTCAGAAATGGAGATGATGAAGATGATCGGGAAGCATAAGAATATCATCAA
CCTGCTGGGGGCCTGCACGCAGGATGGTCCCTTGTATGTCATCGTGGAGTATGCCTCCAAGGGCAACCTG
CGGGAGTACCTGCAGGCCCGGAGGCCCCCAGGGCTGGAATACTGCTACAACCCCAGCCACAACCCAGAGG
AGCAGCTCTCCTCCAAGGACCTGGTGTCCTGCGCCTACCAGGTGGCCCGAGGCATGGAGTATCTGGCCTC
CAAGAAGTGCATACACCGAGACCTGGCAGCCAGGAATGTCCTGGTGACAGAGGACAATGTGATGAAGATA
GCAGACTTTGGCCTCGCACGGGACATTCACCACATCGACTACTATAAAAAGACAACCAACGGCCGACTGC
CTGTGAAGTGGATGGCACCCGAGGCATTATTTGACCGGATCTACACCCACCAGAGTGATGTGTGGTCTTT
CGGGGTGCTCCTGTGGGAGATCTTCACTCTGGGCGGCTCCCCATACCCCGGTGTGCCTGTGGAGGAACTT
TTCAAGCTGCTGAAGGAGGGTCACCGCATGGACAAGCCCAGTAACTGCACCAACGAGCTGTACATGATGA
TGCGGGACTGCTGGCATGCAGTGCCCTCACAGAGACCCACCTTCAAGCAGCTGGTGGAAGACCTGGACCG
CATCGTGGCCTTGACCTCCAACCAGGAGTACCTGGACCTGTCCATGCCCCTGGACCAGTACTCCCCCAGC
TTTCCCGACACCCGGAGCTCTACGTGCTCCTCAGGGGAGGATTCCGTCTTCTCTCATGAGCCGCTGCCCG
AGGAGCCCTGCCTGCCCCGACACCCAGCCCAGCTTGCCAATGGCGGACTCAAACGCCGCTGACTGCCACC
CACACGCCCTCCCCAGACTCCACCGTCAGCTGTAACCCTCACCCACAGCCCCTGCTGGGCCCACCACCTG
TCCGTCCCTGTCCCCTTTCCTGCTGGCAGGAGCCGGCTGCCTACCAGGGGCCTTCCTGTGTGGCCTGCCT
TCACCCCACTCAGCTCACCTCTCCCTCCACCTCCTCTCCACCTGCTGGTGAGAGGTGCAAAGAGGCAGAT
CTTTGCTGCCAGCCACTTCATCCCCTCCCAGATGTTGGACCAACACCCCTCCCTGCCACCAGGCACTGCC
TGGAGGGCAGGGAGTGGGAGCCAATGAACAGGCATGCAAGTGAGAGCTTCCTGAGCTTTCTCCTGTCGGT
TTGGTCTGTTTTGCCTTCACCCATAAGCCCCTCGCACTCTGGTGGCAGGTGCCTTGTCCTCAGGGCTACA
GCAGTAGGGAGGTCAGTGCTTCGTGCCTCGATTGAAGGTGACCTCTGCCCCAGATAGGTGGTGCCAGTGG
CTTATTAATTCCGATACTAGTTTGCTTTGCTGACCAAATGCCTGGTACCAGAGGATGGTGAGGCGAAGGC
CAGGTTGGGGGCAGTGTTGTGGCCCTGGGGCCCAGCCCCAAACTGGGGGCTCTGTATATAGCTATGAAGA
AAACACAAAGTGTATAAATCTGAGTATATATTTACATGTCTTTTTAAAAGGGTCGTTACCAGAGATTTAC
CCATCGGGTAAGATGCTCCTGGTGGCTGGGAGGCATCAGTTGCTATATATTAAAAACAAAAAAGAAAAAA
AAGGAAAATGTTTTTAAAAAGGTCATATATTTTTTGCTACTTTTGCTGTTTTATTTTTTTAAATTATGTT
CTAAACCTATTTTCAGTTTAGGTCCCTCAATAAAAATTGCTGCTGCTTCATTTATCTATGGGCTGTATGA
AAAGGGTGGGAATGTCCACTGGAAAGAAGGGACACCCACGGGCCCTGGGGCTAGGTCTGTCCCGAGGGCA
CCGCATGCTCCCGGCGCAGGTTCCTTGTAACCTCTTCTTCCTAGGTCCTGCACCCAGACCTCACGACGCA
CCTCCTGCCTCTCCGCTGCTTTTGGAAAGTCAGAAAAAGAAGATGTCTGCTTCGAGGGCAGGAACCCCAT
CCATGCAGTAGAGGCGCTGGGCAGAGAGTCAAGGCCCAGCAGCCATCGACCATGGATGGTTTCCTCCAAG
GAAACCGGTGGGGTTGGGCTGGGGAGGGGGCACCTACCTAGGAATAGCCACGGGGTAGAGCTACAGTGAT
TAAGAGGAAAGCAAGGGCGCGGTTGCTCACGCCTGTAATCCCAGCACTTTGGGACACCGAGGTGGGCAGA
TCACTTCAGGTCAGGAGTTTGAGACCAGCCTGGCCAACTTAGTGAAACCCCATCTCTACTAAAAATGCAA
AAATTATCCAGGCATGGTGGCACACGCCTGTAATCCCAGCTCCACAGGAGGCTGAGGCAGAATCCCTTGA
AGCTGGGAGGCGGAGGTTGCAGTGAGCCGAGATTGCGCCATTGCACTCCAGCCTGGGCAACAGAGAAAAC
AAAAAGGAAAACAAATGATGAAGGTCTGCAGAAACTGAAACCCAGACATGTGTCTGCCCCCTCTATGTGG
GCATGGTTTTGCCAGTGCTTCTAAGTGCAGGAGAACATGTCACCTGAGGCTAGTTTTGCATTCAGGTCCC
TGGCTTCGTTTCTTGTTGGTATGCCTCCCCAGATCGTCCTTCCTGTATCCATGTGACCAGACTGTATTTG
TTGGGACTGTCGCAGATCTTGGCTTCTTACAGTTCTTCCTGTCCAAACTCCATCCTGTCCCTCAGGAACG
GGGGGAAAATTCTCCGAATGTTTTTGGTTTTTTGGCTGCTTGGAATTTACTTCTGCCACCTGCTGGTCAT
CACTGTCCTCACTAAGTGGATTCTGGCTCCCCCGTACCTCATGGCTCAAACTACCACTCCTCAGTCGCTA
TATTAAAGCTTATATTTTGCTGGATTACTGCTAAATACAAAAGAAAGTTCAATATGTTTTCATTTCTGTA
GGGAAAATGGGATTGCTGCTTTAAATTTCTGAGCTAGGGATTTTTTGGCAGCTGCAGTGTTGGCGACTAT
TGTAAAATTCTCTTTGTTTCTCTCTGTAAATAGCACCTGCTAACATTACAATTTGTATTTATGTTTAAAG
AAGGCATCATTTGGTGAACAGAACTAGGAAATGAATTTTTAGCTCTTAAAAGCATTTGCTTTGAGACCGC
ACAGGAGTGTCTTTCCTTGTAAAACAGTGATGATAATTTCTGCCTTGGCCCTACCTTGAAGCAATGTTGT
GTGAAGGGATGAAGAATCTAAAAGTCTTCATAAGTCCTTGGGAGAGGTGCTAGAAAAATATAAGGCACTA
TCATAATTACAGTGATGTCCTTGCTGTTACTACTCAAATCACCCACAAATTTCCCCAAAGACTGCGCTAG
CTGTCAAATAAAAGACAGTGAAATTGA
NM_001354870.1 Homo sapiens MYC proto-oncogene, bHLH transcription
factor (MYC), transcript variant 2, mRNA
(SEQ ID NO: 21)
GGAGTTTATTCATAACGCGCTCTCCAAGTATACGTGGCAATGCGTTGCTGGGTTATTTTAATCATTCTAG
GCATCGTTTTCCTCCTTATGCCTCTATCATTCCTCCCTATCTACACTAACATCCCACGCTCTGAACGCGC
GCCCATTAATACCCTTCTTTCCTCCACTCTCCCTGGGACTCTTGATCAAAGCGCGGCCCTTTCCCCAGCC
TTAGCGAGGCGCCCTGCAGCCTGGTACGCGCGTGGCGTGGCGGTGGGCGCGCAGTGCGTTCTCGGTGTGG
AGGGCAGCTGTTCCGCCTGCGATGATTTATACTCACAGGACAAGGATGCGGTTTGTCAAACAGTACTGCT
ACGGAGGAGCAGCAGAGAAAGGGAGAGGGTTTGAGAGGGAGCAAAAGAAAATGGTAGGCGCGCGTAGTTA
ATTCATGCGGCTCTCTTACTCTGTTTACATCCTAGAGCTAGAGTGCTCGGCTGCCCGGCTGAGTCTCCTC
CCCACCTTCCCCACCCTCCCCACCCTCCCCATAAGCGCCCCTCCCGGGTTCCCAAAGCAGAGGGCGTGGG
GGAAAAGAAAAAAGATCCTCTCTCGCTAATCTCCGCCCACCGGCCCTTTATAATGCGAGGGTCTGGACGG
CTGAGGACCCCCGAGCTGTGCTGCTCGCGGCCGCCACCGCCGGGCCCCGGCCGTCCCTGGCTCCCCTCCT
GCCTCGAGAAGGGCAGGGCTTCTCAGAGGCTTGGCGGGAAAAAGAACGGAGGGAGGGATCGCGCTGAGTA
TAAAAGCCGGTTTTCGGGGCTTTATCTAACTCGCTGTAGTAATTCCAGCGAGAGGCAGAGGGAGCGAGCG
GGCGGCCGGCTAGGGTGGAAGAGCCGGGCGAGCAGAGCTGCGCTGCGGGCGTCCTGGGAAGGGAGATCCG
GAGCGAATAGGGGGCTTCGCCTCTGGCCCAGCCCTCCCGCTGATCCCCCAGCCAGCGGTCCGCAACCCTT
GCCGCATCCACGAAACTTTGCCCATAGCAGCGGGCGGGCACTTTGCACTGGAACTTACAACACCCGAGCA
AGGACGCGACTCTCCCGACGCGGGGAGGCTATTCTGCCCATTTGGGGACACTTCCCCGCCGCTGCCAGGA
CCCGCTTCTCTGAAAGGCTCTCCTTGCAGCTGCTTAGACGCTGGATTTTTTTCGGGTAGTGGAAAACCAG
CCTCCCGCGACGATGCCCCTCAACGTTAGCTTCACCAACAGGAACTATGACCTCGACTACGACTCGGTGC
AGCCGTATTTCTACTGCGACGAGGAGGAGAACTTCTACCAGCAGCAGCAGCAGAGCGAGCTGCAGCCCCC
GGCGCCCAGCGAGGATATCTGGAAGAAATTCGAGCTGCTGCCCACCCCGCCCCTGTCCCCTAGCCGCCGC
TCCGGGCTCTGCTCGCCCTCCTACGTTGCGGTCACACCCTTCTCCCTTCGGGGAGACAACGACGGCGGTG
GCGGGAGCTTCTCCACGGCCGACCAGCTGGAGATGGTGACCGAGCTGCTGGGAGGAGACATGGTGAACCA
GAGTTTCATCTGCGACCCGGACGACGAGACCTTCATCAAAAACATCATCATCCAGGACTGTATGTGGAGC
GGCTTCTCGGCCGCCGCCAAGCTCGTCTCAGAGAAGCTGGCCTCCTACCAGGCTGCGCGCAAAGACAGCG
GCAGCCCGAACCCCGCCCGCGGCCACAGCGTCTGCTCCACCTCCAGCTTGTACCTGCAGGATCTGAGCGC
CGCCGCCTCAGAGTGCATCGACCCCTCGGTGGTCTTCCCCTACCCTCTCAACGACAGCAGCTCGCCCAAG
TCCTGCGCCTCGCAAGACTCCAGCGCCTTCTCTCCGTCCTCGGATTCTCTGCTCTCCTCGACGGAGTCCT
CCCCGCAGGGCAGCCCCGAGCCCCTGGTGCTCCATGAGGAGACACCGCCCACCACCAGCAGCGACTCTGA
GGAGGAACAAGAAGATGAGGAAGAAATCGATGTTGTTTCTGTGGAAAAGAGGCAGGCTCCTGGCAAAAGG
TCAGAGTCTGGATCACCTTCTGCTGGAGGCCACAGCAAACCTCCTCACAGCCCACTGGTCCTCAAGAGGT
GCCACGTCTCCACACATCAGCACAACTACGCAGCGCCTCCCTCCACTCGGAAGGACTATCCTGCTGCCAA
GAGGGTCAAGTTGGACAGTGTCAGAGTCCTGAGACAGATCAGCAACAACCGAAAATGCACCAGCCCCAGG
TCCTCGGACACCGAGGAGAATGTCAAGAGGCGAACACACAACGTCTTGGAGCGCCAGAGGAGGAACGAGC
TAAAACGGAGCTTTTTTGCCCTGCGTGACCAGATCCCGGAGTTGGAAAACAATGAAAAGGCCCCCAAGGT
AGTTATCCTTAAAAAAGCCACAGCATACATCCTGTCCGTCCAAGCAGAGGAGCAAAAGCTCATTTCTGAA
GAGGACTTGTTGCGGAAACGACGAGAACAGTTGAAACACAAACTTGAACAGCTACGGAACTCTTGTGCGT
AAGGAAAAGTAAGGAAAACGATTCCTTCTAACAGAAATGTCCTGAGCAATCACCTATGAACTTGTTTCAA
ATGCATGATCAAATGCAACCTCACAACCTTGGCTGAGTCTTGAGACTGAAAGATTTAGCCATAATGTAAA
CTGCCTCAAATTGGACTTTGGGCATAAAAGAACTTTTTTATGCTTACCATCTTTTTTTTTTCTTTAACAG
ATTTGTATTTAAGAATTGTTTTTAAAAAATTTTAAGATTTACACAATGTTTCTCTGTAAATATTGCCATT
AAATGTAAATAACTTTAATAAAACGTTTATAGCAGTTACACAGAATTTCAATCCTAGTATATAGTACCTA
GTATTATAGGTACTATAAACCCTAATTTTTTTTATTTAAGTACATTTTGCTTTTTAAAGTTGATTTTTTT
CTATTGTTTTTAGAAAAAATAAAATAACTGGCAAATATATCATTGAGCCAAATCTTAAGTTGTGAATGTT
TTGTTTCGTTTCTTCCCCCTCCCAACCACCACCATCCCTGTTTGTTTTCATCAATTGCCCCTTCAGAGGG
TGGTCTTAAGAAAGGCAAGAGTTTTCCTCTGTTGAAATGGGTCTGGGGGCCTTAAGGTCTTTAAGTTCTT
GGAGGTTCTAAGATGCTTCCTGGAGACTATGATAACAGCCAGAGTTGACAGTTAGAAGGAATGGCAGAAG
GCAGGTGAGAAGGTGAGAGGTAGGCAAAGGAGATACAAGAGGTCAAAGGTAGCAGTTAAGTACACAAAGA
GGCATAAGGACTGGGGAGTTGGGAGGAAGGTGAGGAAGAAACTCCTGTTACTTTAGTTAACCAGTGCCAG
TCCCCTGCTCACTCCAAACCCAGGAATTCTGCCCAGTTGATGGGGACACGGTGGGAACCAGCTTCTGCTG
CCTTCACAACCAGGCGCCAGTCCTGTCCATGGGTTATCTCGCAAACCCCAGAGGATCTCTGGGAGGAATG
CTACTATTAACCCTATTTCACAAACAAGGAAATAGAAGAGCTCAAAGAGGTTATGTAACTTATCTGTAGC
CACGCAGATAATACAAAGCAGCAATCTGGACCCATTCTGTTCAAAACACTTAACCCTTCGCTATCATGCC
TTGGTTCATCTGGGTCTAATGTGCTGAGATCAAGAAGGTTTAGGACCTAATGGACAGACTCAAGTCATAA
CAATGCTAAGCTCTATTTGTGTCCCAAGCACTCCTAAGCATTTTATCCCTAACTCTACATCAACCCCATG
AAGGAGATACTGTTGATTTCCCCATATTAGAAGTAGAGAGGGAAGCTGAGGCACACAAAGACTCATCCAC
ATGCCCAAGATTCACTGATAGGGAAAAGTGGAAGCGAGATTTGAACCCAGGCTGTTTACTCCTAACCTGT
CCAAGCCACCTCTCAGACGACGGTAGGAATCAGCTGGCTGCTTGTGAGTACAGGAGTTACAGTCCAGTGG
GTTATGTTTTTTAAGTCTCAACATCTAAGCCTGGTCAGGCATCAGTTCCCCTTTTTTTGTGATTTATTTT
GTTTTTATTTTGTTGTTCATTGTTTAATTTTTCCTTTTACAATGAGAAGGTCACCATCTTGACTCCTACC
TTAGCCATTTGTTGAATCAGACTCATGACGGCTCCTGGGAAGAAGCCAGTTCAGATCATAAAATAAAACA
TATTTATTCTTTGTCATGGGAGTCATTATTTTAGAAACTACAAACTCTCCTTGCTTCCATCCTTTTTTAC
ATACTCATGACACATGCTCATCCTGAGTCCTTGAAAAGGTATTTTTGAACATGTGTATTAATTATAAGCC
TCTGAAAACCTATGGCCCAAACCAGAAATGATGTTGATTATATAGGTAAATGAAGGATGCTATTGCTGTT
CTAATTACCTCATTGTCTCAGTCTCAAAGTAGGTCTTCAGCTCCCTGTACTTTGGGATTTTAATCTACCA
CCACCCATAAATCAATAAATAATTACTTTCTTTGA
NM_000314.8 Homo sapiens phosphatase and tensin homolog (PTEN),
transcript variant 1, mRNA
(SEQ ID NO: 22)
GTTCTCTCCTCTCGGAAGCTGCAGCCATGATGGAAGTTTGAGAGTTGAGCCGCTGTGAGGCGAGGCCGGG
CTCAGGCGAGGGAGATGAGAGACGGCGGCGGCCGCGGCCCGGAGCCCCTCTCAGCGCCTGTGAGCAGCCG
CGGGGGCAGCGCCCTCGGGGAGCCGGCCGGCCTGCGGCGGCGGCAGCGGCGGCGTTTCTCGCCTCCTCTT
CGTCTTTTCTAACCGTGCAGCCTCTTCCTCGGCTTCTCCTGAAAGGGAAGGTGGAAGCCGTGGGCTCGGG
CGGGAGCCGGCTGAGGCGCGGCGGCGGCGGCGGCACCTCCCGCTCCTGGAGCGGGGGGGAGAAGCGGCGG
CGGCGGCGGCCGCGGCGGCTGCAGCTCCAGGGGGGGGTCTGAGTCGCCTGTCACCATTTCCAGGGCTGG
GAACGCCGGAGAGTTGGTCTCTCCCCTTCTACTGCCTCCAACACGGCGGCGGCGGCGGCTGGCACATCCA
GGGACCCGGGCCGGTTTTAAACCTCCCGTGCGCCGCCGCCGCACCCCCCGTGGCCCGGGCTCCGGAGGCC
GCCGGCGGAGGCAGCCGTTCGGAGGATTATTCGTCTTCTCCCCATTCCGCTGCCGCCGCTGCCAGGCCTC
TGGCTGCTGAGGAGAAGCAGGCCCAGTCGCTGCAACCATCCAGCAGCCGCCGCAGCAGCCATTACCCGGC
TGCGGTCCAGAGCCAAGCGGCGGCAGAGCGAGGGGCATCAGCTACCGCCAAGTCCAGAGCCATTTCCATC
CTGCAGAAGAAGCCCCGCCACCAGCAGCTTCTGCCATCTCTCTCCTCCTTTTTCTTCAGCCACAGGCTCC
CAGACATGACAGCCATCATCAAAGAGATCGTTAGCAGAAACAAAAGGAGATATCAAGAGGATGGATTCGA
CTTAGACTTGACCTATATTTATCCAAACATTATTGCTATGGGATTTCCTGCAGAAAGACTTGAAGGCGTA
TACAGGAACAATATTGATGATGTAGTAAGGTTTTTGGATTCAAAGCATAAAAACCATTACAAGATATACA
ATCTTTGTGCTGAAAGACATTATGACACCGCCAAATTTAATTGCAGAGTTGCACAATATCCTTTTGAAGA
CCATAACCCACCACAGCTAGAACTTATCAAACCCTTTTGTGAAGATCTTGACCAATGGCTAAGTGAAGAT
GACAATCATGTTGCAGCAATTCACTGTAAAGCTGGAAAGGGACGAACTGGTGTAATGATATGTGCATATT
TATTACATCGGGGCAAATTTTTAAAGGCACAAGAGGCCCTAGATTTCTATGGGGAAGTAAGGACCAGAGA
CAAAAAGGGAGTAACTATTCCCAGTCAGAGGCGCTATGTGTATTATTATAGCTACCTGTTAAAGAATCAT
CTGGATTATAGACCAGTGGCACTGTTGTTTCACAAGATGATGTTTGAAACTATTCCAATGTTCAGTGGCG
GAACTTGCAATCCTCAGTTTGTGGTCTGCCAGCTAAAGGTGAAGATATATTCCTCCAATTCAGGACCCAC
ACGACGGGAAGACAAGTTCATGTACTTTGAGTTCCCTCAGCCGTTACCTGTGTGTGGTGATATCAAAGTA
GAGTTCTTCCACAAACAGAACAAGATGCTAAAAAAGGACAAAATGTTTCACTTTTGGGTAAATACATTCT
TCATACCAGGACCAGAGGAAACCTCAGAAAAAGTAGAAAATGGAAGTCTATGTGATCAAGAAATCGATAG
CATTTGCAGTATAGAGCGTGCAGATAATGACAAGGAATATCTAGTACTTACTTTAACAAAAAATGATCTT
GACAAAGCAAATAAAGACAAAGCCAACCGATACTTTTCTCCAAATTTTAAGGTGAAGCTGTACTTCACAA
AAACAGTAGAGGAGCCGTCAAATCCAGAGGCTAGCAGTTCAACTTCTGTAACACCAGATGTTAGTGACAA
TGAACCTGATCATTATAGATATTCTGACACCACTGACTCTGATCCAGAGAATGAACCTTTTGATGAAGAT
CAGCATACACAAATTACAAAAGTCTGAATTTTTTTTTATCAAGAGGGATAAAACACCATGAAAATAAACT
TGAATAAACTGAAAATGGACCTTTTTTTTTTTAATGGCAATAGGACATTGTGTCAGATTACCAGTTATAG
GAACAATTCTCTTTTCCTGACCAATCTTGTTTTACCCTATACATCCACAGGGTTTTGACACTTGTTGTCC
AGTTGAAAAAAGGTTGTGTAGCTGTGTCATGTATATACCTTTTTGTGTCAAAAGGACATTTAAAATTCAA
TTAGGATTAATAAAGATGGCACTTTCCCGTTTTATTCCAGTTTTATAAAAAGTGGAGACAGACTGATGTG
TATACGTAGGAATTTTTTCCTTTTGTGTTCTGTCACCAACTGAAGTGGCTAAAGAGCTTTGTGATATACT
GGTTCACATCCTACCCCTTTGCACTTGTGGCAACAGATAAGTTTGCAGTTGGCTAAGAGAGGTTTCCGAA
GGGTTTTGCTACATTCTAATGCATGTATTCGGGTTAGGGGAATGGAGGGAATGCTCAGAAAGGAAATAAT
TTTATGCTGGACTCTGGACCATATACCATCTCCAGCTATTTACACACACCTTTCTTTAGCATGCTACAGT
TATTAATCTGGACATTCGAGGAATTGGCCGCTGTCACTGCTTGTTGTTTGCGCATTTTTTTTTAAAGCAT
ATTGGTGCTAGAAAAGGCAGCTAAAGGAAGTGAATCTGTATTGGGGTACAGGAATGAACCTTCTGCAACA
TCTTAAGATCCACAAATGAAGGGATATAAAAATAATGTCATAGGTAAGAAACACAGCAACAATGACTTAA
CCATATAAATGTGGAGGCTATCAACAAAGAATGGGCTTGAAACATTATAAAAATTGACAATGATTTATTA
AATATGTTTTCTCAATTGTAACGACTTCTCCATCTCCTGTGTAATCAAGGCCAGTGCTAAAATTCAGATG
CTGTTAGTACCTACATCAGTCAACAACTTACACTTATTTTACTAGTTTTCAATCATAATACCTGCTGTGG
ATGCTTCATGTGCTGCCTGCAAGCTTCTTTTTTCTCATTAAATATAAAATATTTTGTAATGCTGCACAGA
AATTTTCAATTTGAGATTCTACAGTAAGCGTTTTTTTTCTTTGAAGATTTATGATGCACTTATTCAATAG
CTGTCAGCCGTTCCACCCTTTTGACCTTACACATTCTATTACAATGAATTTTGCAGTTTTGCACATTTTT
TAAATGTCATTAACTGTTAGGGAATTTTACTTGAATACTGAATACATATAATGTTTATATTAAAAAGGAC
ATTTGTGTTAAAAAGGAAATTAGAGTTGCAGTAAACTTTCAATGCTGCACACAAAAAAAAGACATTTGAT
TTTTCAGTAGAAATTGTCCTACATGTGCTTTATTGATTTGCTATTGAAAGAATAGGGTTTTTTTTTTTTT
TTTTTTTTTTTTTTTTAAATGTGCAGTGTTGAATCATTTCTTCATAGTGCTCCCCCGAGTTGGGACTAGG
GCTTCAATTTCACTTCTTAAAAAAAATCATCATATATTTGATATGCCCAGACTGCATACGATTTTAAGCG
GAGTACAACTACTATTGTAAAGCTAATGTGAAGATATTATTAAAAAGGTTTTTTTTTCCAGAAATTTGGT
GTCTTCAAATTATACCTTCACCTTGACATTTGAATATCCAGCCATTTTGTTTCTTAATGGTATAAAATTC
CATTTTCAATAACTTATTGGTGCTGAAATTGTTCACTAGCTGTGGTCTGACCTAGTTAATTTACAAATAC
AGATTGAATAGGACCTACTAGAGCAGCATTTATAGAGTTTGATGGCAAATAGATTAGGCAGAACTTCATC
TAAAATATTCTTAGTAAATAATGTTGACACGTTTTCCATACCTTGTCAGTTTCATTCAACAATTTTTAAA
TTTTTAACAAAGCTCTTAGGATTTACACATTTATATTTAAACATTGATATATAGAGTATTGATTGATTGC
TCATAAGTTAAATTGGTAAAGTTAGAGACAACTATTCTAACACCTCACCATTGAAATTTATATGCCACCT
TGTCTTTCATAAAAGCTGAAAATTGTTACCTAAAATGAAAATCAACTTCATGTTTTGAAGATAGTTATAA
ATATTGTTCTTTGTTACAATTTCGGGCACCGCATATTAAAACGTAACTTTATTGTTCCAATATGTAACAT
GGAGGGCCAGGTCATAAATAATGACATTATAATGGGCTTTTGCACTGTTATTATTTTTCCTTTGGAATGT
GAAGGTCTGAATGAGGGTTTTGATTTTGAATGTTTCAATGTTTTTGAGAAGCCTTGCTTACATTTTATGG
TGTAGTCATTGGAAATGGAAAAATGGCATTATATATATTATATATATAAATATATATTATACATACTCTC
CTTACTTTATTTCAGTTACCATCCCCATAGAATTTGACAAGAATTGCTATGACTGAAAGGTTTTCGAGTC
CTAATTAAAACTTTATTTATGGCAGTATTCATAATTAGCCTGAAATGCATTCTGTAGGTAATCTCTGAGT
TTCTGGAATATTTTCTTAGACTTTTTGGATGTGCAGCAGCTTACATGTCTGAAGTTACTTGAAGGCATCA
CTTTTAAGAAAGCTTACAGTTGGGCCCTGTACCATCCCAAGTCCTTTGTAGCTCCTCTTGAACATGTTTG
CCATACTTTTAAAAGGGTAGTTGAATAAATAGCATCACCATTCTTTGCTGTGGCACAGGTTATAAACTTA
AGTGGAGTTTACCGGCAGCATCAAATGTTTCAGCTTTAAAAAATAAAAGTAGGGTACAAGTTTAATGTTT
AGTTCTAGAAATTTTGTGCAATATGTTCATAACGATGGCTGTGGTTGCCACAAAGTGCCTCGTTTACCTT
TAAATACTGTTAATGTGTCATGCATGCAGATGGAAGGGGTGGAACTGTGCACTAAAGTGGGGGCTTTAAC
TGTAGTATTTGGCAGAGTTGCCTTCTACCTGCCAGTTCAAAAGTTCAACCTGTTTTCATATAGAATATAT
ATACTAAAAAATTTCAGTCTGTTAAACAGCCTTACTCTGATTCAGCCTCTTCAGATACTCTTGTGCTGTG
CAGCAGTGGCTCTGTGTGTAAATGCTATGCACTGAGGATACACAAAAATACCAATATGATGTGTACAGGA
TAATGCCTCATCCCAATCAGATGTCCATTTGTTATTGTGTTTGTTAACAACCCTTTATCTCTTAGTGTTA
TAAACTCCACTTAAAACTGATTAAAGTCTCATTCTTGTCATTGTGTGGGTGTTTTATTAAATGAGAGTTT
ATAATTCAAATTGCTTAAGTCCATTGAAGTTTTAATTAATGGGCAGCCAAATGTGAATACAAAGTTTTCA
GTTTTTTTTTTTCCTGCTGTCCTTCAAAGCCTACTGTTTAAAAAAAAAAAAAAAAAAAAACATGGCCTGA
GAGTAGAGTATCTGTCTACTCATGTTTAATTAAGGAAAAACACTTATTTTTAGGGCTTTAGTCATCACTT
CATAAATTGTATAAGCACATTAAATAGCGTTCTAGTCCTGAAAAAGTCCAAGATTCTTAGAAAATTGTGC
ATATTTTTATTATGACAGATGTTTGAAGATAATTCCCCAGAATGGATTTGATACTTTAGATTTCAATTTT
GTGGCTTTTGTCTATTATTCTGTACTCTGCCATCAGCATATGGAAAGCTTCATTTACTCATCATGACTTG
TGCCATATAAAAATTGATATTTCGGAATAGTCTAAAGGACTTTTTGTACTTGAATTTAATCATGTTGTTT
CTAATATTCTTAAAAGCTTGAAGACTAAAGCATATCCTTTCAACAAAGCATAGTAAGGTAATAAGAAAGT
GTAGTTTGTACAAGTGTTAAAAAAATAAAGTAGACAATGTTACAGTGGGACTTATTATTTCAAGTTTACA
TTTTCTCCATGTAATTTTTTAAAAAGTAAATGAAAAAATGTGCAATAATGTAAAATATGAAGTGTATGTG
TACACACATTTTATTTTTCGGTATCTTGGGTATACGTATGGTTGAAAACTATACTGGAGTCTAAAAGTAT
TCTAATTTATAAGAAGACATTTTGGTGATGTTTGAAAAATAGAAATGTGCTAGTTTTGTTTTTATATCAT
GTCCTTTGTACGTTGTAATATGAGCTGGCTTGGTTCAGTAAATGCCATCACCATTTCCATTGAGAATTTA
AAACTCACCAGTGTTTAATATGCAGGCTTCCAAAGGCTTATGAAAAAAATCAAGACCCTTAAATCTAGTT
AATTTGCTGCTAACATGAAACTCTTTGGTTCTTTTATTTTTGCCAGATAATTAGACACACATCTAAAGCT
TAGTCTTAAATGGCTTAAGTGTAGCTATTGATTAGTGCTGTTGCTAGTTCAGAAAGAAATGTTTGTGAAT
GGAAACAAGAATATTCAGTCCAAACTGTTGTAAGGACAGTACCTGAAAACCAGGAAACAGGATAATGGAA
AAAGTCTTTTAAAGATGAAATGTTGGAGCCAACTTTCTTATAGAATTAATTGTATGTGGCTATAGAAAGC
CTAATGATTGTTGCTTATTTTTGAGAGCATATTATTCTTTTATGACCATAATCTTGCTGTTTTTCCATCT
TCCAAAAGATCTTCCTTCTAATATGTATATCAGAATGTGGGTAGCCAGTCAGACAAATTCATATTGGTTG
GTAGCTTTAAAAAGTTTGTAATGTGAAGACAGGAAAGGACAAAATAGTTTGCTTTGGTGGTAGTACTCTG
GTTGTTAAGCTAGGTATTTTGAGACTACTTCCCCATCACAACAACAATAAAATAATCACTCATAATCCTA
TCACCTGGAGACATAGCCATCGTTAATATGTTAGTGACTATACAATCATGTTTTCTTCTGTATATCCATG
TATATTCTTTAAAAATGAAATTTATACTGTACCTGATCTCAAAGCTTTTTAGCTTAGTATATCTGTCATG
AATTTGTAGGATGTTCCATTGCATCAGAAAACGGACAGTGATTTGATTACTTTCTAATGCCACAGATGCA
GATTACATGTAGTTATTGAGAATCCTTTCGAATTCAGTGGCTTAATCATGAATGTCTAAATATTGTTGAC
ATTAGGATGATACATGTAAATTAAAGTTACATTTGTTTAGCATAGACAAGCTTAACATTGTAGATGTTTC
TCTTCAAAAATCATCTTAAACATTTGCATTTGGAATTGTGTTAAATAGAATGTGTGAAACACTGTATTAG
TAAACTTCATCACCTTTCTACTTCCTTATAGTTTGAACTTTTCAGTTTTTGTAGTTCCCAAACAGTTGCT
CAATTTAGAGCAAATTAATTTAACACCTGCCAAAAAAAGGCTGCTGTTGGCTTATCAGTTGTCTTTAAAT
TCAAATGCTCATGTGACTTTTATCACATCAAAAAATATTTCATTAATGATTCACCTTTAGCTCTGAAAAT
TACCGCGTTTAGTAATTATAGTGGGCTTATAAAAACATGCAACTCTTTTTGATAGTTATTTGAGAATTTT
GGTGAAAAATATTTAGCTGAGGGCAGTATAGAACTTATAAACCAATATATTGATATTTTTAAAACATTTT
TACATATAAGTAAACTGCCATCTTTGAGCATAACTACATTTAAAAATAAAGCTGCATATTTTTAAATCAA
GTGTTTAACAAGAATTTATATTTTTTATTTTTTAAAATTAAAAATAATTTATATTTCCTCTGTTGCATGA
GGATTCTCATCTGTGCTTATAATGGTTAGAGATTTTATTTGTGTGGAATGAAGTGAGGCTTGTAGTCATG
GTTCTAGTGTTTCAGTTTGCCAAGTCTGTTTACTGCAGTGAAATTCATCAAATGTTTCAGTGTGGTTTTC
TGTAGCCTATCATTTACTGGCTATTTTTTTATGTACACCTTTAGGATTTTCTGCCTACTCTATCCAGTTG
TCCAAATGATATCCTACATTTTACAAATGCCCTTTCAGTTTCTATTTTCTTTTTCCATTAAATTGCCCTC
ATGTCCTAATGTGCAGTTTGTAAGTGTGTGTGTGTGTGTCTGTGTGTGTGTGAATTTGATTTTCAAGAGT
GCTAGACTTCCAATTTGAGAGATTAAATAATTTAATTCAGGCAAACATTTTTCATTGGAATTTCACAGTT
CATTGTAATGAAAATGTTAATCCTGGATGACCTTTGACATACAGTAATGAATCTTGGATATTAATGAATT
TGTTAGTAGCATCTTGATGTGTGTTTTAATGAGTTATTTTCAAAGTTGTGCATTAAACCAAAGTTGGCAT
ACTGGAAGTGTTTATATCAAGTTCCATTTGGCTACTGATGGACAAAAAATAGAAATGCCTTCCTATGGAG
AGTATTTTTCCTTTAAAAAATTAAAAAGGTTAATTATTTTGACTA
NM_001285439.2 Homo sapiens RPTOR independent companion of MTOR
complex 2 (RICTOR), transcript variant 2, mRNA
(SEQ ID NO: 23)
GTTGTGACTGAAACCCGTCAATATGGCGGCGATCGGCCGCGGCCGCTCTCTGAAGAACCTCCGAGTACGA
GGGCGGAATGACAGCGGCGAGGAGAACGTCCCGCTGGATCTGACCCGAGAACCTTCTGATAACTTAAGAG
AGATTCTCCAAAATGTGGCCAGATTGCAGGGAGTATCAAATATGAGAAAGCTAGGCCATCTGAATAACTT
TACTAAGCTTCTTTGTGATATTGGCCACAGTGAAGAAAAACTGGGCTTTCACTATGAGGATATCATAATT
TGTTTGCGGTTAGCTTTATTAAATGAAGCAAAAGAAGTGCGAGCAGCAGGGCTACGAGCGCTTCGATATC
TCATCCAAGACTCCAGTATTCTCCAGAAGGTGCTAAAATTGAAAGTGGACTATTTAATAGCTAGGTGCAT
TGACATACAACAGAGCAACGAGGTAGAGAGGACACAAGCACTTCGATTAGTCAGAAAGATGATTACTGTG
AATGCTTCCTTGTTTCCTAGTTCTGTGACCAACTCATTAATTGCAGTTGGAAATGATGGACTTCAAGAAA
GAGACAGAATGGTCCGAGCATGCATTGCCATTATCTGTGAACTAGCACTTCAGAATCCAGAGGTGGTGGC
CCTTCGAGGTGGACTAAACACCATCTTGAAAAATGTGATCGATTGCCAATTAAGTCGAATAAATGAGGCC
CTAATTACTACAATTTTGCACCTTCTTAATCATCCAAAGACTCGACAGTATGTGCGAGCTGATGTAGAAT
TAGAGAGAATTTTAGCACCCTATACTGATTTTCACTACAGACATAGTCCAGATACAGCTGAAGGACAGCT
CAAAGAAGACAGAGAAGCACGATTTCTAGCCAGTAAAATGGGAATCATAGCAACATTCCGATCATGGGCA
GGTATTATTAATTTATGTAAACCTGGAAATTCTGGGATCCAGTCTCTAATAGGAGTACTTTGCATACCAA
ATATGGAAATAAGGCGAGGTCTACTTGAAGTGCTTTATGATATATTTCGTCTTCCTCTACCTGTTGTGAC
TGAGGAGTTCATAGAAGCACTACTCAGTGTAGATCCAGGGAGGTTCCAAGACAGTTGGAGGCTTTCAGAT
GGCTTTGTGGCAGCTGAGGCAAAAACTATTCTTCCTCATCGTGCCAGATCCAGGCCAGACCTCATGGATA
ATTATTTGGCACTGATACTCTCTGCATTTATTCGTAATGGACTTTTAGAGGGTCTAGTTGAAGTGATAAC
AAACAGTGATGATCATATCTCAGTTAGAGCTACCATCCTTTTAGGAGAGCTTTTACATATGGCAAACACA
ATTCTTCCTCATTCACATAGCCATCATTTACACTGCTTGCCAACCCTAATGAATATGGCTGCATCCTTTG
ATATCCCCAAGGAAAAGAGACTGCGAGCCAGTGCAGCCTTGAACTGTTTAAAACGCTTCCATGAAATGAA
GAAACGAGGACCTAAGCCTTATAGTCTTCATTTAGACCACATTATTCAGAAAGCAATTGCAACACACCAG
AAACGGGATCAGTATCTCCGAGTTCAGAAAGATATATTTATCCTTAAGGATACAGAGGAAGCTCTTTTAA
TTAACCTTAGAGATAGCCAAGTCCTTCAACATAAAGAGAATCTTGAATGGAATTGGAATCTTATAGGGAC
CATTCTTAAGTGGCCAAATGTAAATCTAAGAAACTATAAAGATGAACAGTTACACAGGTTTGTACGAAGA
CTACTTTATTTTTACAAGCCCAGCAGTAAATTATATGCCAACCTGGATCTGGATTTTGCCAAGGCCAAAC
AGCTCACGGTTGTAGGTTGCCAGTTTACAGAATTTCTTCTTGAATCTGAAGAGGATGGGCAAGGCTACTT
AGAAGATCTAGTAAAGGATATTGTTCAGTGGCTCAATGCTTCATCTGGAATGAAACCCGAAAGAAGTCTT
CAAAATAATGGTTTATTGACCACCCTTAGTCAACACTACTTTTTATTTATTGGAACACTTTCTTGCCACC
CTCATGGAGTTAAAATGCTGGAAAAATGCAGTGTATTTCAGTGTCTCCTTAATCTTTGCTCCTTGAAAAA
CCAAGATCACTTGCTAAAACTTACTGTTTCTAGCTTGGACTATAGCAGAGATGGATTGGCTAGAGTCATC
CTTTCCAAAATTTTAACTGCAGCTACTGATGCCTGCAGACTCTATGCAACAAAACATTTAAGGGTATTAT
TGAGAGCTAATGTTGAATTCTTTAATAATTGGGGAATTGAGTTGTTAGTGACCCAGCTACATGATAAAAA
CAAAACGATTTCCTCTGAAGCTCTTGATATCCTCGATGAAGCATGTGAAGACAAGGCCAATCTTCATGCT
CTCATTCAGATGAAACCAGCGTTATCCCACCTTGGAGACAAGGGTTTGCTTCTCCTGCTGAGATTTCTCT
CCATTCCAAAAGGATTTTCCTATCTGAATGAAAGAGGTTATGTAGCAAAACAATTGGAAAAGTGGCACAG
GGAATACAACTCCAAATATGTTGACTTGATTGAGGAACAACTCAATGAAGCACTTACTACTTACCGGAAG
CCTGTTGATGGTGATAACTATGTTCGTCGGAGTAACCAAAGATTACAGCGTCCTCACGTCTACCTGCCTA
TACACCTTTATGGACAACTAGTACACCATAAAACAGGCTGCCATTTGTTGGAAGTACAGAATATTATTAC
AGAACTCTGTCGTAATGTTCGTACACCAGATTTGGATAAGTGGGAAGAAATTAAAAAACTGAAAGCATCT
CTTTGGGCCTTGGGAAATATCGGCTCATCAAATTGGGGTCTCAATTTGCTACAGGAAGAAAACGTGATTC
CAGATATACTAAAACTTGCAAAACAGTGTGAAGTTCTTTCCATCAGAGGGACCTGTGTATATGTACTTGG
GCTCATAGCTAAAACCAAACAAGGCTGTGATATTCTAAAATGTCACAACTGGGATGCTGTGAGGCATAGT
CGCAAACATCTGTGGCCAGTGGTTCCAGATGATGTGGAACAACTCTGTAATGAACTTTCATCTATCCCAA
GCACTCTAAGTTTGAACTCGGAGTCAACCAGCTCTAGACATAATAGTGAAAGTGAATCTGTGCCATCGAG
TATGTTCATATTGGAGGATGACCGGTTTGGCAGCAGCTCTACTAGCACATTTTTCCTTGATATCAATGAA
GATACAGAGCCAACATTTTATGACCGATCTGGACCCATAAAGGATAAAAATTCATTCCCTTTCTTTGCTT
CTAGTAAACTTGTGAAGAATCGTATCTTAAATTCGCTTACTTTGCCTAACAAAAAACATCGTAGTAGCAG
TGATCCAAAAGGAGGGAAATTATCATCTGAAAGTAAGACAAGCAACAGGCGAATCAGAACACTTACGGAG
CCCAGTGTTGATTTTAATCATAGTGATGATTTTACACCCATATCCACTGTACAGAAAACATTACAATTAG
AGACTTCATTTATGGGGAATAAGCACATTGAAGACACTGGTAGTACACCAAGCATTGGAGAAAATGACTT
AAAATTCACCAAGAATTTTGGTACAGAGAATCACAGAGAAAATACAAGCCGAGAGAGGTTAGTAGTAGAA
AGTTCAACGAGCTCACATATGAAGATACGTAGCCAAAGTTTCAATACAGACACTACAACAAGTGGCATAA
GTTCAATGAGCTCAAGTCCTTCACGAGAGACAGTAGGTGTAGATGCTACAACTATGGACACAGACTGTGG
AAGCATGAGTACTGTGGTAAGTACTAAAACTATTAAGACAAGCCACTATTTGACGCCACAGTCTAACCAT
CTGTCTCTCTCCAAATCAAATTCGGTGTCCCTGGTGCCTCCAGGTTCTTCTCATACGCTTCCTAGAAGAG
CACAGTCCCTTAAAGCACCCTCTATTGCTACAATTAAAAGTCTAGCAGATTGTAACTTTAGTTACACAAG
TTCTAGAGATGCTTTTGGCTATGCTACACTGAAAAGACTACAGCAACAAAGAATGCATCCATCCTTATCT
CACTCTGAAGCTTTGGCATCTCCAGCAAAAGATGTGCTATTTACTGATACCATCACCATGAAGGCCAACA
GTTTTGAGTCCAGATTAACACCAAGCAGGATCGATTTTAAAAAGAAGCATGTCGGGGGAATCAGGAGCTT
AAGACCTACAATAACAAACAACCTTTTCAGGTTCATGAAAGCCTTAAGTTATGCATCATTAGATAAAGAA
GATTTATTGAGTCCTATTAATCAAAATACCCTGCAACGATCTTCCTCAGTGCGGTCCATGGTGTCCAGTG
CCACATATGGGGGTTCAGATGATTACATTGGTCTTGCTCTCCCGGTGGATATAAATGATATATTCCAGGT
AAAGGATATTCCCTATTTTCAGACAAAAAACATACCACCACATGATGATCGAGGTGCAAGAGCATTTGCC
CATGATGCAGGAGGTCTTCCATCTGGAACTGGAGGTCTTGTAAAAAATTCTTTTCACTTGCTACGACAGC
AGATGAGTCTTACGGAAATAATGAATTCAATCCATTCAGATGCCTCTCTGTTTTTAGAAAGTACAGAAGA
CACTGGACTACAGGAACATACAGATGATAACTGCCTTTATTGTGTCTGTATTGAAATTCTGGGTTTCCAG
CCCAGCAACCAACTGAGTGCAATATGTAGTCATTCAGACTTTCAAGATATTCCATATTCTGATTGGTGTG
AGCAGACTATCCATAATCCTTTAGAAGTGGTTCCCTCTAAGTTTTCGGGGATTTCTGGATGCAGTGATGG
GGTGTCTCAAGAAGGCTCAGCTAGCAGCACCAAAAGCACAGAATTGTTACTAGGTGTTAAAACAATTCCA
GATGATACACCAATGTGCCGTATACTCCTTCGCAAAGAAGTTCTAAGATTAGTCATTAATTTGAGTAGTT
CAGTTTCAACTAAATGTCATGAGACTGGGCTTTTAACAATTAAGGAGAAGTATCCTCAAACATTTGATGA
CATATGCCTTTACTCTGAGGTTTCCCATTTGCTGTCACACTGCACATTCAGACTTCCGTGTCGGAGGTTC
ATACAAGAATTATTTCAAGATGTACAGTTTCTACAAATGCATGAAGAAGCAGAGGCTGTGTTGGCAACAC
CACCAAAGCAACCTATAGTTGATACATCTGCTGAATCCTGACCTCATATTTATGATGGATATAGATACAT
ACTATATATATTCATATTTGTGGATTTCCTAAAAGCCTCAGAAAATACGACTGACTAGGCAGCAAAGACA
GGAGTATCTTCTGTACACTGTTCCGCAGTTACTGGTACATGAACAGTTGGAACTGCTGACTTTCCTAACC
AAAACAACTTCCTTCTCTCCTTTGTTGAGCCTTTTGAGGGGTTCATGATTCATTACCACAGTTTTAAGAG
TTTCAGTTACCATTGTATGCAAGAGCCAAGCACTGAATACCTACATAGGTTTTCTATTTTCTTTCATTTT
AAAAGCATAATGACAGTGGAACAATAATGGGATATGCAGAAGCACCCTTCACAAGTTATTTCTGAATGAT
TTTTAGGGTAAATAATACAGATGCCTTGTTTGTTAACTAACTTGTGGAAAGCAGGAATCAGTGTCTCTAA
GGCTGCATCCTATTACCACAATGGGGTGTGCTATAACTGCTGGTATTAGAGAGGGAACTTTGGCCCTTTC
ACGTTTTTCTTAATGTTTGTAACACTACTTCAGAGGTTTATAACCTCAAAGCAGAAGAAGAGCCTCAACA
ACCCGGGACTTATAAGTTATTTTTATGTTACTAGACTTGCATAAAGATTCTTGTTTTCCAACTCTTCATT
TTGTTGCAATGTGTTATTACAGGATATATGAACCAATTAAGGTTTTTCACTACAGTTCTTGAATAAAATT
TAAAAATCATTTTTTATTTTAATTAAAAATATTTCCCATTTATAGAATGCATATATTTGCAATGGACTTC
CACTTTCATCAACTTTCCATCTCATCGCTTTAAACAGGAACTTGAACAAGCACTGTTAGTTTAGACCTAA
AGGATAGGAAAGCATTAAATAATACTTTGGATCTCCTGAGGAAAAGATAAGTTTGCTTGCAATTTACACA
TTCCATGGGGAAAGAAGAGCCATATTTCCTTAAAAAAAACATTAATAAAGCTTGTTATTGAGAAAAATTG
TAGTGAAAAGCCTTAAGTACCAAATTTTAAAGCAGCAGTAACTTAATTTTTATATCAGTGTTTTTGTTTT
GCACAAACTAAATGCAGTGGTAGGTGGGTTTATGAGTATATTAATTGCCTTTATCCATTTGTGAAGTTAA
GTTGATGAGGGCAAGGTTTTTGTTTGTTTAATTTGTATATGTCTAAAGGTATTTGGAACTTTTTACAGGA
ATTAAACATATATGCAAATTTGTATATAAAAATAGCATGGCCATCATTTGAATGCTTGTAAATGAAAGGA
TTATCTTTTTTGAGATCTATATATAAATAGAAATAGAAAATCCAGCTGGACTGATTAGGATTCTTTTTTA
ATTCATTTGTGTATAACATTTTTATTACAATTACACATCAGTTTTGACACAGTCATAGCAACATTAATAT
TTTCCCATGATGCAGATCCTTTTTGTAATGGGCTTGTTCTTTGAGATCTCTGTAAAGAACCCTGTGAACT
AGAAAACATAACTCACAGAGATACTTTTTTAAAAAATTTATTTACTGGAACTGAAAGTTCCAGTTGGGAT
GAAGCATTTCATCTCACTTCATAACACCTCTTTGACTGCACTTCAGTGAATTGTTCTTATGTGCACTGTG
TAGCAACTTACATTATAACAAAGCAGATAAGGGCTGTAAGCTGCTGCTTATGTTGAAAAGTGGTTCTTCA
GATTTTCTCTCATAAAATCCAGTTGAAGATAAATAATTTTTTTATACTTTATCACTGAACCCAAGTGTTT
ATTTAAATGTCAACAGTACTTCTAAGAACGTTGCCTGTCATCGTGGTCTTTGGTCTTGGATAACTAAACT
GCCTTTCCAGAGAACCAAATGTCAGAGTTACTAGACCAAATAGTGGTTAAAACCTCCAAAGGAAGTAATG
TAATCTTATTCATAATGGGATTAACATATTTTAGACATTCATTTTAAACACTACCTCAGTTAATATAGAG
TATAAAAATCTGTGGTTTAATCCCTCAAAAGTTAACAGTAATTTTTTTTTTGTCTTACACACACACACAC
CCCCTCCCCCACCATCACTATCCCTGTACCCTCACCTTGGTCATCTATCCTGAAATAAGGCTTAGTTAGT
ATTGGCCTGAATGTTTTGTGTTTTTTTTTTTGTTTTTTTTTTTTACTGTTACTTTGAAAAATATGTATGT
ATACCTTATCATATCTGCCTATATCACTTACTTTGGGGAGATACTCAGAGCTTTGTGGTTATCAGTATAC
TAAAAAAAAAAAAAAGTCTACGCTTAAATTTATAGTGCTATTTGGTTTCTCCATGATTTCACTGACAGGT
CTAATACATTTTCTTTGAGTACTTGTTTGTAAAAAGTAGACTTTATGGTGAAAAATACATGCAGTGCCAA
GTGATTAACTTAAGTGTTTAAAAATATTAAATTATAGCAGAAGAGGTTAGGAATGATATCAGCAGTAATA
GAAATAATTGAGAAAATCATCTATAAATAATAGATATTACAGACTATAGAATACCAAAATAATGTCAATA
CTGTAGTTTTTAAAGATTTTAGGATTAATCTTAGTCCATATAAATTTGTACTATTGGTAATTATTGAATA
ATTGGGAGGAATCTGGGCAGTTGTGCTGGTTGTAAACTATGAATTTCTAATCGTAAAGTGAATTGTTATT
TCTAATTGAACTTTTTTTCAAGAACAGATTTCAGCCTCACATACTAAGTAAATACTGATAAATAAGGAAA
TTAGAAATTTAGTATTCATAATTAAATATGCTCTAAAATTTCCTATACTTTTATTTCCTGTTTATTCTTA
GGTAGATTGGAAGGGGGAAACAGTCTGTTCTCCCTAATTAAATTTTTTCTAATAACGATTAGTAGAATAT
GGACATTCTATATGACAGTGACATTAAAAGAGGCTCTTTGGAAGTATATACATTATTAACATAATGTGTA
CAAGTCCTTTTGAAATGACAACTTTAATGGGTTTCAGCTCTTTTATCTAGAGCTTGAGATAATTCAAGCT
GAGTTTTTCAGGGCATATCACAACGGCCAAGTGTTCAGCAGTGGGATATCAATGCTTATTTACATTTTCC
TACTGCTATTTATATAAAATGTTATTCCATTCAGAGGATGCCTTTTATCCCCACATTAAAGCACAGATCA
TTAAGCAATAAAAACCAAATTGTCTGTCATTCAAATTATAACTGCAGTTATTTTTGCATGGTAAGAGTGA
GGTGCTAATTTTGTGTGAGATGAACTTTGTAAACTACTTTGGGAAATGTTCTTTGGAAGTAAGGTTTTTT
CTCCTTTAGTCTTATGCTTCCACTTTTGTCTCAGATTCACAATCCATTAAAACATGGGGAAAAAAGAAAA
GGTAAAATTGAGAGACTTTTGTTAGAGGAGCTATTTGGAATGAACCAACATTTCAGATTTTCCAAAATGT
AAGTTAGGAAGTCTCCATTGTCTCTGCATTAACAAAATACACTGTTACTATCTTAATCTCAAGAGTGTCA
TTACAGTGAGAATCTCATTTAAAAGCATACCAGTGAAATTAATAGCAGTGCTTATCAAAGAACACTGAAA
TCTGTGAGAATCTTTCTAGGAGCATTCTTTTCTTCTTTTAGTTCCAAGTTCCAGGGTATTTTTCATTCCT
AGTAGGTTTATATGACTCACAGAATGTGGACTTTTTTCCTGTTTGGAGTATTTTTGTAATGTAAGTATCG
GATAGCTGCACCACAGCATGCATAAATTGCACATTTTGTTTTACTTTCTTTATAGAATATTTAATTTCAA
AAATATAATTTATGCCAAAAAAAGCATACCTTTCAATTTTGCTACTTGGTTGATTTAGCACAAAATGCAA
AGTCTTGGGGCAGAGAGGGGGAGTGAAAAAAATTTTATAGGTAATTGTTACAAAAATACCTGTCAGAAAC
CCTAAAGCTGCATTGTAAAACAAATGGTGTAAACTAGTTTTGAAAAGTGGTAAGGAATTGTGAAAAAAAT
CTCAGACTTAATGCTCTCTAACCACATGAGTTTCTTCTTTTTTATTTAGTAATACGCTGCTACATATTTG
GAGGTTCTGGTGTTTGTAGGTCACTGAACAGACATTGAAATCTGATTTATATTGTATAACTGTAACATAG
AAAGAAAAAGTATTTATATTTTTTCTGTAAGAATATTTCATTGAGTTGTGTATAATTTAAATAAGATTTG
TCCCCAAATGGTTTTGCTCACCTTGATTTTTTTTGTTGTGATTTTCTTGTTTTTGTATAATGTGTATAGT
TTATGTCAAGGGCATTAAAAGCCTCCTGAAGCATAATCTTATCAAAGGGATACATTGTTAATAAAATGTA
CTTAAAATTCTTAAA

Primers or probes can be designed so that they hybridize under stringent conditions to mutant nucleotide sequences of AKT1, ALK, B2M, BRAF, EGFR, ERBB2 (HER2), FGFR2, FGFR3, KEAP1, KRAS, MAP2K1 (MEK1), MET, NRAS, PIK3CA, RET, ROS1, STK11, TP53, NTRK1, FGFR1, MYC, PTEN, and RICTOR, but not to the respective wild-type nucleotide sequences. Primers or probes can also be prepared that are complementary and specific for the wild-type nucleotide sequence of AKT1, ALK, B2M, BRAF, EGFR, ERBB2 (HER2), FGFR2, FGFR3, KEAP1, KRAS, MAP2K1 (MEK1), MET, NRAS, PIK3CA, RET, ROS1, STK11, TP53, NTRK1, FGFR1, MYC, PTEN, and RICTOR, but not to any of the corresponding mutant nucleotide sequences. In some embodiments, the mutant nucleotide sequences of AKT1, ALK, B2M, BRAF, EGFR, ERBB2 (HER2), FGFR2, FGFR3, KEAP1, KRAS, MAP2K1 (MEK1), MET, NRAS, PIK3CA, RET, ROS1, STK11, TP53, NTRK1, FGFR1, MYC, PTEN, and RICTOR may be a frameshift mutation, a missense mutation, a deletion, an insertion, a nonsense mutation, an inversion, a translocation, a duplication, or a CNV that results in the altered expression and/or activity of AKT1, ALK, B2M, BRAF, EGFR, ERBB2 (HER2), FGFR2, FGFR3, KEAP1, KRAS, MAP2K1 (MEK1), MET, NRAS, PIK3CA, RET, ROS1, STK11, TP53, NTRK1, FGFR1, MYC, PTEN, and RICTOR.

In some embodiments, detection can occur through any of a variety of mobility dependent analytical techniques based on the differential rates of migration between different nucleic acid sequences. Exemplary mobility-dependent analysis techniques include electrophoresis, chromatography, mass spectroscopy, sedimentation, gradient centrifugation, field-flow fractionation, multi-stage extraction techniques, and the like. In some embodiments, mobility probes can be hybridized to amplification products, and the identity of the target nucleic acid sequence determined via a mobility dependent analysis technique of the eluted mobility probes, as described in Published PCT Applications WO04/46344 and WO01/92579. In some embodiments, detection can be achieved by various microarrays and related software such as the Applied Biosystems Array System with the Applied Biosystems 1700 Chemiluminescent Microarray Analyzer and other commercially available array systems available from Affymetrix, Agilent, Illumina, and Amersham Biosciences, among others (see also Gerry et al., J. Mol. Biol. 292:251-62, 1999; De Bellis et al., Minerva Biotec 14:247-52, 2002; and Stears et al., Nat. Med. 9:14045, including supplements, 2003).

It is also understood that detection can comprise reporter groups that are incorporated into the reaction products, either as part of labeled primers or due to the incorporation of labeled dNTPs during an amplification, or attached to reaction products, for example but not limited to, via hybridization tag complements comprising reporter groups or via linker arms that are integral or attached to reaction products. In some embodiments, unlabeled reaction products may be detected using mass spectrometry.

### NGS Platforms

In some embodiments, high throughput, massively parallel sequencing employs sequencing-by-synthesis with reversible dye terminators. In other embodiments, sequencing is performed via sequencing-by-ligation. In yet other embodiments, sequencing is single molecule sequencing. Examples of Next Generation Sequencing techniques include, but are not limited to pyrosequencing, Reversible dye-terminator sequencing, SOLID sequencing, Ion semiconductor sequencing, Helioscope single molecule sequencing etc.

The Ion Torrent™ (Life Technologies, Carlsbad, CA) amplicon sequencing system employs a flow-based approach that detects pH changes caused by the release of hydrogen ions during incorporation of unmodified nucleotides in DNA replication. For use with this system, a sequencing library is initially produced by generating DNA fragments flanked by sequencing adapters. In some embodiments, these fragments can be clonally amplified on particles by emulsion PCR. The particles with the amplified template are then placed in a silicon semiconductor sequencing chip. During replication, the chip is flooded with one nucleotide after another, and if a nucleotide complements the DNA molecule in a particular microwell of the chip, then it will be incorporated. A proton is naturally released when a nucleotide is incorporated by the polymerase in the DNA molecule, resulting in a detectable local change of pH. The pH of the solution then changes in that well and is detected by the ion sensor. If homopolymer repeats are present in the template sequence, multiple nucleotides will be incorporated in a single cycle. This leads to a corresponding number of released hydrogens and a proportionally higher electronic signal.

The 454™ GS FLX™ sequencing system (Roche, Germany), employs a light-based detection methodology in a large-scale parallel pyrosequencing system. Pyrosequencing uses DNA polymerization, adding one nucleotide species at a time and detecting and quantifying the number of nucleotides added to a given location through the light emitted by the release of attached pyrophosphates. For use with the 454™ system, adapter-ligated DNA fragments are fixed to small DNA-capture beads in a water-in-oil emulsion and amplified by PCR (emulsion PCR). Each DNA-bound bead is placed into a well on a picotiter plate and sequencing reagents are delivered across the wells of the plate. The four DNA nucleotides are added sequentially in a fixed order across the picotiter plate device during a sequencing run. During the nucleotide flow, millions of copies of DNA bound to each of the beads are sequenced in parallel. When a nucleotide complementary to the template strand is added to a well, the nucleotide is incorporated onto the existing DNA strand, generating a light signal that is recorded by a CCD camera in the instrument.

Sequencing technology based on reversible dye-terminators: DNA molecules are first attached to primers on a slide and amplified so that local clonal colonies are formed. Four types of reversible terminator bases (RT-bases) are added, and non-incorporated nucleotides are washed away. Unlike pyrosequencing, the DNA can only be extended one nucleotide at a time. A camera takes images of the fluorescently labeled nucleotides, then the dye along with the terminal 3′ blocker is chemically removed from the DNA, allowing the next cycle.

Helicos's single-molecule sequencing uses DNA fragments with added polyA tail adapters, which are attached to the flow cell surface. At each cycle, DNA polymerase and a single species of fluorescently labeled nucleotide are added, resulting in template-dependent extension of the surface-immobilized primer-template duplexes. The reads are performed by the Helioscope sequencer. After acquisition of images tiling the full array, chemical cleavage and release of the fluorescent label permits the subsequent cycle of extension and imaging.

Sequencing by synthesis (SBS), like the “old style” dye-termination electrophoretic sequencing, relies on incorporation of nucleotides by a DNA polymerase to determine the base sequence. A DNA library with affixed adapters is denatured into single strands and grafted to a flow cell, followed by bridge amplification to form a high-density array of spots onto a glass chip. Reversible terminator methods use reversible versions of dye-terminators, adding one nucleotide at a time, detecting fluorescence at each position by repeated removal of the blocking group to allow polymerization of another nucleotide. The signal of nucleotide incorporation can vary with fluorescently labeled nucleotides, phosphate-driven light reactions and hydrogen ion sensing having all been used. Examples of SBS platforms include Illumina GA and HiSeq 2000. The MiSeq® personal sequencing system (Illumina, Inc.) also employs sequencing by synthesis with reversible terminator chemistry.

In contrast to the sequencing by synthesis method, the sequencing by ligation method uses a DNA ligase to determine the target sequence. This sequencing method relies on enzymatic ligation of oligonucleotides that are adjacent through local complementarity on a template DNA strand. This technology employs a partition of all possible oligonucleotides of a fixed length, labeled according to the sequenced position. Oligonucleotides are annealed and ligated and the preferential ligation by DNA ligase for matching sequences results in a dinucleotide encoded color space signal at that position (through the release of a fluorescently labeled probe that corresponds to a known nucleotide at a known position along the oligo). This method is primarily used by Life Technologies' SOLID™ sequencers. Before sequencing, the DNA is amplified by emulsion PCR. The resulting beads, each containing only copies of the same DNA molecule, are deposited on a solid planar substrate.

SMRT™ sequencing is based on the sequencing by synthesis approach. The DNA is synthesized in zero-mode wave-guides (ZMWs)-small well-like containers with the capturing tools located at the bottom of the well. The sequencing is performed with use of unmodified polymerase (attached to the ZMW bottom) and fluorescently labeled nucleotides flowing freely in the solution. The wells are constructed in a way that only the fluorescence occurring at the bottom of the well is detected. The fluorescent label is detached from the nucleotide at its incorporation into the DNA strand, leaving an unmodified DNA strand.

Methods for Predicting the Risk of VTE Using ctDNA as a Biomarker

### Pan Cancer

In one aspect, the present disclosure provides a method for preventing cancer associated thromboembolism (CAT) in a cancer patient in need thereof comprising (a) detecting ctDNA molecules in a biological sample obtained from the cancer patient, wherein the ctDNA molecules are detected at a variant allele fraction (VAF) detection limit of at least 0.1%-0.5% and (b) administering to the cancer patient an effective amount of anticoagulant therapy.

In another aspect, the present disclosure provides a method for preventing cancer associated thromboembolism (CAT) in a cancer patient in need thereof comprising administering to the cancer patient an effective amount of anticoagulant therapy, wherein a biological sample obtained from the cancer patient comprises detectable ctDNA molecules, wherein the ctDNA molecules are detected at a variant allele fraction (VAF) detection limit of at least 0.1%-0.5%.

Additionally or alternatively, in some embodiments of the methods disclosed herein, the ctDNA molecules are detected at a VAF detection limit of from about 0.1% to about 0.5%, from about 0.5% to about 2%, from about 2% to about 10% or from about 10% to about 99%. In certain embodiments, the ctDNA molecules are detected at a VAF detection limit of about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%.

In any of the preceding embodiments of the methods disclosed herein, the cancer patient is diagnosed with or suffers from a cancer selected from the group consisting of non-small cell lung cancer, breast cancer, pancreatic cancer, melanoma, retinoblastoma, prostate cancer, esophagogastric cancer, histiocytosis, germ cell tumor, endometrial cancer, small cell lung cancer, soft tissue sarcoma, Gastrointestinal Stromal Tumor, ovarian cancer, mature B-Cell neoplasms, small bowel cancer, renal cell carcinoma, thyroid cancer, ampullary cancer, appendiceal cancer, sellar tumor, uterine sarcoma, bone cancer, non-melanoma skin cancer, cervical cancer, mesothelioma, glioma, thymic tumor, gastrointestinal neuroendocrine tumor, salivary gland cancer, sex cord stromal tumor, anal cancer, mature T and NK neoplasms, peritoneal cancer, Head and neck cancer, choroid plexus tumor, leukemia, primary CNS melanocytic tumors, Myelodysplastic Syndromes, Peripheral Nervous System, mastocytosis, Wilms tumor, lymphatic cancer, vaginal cancer, Hodgkin lymphoma, adrenocortical carcinoma, brain tumors, embryonal tumors and Non-Hodgkin lymphoma. The cancer may be a Stage 1, Stage 2, Stage 3, or Stage 4 cancer. Additionally or alternatively, in some embodiments, the cancer patient has a Khorana Score ≥2 or <2 and/or has one or more organ sites of metastasis.

Additionally or alternatively, in some embodiments of the methods disclosed herein, the ctDNA molecules comprise one or more mutations (e.g., SNVs) in at least one cancer associated gene selected from the group consisting of AKT1, ALK, APC, AR, ARAF, ARIDIA, ARID2, ATM, B2M, BCL2, BCOR, BRAF, BRCA1, BRCA2, CARD11, CBFB, CCND1, CDH1, CDK4, CDKN2A, CIC, CREBBP, CTCF, CTNNB1, DICER1, DIS3, DNMT3A, EGFR, EIF1AX, EP300, ERBB2, ERBB3, ERCC2, ESR1, EZH2, FBXW7, FGFR1, FGFR2, FGFR3, FGFR4, FLT3, FOXA1, FOXL2, FOXO1, FUBP1, GATA3, GNA11, GNAQ, GNAS, H3F3A, HIST1H3B, HRAS, IDH1, IDH2, IKZF1, INPPL1, JAK1, KDM6A, KEAP1, KIT, KNSTRN, KRAS, MAP2K1, MAPK1, MAX, MED12, MET, MLH1, MSH2, MSH3, MSH6, MTOR, MYC, MYCN, MYD88, MYOD1, NF1, NFE2L2, NOTCH1, NRAS, NTRK1, NTRK2, NTRK3, NUP93, PAK7, PDGFRA, PIK3CA, PIK3CB, PIK3R1, PIK3R2, PMS2, POLE, PPP2RIA, PPP6C, PRKCI, PTCH1, PTEN, PTPN11, RAC1, RAF1, RB1, RET, RHOA, RIT1, ROS1, RRAS2, RXRA, SETD2, SF3B1, SMAD3, SMAD4, SMARCA4, SMARCB1, SOS1, SPOP, STAT3, STK11, STK19, TCF7L2, TGFBR1, TGFBR2, TP53, TP63, TSC1, TSC2, U2AF1, VHL, XPO1, and TERT. In certain embodiments, the ctDNA molecules comprise 2-20 mutations in the at the least one cancer associated gene.

In any and all embodiments of the methods disclosed herein, the ctDNA molecules comprise one or more rearrangements in at least one cancer associated gene selected from the group consisting of ALK, BRAF, EGFR, ETV6, FGFR2, FGFR3, MET, NTRK1, RET and ROS1. The one or more rearrangements may comprise indels, CNVs, and/or gene fusions. Additionally or alternatively, in some embodiments, the ctDNA molecules comprise 2-20 rearrangements in the at the least one cancer associated gene.

In any of the preceding embodiments of the methods disclosed herein, the biological sample is whole blood, serum or plasma. In some embodiments, the biological sample has a cfDNA concentration ranging from about 3 pg/μL to 5.5 ng/μL. In some embodiments, the biological sample has a cfDNA concentration of about 3 pg/μL, about 4 pg/μL, about 5 pg/μL, about 6 pg/μL, about 7 pg/μL, about 8 pg/μL, about 9 pg/μL, about 10 pg/μL, about 15 pg/μL, about 20 pg/μL, about 25 pg/μL, about 30 pg/μL, about 35 pg/μL, about 40 pg/μL, about 45 pg/μL, about 50 pg/μL, about 55 pg/μL, about 60 pg/μL, about 65 pg/μL, about 70 pg/μL, about 75 pg/μL, about 80 pg/μL, about 85 pg/μL, about 90 pg/μL, about 100 pg/μL, about 125 pg/μL, about 150 pg/μL, about 175 pg/μL, about 200 pg/μL, about 225 pg/μL, about 250 pg/μL, about 275 pg/μL, about 300 pg/μL, about 325 pg/μL, about 350 pg/μL, about 375 pg/μL, about 400 pg/μL, about 425 pg/μL, about 450 pg/μL, about 475 pg/μL, about 500 pg/μL, about 525 pg/μL, about 550 pg/μL, about 575 pg/μL, about 600 pg/μL, about 625 pg/μL, about 650 pg/μL, about 675 pg/μL, about 700 pg/μL, about 725 pg/μL, about 750 pg/μL, about 775 pg/μL, about 800 pg/μL, about 825 pg/μL, about 850 pg/μL, about 875 pg/μL, about 900 pg/μL, about 925 pg/μL, about 950 pg/μL, about 975 pg/μL, about 1 ng/μL, about 1.25 ng/μL, about 1.5 ng/μL, about 1.75 ng/μL, about 2 ng/μL, about 2.25 ng/μL, about 2.5 ng/μL, about 2.75 ng/μL, about 3 ng/μL, about 3.25 ng/μL, about 3.5 ng/μL, about 3.75 ng/μL, about 4 ng/μL, about 4.25 ng/μL, about 4.5 ng/μL, about 4.75 ng/μL, about 5 ng/μL, about 5.25 ng/μL, or about 5.5 ng/μL.

Additionally or alternatively, in some embodiments, the anticoagulant therapy comprises one or more of apixaban, betrixaban, dabigatran, edoxaban, fondaparinux, heparin, rivaroxaban, warfarin, Xa inhibitors, statins, or enoxaparin. Examples of statins include, but are not limited to atorvastatin, fluvastatin, lovastatin, pitavastatin, pravastatin, rosuvastatin, and simvastatin.

In any of the foregoing embodiments of the methods disclosed herein, the cancer patient is chemotherapy-naïve or has received/is receiving systemic chemotherapy. Systemic chemotherapy may comprise one or more of alkylating agents, antibiotics, antimetabolites, antimitotics, cyclin-dependent kinase inhibitors, epidermal growth factor receptor inhibitors, multikinase inhibitors, PARP inhibitors, platinum-based agents, selective estrogen receptor modulators (SERM), or VEGF inhibitors. Examples of chemotherapeutic agents include, but are not limited to, alkylating agents, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, bisphosphonate therapy agents and targeted biological therapy agents (e.g., therapeutic peptides described in U.S. Pat. No. 6,306,832, WO 2012007137, WO 2005000889, WO 2010096603 etc.). In some embodiments, the at least one additional therapeutic agent is a chemotherapeutic agent. Specific chemotherapeutic agents include, but are not limited to, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), bevacizumab, oxaliplatin, melphalan, etoposide, mechlorethamine, bleomycin, microtubule poisons, annonaceous acetogenins, or combinations thereof.

Additionally or alternatively, in some embodiments of the methods disclosed herein, the cancer patient is immunotherapy-naïve or has received/is receiving immunotherapy. Examples of immunotherapy include, but are not limited to, anti-PD-1 antibody, anti-PD-L1 antibody, anti-PD-L2 antibody, anti-CTLA-4 antibody, anti-TIM3 antibody, anti-4-1BB antibody, anti-CD73 antibody, anti-GITR antibody, and anti-LAG-3 antibody.

Additionally or alternatively, in certain embodiments of the methods disclosed herein, the cancer patient is radiotherapy-naïve or has received/is receiving radiotherapy. The radiotherapy may comprise external radiotherapy, radiotherapy implants (brachytherapy), pre-targeted radioimmunotherapy, radiotherapy injections, radioisotope therapy, or intrabeam radiotherapy.

In any and all embodiments of the methods disclosed herein, the CAT is pulmonary embolism or lower extremity deep vein thrombosis (DVT). In some embodiments, lower extremity DVT includes thrombi involving a common iliac vein, an external iliac vein, a common femoral vein, a superficial femoral vein, a deep femoral vein, a popliteal vein, a peroneal vein, an anterior tibial vein, a posterior tibial vein, or a deep calf vein.

### Lung Cancer

In one aspect, the present disclosure provides a method for preventing cancer associated thromboembolism (CAT) in a lung cancer patient in need thereof comprising detecting ctDNA molecules in a biological sample obtained from the lung cancer patient, wherein the ctDNA molecules comprise at least one alteration in at least one cancer-associated gene selected from the group consisting of AKT1, ALK, B2M, BRAF, EGFR, ERBB2 (HER2), FGFR2, FGFR3, KEAP1, KRAS, MAP2K1 (MEK1), MET, NRAS, PIK3CA, RET, ROS1, STK11, TP53, NTRK1, FGFR1, MYC, PTEN, and RICTOR; and administering to the lung cancer patient an effective amount of anticoagulant therapy. The lung cancer may be non-small cell lung cancer (NSCLC) or small cell lung cancer (SCLC). In some embodiments, the lung cancer is Stage 1, Stage 2, Stage 3, or Stage 4.

In another aspect, the present disclosure provides a method for preventing cancer associated thromboembolism (CAT) in a lung cancer patient in need thereof comprising administering to the lung cancer patient an effective amount of anticoagulant therapy, wherein a biological sample obtained from the lung cancer patient comprises detectable ctDNA molecules comprising at least one alteration in at least one cancer-associated gene selected from the group consisting of AKT1, ALK, B2M, BRAF, EGFR, ERBB2 (HER2), FGFR2, FGFR3, KEAP1, KRAS, MAP2K1 (MEK1), MET, NRAS, PIK3CA, RET, ROS1, STK11, TP53, NTRK1, FGFR1, MYC, PTEN, and RICTOR. The lung cancer may be non-small cell lung cancer (NSCLC) or small cell lung cancer (SCLC). In certain embodiments, the lung cancer is Stage 1, Stage 2, Stage 3, or Stage 4.

Additionally or alternatively, in some embodiments, the anticoagulant therapy comprises one or more of apixaban, betrixaban, dabigatran, edoxaban, fondaparinux, heparin, rivaroxaban, warfarin, Xa inhibitors, statins, or enoxaparin. Examples of statins include, but are not limited to atorvastatin, fluvastatin, lovastatin, pitavastatin, pravastatin, rosuvastatin, and simvastatin.

In any of the preceding embodiments of the methods disclosed herein, the lung cancer patient has a Khorana Score <2 or ≥2. Additionally or alternatively, in certain embodiments, the at least one alteration is a SNV, an indel, a CNV, or a gene fusion.

Additionally or alternatively, in some embodiments of the methods disclosed herein, the at least one alteration is detected at a variant allele fraction (VAF) detection limit of 0.1%-0.5%. In certain embodiments, the detected ctDNA molecules comprise one alteration in the at the least one cancer associated gene. In other embodiments, the detected ctDNA molecules comprise 2-20 alterations in the at the least one cancer associated gene. Additionally or alternatively, in some embodiments of the methods disclosed herein, the ctDNA molecules are detected via polymerase chain reaction (PCR), real-time quantitative PCR (qPCR), droplet digital PCR (ddPCR), Reverse transcriptase-PCR (RT-PCR), microarray, RNA-Seq, or next-generation sequencing. In any of the preceding embodiments of the methods disclosed herein, the biological sample is whole blood, serum or plasma.

In any of the foregoing embodiments of the methods disclosed herein, the lung cancer patient is chemotherapy-naïve or has received/is receiving systemic chemotherapy. Systemic chemotherapy may comprise one or more of alkylating agents, antibiotics, antimetabolites, antimitotics, cyclin-dependent kinase inhibitors, epidermal growth factor receptor inhibitors, multikinase inhibitors, PARP inhibitors, platinum-based agents, selective estrogen receptor modulators (SERM), or VEGF inhibitors. Examples of chemotherapeutic agents include, but are not limited to, alkylating agents, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, bisphosphonate therapy agents and targeted biological therapy agents (e.g., therapeutic peptides described in U.S. Pat. No. 6,306,832, WO 2012007137, WO 2005000889, WO 2010096603 etc.). In some embodiments, the at least one additional therapeutic agent is a chemotherapeutic agent. Specific chemotherapeutic agents include, but are not limited to, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), bevacizumab, oxaliplatin, melphalan, etoposide, mechlorethamine, bleomycin, microtubule poisons, annonaceous acetogenins, or combinations thereof.

Additionally or alternatively, in some embodiments of the methods disclosed herein, the lung cancer patient is immunotherapy-naïve or has received/is receiving immunotherapy. Examples of immunotherapy include, but are not limited to, anti-PD-1 antibody, anti-PD-L1 antibody, anti-PD-L2 antibody, anti-CTLA-4 antibody, anti-TIM3 antibody, anti-4-1BB antibody, anti-CD73 antibody, anti-GITR antibody, and anti-LAG-3 antibody.

Additionally or alternatively, in certain embodiments of the methods disclosed herein, the lung cancer patient is radiotherapy-naïve or has received/is receiving radiotherapy. The radiotherapy may comprise external radiotherapy, radiotherapy implants (brachytherapy), pre-targeted radioimmunotherapy, radiotherapy injections, radioisotope therapy, or intrabeam radiotherapy.

In any and all embodiments of the methods disclosed herein, the CAT is pulmonary embolism or lower extremity deep vein thrombosis (DVT). In some embodiments, lower extremity DVT includes thrombi involving a common iliac vein, an external iliac vein, a common femoral vein, a superficial femoral vein, a deep femoral vein, a popliteal vein, a peroneal vein, an anterior tibial vein, a posterior tibial vein, or a deep calf vein.

Additionally or alternatively, in certain embodiments of the methods disclosed herein, the at least one alteration comprises a SNV and/or an indel in one or more of AKT1, ALK, B2M, BRAF, EGFR, ERBB2 (HER2), FGFR2, FGFR3, KEAP1, KRAS, MAP2K1 (MEK1), MET, NRAS, PIK3CA, RET, ROS1, STK11 and TP53. In some embodiments of the methods disclosed herein, the at least one alteration comprises a gene fusion in one or more of ALK, EGFR, FGFR2, FGFR3, NTRK1, RET, and ROS1. Additionally or alternatively, in some embodiments, the at least one alteration comprises a CNV in one or more of B2M, EGFR, ERBB2 (HER2), FGFR1, KRAS, MET, MYC, NTRK1, PIK3CA, PTEN, RICTOR, STK11, and TP53.

### Systems, Devices, and Methods for Predicting the Risk of VTE Across Multiple Cancer Types

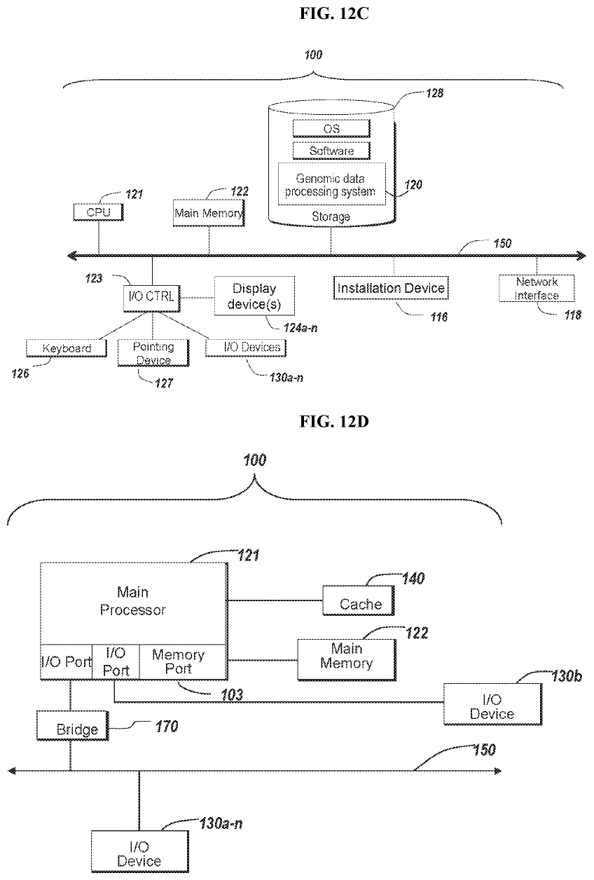

Aspects of the operating environment as well as associated system components (e.g., hardware elements) in connection with various embodiments of the methods and systems described herein will now be discussed. Referring to FIG. 12A, an embodiment of a network environment is depicted. In brief overview, the network environment includes one or more clients 102a-102n (also generally referred to as local machine(s) 102, client(s) 102, client node(s) 102, client machine(s) 102, client computer(s) 102, client device(s) 102, endpoint(s) 102, or endpoint node(s) 102) in communication with one or more servers 106a-106n (also generally referred to as server(s) 106, node 106, or remote machine(s) 106) via one or more networks 104. In some embodiments, a client 102 has the capacity to function as both a client node seeking access to resources provided by a server and as a server providing access to hosted resources for other clients 102a-102n.

Although FIG. 12A shows a network 104 between the clients 102 and the servers 106, the clients 102 and the servers 106 may be on the same network 104. In some embodiments, there are multiple networks 104 between the clients 102 and the servers 106. In one of these embodiments, a network 104′ (not shown) may be a private network and a network 104 may be a public network. In another of these embodiments, a network 104 may be a private network and a network 104′ a public network. In still another of these embodiments, networks 104 and 104′ may both be private networks.

The network 104 may be connected via wired or wireless links. Wired links may include Digital Subscriber Line (DSL), coaxial cable lines, or optical fiber lines. The wireless links may include BLUETOOTH, Wi-Fi, Worldwide Interoperability for Microwave Access (WiMAX), an infrared channel or satellite band. The wireless links may also include any cellular network standards used to communicate among mobile devices, including standards that qualify as 1G, 2G, 3G, 4G, or 5G. The network standards may qualify as one or more generation of mobile telecommunication standards by fulfilling a specification or standards such as the specifications maintained by International Telecommunication Union. The 3G standards, for example, may correspond to the International Mobile Telecommunications-2000 (IMT-2000) specification, and the 4G standards may correspond to the International Mobile Telecommunications Advanced (IMT-Advanced) specification. Examples of cellular network standards include AMPS, GSM, GPRS, UMTS, LTE, LTE Advanced, Mobile WiMAX, and WiMAX-Advanced. Cellular network standards may use various channel access methods e.g. FDMA, TDMA, CDMA, or SDMA. In some embodiments, different types of data may be transmitted via different links and standards. In other embodiments, the same types of data may be transmitted via different links and standards.

The network 104 may be any type and/or form of network. The geographical scope of the network 104 may vary widely and the network 104 can be a body area network (BAN), a personal area network (PAN), a local-area network (LAN), e.g. Intranet, a metropolitan area network (MAN), a wide area network (WAN), or the Internet. The topology of the network 104 may be of any form and may include, e.g., any of the following: point-to-point, bus, star, ring, mesh, or tree. The network 104 may be an overlay network which is virtual and sits on top of one or more layers of other networks 104′. The network 104 may be of any such network topology as known to those ordinarily skilled in the art capable of supporting the operations described herein. The network 104 may utilize different techniques and layers or stacks of protocols, including, e.g., the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including, e.g., IPV6), or the link layer. The network 104 may be a type of a broadcast network, a telecommunications network, a data communication network, or a computer network.

In some embodiments, the system may include multiple, logically-grouped servers 106. In one of these embodiments, the logical group of servers may be referred to as a server farm 38 or a machine farm 38. In another of these embodiments, the servers 106 may be geographically dispersed. In other embodiments, a machine farm 38 may be administered as a single entity. In still other embodiments, the machine farm 38 includes a plurality of machine farms 38. The servers 106 within each machine farm 38 can be heterogeneous-one or more of the servers 106 or machines 106 can operate according to one type of operating system platform (e.g., WINDOWS NT, manufactured by Microsoft Corp. of Redmond, Washington), while one or more of the other servers 106 can operate on according to another type of operating system platform (e.g., Unix, Linux, or Mac OS X).

In one embodiment, servers 106 in the machine farm 38 may be stored in high-density rack systems, along with associated storage systems, and located in an enterprise data center. In this embodiment, consolidating the servers 106 in this way may improve system manageability, data security, the physical security of the system, and system performance by locating servers 106 and high performance storage systems on localized high performance networks. Centralizing the servers 106 and storage systems and coupling them with advanced system management tools allows more efficient use of server resources.

The servers 106 of each machine farm 38 do not need to be physically proximate to another server 106 in the same machine farm 38. Thus, the group of servers 106 logically grouped as a machine farm 38 may be interconnected using a wide-area network (WAN) connection or a metropolitan-area network (MAN) connection. For example, a machine farm 38 may include servers 106 physically located in different continents or different regions of a continent, country, state, city, campus, or room. Data transmission speeds between servers 106 in the machine farm 38 can be increased if the servers 106 are connected using a local-area network (LAN) connection or some form of direct connection. Additionally, a heterogeneous machine farm 38 may include one or more servers 106 operating according to a type of operating system, while one or more other servers 106 execute one or more types of hypervisors rather than operating systems. In these embodiments, hypervisors may be used to emulate virtual hardware, partition physical hardware, virtualize physical hardware, and execute virtual machines that provide access to computing environments, allowing multiple operating systems to run concurrently on a host computer. Native hypervisors may run directly on the host computer. Hypervisors may include VMware ESX/ESXi, manufactured by VMWare, Inc., of Palo Alto, California; the Xen hypervisor, an open source product whose development is overseen by Citrix Systems, Inc.; the HYPER-V hypervisors provided by Microsoft or others. Hosted hypervisors may run within an operating system on a second software level. Examples of hosted hypervisors may include VMware Workstation and VIRTUALBOX.

Management of the machine farm 38 may be de-centralized. For example, one or more servers 106 may comprise components, subsystems and modules to support one or more management services for the machine farm 38. In one of these embodiments, one or more servers 106 provide functionality for management of dynamic data, including techniques for handling failover, data replication, and increasing the robustness of the machine farm 38. Each server 106 may communicate with a persistent store and, in some embodiments, with a dynamic store.

Server 106 may be a file server, application server, web server, proxy server, appliance, network appliance, gateway, gateway server, virtualization server, deployment server, SSL VPN server, or firewall. In one embodiment, the server 106 may be referred to as a remote machine or a node. In another embodiment, a plurality of nodes 290 may be in the path between any two communicating servers.

Referring to FIG. 12B, a cloud computing environment is depicted. A cloud computing environment may provide client 102 with one or more resources provided by a network environment. The cloud computing environment may include one or more clients 102a-102n, in communication with the cloud 108 over one or more networks 104. Clients 102 may include, e.g., thick clients, thin clients, and zero clients. A thick client may provide at least some functionality even when disconnected from the cloud 108 or servers 106. A thin client or a zero client may depend on the connection to the cloud 108 or server 106 to provide functionality. A zero client may depend on the cloud 108 or other networks 104 or servers 106 to retrieve operating system data for the client device. The cloud 108 may include back end platforms, e.g., servers 106, storage, server farms or data centers.

The cloud 108 may be public, private, or hybrid. Public clouds may include public servers 106 that are maintained by third parties to the clients 102 or the owners of the clients. The servers 106 may be located off-site in remote geographical locations as disclosed above or otherwise. Public clouds may be connected to the servers 106 over a public network. Private clouds may include private servers 106 that are physically maintained by clients 102 or owners of clients. Private clouds may be connected to the servers 106 over a private network 104. Hybrid clouds 108 may include both the private and public networks 104 and servers 106.

The cloud 108 may also include a cloud based delivery, e.g. Software as a Service (SaaS) 110, Platform as a Service (PaaS) 112, and Infrastructure as a Service (IaaS) 114. IaaS may refer to a user renting the use of infrastructure resources that are needed during a specified time period. IaaS providers may offer storage, networking, servers or virtualization resources from large pools, allowing the users to quickly scale up by accessing more resources as needed. Examples of IaaS can include infrastructure and services (e.g., EG-32) provided by OVH HOSTING of Montreal, Quebec, Canada, AMAZON WEB SERVICES provided by Amazon.com, Inc., of Seattle, Washington, RACKSPACE CLOUD provided by Rackspace US, Inc., of San Antonio, Texas, Google Compute Engine provided by Google Inc. of Mountain View, California, or RIGHTSCALE provided by RightScale, Inc., of Santa Barbara, California. PaaS providers may offer functionality provided by IaaS, including, e.g., storage, networking, servers or virtualization, as well as additional resources such as, e.g., the operating system, middleware, or runtime resources. Examples of PaaS include WINDOWS AZURE provided by Microsoft Corporation of Redmond, Washington, Google App Engine provided by Google Inc., and HEROKU provided by Heroku, Inc. of San Francisco, California. SaaS providers may offer the resources that PaaS provides, including storage, networking, servers, virtualization, operating system, middleware, or runtime resources. In some embodiments, SaaS providers may offer additional resources including, e.g., data and application resources. Examples of SaaS include GOOGLE APPS provided by Google Inc., SALESFORCE provided by Salesforce.com Inc. of San Francisco, California, or OFFICE 365 provided by Microsoft Corporation. Examples of SaaS may also include data storage providers, e.g. DROPBOX provided by Dropbox, Inc. of San Francisco, California, Microsoft SKYDRIVE provided by Microsoft Corporation, Google Drive provided by Google Inc., or Apple ICLOUD provided by Apple Inc. of Cupertino, California.

Clients 102 may access IaaS resources with one or more IaaS standards, including, e.g., Amazon Elastic Compute Cloud (EC2), Open Cloud Computing Interface (OCCI), Cloud Infrastructure Management Interface (CIMI), or OpenStack standards. Some IaaS standards may allow clients access to resources over HTTP, and may use Representational State Transfer (REST) protocol or Simple Object Access Protocol (SOAP). Clients 102 may access PaaS resources with different PaaS interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that may be built on REST, HTTP, XML, or other protocols. Clients 102 may access SaaS resources through the use of web-based user interfaces, provided by a web browser (e.g. GOOGLE CHROME, Microsoft INTERNET EXPLORER, or Mozilla Firefox provided by Mozilla Foundation of Mountain View, California). Clients 102 may also access SaaS resources through smartphone or tablet applications, including, e.g., Salesforce Sales Cloud, or Google Drive app. Clients 102 may also access SaaS resources through the client operating system, including, e.g., Windows file system for DROPBOX.

In some embodiments, access to IaaS, PaaS, or SaaS resources may be authenticated. For example, a server or authentication server may authenticate a user via security certificates, HTTPS, or API keys. API keys may include various encryption standards such as, e.g., Advanced Encryption Standard (AES). Data resources may be sent over Transport Layer Security (TLS) or Secure Sockets Layer (SSL).

The client 102 and server 106 may be deployed as and/or executed on any type and form of computing device, e.g. a computer, network device or appliance capable of communicating on any type and form of network and performing the operations described herein. FIGS. 12C and 12D depict block diagrams of a computing device 100 useful for practicing an embodiment of the client 102 or a server 106. As shown in FIGS. 12C and 12D, each computing device 100 includes a central processing unit 121, and a main memory unit 122. As shown in FIG. 12C, a computing device 100 may include a storage device 128, an installation device 116, a network interface 118, an I/O controller 123, display devices 124a-124n, a keyboard 126 and a pointing device 127, e.g. a mouse. The storage device 128 may include, without limitation, an operating system, software, and a software of a genomic data processing system 120. As shown in FIG. 12D, each computing device 100 may also include additional optional elements, e.g. a memory port 103, a bridge 170, one or more input/output devices 130a-130n (generally referred to using reference numeral 130), and a cache memory 140 in communication with the central processing unit 121.

The central processing unit 121 is any logic circuitry that responds to and processes instructions fetched from the main memory unit 122. In many embodiments, the central processing unit 121 is provided by a microprocessor unit, e.g.: those manufactured by Intel Corporation of Mountain View, California; those manufactured by Motorola Corporation of Schaumburg, Illinois; the ARM processor and TEGRA system on a chip (SoC) manufactured by Nvidia of Santa Clara, California; the POWER7 processor, those manufactured by International Business Machines of White Plains, New York; or those manufactured by Advanced Micro Devices of Sunnyvale, California. The computing device 100 may be based on any of these processors, or any other processor capable of operating as described herein. The central processing unit 121 may utilize instruction level parallelism, thread level parallelism, different levels of cache, and multi-core processors. A multi-core processor may include two or more processing units on a single computing component. Examples of multi-core processors include the AMD PHENOM IIX2, INTEL CORE i5 and INTEL CORE i7.

Main memory unit or memory device 122 may include one or more memory chips capable of storing data and allowing any storage location to be directly accessed by the microprocessor 121. Main memory unit or device 122 may be volatile and faster than storage 128 memory. Main memory units or devices 122 may be Dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 122 or the storage 128 may be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 122 may be based on any of the above described memory chips, or any other available memory chips capable of operating as described herein. In the embodiment shown in FIG. 12C, the processor 121 communicates with main memory 122 via a system bus 150 (described in more detail below). FIG. 12D depicts an embodiment of a computing device 100 in which the processor communicates directly with main memory 122 via a memory port 103. For example, in FIG. 12D the main memory 122 may be DRDRAM.

FIG. 12D depicts an embodiment in which the main processor 121 communicates directly with cache memory 140 via a secondary bus, sometimes referred to as a backside bus. In other embodiments, the main processor 121 communicates with cache memory 140 using the system bus 150. Cache memory 140 typically has a faster response time than main memory 122 and is typically provided by SRAM, BSRAM, or EDRAM. In the embodiment shown in FIG. 12D, the processor 121 communicates with various I/O devices 130 via a local system bus 150. Various buses may be used to connect the central processing unit 121 to any of the I/O devices 130, including a PCI bus, a PCI-X bus, or a PCI-Express bus, or a NuBus. For embodiments in which the I/O device is a video display 124, the processor 121 may use an Advanced Graphics Port (AGP) to communicate with the display 124 or the I/O controller 123 for the display 124. FIG. 12D depicts an embodiment of a computer 100 in which the main processor 121 communicates directly with I/O device 130b or other processors 121′ via HYPERTRANSPORT, RAPIDIO, or INFINIBAND communications technology. FIG. 12D also depicts an embodiment in which local busses and direct communication are mixed: the processor 121 communicates with I/O device 130a using a local interconnect bus while communicating with I/O device 130b directly.

A wide variety of I/O devices 130a-130n may be present in the computing device 100. Input devices may include keyboards, mice, trackpads, trackballs, touchpads, touch mice, multi-touch touchpads and touch mice, microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. Output devices may include video displays, graphical displays, speakers, headphones, inkjet printers, laser printers, and 3D printers.

Devices 130a-130n may include a combination of multiple input or output devices, including, e.g., Microsoft KINECT, Nintendo Wiimote for the WII, Nintendo WII U GAMEPAD, or Apple IPHONE. Some devices 130a-130n allow gesture recognition inputs through combining some of the inputs and outputs. Some devices 130a-130n provides for facial recognition which may be utilized as an input for different purposes including authentication and other commands. Some devices 130a-130n provides for voice recognition and inputs, including, e.g., Microsoft KINECT, SIRI for IPHONE by Apple, Google Now or Google Voice Search.

Additional devices 130a-130n have both input and output capabilities, including, e.g., haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, may have larger surfaces, such as on a table-top or on a wall, and may also interact with other electronic devices. Some I/O devices 130a-130n, display devices 124a-124n or group of devices may be augment reality devices. The I/O devices may be controlled by an I/O controller 123 as shown in FIG. 12C. The I/O controller may control one or more I/O devices, such as, e.g., a keyboard 126 and a pointing device 127, e.g., a mouse or optical pen. Furthermore, an I/O device may also provide storage and/or an installation medium 116 for the computing device 100. In still other embodiments, the computing device 100 may provide USB connections (not shown) to receive handheld USB storage devices. In further embodiments, an I/O device 130 may be a bridge between the system bus 150 and an external communication bus, e.g. a USB bus, a SCSI bus, a FireWire bus, an Ethernet bus, a Gigabit Ethernet bus, a Fibre Channel bus, or a Thunderbolt bus.

In some embodiments, display devices 124a-124n may be connected to I/O controller 123. Display devices may include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays may use, e.g. stereoscopy, polarization filters, active shutters, or autostereoscopy. Display devices 124a-124n may also be a head-mounted display (HMD). In some embodiments, display devices 124a-124n or the corresponding I/O controllers 123 may be controlled through or have hardware support for OPENGL or DIRECTX API or other graphics libraries.

In some embodiments, the computing device 100 may include or connect to multiple display devices 124a-124n, which each may be of the same or different type and/or form. As such, any of the I/O devices 130a-130n and/or the I/O controller 123 may include any type and/or form of suitable hardware, software, or combination of hardware and software to support, enable or provide for the connection and use of multiple display devices 124a-124n by the computing device 100. For example, the computing device 100 may include any type and/or form of video adapter, video card, driver, and/or library to interface, communicate, connect or otherwise use the display devices 124a-124n. In one embodiment, a video adapter may include multiple connectors to interface to multiple display devices 124a-124n. In other embodiments, the computing device 100 may include multiple video adapters, with each video adapter connected to one or more of the display devices 124a-124n. In some embodiments, any portion of the operating system of the computing device 100 may be configured for using multiple displays 124a-124n. In other embodiments, one or more of the display devices 124a-124n may be provided by one or more other computing devices 100a or 100b connected to the computing device 100, via the network 104. In some embodiments software may be designed and constructed to use another computer's display device as a second display device 124a for the computing device 100. For example, in one embodiment, an Apple iPad may connect to a computing device 100 and use the display of the device 100 as an additional display screen that may be used as an extended desktop. One ordinarily skilled in the art will recognize and appreciate the various ways and embodiments that a computing device 100 may be configured to have multiple display devices 124a-124n.

Referring again to FIG. 12C, the computing device 100 may comprise a storage device 128 (e.g. one or more hard disk drives or redundant arrays of independent disks) for storing an operating system or other related software, and for storing application software programs such as any program related to the software for the genomic data processing system 120. Examples of storage device 128 include, e.g., hard disk drive (HDD); optical drive including CD drive, DVD drive, or BLU-RAY drive; solid-state drive (SSD); USB flash drive; or any other device suitable for storing data. Some storage devices may include multiple volatile and non-volatile memories, including, e.g., solid state hybrid drives that combine hard disks with solid state cache. Some storage device 128 may be non-volatile, mutable, or read-only. Some storage device 128 may be internal and connect to the computing device 100 via a bus 150. Some storage devices 128 may be external and connect to the computing device 100 via an I/O device 130 that provides an external bus. Some storage device 128 may connect to the computing device 100 via the network interface 118 over a network 104, including, e.g., the Remote Disk for MACBOOK AIR by Apple. Some client devices 100 may not require a non-volatile storage device 128 and may be thin clients or zero clients 102. Some storage device 128 may also be used as an installation device 116, and may be suitable for installing software and programs. Additionally, the operating system and the software can be run from a bootable medium, for example, a bootable CD, e.g. KNOPPIX, a bootable CD for GNU/Linux that is available as a GNU/Linux distribution from knoppix.net.

Client device 100 may also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc. An application distribution platform may facilitate installation of software on a client device 102. An application distribution platform may include a repository of applications on a server 106 or a cloud 108, which the clients 102a-102n may access over a network 104. An application distribution platform may include application developed and provided by various developers. A user of a client device 102 may select, purchase and/or download an application via the application distribution platform.

Furthermore, the computing device 100 may include a network interface 118 to interface to the network 104 through a variety of connections including, but not limited to, standard telephone lines LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections). In one embodiment, the computing device 100 communicates with other computing devices 100′ via any type and/or form of gateway or tunneling protocol e.g. Secure Socket Layer (SSL) or Transport Layer Security (TLS), or the Citrix Gateway Protocol manufactured by Citrix Systems, Inc. of Ft. Lauderdale, Florida. The network interface 118 may comprise a built-in network adapter, network interface card, PCMCIA network card, EXPRESSCARD network card, card bus network adapter, wireless network adapter, USB network adapter, modem or any other device suitable for interfacing the computing device 100 to any type of network capable of communication and performing the operations described herein.

A computing device 100 of the sort depicted in FIGS. 12B and 12C may operate under the control of an operating system, which controls scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 2000, WINDOWS Server 2022, WINDOWS CE, WINDOWS Phone, WINDOWS XP, WINDOWS VISTA, and WINDOWS 7, WINDOWS RT, WINDOWS 8, and WINDOWS 10, all of which are manufactured by Microsoft Corporation of Redmond, Washington; MAC OS and iOS, manufactured by Apple, Inc. of Cupertino, California; and Linux, a freely-available operating system, e.g. Linux Mint distribution (“distro”) or Ubuntu, distributed by Canonical Ltd. of London, United Kingdom; or Unix or other Unix-like derivative operating systems; and Android, designed by Google, of Mountain View, California, among others. Some operating systems, including, e.g., the CHROME OS by Google, may be used on zero clients or thin clients, including, e.g., CHROMEBOOKS.

The computer system 100 can be any workstation, telephone, desktop computer, laptop or notebook computer, netbook, ULTRABOOK, tablet, server, handheld computer, mobile telephone, smartphone or other portable telecommunications device, media playing device, a gaming system, mobile computing device, or any other type and/or form of computing, telecommunications or media device that is capable of communication. The computer system 100 has sufficient processor power and memory capacity to perform the operations described herein. The computer system 100 can be of any suitable size, such as a standard desktop computer or a Raspberry Pi 4 manufactured by Raspberry Pi Foundation, of Cambridge, United Kingdom. In some embodiments, the computing device 100 may have different processors, operating systems, and input devices consistent with the device. The Samsung GALAXY smartphones, e.g., operate under the control of Android operating system developed by Google, Inc. GALAXY smartphones receive input via a touch interface.

In some embodiments, the computing device 100 is a gaming system. For example, the computer system 100 may comprise a PLAYSTATION 3, or PERSONAL PLAYSTATION PORTABLE (PSP), or a PLAYSTATION VITA device manufactured by the Sony Corporation of Tokyo, Japan, a NINTENDO DS, NINTENDO 3DS, NINTENDO WII, or a NINTENDO WII U device manufactured by Nintendo Co., Ltd., of Kyoto, Japan, an XBOX 360 device manufactured by the Microsoft Corporation of Redmond, Washington.

In some embodiments, the computing device 100 is a digital audio player such as the Apple IPOD, IPOD Touch, and IPOD NANO lines of devices, manufactured by Apple Computer of Cupertino, California. Some digital audio players may have other functionality, including, e.g., a gaming system or any functionality made available by an application from a digital application distribution platform. For example, the IPOD Touch may access the Apple App Store. In some embodiments, the computing device 100 is a portable media player or digital audio player supporting file formats including, but not limited to, MP3, WAV, M4A/AAC, WMA Protected AAC, AIFF, Audible audiobook, Apple Lossless audio file formats and .mov, .m4v, and .mp4 MPEG-4 (H.264/MPEG-4 AVC) video file formats.

In some embodiments, the computing device 100 is a tablet e.g. the IPAD line of devices by Apple; GALAXY TAB family of devices by Samsung; or KINDLE FIRE, by Amazon.com, Inc. of Seattle, Washington. In other embodiments, the computing device 100 is an eBook reader, e.g. the KINDLE family of devices by Amazon.com, or NOOK family of devices by Barnes & Noble, Inc. of New York City, New York.

In some embodiments, the communications device 102 includes a combination of devices, e.g. a smartphone combined with a digital audio player or portable media player. For example, one of these embodiments is a smartphone, e.g. the IPHONE family of smartphones manufactured by Apple, Inc.; a Samsung GALAXY family of smartphones manufactured by Samsung, Inc.; or a Motorola DROID family of smartphones. In yet another embodiment, the communications device 102 is a laptop or desktop computer equipped with a web browser and a microphone and speaker system, e.g. a telephony headset. In these embodiments, the communications devices 102 are web-enabled and can receive and initiate phone calls. In some embodiments, a laptop or desktop computer is also equipped with a webcam or other video capture device that enables video chat and video call.

In some embodiments, the status of one or more machines 102, 106 in the network 104 are monitored, generally as part of network management. In one of these embodiments, the status of a machine may include an identification of load information (e.g., the number of processes on the machine, CPU and memory utilization), of port information (e.g., the number of available communication ports and the port addresses), or of session status (e.g., the duration and type of processes, and whether a process is active or idle). In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

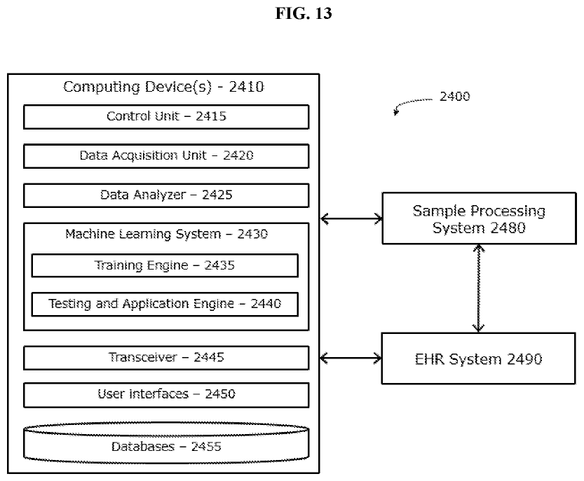

Referring to FIG. 13, in various embodiments, a system 2400 may include a computing device 2410 (or multiple computing devices, co-located or remote to each other), a sample processing system 2480, and an electronic health record (EHR) system 2490. In various embodiments, computing device 2410 (or components thereof) may be integrated with the sample processing system 2480 (or components thereof) and/or EHR system 2490 (or components thereof). In various embodiments, the sample processing system 2480 may include, may be, or may employ, in situ hybridization, PCR, Next-generation sequencing, Northern blotting, microarray, dot or slot blots, FISH, Western blotting, ELISA, colorimetric dye binding assays, complete blood count (CBC) panels, FACs, electrophoresis, chromatography, and/or mass spectroscopy on such biological sample as blood, plasma, serum, and/or tissue and/or Whole-body MRI and PET-CT scans of a subject. For example, in certain embodiments, the sample processing system 2490 may be or may include a Next-generation sequencer. In various embodiments, the EHR system 2490 may include, may be, or may employ, various computing devices that include health records of patients and study subjects (including devices of hospitals, clinics, healthcare practitioners, etc.), obtained from various sources, such as entries by healthcare practitioners, sample processing system 2480, university and hospital systems, government agency systems, etc.

In various embodiments, the computing device 2410 (or multiple computing devices) may be used to control, and receive signals acquired via, components of sample processing system 2480. The computing device 2410 may include one or more processors and one or more volatile and non-volatile memories for storing computing code and data that are captured, acquired, recorded, and/or generated. The computing device 2410 may include a control unit 2415 that in certain embodiments may be configured to exchange control signals with sample processing system 2480, allowing the computing device 2410 to be used to control, for example, processing of samples and/or scans and/or delivery of data generated and/or acquired through processing of samples and/or scans.

In various embodiments, computing device 2410 may include a data acquisition unit 2420 that may be configured to exchange control signals, or otherwise communicate, with sample processing system 2480 (or components thereof) and/or EHR system 2490, allowing the computing device 2410 to be used to control the capture of physiological data and/or signals via sensors of the sample processing system 2480, retrieve data or signals (e.g., from sample processing system 2480, EHR system 2490, and/or memory devices where data is stored), and direct transfer of data or signals (e.g., to sample processing system 2490 as feedback thereto, to EHR system 2490, to memory for storage, and/or to other systems or devices).

In various embodiment, a data analyzer 2425 may direct analysis of the data and signals, and output analysis results. Data analyzer 2425 may be used, for example, to transform raw data captured or obtained via sample processing system 2480 and/or EHR system 2490, and may employ pre-processing procedures involved in generating a training dataset. For example, in some implementations, data may be generated as a multi-dimensional array or vector with values representing, and to prevent the machine learning system from overemphasizing certain readings, values may be normalized to a predetermined range (e.g. 0-1, 0-100, or any other such range). The normalization may comprise linear rescaling, or may be a more complex function. In some implementations, dimension reduction may be performed to reduce large and sparse arrays or vectors. In some implementations, feature recognition may be performed to select a subset of features for further analysis, such as principal component analysis.

In various embodiments, a machine learning system 2430 may be used to implement various machine learning functionality discussed herein. Machine learning system 2430 may include a training engine 2435 configured to train predictive models using, for example, data obtained from or via data acquisition unit 2420 and/or processed data obtained from or via data analyzer 2425. The training engine 2435 may, for example, generate or obtain training datasets from or via data analyzer 2425 and may perform validation of datasets. The training engine 2435 may comprise a feature analyzer used to evaluate features by, for example, quantifying the impact of each feature on the developed model. Such a feature analyzer may, for example, uncover clinically important features that were globally predictive of the outcome, and may determine, for example, contributions of all features, or the top features (e.g., the top 2, top 5, top 10, top 15, top 20, top 25, top 30, etc.) on individual predictions. Features may be selected based on a threshold, such a percent contribution to predicting a medical condition, such as 0.5%, 1%, 2%, 5%, 10%, etc. A testing and application engine 2440 may be configured to test and apply models trained via training engine 2435 to, for example, study subject and/or patient data from data acquisition unit 2420 and/or data analyzer 2425.

In various embodiments, a transceiver 2445 allows the computing device 2410 to exchange readings, control commands, and/or other data with sample processing system 2480 (or components thereof) and/or EHR system 2490 (or components thereof). The transceiver 2445 may additionally or alternatively include a network interface permitting the computing device 2410 to communicate with other remote devices and systems via, for example, a telecommunications network such as the internet. One or more user interfaces 2450 allow the computing device 2410 to receive user inputs (e.g., via a keyboard, touchscreen, microphone, camera, etc.) and provide outputs (e.g., via a touchscreen or other display screen, audio speakers, haptic devices, etc.). A display screen may be employed, for example, to provide real time or near real time waveforms or other readings or measurements obtained via sensors being used to capture physiological data from subjects and patients. The computing device 2410 may additionally include one or more databases 2455 (stored in, e.g., one or more computer-readable non-volatile memory devices) for storing, for example, data and analyses obtained from or via data acquisition unit 2420, data analyzer 2425, machine learning system 2430 (e.g., training engine 2435 and/or testing and application engine 2440), sample processing system 2480, and/or EHR system 2490. In some implementations, database 2455 (or portions thereof) may alternatively or additionally be part of another computing device that is co-located or remote and in communication with computing device 2410, sample processing system 2480 (or components thereof), and/or EHR system 2490.

In one aspect, the present disclosure provides a method of training a machine learning classifier for estimating risk of cancer-associated venous thromboembolism (VTE) in cancer patients comprising: (a) receiving data on a cohort of subjects, the subjects in the cohort having a plurality of cancer types; (b) generating a training dataset based on the received data, wherein the training dataset comprises a plurality of features for each subject in the cohort, wherein the plurality of features comprises (i) cell free DNA concentration, (ii) maximum ctDNA VAF, (iii) ctDNA alterations in at least one cancer associated gene, and (iv) cancer type; and (c) applying a machine learning method to the training dataset to develop the machine learning classifier for estimating risk of cancer-associated VTE in cancer patients, wherein applying the machine learning method comprises: applying a machine learning technique to the training dataset; performing hyperparameter optimization to identify one or more machine learning models with an accuracy that exceeds an accuracy threshold for the classifier; and determining an optimal operating-point threshold based on optimization of sensitivity and specificity of the receiver operating characteristic (ROC) curves for the training dataset; wherein the classifier is configured to receive the plurality of features for cancer patients and generate predictors for risk of cancer-associated VTE in cancer patients. The subjects in the cohort may be chemotherapy-naïve or may have received systemic chemotherapy. Additionally or alternatively, in certain embodiments, the plurality of cancer types are selected from the group consisting of non-small cell lung cancer, breast cancer, pancreatic cancer, melanoma, retinoblastoma, prostate cancer, esophagogastric cancer, histiocytosis, germ cell tumor, endometrial cancer, small cell lung cancer, soft tissue sarcoma, Gastrointestinal Stromal Tumor, ovarian cancer, mature B-Cell neoplasms, small bowel cancer, renal cell carcinoma, thyroid cancer, ampullary cancer, appendiceal cancer, sellar tumor, uterine sarcoma, bone cancer, non-melanoma skin cancer, cervical cancer, mesothelioma, glioma, thymic tumor, gastrointestinal neuroendocrine tumor, salivary gland cancer, sex cord stromal tumor, anal cancer, mature T and NK neoplasms, peritoneal cancer, Head and neck cancer, choroid plexus tumor, leukemia, primary CNS melanocytic tumors, Myelodysplastic Syndromes, Peripheral Nervous System, mastocytosis, Wilms tumor, lymphatic cancer, vaginal cancer, Hodgkin lymphoma, adrenocortical carcinoma, brain tumors, embryonal tumors and Non-Hodgkin lymphoma.

The machine learning technique may model survival outcomes with competing risks. In some embodiments, the machine learning technique is a random forest technique, and the one or more machine learning models are random forest models. Additionally or alternatively, in certain embodiments, the machine learning classifier is an ensemble learning random forest classifier. Additionally or alternatively, in some embodiments, performing the hyperparameter optimization comprises performing an exhaustive grid search technique.

Additionally or alternatively, in some embodiments of the methods disclosed herein, the at least one cancer associated gene is selected from the group consisting of AKT1, ALK, APC, AR, ARAF, ARIDIA, ARID2, ATM, B2M, BCL2, BCOR, BRAF, BRCA1, BRCA2, CARD11, CBFB, CCND1, CDH1, CDK4, CDKN2A, CIC, CREBBP, CTCF, CTNNB1, DICER1, DIS3, DNMT3A, EGFR, EIF1AX, EP300, ERBB2, ERBB3, ERCC2, ESR1, EZH2, FBXW7, FGFR1, FGFR2, FGFR3, FGFR4, FLT3, FOXA1, FOXL2, FOXO1, FUBP1, GATA3, GNA11, GNAQ, GNAS, H3F3A, HIST1H3B, HRAS, IDH1, IDH2, IKZF1, INPPL1, JAK1, KDM6A, KEAP1, KIT, KNSTRN, KRAS, MAP2K1, MAPK1, MAX, MED12, MET, MLH1, MSH2, MSH3, MSH6, MTOR, MYC, MYCN, MYD88, MYOD1, NF1, NFE2L2, NOTCH1, NRAS, NTRK1, NTRK2, NTRK3, NUP93, PAK7, PDGFRA, PIK3CA, PIK3CB, PIK3R1, PIK3R2, PMS2, POLE, PPP2RIA, PPP6C, PRKCI, PTCH1, PTEN, PTPN11, RAC1, RAF1, RB1, RET, RHOA, RIT1, ROS1, RRAS2, RXRA, SETD2, SF3B1, SMAD3, SMAD4, SMARCA4, SMARCB1, SOS1, SPOP, STAT3, STK11, STK19, TCF7L2, TGFBR1, TGFBR2, TP53, TP63, TSC1, TSC2, U2AF1, VHL, XPO1, and TERT.

Additionally or alternatively, in some embodiments of the methods disclosed herein, the plurality of features further comprises platelet count, hemoglobin levels, leukocyte counts, body mass index (BMI), administration of chemotherapy, age, time from cancer diagnosis, race, and metastatic sites of disease. In certain embodiments, the metastatic sites of disease comprise one or more of adrenal gland, bone, brain, liver, lung, lymph, and pleura.

In any of the preceding embodiments, the method further comprises applying the classifier to data on a cancer patient to generate a predictor, and determining whether the cancer patient is at risk for cancer-associated VTE based on the predictor and the operating-point threshold. In some embodiments, the predictor comprises a cumulative incidence function (CIF) for cancer-associated VTE.

In any of the foregoing embodiments, the method further comprises administering an effective amount of anticoagulant therapy to the cancer patient predicted to be at risk for cancer-associated VTE based on the predictor and the operating-point threshold. Examples of anticoagulant therapy include, but are not limited to, apixaban, betrixaban, dabigatran, edoxaban, fondaparinux, heparin, rivaroxaban, warfarin, Xa inhibitors, statins, and enoxaparin. Examples of statins include, but are not limited to atorvastatin, fluvastatin, lovastatin, pitavastatin, pravastatin, rosuvastatin, and simvastatin.

In some embodiments, the cancer patient is chemotherapy-naïve or has received/is receiving systemic chemotherapy.

In one aspect, the present disclosure provides a method of estimating risk of cancer-associated venous thromboembolism (VTE) in a cancer patient using a machine learning classifier, the method comprising: receiving patient data corresponding to a plurality of features for the cancer patient; applying the machine learning classifier to the patient data to generate a predictor; and determining whether the cancer patient is at risk for cancer-associated VTE based on the predictor and an operating-point threshold, wherein the machine learning classifier is trained by: (a) receiving cohort data on a cohort of subjects, the subjects in the cohort having a plurality of cancer types; (b) generating a training dataset based on the received cohort data, wherein the training dataset comprises the plurality of features for each subject in the cohort, wherein the plurality of features comprises (i) cell free DNA concentration, (ii) maximum ctDNA VAF, (iii) ctDNA alterations in at least one cancer associated gene, and (iv) cancer type; and (c) applying a machine learning method to the training dataset to develop the machine learning classifier for estimating risk of cancer-associated VTE, wherein applying the machine learning method comprises: applying a machine learning technique to the training dataset; performing hyperparameter optimization to identify one or more machine learning models with an accuracy that exceeds an accuracy threshold for the machine learning classifier; and determining the optimal operating-point threshold based on optimization of sensitivity and specificity of the receiver operating characteristic (ROC) curves for the training dataset; wherein the machine learning classifier is configured to receive the plurality of features for cancer patients and generate predictors for risk of cancer-associated VTE in cancer patients. In some embodiments, the method further comprises administering an effective amount of anticoagulant therapy to the cancer patient predicted to be at risk for cancer-associated VTE based on the predictor and the operating-point threshold. Examples of anticoagulant therapy include, but are not limited to, apixaban, betrixaban, dabigatran, edoxaban, fondaparinux, heparin, rivaroxaban, warfarin, Xa inhibitors, statins, and enoxaparin. Examples of statins include, but are not limited to atorvastatin, fluvastatin, lovastatin, pitavastatin, pravastatin, rosuvastatin, and simvastatin. Additionally or alternatively, in some embodiments, the predictor comprises a cumulative incidence function (CIF) for cancer-associated VTE. The subjects in the cohort may be chemotherapy-naïve or may have received systemic chemotherapy. In any of the preceding embodiments of the methods disclosed herein, one or more of the plurality of features for the cancer patient are determined by assaying blood and/or sequencing tumor DNA.

Additionally or alternatively, in certain embodiments, the plurality of cancer types are selected from the group consisting of non-small cell lung cancer, breast cancer, pancreatic cancer, melanoma, retinoblastoma, prostate cancer, esophagogastric cancer, histiocytosis, germ cell tumor, endometrial cancer, small cell lung cancer, soft tissue sarcoma, Gastrointestinal Stromal Tumor, ovarian cancer, mature B-Cell neoplasms, small bowel cancer, renal cell carcinoma, thyroid cancer, ampullary cancer, appendiceal cancer, sellar tumor, uterine sarcoma, bone cancer, non-melanoma skin cancer, cervical cancer, mesothelioma, glioma, thymic tumor, gastrointestinal neuroendocrine tumor, salivary gland cancer, sex cord stromal tumor, anal cancer, mature T and NK neoplasms, peritoneal cancer, Head and neck cancer, choroid plexus tumor, leukemia, primary CNS melanocytic tumors, Myelodysplastic Syndromes, Peripheral Nervous System, mastocytosis, Wilms tumor, lymphatic cancer, vaginal cancer, Hodgkin lymphoma, adrenocortical carcinoma, brain tumors, embryonal tumors and Non-Hodgkin lymphoma.

The machine learning technique may model survival outcomes with competing risks. In some embodiments, the machine learning technique is a random forest technique, and the one or more machine learning models are random forest models. Additionally or alternatively, in certain embodiments, the machine learning classifier is an ensemble learning random forest classifier. Additionally or alternatively, in some embodiments, performing the hyperparameter optimization comprises performing an exhaustive grid search technique.

Additionally or alternatively, in some embodiments of the methods disclosed herein, the plurality of features further comprises platelet count, hemoglobin levels, leukocyte counts, body mass index (BMI), administration of chemotherapy, age, time from cancer diagnosis, race, and metastatic sites of disease.

Additionally or alternatively, in some embodiments of the methods disclosed herein, the at least one cancer associated gene is selected from the group consisting of AKT1, ALK, APC, AR, ARAF, ARIDIA, ARID2, ATM, B2M, BCL2, BCOR, BRAF, BRCA1, BRCA2, CARD11, CBFB, CCND1, CDH1, CDK4, CDKN2A, CIC, CREBBP, CTCF, CTNNB1, DICER1, DIS3, DNMT3A, EGFR, EIF1AX, EP300, ERBB2, ERBB3, ERCC2, ESR1, EZH2, FBXW7, FGFR1, FGFR2, FGFR3, FGFR4, FLT3, FOXA1, FOXL2, FOXO1, FUBP1, GATA3, GNA11, GNAQ, GNAS, H3F3A, HIST1H3B, HRAS, IDH1, IDH2, IKZF1, INPPL1, JAK1, KDM6A, KEAP1, KIT, KNSTRN, KRAS, MAP2K1, MAPK1, MAX, MED12, MET, MLH1, MSH2, MSH3, MSH6, MTOR, MYC, MYCN, MYD88, MYOD1, NF1, NFE2L2, NOTCH1, NRAS, NTRK1, NTRK2, NTRK3, NUP93, PAK7, PDGFRA, PIK3CA, PIK3CB, PIK3R1, PIK3R2, PMS2, POLE, PPP2RIA, PPP6C, PRKCI, PTCH1, PTEN, PTPN11, RAC1, RAF1, RB1, RET, RHOA, RIT1, ROS1, RRAS2, RXRA, SETD2, SF3B1, SMAD3, SMAD4, SMARCA4, SMARCB1, SOS1, SPOP, STAT3, STK11, STK19, TCF7L2, TGFBR1, TGFBR2, TP53, TP63, TSC1, TSC2, U2AF1, VHL, XPO1, and TERT.

In some embodiments, the cancer patient is chemotherapy-naïve or has received/is receiving systemic chemotherapy.

In any and all embodiments of the methods disclosed herein, one or more of the plurality of features for each subject in the cohort are determined by assaying blood and/or sequencing tumor DNA.

In any and all embodiments of the methods disclosed herein, the cancer-associated VTE is pulmonary embolism or lower extremity deep vein thrombosis (DVT), optionally wherein lower extremity DVT includes thrombi involving a common iliac vein, an external iliac vein, a common femoral vein, a superficial femoral vein, a deep femoral vein, a popliteal vein, a peroneal vein, an anterior tibial vein, a posterior tibial vein, or a deep calf vein.

In another aspect, the present disclosure provides a machine learning system for training a machine learning classifier for estimating risk of cancer-associated venous thromboembolism (VTE) in cancer patients, the system comprising a processor and a memory with instructions which, when executed by the processor, cause the processor to: (a) receive data on a cohort of subjects, the subjects in the cohort having a plurality of cancer types; (b) generate a training dataset based on the received data, wherein the training dataset comprises a plurality of features for each subject in the cohort, wherein the plurality of features comprises (i) cell free DNA concentration, (ii) maximum ctDNA VAF, (iii) ctDNA alterations in at least one cancer associated gene, and (iv) cancer type; and (c) apply a machine learning method to the training dataset to develop the machine learning classifier for estimating risk of cancer-associated VTE in cancer patients; wherein applying the machine learning method comprises: applying a machine learning technique to the training dataset; performing hyperparameter optimization to identify one or more machine learning models with an accuracy that exceeds an accuracy threshold for the machine learning classifier; and determining an optimal operating-point threshold based on optimization of sensitivity and specificity of the receiver operating characteristic (ROC) curves for the training dataset; wherein the machine learning classifier is configured to receive the plurality of features for cancer patients and generate predictors for risk of cancer-associated VTE in cancer patients. The subjects in the cohort may be chemotherapy-naïve or may have received systemic chemotherapy.

The machine learning technique may model survival outcomes with competing risks. In some embodiments, the machine learning technique is a random forest technique, and the one or more machine learning models are random forest models. Additionally or alternatively, in certain embodiments, the machine learning classifier is an ensemble learning random forest classifier.

Additionally or alternatively, in some embodiments, performing the hyperparameter optimization comprises performing an exhaustive grid search technique.

Additionally or alternatively, in some embodiments of the systems disclosed herein, the at least one cancer associated gene is selected from the group consisting of AKT1, ALK, APC, AR, ARAF, ARIDIA, ARID2, ATM, B2M, BCL2, BCOR, BRAF, BRCA1, BRCA2, CARD11, CBFB, CCND1, CDH1, CDK4, CDKN2A, CIC, CREBBP, CTCF, CTNNB1, DICER1, DIS3, DNMT3A, EGFR, EIF1AX, EP300, ERBB2, ERBB3, ERCC2, ESR1, EZH2, FBXW7, FGFR1, FGFR2, FGFR3, FGFR4, FLT3, FOXA1, FOXL2, FOXO1, FUBP1, GATA3, GNA11, GNAQ, GNAS, H3F3A, HIST1H3B, HRAS, IDH1, IDH2, IKZF1, INPPL1, JAK1, KDM6A, KEAP1, KIT, KNSTRN, KRAS, MAP2K1, MAPK1, MAX, MED12, MET, MLH1, MSH2, MSH3, MSH6, MTOR, MYC, MYCN, MYD88, MYOD1, NF1, NFE2L2, NOTCH1, NRAS, NTRK1, NTRK2, NTRK3, NUP93, PAK7, PDGFRA, PIK3CA, PIK3CB, PIK3R1, PIK3R2, PMS2, POLE, PPP2RIA, PPP6C, PRKCI, PTCH1, PTEN, PTPN11, RAC1, RAF1, RB1, RET, RHOA, RIT1, ROS1, RRAS2, RXRA, SETD2, SF3B1, SMAD3, SMAD4, SMARCA4, SMARCB1, SOS1, SPOP, STAT3, STK11, STK19, TCF7L2, TGFBR1, TGFBR2, TP53, TP63, TSC1, TSC2, U2AF1, VHL, XPO1, and TERT.

Additionally or alternatively, in some embodiments of the systems disclosed herein, the plurality of features further comprises platelet count, hemoglobin levels, leukocyte counts, body mass index (BMI), administration of chemotherapy, age, time from cancer diagnosis, race, and metastatic sites of disease. Metastatic sites of disease may comprise one or more of adrenal gland, bone, brain, liver, lung, lymph, and pleura.

Additionally or alternatively, in certain embodiments of the systems disclosed herein, the plurality of cancer types are selected from the group consisting of non-small cell lung cancer, breast cancer, pancreatic cancer, melanoma, retinoblastoma, prostate cancer, esophagogastric cancer, histiocytosis, germ cell tumor, endometrial cancer, small cell lung cancer, soft tissue sarcoma, Gastrointestinal Stromal Tumor, ovarian cancer, mature B-Cell neoplasms, small bowel cancer, renal cell carcinoma, thyroid cancer, ampullary cancer, appendiceal cancer, sellar tumor, uterine sarcoma, bone cancer, non-melanoma skin cancer, cervical cancer, mesothelioma, glioma, thymic tumor, gastrointestinal neuroendocrine tumor, salivary gland cancer, sex cord stromal tumor, anal cancer, mature T and NK neoplasms, peritoneal cancer, Head and neck cancer, choroid plexus tumor, leukemia, primary CNS melanocytic tumors, Myelodysplastic Syndromes, Peripheral Nervous System, mastocytosis, Wilms tumor, lymphatic cancer, vaginal cancer, Hodgkin lymphoma, adrenocortical carcinoma, brain tumors, embryonal tumors and Non-Hodgkin lymphoma.

In any of the preceding embodiments of the systems described herein, the instructions further cause the processor to apply the machine learning classifier to data on a cancer patient to generate a predictor, and determine whether the cancer patient is at risk for cancer-associated VTE based on the predictor and the operating-point threshold. In some embodiments, the predictor comprises a cumulative incidence function (CIF) for cancer-associated VTE.

In any of the foregoing embodiments of the systems described herein, the instructions further cause the processor to recommend an anticoagulant therapy to the cancer patient predicted to be at risk for cancer-associated VTE based on the predictor and the operating-point threshold. Examples of anticoagulant therapy include, but are not limited to, apixaban, betrixaban, dabigatran, edoxaban, fondaparinux, heparin, rivaroxaban, warfarin, Xa inhibitors, statins, and enoxaparin. Examples of statins include, but are not limited to atorvastatin, fluvastatin, lovastatin, pitavastatin, pravastatin, rosuvastatin, and simvastatin.

In some embodiments, the cancer patient is chemotherapy-naïve or has received/is receiving systemic chemotherapy.

In yet another aspect, the present disclosure provides a computing system for estimating risk of cancer-associated venous thromboembolism (VTE) in a cancer patient, the computing system comprising a processor and a memory with instructions which, when executed by the processor, cause the processor to: receive patient data corresponding to a plurality of features for the cancer patient; apply a machine learning classifier to the patient data to generate a predictor; and determine whether the cancer patient is at risk for cancer-associated VTE based on the predictor and an operating-point threshold, wherein the classifier is trained by: (a) receiving cohort data on a cohort of subjects, the subjects in the cohort having a plurality of cancer types; (b) generating a training dataset based on the received cohort data, wherein the training dataset comprises the plurality of features for each subject in the cohort, wherein the plurality of features comprises (i) cell free DNA concentration, (ii) maximum ctDNA VAF, (iii) ctDNA alterations in at least one cancer associated gene, and (iv) cancer type; and (c) applying a machine learning method to the training dataset to develop the machine learning classifier for estimating risk of cancer-associated VTE, wherein applying the machine learning method comprises: applying a machine learning technique to the training dataset; performing hyperparameter optimization to identify one or more machine learning models with an accuracy that exceeds an accuracy threshold for the machine learning classifier; and determining the optimal operating-point threshold based on optimization of sensitivity and specificity of the receiver operating characteristic (ROC) curves for the training dataset; wherein the machine learning classifier is configured to receive the plurality of features for cancer patients and generate predictors for risk of cancer-associated VTE in cancer patients.

The machine learning technique may model survival outcomes with competing risks. In some embodiments, the machine learning technique is a random forest technique, and the one or more machine learning models are random forest models. Additionally or alternatively, in certain embodiments, the machine learning classifier is an ensemble learning random forest classifier.

Additionally or alternatively, in some embodiments, performing the hyperparameter optimization comprises performing an exhaustive grid search technique.

Additionally or alternatively, in some embodiments of the systems disclosed herein, the plurality of features further comprises platelet count, hemoglobin levels, leukocyte counts, body mass index (BMI), administration of chemotherapy, age, time from cancer diagnosis, race, and metastatic sites of disease.

In certain embodiments, the at least one cancer associated gene is selected from the group consisting of AKT1, ALK, APC, AR, ARAF, ARIDIA, ARID2, ATM, B2M, BCL2, BCOR, BRAF, BRCA1, BRCA2, CARD11, CBFB, CCND1, CDH1, CDK4, CDKN2A, CIC, CREBBP, CTCF, CTNNB1, DICER1, DIS3, DNMT3A, EGFR, EIF1AX, EP300, ERBB2, ERBB3, ERCC2, ESR1, EZH2, FBXW7, FGFR1, FGFR2, FGFR3, FGFR4, FLT3, FOXA1, FOXL2, FOXO1, FUBP1, GATA3, GNA11, GNAQ, GNAS, H3F3A, HIST1H3B, HRAS, IDH1, IDH2, IKZF1, INPPL1, JAK1, KDM6A, KEAP1, KIT, KNSTRN, KRAS, MAP2K1, MAPK1, MAX, MED12, MET, MLH1, MSH2, MSH3, MSH6, MTOR, MYC, MYCN, MYD88, MYOD1, NF1, NFE2L2, NOTCH1, NRAS, NTRK1, NTRK2, NTRK3, NUP93, PAK7, PDGFRA, PIK3CA, PIK3CB, PIK3R1, PIK3R2, PMS2, POLE, PPP2R1A, PPP6C, PRKCI, PTCH1, PTEN, PTPN11, RAC1, RAF1, RB1, RET, RHOA, RIT1, ROS1, RRAS2, RXRA, SETD2, SF3B1, SMAD3, SMAD4, SMARCA4, SMARCB1, SOS1, SPOP, STAT3, STK11, STK19, TCF7L2, TGFBR1, TGFBR2, TP53, TP63, TSC1, TSC2, U2AF1, VHL, XPO1, and TERT

In any of the preceding embodiments of the systems described herein, the instructions further cause the processor to recommend an anticoagulant therapy to the cancer patient predicted to be at risk for cancer-associated VTE based on the predictor and the operating-point threshold. In some embodiments, the predictor comprises a cumulative incidence function (CIF) for cancer-associated VTE. Examples of anticoagulant therapy include, but are not limited to, apixaban, betrixaban, dabigatran, edoxaban, fondaparinux, heparin, rivaroxaban, warfarin, Xa inhibitors, statins, and enoxaparin. Examples of statins include, but are not limited to atorvastatin, fluvastatin, lovastatin, pitavastatin, pravastatin, rosuvastatin, and simvastatin.

Additionally or alternatively, in certain embodiments of the systems disclosed herein, the plurality of cancer types are selected from the group consisting of non-small cell lung cancer, breast cancer, pancreatic cancer, melanoma, retinoblastoma, prostate cancer, esophagogastric cancer, histiocytosis, germ cell tumor, endometrial cancer, small cell lung cancer, soft tissue sarcoma, Gastrointestinal Stromal Tumor, ovarian cancer, mature B-Cell neoplasms, small bowel cancer, renal cell carcinoma, thyroid cancer, ampullary cancer, cervical cancer, mesothelioma, glioma, thymic tumor, gastrointestinal neuroendocrine tumor, salivary gland cancer, sex cord stromal tumor, anal cancer, mature T and NK neoplasms, peritoneal cancer, Head and neck cancer, choroid plexus tumor, leukemia, primary CNS melanocytic tumors, Myelodysplastic Syndromes, Peripheral Nervous System, mastocytosis, Wilms tumor, lymphatic cancer, vaginal cancer, Hodgkin lymphoma, adrenocortical carcinoma, brain tumors, embryonal tumors and Non-Hodgkin lymphoma.

In some embodiments, the cancer patient is chemotherapy-naïve or has received/is receiving systemic chemotherapy.

In any and all embodiments of the systems disclosed herein, one or more of the plurality of features for each subject in the cohort are determined by assaying blood and/or sequencing tumor DNA.

In one aspect, the present disclosure provides a non-transitory computer-readable storage medium comprising instructions which, when executed by a processor of a machine learning system, configure the machine learning system to train a machine learning classifier to estimate risk of cancer-associated venous thromboembolism (VTE) in cancer patients, wherein the instructions are configured to cause the processor to: (a) receive data on a cohort of subjects, the subjects in the cohort having a plurality of cancer types; (b) generate a training dataset based on the received data, wherein the training dataset comprises a plurality of features for each subject in the cohort, the plurality of features comprising (i) cell free DNA concentration, (ii) maximum ctDNA VAF, (iii) ctDNA alterations in at least one cancer associated gene, and (iv) cancer type; and (c) apply a machine learning method to the training dataset to develop the machine learning classifier for estimating risk of cancer-associated VTE in cancer patients; wherein applying the machine learning method comprises: applying a machine learning technique to the training dataset; performing hyperparameter optimization to identify one or more machine learning models with an accuracy that exceeds an accuracy threshold for the machine learning classifier; and determining an optimal operating-point threshold based on optimization of sensitivity and specificity of the receiver operating characteristic (ROC) curves for the training dataset; wherein the machine learning classifier is configured to receive the plurality of features for cancer patients and generate predictors for risk of cancer-associated VTE in cancer patients. The subjects in the cohort may be chemotherapy-naïve or may have received systemic chemotherapy.

The machine learning technique may model survival outcomes with competing risks. In some embodiments, the machine learning technique is a random forest technique, and the one or more machine learning models are random forest models. Additionally or alternatively, in certain embodiments, the machine learning classifier is an ensemble learning random forest classifier.

Additionally or alternatively, in some embodiments, performing the hyperparameter optimization comprises performing an exhaustive grid search technique.

Additionally or alternatively, in some embodiments of the computer-readable storage medium disclosed herein, the at least one cancer associated gene is selected from the group consisting of AKT1, ALK, APC, AR, ARAF, ARIDIA, ARID2, ATM, B2M, BCL2, BCOR, BRAF, BRCA1, BRCA2, CARD11, CBFB, CCND1, CDH1, CDK4, CDKN2A, CIC, CREBBP, CTCF, CTNNB1, DICER1, DIS3, DNMT3A, EGFR, EIF1AX, EP300, ERBB2, ERBB3, ERCC2, ESR1, EZH2, FBXW7, FGFR1, FGFR2, FGFR3, FGFR4, FLT3, FOXA1, FOXL2, FOXO1, FUBP1, GATA3, GNA11, GNAQ, GNAS, H3F3A, HIST1H3B, HRAS, IDH1, IDH2, IKZF1, INPPL1, JAK1, KDM6A, KEAP1, KIT, KNSTRN, KRAS, MAP2K1, MAPK1, MAX, MED12, MET, MLH1, MSH2, MSH3, MSH6, MTOR, MYC, MYCN, MYD88, MYOD1, NF1, NFE2L2, NOTCH1, NRAS, NTRK1, NTRK2, NTRK3, NUP93, PAK7, PDGFRA, PIK3CA, PIK3CB, PIK3R1, PIK3R2, PMS2, POLE, PPP2RIA, PPP6C, PRKCI, PTCH1, PTEN, PTPN11, RAC1, RAF1, RB1, RET, RHOA, RIT1, ROS1, RRAS2, RXRA, SETD2, SF3B1, SMAD3, SMAD4, SMARCA4, SMARCB1, SOS1, SPOP, STAT3, STK11, STK19, TCF7L2, TGFBR1, TGFBR2, TP53, TP63, TSC1, TSC2, U2AF1, VHL, XPO1, and TERT.

Additionally or alternatively, in some embodiments of the computer-readable storage medium disclosed herein, the plurality of features further comprises platelet count, hemoglobin levels, leukocyte counts, body mass index (BMI), administration of chemotherapy, age, time from cancer diagnosis, race, and metastatic sites of disease. Metastatic sites of disease may comprise one or more of adrenal gland, bone, brain, liver, lung, lymph, and pleura.

In any of the preceding embodiments of the computer-readable storage medium described herein, the instructions further cause the processor to apply the machine learning classifier to data on a cancer patient to generate a predictor, and determine whether the cancer patient is at risk for cancer-associated VTE based on the predictor and the operating-point threshold. In some embodiments, the predictor comprises a cumulative incidence function (CIF) for cancer-associated VTE.

Additionally or alternatively, in certain embodiments of the computer-readable storage medium disclosed herein, the plurality of cancer types are selected from the group consisting of non-small cell lung cancer, breast cancer, pancreatic cancer, melanoma, retinoblastoma, prostate cancer, esophagogastric cancer, histiocytosis, germ cell tumor, endometrial cancer, small cell lung cancer, soft tissue sarcoma, Gastrointestinal Stromal Tumor, ovarian cancer, mature B-Cell neoplasms, small bowel cancer, renal cell carcinoma, thyroid cancer, ampullary cancer, appendiceal cancer, sellar tumor, uterine sarcoma, bone cancer, non-melanoma skin cancer, cervical cancer, mesothelioma, glioma, thymic tumor, gastrointestinal neuroendocrine tumor, salivary gland cancer, sex cord stromal tumor, anal cancer, mature T and NK neoplasms, peritoneal cancer, Head and neck cancer, choroid plexus tumor, leukemia, primary CNS melanocytic tumors, Myelodysplastic Syndromes, Peripheral Nervous System, mastocytosis, Wilms tumor, lymphatic cancer, vaginal cancer, Hodgkin lymphoma, adrenocortical carcinoma, brain tumors, embryonal tumors and Non-Hodgkin lymphoma.

In any of the preceding embodiments of the computer-readable storage medium described herein, the instructions further cause the processor to recommend an anticoagulant therapy to the cancer patient predicted to be at risk for cancer-associated VTE based on the predictor and the operating-point threshold. Examples of anticoagulant therapy include, but are not limited to, apixaban, betrixaban, dabigatran, edoxaban, fondaparinux, heparin, rivaroxaban, warfarin, Xa inhibitors, statins, and enoxaparin. Examples of statins include, but are not limited to atorvastatin, fluvastatin, lovastatin, pitavastatin, pravastatin, rosuvastatin, and simvastatin.

In some embodiments, the cancer patient is chemotherapy-naïve or has received/is receiving systemic chemotherapy.

In another aspect, the present disclosure provides a non-transitory computer-readable storage medium comprising instructions which, when executed by a processor of a computing system, configure the computing system to estimate risk of cancer-associated venous thromboembolism (VTE) in a cancer patient, wherein the instructions are configured to cause the processor to: receive patient data corresponding to a plurality of features for the cancer patient; apply a machine learning classifier to the patient data to generate a predictor; and determine whether the cancer patient is at risk for cancer-associated VTE based on the predictor and an operating-point threshold, wherein the classifier is trained by: (a) receiving cohort data on a cohort of subjects, the subjects in the cohort having a plurality of cancer types; (b) generating a training dataset based on the received cohort data, wherein the training dataset comprises the plurality of features for each subject in the cohort, wherein the plurality of features comprises (i) cell free DNA concentration, (ii) maximum ctDNA VAF, (iii) ctDNA alterations in at least one cancer associated gene, and (iv) cancer type; and (c) applying a machine learning method to the training dataset to develop the machine learning classifier for estimating risk of cancer-associated VTE, wherein applying the machine learning method comprises: applying a machine learning technique to the training dataset; performing hyperparameter optimization to identify one or more machine learning models with an accuracy that exceeds an accuracy threshold for the machine learning classifier; and determining the optimal operating-point threshold based on optimization of sensitivity and specificity of the receiver operating characteristic (ROC) curves for the training dataset; wherein the machine learning classifier is configured to receive the plurality of features for cancer patients and generate predictors for risk of cancer-associated VTE in cancer patients.

The machine learning technique may model survival outcomes with competing risks. In some embodiments, the machine learning technique is a random forest technique, and the one or more machine learning models are random forest models. Additionally or alternatively, in certain embodiments, the machine learning classifier is an ensemble learning random forest classifier.

Additionally or alternatively, in some embodiments, performing the hyperparameter optimization comprises performing an exhaustive grid search technique.

Additionally or alternatively, in some embodiments of the computer-readable storage medium disclosed herein, the at least one cancer associated gene is selected from the group consisting of AKT1, ALK, APC, AR, ARAF, ARIDIA, ARID2, ATM, B2M, BCL2, BCOR, BRAF, BRCA1, BRCA2, CARD11, CBFB, CCND1, CDH1, CDK4, FGFR4, FLT3, FOXA1, FOXL2, FOXO1, FUBP1, GATA3, GNA11, GNAQ, GNAS, H3F3A, HIST1H3B, HRAS, IDH1, IDH2, IKZF1, INPPL1, JAK1, KDM6A, KEAP1, KIT, KNSTRN, KRAS, MAP2K1, MAPK1, MAX, MED12, MET, MLH1, MSH2, MSH3, MSH6, MTOR, MYC, MYCN, MYD88, MYOD1, NF1, NFE2L2, NOTCH1, NRAS, NTRK1, NTRK2, NTRK3, NUP93, PAK7, PDGFRA, PIK3CA, PIK3CB, PIK3R1, PIK3R2, PMS2, POLE, PPP2RIA, PPP6C, PRKCI, PTCH1, PTEN, PTPN11, RAC1, RAF1, RB1, RET, RHOA, RIT1, ROS1, RRAS2, RXRA, SETD2, SF3B1, SMAD3, SMAD4, SMARCA4, SMARCB1, SOS1, SPOP, STAT3, STK11, STK19, TCF7L2, TGFBR1, TGFBR2, TP53, TP63, TSC1, TSC2, U2AF1, VHL, XPO1, and TERT.

Additionally or alternatively, in some embodiments of the computer-readable storage medium disclosed herein, the plurality of features further comprises platelet count, hemoglobin levels, leukocyte counts, body mass index (BMI), administration of chemotherapy, age, time from cancer diagnosis, race, and metastatic sites of disease.

In any of the preceding embodiments of the computer-readable storage medium described herein, the instructions further cause the processor to recommend an anticoagulant therapy to the cancer patient predicted to be at risk for cancer-associated VTE based on the predictor and the operating-point threshold. In some embodiments, the predictor comprises a cumulative incidence function (CIF) for cancer-associated VTE. Examples of anticoagulant therapy include, but are not limited to, apixaban, betrixaban, dabigatran, edoxaban, fondaparinux, heparin, rivaroxaban, warfarin, Xa inhibitors, statins, and enoxaparin. Examples of statins include, but are not limited to atorvastatin, fluvastatin, lovastatin, pitavastatin, pravastatin, rosuvastatin, and simvastatin.

Additionally or alternatively, in certain embodiments of the computer-readable storage medium disclosed herein, the plurality of cancer types are selected from the group consisting of non-small cell lung cancer, breast cancer, pancreatic cancer, melanoma, retinoblastoma, prostate cancer, esophagogastric cancer, histiocytosis, germ cell tumor, endometrial cancer, small cell lung cancer, soft tissue sarcoma, Gastrointestinal Stromal Tumor, ovarian cancer, mature B-Cell neoplasms, small bowel cancer, renal cell carcinoma, thyroid cancer, ampullary cancer, appendiceal cancer, sellar tumor, uterine sarcoma, bone cancer, non-melanoma skin cancer, cervical cancer, mesothelioma, glioma, thymic tumor, gastrointestinal neuroendocrine tumor, salivary gland cancer, sex cord stromal tumor, anal plexus tumor, leukemia, primary CNS melanocytic tumors, Myelodysplastic Syndromes, Peripheral Nervous System, mastocytosis, Wilms tumor, lymphatic cancer, vaginal cancer, Hodgkin lymphoma, adrenocortical carcinoma, brain tumors, embryonal tumors and Non-Hodgkin lymphoma.

In some embodiments, the cancer patient is chemotherapy-naïve or has received/is receiving systemic chemotherapy.

In any of the preceding embodiments of the computer-readable storage medium disclosed herein, one or more of the plurality of features for the cancer patient are determined by assaying blood and/or sequencing tumor DNA.

### EXAMPLES

The present technology is further illustrated by the following Examples, which should not be construed as limiting in any way.

### Example 1: Materials and Experimental Methods

Patients. Adults with stage IV or recurrent NSCLC and either no known driver mutation pre-enrollment or progression of disease following targeted therapy were eligible for ctDNA sequencing at the provider's discretion. Patients also required clinical annotation based on previous cohort requirements (Mantha et al Blood 2021).

ctDNA Sequencing. Blood samples were sent for plasma sequencing by the ctDx Lung Assay (Resolution Bioscience, Agilent Technologies), a hybrid capture next-generation sequencing assay with a variant allele fraction (VAF) detection limit of 0.1%-0.5%. Detection of any copy number alteration or mutation that passed a standard germline filtering protocol (Jee et al ASCO 2021) resulted in a label of ctDNA being detected in that plasma sample. Genes/alterations included in the panel are the following:

| SNV/Indel | Fusions | CNV |
|---|---|---|
| AKT1 | ALK | B2M |
| ALK | EGFR | EGFR |
| B2M | FGFR2 | ERBB2 (HER2) |
| BRAF | FGFR3 | FGFR1 |
| EGFR | NTRK1 | KRAS |
| ERBB2 (HER2) | RET | MET |
| FGFR2 | ROS1 | MYC |
| FGFR3 | | NTRK1 |
| KEAP1 | | PIK3CA |
| KRAS | | PTEN |
| MAP2K1 (MEK1) | | RICTOR |
| MET | | STK11 |
| NRAS | | TP53 |
| PIK3CA | | |
| RET | | |
| ROS1 | | |
| STK11 | | |
| TP53 | | |

Clinical annotation. CAT events were abstracted from the clinical chart using a previously validated process (Mantha et al, Blood 137 (15): 2103-2113 (2021)). Khorana score parameters were obtained from pre-chemotherapy laboratory and BMI values as previously described (Khorana et al., Blood 111 (10): 4902-7 (2008)).

Statistical analysis. Time-to-event analyses were performed from time of ctDNA blood draw to time of CAT event or last follow-up (right censorship). Risk of CAT between cohorts were compared using Cox proportional hazards models.

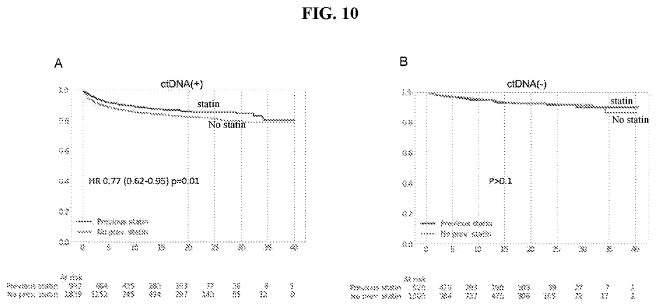

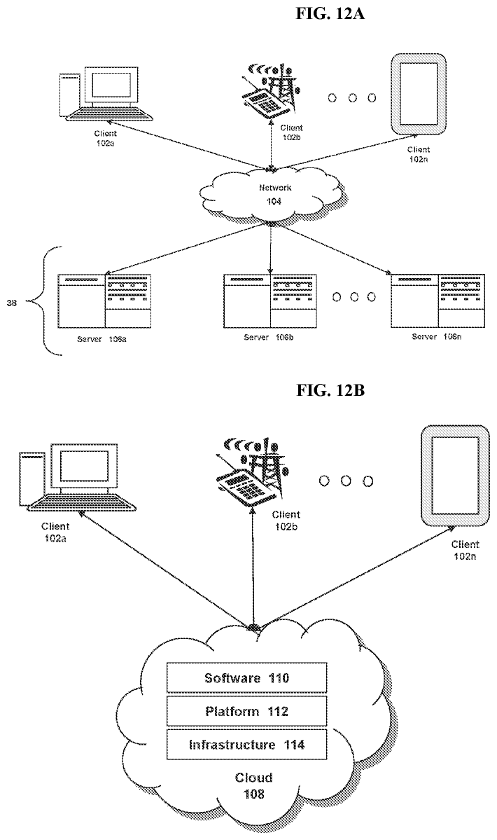

Machine learning model details. We implemented random survival forest (RSF; Ishwaran et al The Annals of Applied Statistics 2008) models to predict time to CAT. Models were implemented in python using the sksurv library. We implemented two versions of the RSF model. In the first, input variables included cancer type (i.e. the cancer types in FIG. 11 as one-hot encoded variables) as well as liquid biopsy-related parameters (i.e. logiocfDNA concentration and max VAF as continuous variables, and presence or absence of any of the listed MSK-ACCESS genes as one-hot encoded variables). In the second, the aforementioned variables were included as well as Khorana score components (platelet count, hemoglobin level, leukocyte count, BMI, and receipt of chemotherapy), demographics (age and time since diagnosis as a continuous variable as well as White, Black, Asian, or Other race as one-hot encoded variables), and metastatic sites of disease (adrenal, bone, brain, liver, lung, lymph, pleura, and other as one-hot encoded variables). Models were trained and validated using 5-fold cross validation. The primary metric of success was the c-index. The first model achieved a c-index of 0.73 (95% CI 0.70-0.76) and the second achieved a c-index of 0.75 (0.72-0.78). These models outperformed those based on Khorana score, metastatic sites, or demographics alone including within cancer subtypes (FIG. 8B, “Liquid Biopsy”=model 1, “All”=model 2) and successfully risk-stratified patients CAT (FIG. 8D).

### Example 2: CtDNA Biomarker Accurately Predicts Cancer-Associated Thromboembolism in Lung Cancer Patients

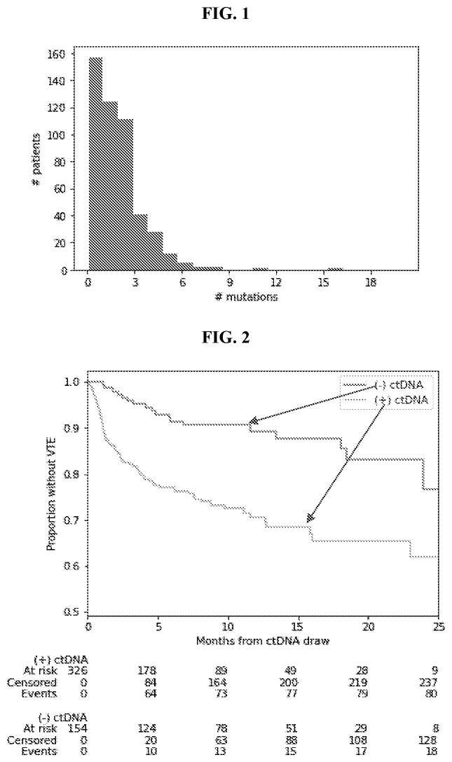

A total of 480 patients were analyzed. Of these 480 patients, 157 had no detectable ctDNA (i.e. no ctDNA alterations). Among patients with detectable ctDNA, most patients had only one ctDNA alteration (FIG. 1).

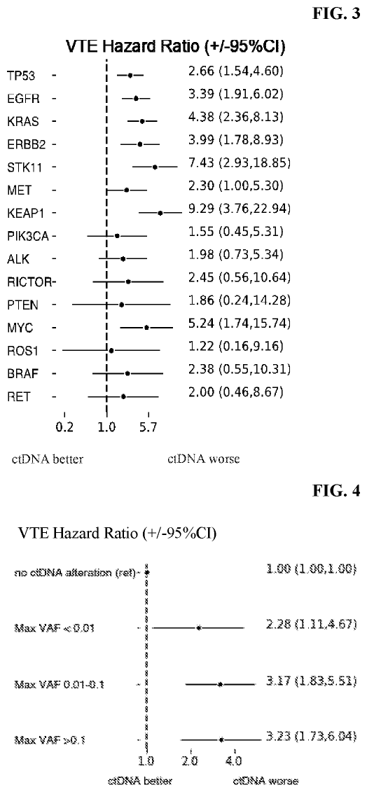

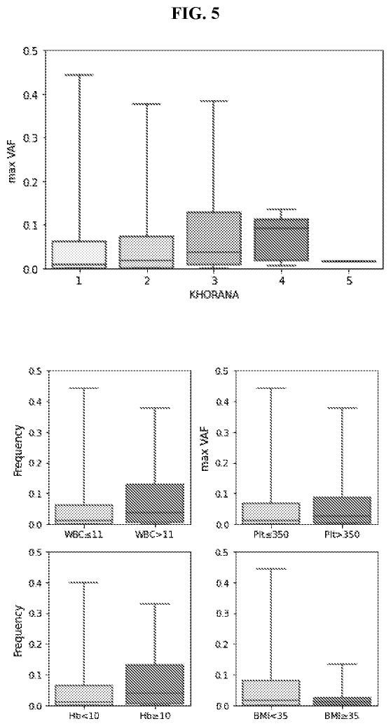

FIG. 2 demonstrates that patients with ctDNA alterations had higher risk of CAT than those without (HR 2.9, 95% CI 1.8-4.9). In subgroup analyses in which only alterations in specific, individual genes are considered (with at least 8 patients with ctDNA mutations in that gene), trends toward higher CAT rates were observed for all genes considered relative to the ctDNA(−) group, supporting the notion that a diverse gene panel increases the sensitivity of the assay for patients at risk for CAT. See FIG. 3.

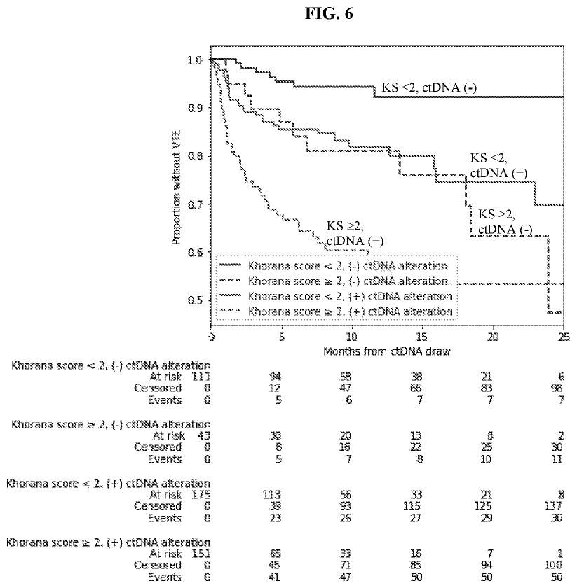

As shown in FIG. 4, there was a trend toward higher rates of CAT with higher ctDNA VAF, although any above the limit of detection (LOD) with this assay resulted in higher rates of CAT than the ctDNA(−) group.

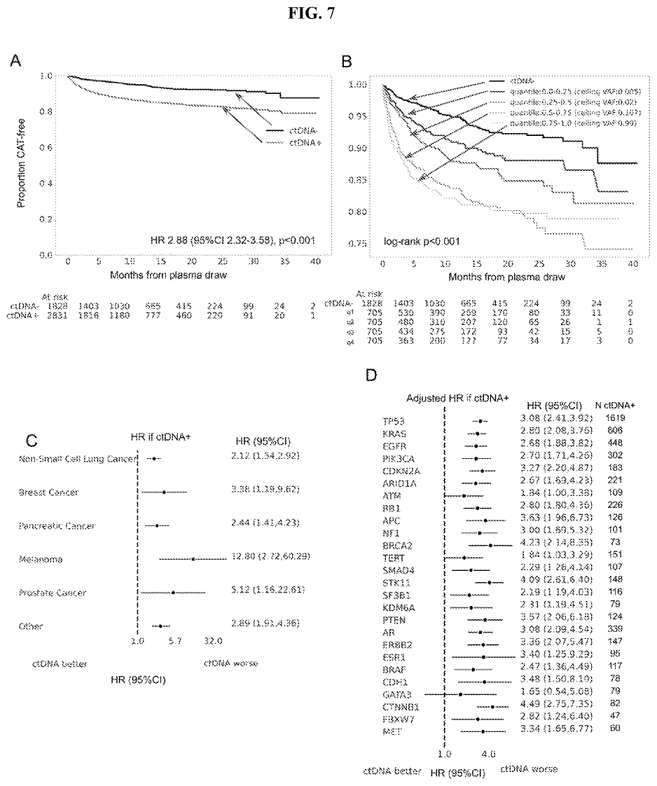

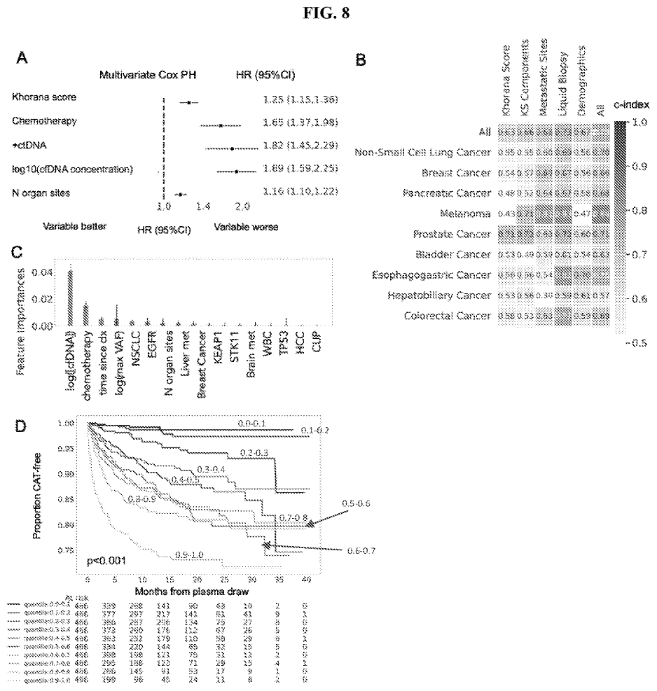

Surprisingly, ctDNA levels did not correlate with Khorana Score (R=0.18, p<0.001) or its individual components. See FIG. 5. Moreover, ctDNA predicts CAT risk in a manner that is orthogonal to the Khorana Score (FIG. 6). These results demonstrate a means for risk-stratifying patients for CAT based on the results of ctDNA panel sequencing using a prespecified gene panel and a LOD of 0.1%-0.5%.

### Example 3: CtDNA Biomarker Accurately Predicts Cancer-Associated Thromboembolism in Additional Cancer Types

### Patients and Methods

A single-center, pan-cancer observational study including patients who underwent ctDNA sequencing with MSK-ACCESS, a NY State-approved, 129-gene assay (N=4,659, breakdown by cancer type included in FIG. 11) was conducted. It was hypothesized that ctDNA detection would be associated with higher rates of CAT while controlling for cancer type and genomic content. It was further hypothesized that the inclusion of data from ctDNA sequencing assays in multivariable machine learning models including cell-free (cf) DNA concentrations, Khorana score components, and other features would improve CAT prediction. The ability of ctDNA as a predictive biomarker for prophylactic anticoagulation using nonrandomized, real-world evidence was assessed.

### Results

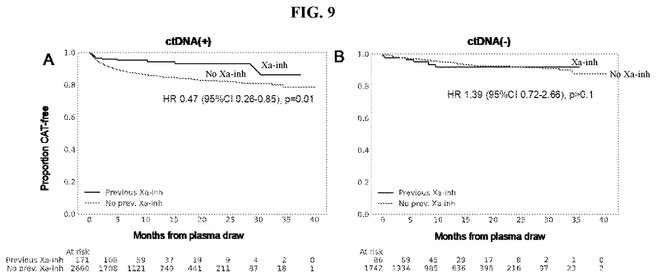

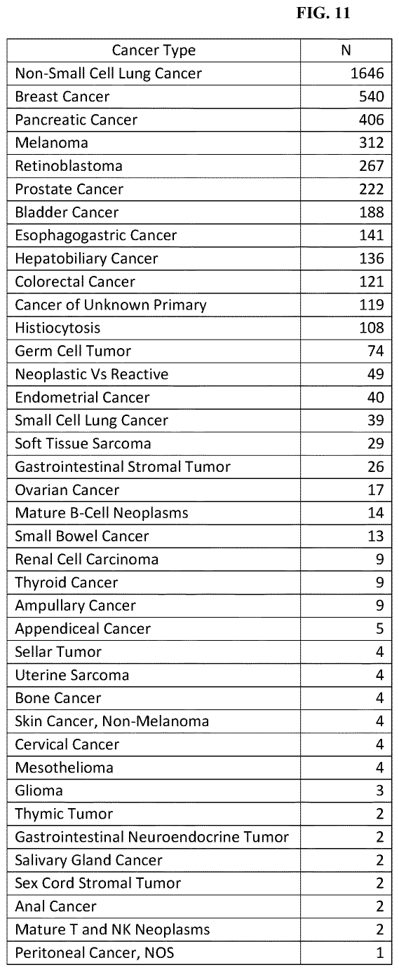

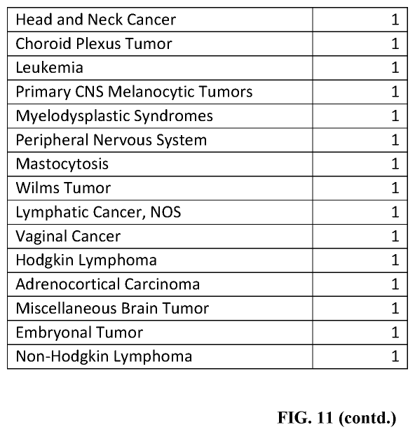

ctDNA detection was associated with CAT (HR 2.88, 95% CI 2.32-3.58) in a dose-dependent manner (FIGS. 7A-7B). This association was observed across multiple cancer types and regardless of detected gene alterations (FIGS. 7C-7D). ctDNA and cfDNA concentration were predictive of CAT independent of each other and other CAT-related variables including Khorana score and number of organ sites of metastasis (FIGS. 8A-8B). Patients receiving pre-existing anticoagulant agents had lower rates of CAT if ctDNA was detected (HR 0.60 95% CI 0.38-0.92) but not if ctDNA was undetected (FIGS. 9A-9B). Patients receiving pre-existing statins also had lower rates of CAT if ctDNA was detected but not if ctDNA was undetected (FIGS. 10A-10B).

Random survival forests (python, sksurv) from time of plasma draw (for ctDNA) to CAT or last follow-up were 5-fold trained and cross validated across all patients with MSK-ACCESS (N=4,659). The probability of CAT at 6 months was computed for all patients in the respective validation sets. Patients in the validation set who either had CAT within 6 months of plasma draw or were confirmed CAT-free for at least 6 months were used as labels to generate the receiver operating curve (shown in FIG. 12) and to compute the area under the curve (AUC) as well as sensitivity and specificity for optimal cut points.

The sensitivity/specificity metrics for the three models Khorana Score, Liquid biopsy and combined are shown below:
Khorana Score (Sensitivity: 0.658, Specificity: 0.585)
Liquid Biopsy (Sensitivity: 0.698, Specificity: 0.697)
All (Sensitivity: 0.705, Specificity: 0.703)

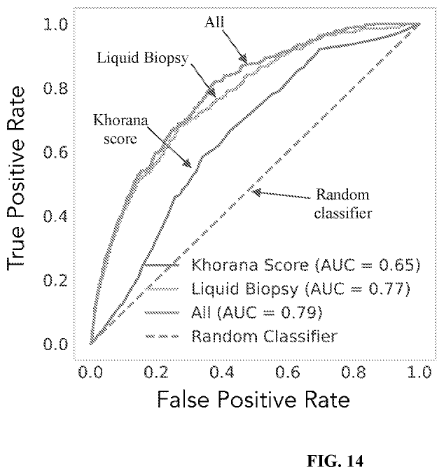

The AUCs of the three models are reported in FIG. 14.

### Conclusion

ctDNA is an independent prognostic biomarker for CAT and may help identify patients who may benefit from prophylactic anticoagulation in a pan-cancer setting.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for preventing cancer associated thromboembolism (CAT) in a cancer patient in need thereof comprising
(I)
a. detecting ctDNA molecules in a biological sample obtained from the cancer patient, wherein the ctDNA molecules are detected at a variant allele fraction (VAF) detection limit of at least 0.1%-0.5% and
b. administering to the cancer patient an effective amount of anticoagulant therapy; or
(II)
administering to the cancer patient an effective amount of anticoagulant therapy, wherein a biological sample obtained from the cancer patient comprises detectable ctDNA molecules, wherein the ctDNA molecules are detected at a variant allele fraction (VAF) detection limit of at least 0.1%-0.5%.

2. (canceled)

3. The method of claim 1, wherein the ctDNA molecules are detected at a VAF detection limit of from about 0.1% to about 0.5%, or from about 0.5% to about 2%, or from about 2% to about 10%, or from about 10% to about 99%.

4. (canceled)

5. (canceled)

6. (canceled)

7. The method of claim 1, wherein the cancer patient is diagnosed with or suffers from a cancer selected from the group consisting of non-small cell lung cancer, breast cancer, pancreatic cancer, melanoma, retinoblastoma, prostate cancer, esophagogastric cancer, histiocytosis, germ cell tumor, endometrial cancer, small cell lung cancer, soft tissue sarcoma, Gastrointestinal Stromal Tumor, ovarian cancer, mature B-Cell neoplasms, small bowel cancer, renal cell carcinoma, thyroid cancer, ampullary cancer, appendiceal cancer, sellar tumor, uterine sarcoma, bone cancer, non-melanoma skin cancer, cervical cancer, mesothelioma, glioma, thymic tumor, gastrointestinal neuroendocrine tumor, salivary gland cancer, sex cord stromal tumor, anal cancer, mature T and NK neoplasms, peritoneal cancer, Head and neck cancer, choroid plexus tumor, leukemia, primary CNS melanocytic tumors, Myelodysplastic Syndromes, Peripheral Nervous System, mastocytosis, Wilms tumor, lymphatic cancer, vaginal cancer, Hodgkin lymphoma, adrenocortical carcinoma, brain tumors, embryonal tumors and Non-Hodgkin lymphoma, optionally wherein the cancer is Stage 1, Stage 2, Stage 3, or Stage 4.

8. The method of claim 1, wherein the ctDNA molecules comprise one or more mutations in at least one cancer associated gene selected from the group consisting of AKT1, ALK, APC, AR, ARAF, ARIDIA, ARID2, ATM, B2M, BCL2, BCOR, BRAF, BRCA1, BRCA2, CARD11, CBFB, CCND1, CDH1, CDK4, CDKN2A, CIC, CREBBP, CTCF, CTNNB1, DICER1, DIS3, DNMT3A, EGFR, EIF1AX, EP300, ERBB2, ERBB3, ERCC2, ESR1, EZH2, FBXW7, FGFR1, FGFR2, FGFR3, FGFR4, FLT3, FOXA1, FOXL2, FOXO1, FUBP1, GATA3, GNA11, GNAQ, GNAS, H3F3A, HIST1H3B, HRAS, IDH1, IDH2, IKZF1, INPPL1, JAK1, KDM6A, KEAP1, KIT, KNSTRN, KRAS, MAP2K1, MAPK1, MAX, MED12, MET, MLH1, MSH2, MSH3, MSH6, MTOR, MYC, MYCN, MYD88, MYOD1, NF1, NFE2L2, NOTCH1, NRAS, NTRK1, NTRK2, NTRK3, NUP93, PAK7, PDGFRA, PIK3CA, PIK3CB, PIK3R1, PIK3R2, PMS2, POLE, PPP2RIA, PPP6C, PRKCI, PTCH1, PTEN, PTPN11, RAC1, RAF1, RB1, RET, RHOA, RIT1, ROS1, RRAS2, RXRA, SETD2, SF3B1, SMAD3, SMAD4, SMARCA4, SMARCB1, SOS1, SPOP, STAT3, STK11, STK19, TCF7L2, TGFBR1, TGFBR2, TP53, TP63, TSC1, TSC2, U2AF1, VHL, XPO1, and TERT, optionally wherein the ctDNA molecules comprise 2-20 mutations in the at the least one cancer associated gene.

9. (canceled)

10. The method of claim 1, wherein the ctDNA molecules comprise one or more rearrangements in at least one cancer associated gene selected from the group consisting of ALK, BRAF, EGFR, ETV6, FGFR2, FGFR3, MET, NTRK1, RET and ROS1, optionally wherein the one or more rearrangements comprise indels, CNVs, and/or gene fusions or wherein the ctDNA molecules comprise 2-20 rearrangements in the at the least one cancer associated gene.

11. (canceled)

12. (canceled)

13. The method of claim 1, wherein the cancer patient has a Khorana Score ≥2 or <2; or
wherein the cancer patient has one or more organ sites of metastasis; or
wherein the biological sample is whole blood, serum or plasma; or
wherein the biological sample has a cfDNA concentration ranging from about 3 pg/μL to 5.5 ng/μL; or
wherein the anticoagulant therapy comprises one or more of apixaban, betrixaban, dabigatran, edoxaban, fondaparinux, heparin, rivaroxaban, warfarin, Xa inhibitors, a statin, or enoxaparin, optionally wherein the statin is selected from the group consisting of atorvastatin, fluvastatin, lovastatin, pitavastatin, pravastatin, rosuvastatin, and simvastatin; or
wherein the cancer patient is chemotherapy-naïve or has received/is receiving systemic chemotherapy, optionally wherein the systemic chemotherapy comprises one or more of an alkylating agent, an antibiotic, an antimetabolite, an antimitotic, a cyclin-dependent kinase inhibitor, an epidermal growth factor receptor inhibitor, a multikinase inhibitor, a PARP inhibitor, a platinum-based agent, a selective estrogen receptor modulator (SERM), or a VEGF inhibitor; or
wherein the cancer patient is immunotherapy-naïve or has received/is receiving immunotherapy, optionally wherein the immunotherapy comprises one or more of anti-PD-1 antibody, anti-PD-L1 antibody, anti-PD-L2 antibody, anti-CTLA-4 antibody, anti-TIM3 antibody, anti-4-1BB antibody, anti-CD73 antibody, anti-GITR antibody, and anti-LAG-3 antibody; or
wherein the cancer patient is radiotherapy-naïve or has received/is receiving radiotherapy, optionally wherein the radiotherapy comprises external radiotherapy, radiotherapy implants (brachytherapy), pre-targeted radioimmunotherapy, radiotherapy injections, radioisotope therapy, or intrabeam radiotherapy; or
wherein the CAT is pulmonary embolism or lower extremity deep vein thrombosis (DVT), optionally wherein lower extremity DVT includes thrombi involving a common iliac vein, an external iliac vein, a common femoral vein, a superficial femoral vein, a deep femoral vein, a popliteal vein, a peroneal vein, an anterior tibial vein, a posterior tibial vein, or a deep calf vein.

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. A method for preventing cancer associated thromboembolism (CAT) in a lung cancer patient in need thereof comprising
(I)
a. detecting ctDNA molecules in a biological sample obtained from the lung cancer patient, wherein the ctDNA molecules comprise at least one alteration in at least one cancer-associated gene selected from the group consisting of AKT1, ALK, B2M, BRAF, EGFR, ERBB2 (HER2), FGFR2, FGFR3, KEAP1, KRAS, MAP2K1 (MEK1), MET, NRAS, PIK3CA, RET, ROS1, STK11, TP53, NTRK1, FGFR1, MYC, PTEN, and RICTOR; and
b. administering to the lung cancer patient an effective amount of anticoagulant therapy; or
(II)
administering to the lung cancer patient an effective amount of anticoagulant therapy,
wherein a biological sample obtained from the lung cancer patient comprises detectable ctDNA molecules comprising at least one alteration in at least one cancer-associated gene selected from the group consisting of AKT1, ALK, B2M, BRAF, EGFR, ERBB2 (HER2), FGFR2, FGFR3, KEAP1, KRAS, MAP2K1 (MEK1), MET, NRAS, PIK3CA, RET, ROS1, STK11, TP53, NTRK1, FGFR1, MYC, PTEN, and RICTOR.

26. (canceled)

27. The method of claim 25, wherein the anticoagulant therapy comprises one or more of apixaban, betrixaban, dabigatran, edoxaban, fondaparinux, heparin, rivaroxaban, warfarin, Xa inhibitors, a statin, or enoxaparin, optionally wherein the statin is selected from the group consisting of atorvastatin, fluvastatin, lovastatin, pitavastatin, pravastatin, rosuvastatin, and simvastatin; or
wherein the lung cancer patient has a Khorana Score <2 or ≥2; or
wherein the at least one alteration is a SNV, an indel, a CNV, or a gene fusion; or
wherein the at least one alteration is detected at a variant allele fraction (VAF) detection limit of 0.1%-0.5%; or
wherein the lung cancer is non-small cell lung cancer (NSCLC) or small cell lung cancer (SCLC); or
wherein the detected ctDNA molecules comprise one alteration in the at the least one cancer associated gene; or
wherein the detected ctDNA molecules comprise 2-20 alterations in the at the least one cancer associated gene; or
wherein the ctDNA molecules are detected via polymerase chain reaction (PCR), real-time quantitative PCR (qPCR), droplet digital PCR (ddPCR), Reverse transcriptase-PCR (RT-PCR), microarray, RNA-Seq, or next-generation sequencing; or
wherein the biological sample is whole blood, serum or plasma; or
wherein the lung cancer patient is chemotherapy-naïve or has received/is receiving systemic chemotherapy, optionally wherein the systemic chemotherapy comprises one or more of an alkylating agent, an antibiotic, an antimetabolite, an antimitotic, a cyclin-dependent kinase inhibitor, an epidermal growth factor receptor inhibitor, a multikinase inhibitor, a PARP inhibitor, a platinum-based agent, a selective estrogen receptor modulator (SERM), or a VEGF inhibitor; or
wherein the lung cancer patient is immunotherapy-naïve or has received/is receiving immunotherapy, optionally wherein the immunotherapy comprises one or more of anti-PD-1 antibody, anti-PD-L1 antibody, anti-PD-L2 antibody, anti-CTLA-4 antibody, anti-TIM3 antibody, anti-4-1BB antibody, anti-CD73 antibody, anti-GITR antibody, and anti-LAG-3 antibody; or
wherein the lung cancer patient is radiotherapy-naïve or has received/is receiving radiotherapy, optionally wherein the radiotherapy comprises external radiotherapy, radiotherapy implants (brachytherapy), pre-targeted radioimmunotherapy, radiotherapy injections, radioisotope therapy, or intrabeam radiotherapy; or
wherein the lung cancer is Stage 1, Stage 2, Stage 3, or Stage 4; or
wherein the CAT is pulmonary embolism or lower extremity deep vein thrombosis (DVT), optionally wherein lower extremity DVT includes thrombi involving a common iliac vein, an external iliac vein, a common femoral vein, a superficial femoral vein, a deep femoral vein, a popliteal vein, a peroneal vein, an anterior tibial vein, a posterior tibial vein, or a deep calf vein; or
wherein the at least one alteration comprises a SNV and/or an indel in one or more of AKT1, ALK, B2M, BRAF, EGFR, ERBB2 (HER2), FGFR2, FGFR3, KEAP1, KRAS, MAP2K1 (MEK1), MET, NRAS, PIK3CA, RET, ROS1, STK11 and TP53; or
wherein the at least one alteration comprises a gene fusion in one or more of ALK, EGFR, FGFR2, FGFR3, NTRK1, RET, and ROS1; or
wherein the at least one alteration comprises a CNV in one or more of B2M, EGFR, ERBB2 (HER2), FGFR1, KRAS, MET, MYC, NTRK1, PIK3CA, PTEN, RICTOR, STK11, and TP53.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. A method of training a machine learning classifier for estimating risk of cancer-associated venous thromboembolism (VTE) in cancer patients, comprising:
a. receiving data on a cohort of subjects, the subjects in the cohort having a plurality of cancer types;
b. generating a training dataset based on the received data, the training dataset comprising a plurality of features for each subject in the cohort, the plurality of features comprising (i) cell free DNA concentration, (ii) maximum ctDNA VAF, (iii) ctDNA alterations in at least one cancer associated gene, and (iv) cancer type; and
c. applying a machine learning method to the training dataset to develop the machine learning classifier for estimating risk of cancer-associated VTE in cancer patients,
wherein applying the machine learning method comprises:
applying a machine learning technique to the training dataset;
performing hyperparameter optimization to identify one or more machine learning models with an accuracy that exceeds an accuracy threshold for the classifier; and
determining an optimal operating-point threshold based on optimization of sensitivity and specificity of the receiver operating characteristic (ROC) curves for the training dataset;
wherein the classifier is configured to receive the plurality of features for cancer patients and generate predictors for risk of cancer-associated VTE in cancer patients.

52. The method of claim 51, wherein the at least one cancer associated gene is selected from the group consisting of AKT1, ALK, APC, AR, ARAF, ARIDIA, ARID2, ATM, B2M, BCL2, BCOR, BRAF, BRCA1, BRCA2, CARD11, CBFB, CCND1, CDH1, CDK4, CDKN2A, CIC, CREBBP, CTCF, CTNNB1, DICER1, DIS3, DNMT3A, EGFR, EIF1AX, EP300, ERBB2, ERBB3, ERCC2, ESR1, EZH2, FBXW7, FGFR1, FGFR2, FGFR3, FGFR4, FLT3, FOXA1, FOXL2, FOX01, FUBP1, GATA3, GNA11, GNAQ, GNAS, H3F3A, HIST1H3B, HRAS, IDH1, IDH2, IKZF1, INPPL1, JAK1, KDM6A, KEAP1, KIT, KNSTRN, KRAS, MAP2K1, MAPK1, MAX, MED12, MET, MLH1, MSH2, MSH3, MSH6, MTOR, MYC, MYCN, MYD88, MYOD1, NF1, NFE2L2, NOTCH1, NRAS, NTRK1, NTRK2, NTRK3, NUP93, PAK7, PDGFRA, PIK3CA, PIK3CB, PIK3R1, PIK3R2, PMS2, POLE, PPP2RIA, PPP6C, PRKCI, PTCH1, PTEN, PTPN11, RAC1, RAF1, RB1, RET, RHOA, RIT1, ROS1, RRAS2, RXRA, SETD2, SF3B1, SMAD3, SMAD4, SMARCA4, SMARCB1, SOS1, SPOP, STAT3, STK11, STK19, TCF7L2, TGFBR1, TGFBR2, TP53, TP63, TSC1, TSC2, U2AF1, VHL, XPO1, and TERT or
wherein the plurality of cancer types are selected from the group consisting of non-small cell lung cancer, breast cancer, pancreatic cancer, melanoma, retinoblastoma, prostate cancer, esophagogastric cancer, histiocytosis, germ cell tumor, endometrial cancer, small cell lung cancer, soft tissue sarcoma, Gastrointestinal Stromal Tumor, ovarian cancer, mature B-Cell neoplasms, small bowel cancer, renal cell carcinoma, thyroid cancer, ampullary cancer, appendiceal cancer, sellar tumor, uterine sarcoma, bone cancer, non-melanoma skin cancer, cervical cancer, mesothelioma, glioma, thymic tumor, gastrointestinal neuroendocrine tumor, salivary gland cancer, sex cord stromal tumor, anal cancer, mature T and NK neoplasms, peritoneal cancer, Head and neck cancer, choroid plexus tumor, leukemia, primary CNS melanocytic tumors, Myelodysplastic Syndromes, Peripheral Nervous System, mastocytosis, Wilms tumor, lymphatic cancer, vaginal cancer, Hodgkin lymphoma, adrenocortical carcinoma, brain tumors, embryonal tumors and Non-Hodgkin lymphoma; or
wherein the subjects in the cohort are chemotherapy-naïve or have received systemic chemotherapy.

53. The method of claim 51, wherein the plurality of features further comprises platelet count, hemoglobin levels, leukocyte counts, body mass index (BMI), administration of chemotherapy, age, time from cancer diagnosis, race, and metastatic sites of disease, optionally wherein the metastatic sites of disease comprise one or more of adrenal gland, bone, brain, liver, lung, lymph, and pleura.

54. (canceled)

55. The method of claim 51, wherein the machine learning technique is a random forest technique, and wherein the one or more machine learning models are random forest models; or
wherein the machine learning classifier is an ensemble learning random forest classifier; or
wherein the machine learning technique models survival outcomes with competing risks; or
wherein performing the hyperparameter optimization comprises performing an exhaustive grid search technique.

56. (canceled)

57. (canceled)

58. (canceled)

59. The method of claim 51, further comprising applying the classifier to data on a cancer patient to generate a predictor, and determining whether the cancer patient is at risk for cancer-associated VTE based on the predictor and the operating-point threshold, optionally wherein the predictor comprises a cumulative incidence function (CIF) for cancer-associated VTE.

60. (canceled)

61. The method of claim 59, further comprising administering an effective amount of anticoagulant therapy to the cancer patient predicted to be at risk for cancer-associated VTE based on the predictor and the operating-point threshold, optionally wherein the anticoagulant therapy comprises one or more of apixaban, betrixaban, dabigatran, edoxaban, fondaparinux, heparin, rivaroxaban, warfarin, Xa inhibitors, a statin, or enoxaparin, optionally wherein the statin is selected from the group consisting of atorvastatin, fluvastatin, lovastatin, pitavastatin, pravastatin, rosuvastatin, and simvastatin; or
wherein the cancer patient is chemotherapy-naïve or has received/is receiving systemic chemotherapy

62. (canceled)

63. (canceled)

64. (canceled)

65. (canceled)

66. A method of estimating risk of cancer-associated venous thromboembolism (VTE) in a cancer patient using a machine learning classifier, the method comprising:
a. receiving patient data corresponding to a plurality of features for the cancer patient;
b. applying the machine learning classifier to the patient data to generate a predictor; and
c. determining whether the cancer patient is at risk for cancer-associated VTE based on the predictor and an operating-point threshold, wherein the machine learning classifier is trained by:
i. receiving cohort data on a cohort of subjects, the subjects in the cohort having a plurality of cancer types;
ii. generating a training dataset based on the received cohort data, the training dataset comprising the plurality of features for each subject in the cohort, the plurality of features comprising (i) cell free DNA concentration, (ii) maximum ctDNA VAF, (iii) ctDNA alterations in at least one cancer associated gene, and (iv) cancer type; and
iii. applying a machine learning method to the training dataset to develop the machine learning classifier for estimating risk of cancer-associated VTE,
wherein applying the machine learning method comprises:
applying a machine learning technique to the training dataset;
performing hyperparameter optimization to identify one or more machine learning models with an accuracy that exceeds an accuracy threshold for the machine learning classifier; and
determining the optimal operating-point threshold based on optimization of sensitivity and specificity of the receiver operating characteristic (ROC) curves for the training dataset;
wherein the machine learning classifier is configured to receive the plurality of features for cancer patients and generate predictors for risk of cancer-associated VTE in cancer patients.

67. The method of claim 66, further comprising administering an effective amount of anticoagulant therapy to the cancer patient predicted to be at risk for cancer-associated VTE based on the predictor and the operating-point threshold, optionally wherein the predictor comprises a cumulative incidence function (CIF) for cancer-associated VTE; or
wherein the plurality of cancer types are selected from the group consisting of non-small cell lung cancer, breast cancer, pancreatic cancer, melanoma, retinoblastoma, prostate cancer, esophagogastric cancer, histiocytosis, germ cell tumor, endometrial cancer, small cell lung cancer, soft tissue sarcoma, Gastrointestinal Stromal Tumor, ovarian cancer, mature B-Cell neoplasms, small bowel cancer, renal cell carcinoma, thyroid cancer, ampullary cancer, appendiceal cancer, sellar tumor, uterine sarcoma, bone cancer, non-melanoma skin cancer, cervical cancer, mesothelioma, glioma, thymic tumor, gastrointestinal neuroendocrine tumor, salivary gland cancer, sex cord stromal tumor, anal cancer, mature T and NK neoplasms, peritoneal cancer, Head and neck cancer, choroid plexus tumor, leukemia, primary CNS melanocytic tumors, Myelodysplastic Syndromes, Peripheral Nervous System, mastocytosis, Wilms tumor, lymphatic cancer, vaginal cancer, Hodgkin lymphoma, adrenocortical carcinoma, brain tumors, embryonal tumors and Non-Hodgkin lymphoma; or
wherein one or more of the plurality of features for the cancer patient are determined by assaying blood and/or sequencing tumor DNA.

68. (canceled)

69. The method of claim 66, wherein the at least one cancer associated gene is selected from the group consisting of AKT1, ALK, APC, AR, ARAF, ARIDIA, ARID2, ATM, B2M, BCL2, BCOR, BRAF, BRCA1, BRCA2, CARD11, CBFB, CCND1, CDH1, CDK4, CDKN2A, CIC, CREBBP, CTCF, CTNNB1, DICER1, DIS3, DNMT3A, EGFR, EIF1AX, EP300, ERBB2, ERBB3, ERCC2, ESR1, EZH2, FBXW7, FGFR1, FGFR2, FGFR3, FGFR4, FLT3, FOXA1, FOXL2, FOXO1, FUBP1, GATA3, GNA11, GNAQ, GNAS, H3F3A, HIST1H3B, HRAS, IDH1, IDH2, IKZF1, INPPL1, JAK1, KDM6A, KEAP1, KIT, KNSTRN, KRAS, MAP2K1, MAPK1, MAX, MED12, MET, MLH1, MSH2, MSH3, MSH6, MTOR, MYC, MYCN, MYD88, MYOD1, NF1, NFE2L2, NOTCH1, NRAS, NTRK1, NTRK2, NTRK3, NUP93, PAK7, PDGFRA, PIK3CA, PIK3CB, PIK3R1, PIK3R2, PMS2, POLE, PPP2R1A, PPP6C, PRKCI, PTCH1, PTEN, PTPN11, RAC1, RAF1, RB1, RET, RHOA, RIT1, ROS1, RRAS2, RXRA, SETD2, SF3B1, SMAD3, SMAD4, SMARCA4, SMARCB1, SOS1, SPOP, STAT3, STK11, STK19, TCF7L2, TGFBR1, TGFBR2, TP53, TP63, TSC1, TSC2, U2AF1, VHL, XPO1, and TERT.

70. The method of claim 66, wherein the plurality of features further comprises platelet count, hemoglobin levels, leukocyte counts, body mass index (BMI), administration of chemotherapy, age, time from cancer diagnosis, race, and metastatic sites of disease, optionally wherein the metastatic sites of disease comprise one or more of adrenal gland, bone, brain, liver, lung, lymph, and pleura; or
wherein the machine learning technique is a random forest technique, and wherein the one or more machine learning models are random forest models; or
wherein the machine learning classifier is an ensemble learning random forest classifier; or
wherein the machine learning technique models survival outcomes with competing risks
wherein performing the hyperparameter optimization comprises performing an exhaustive grid search technique; or
wherein the anticoagulant therapy comprises one or more of apixaban, betrixaban, dabigatran, edoxaban, fondaparinux, heparin, rivaroxaban, warfarin, Xa inhibitors, a statin, or enoxaparin, optionally wherein the statin is selected from the group consisting of atorvastatin, fluvastatin, lovastatin, pitavastatin, pravastatin, rosuvastatin, and simvastatin.

71. (canceled)

72. (canceled)

73. (canceled)

74. (canceled)

75. (canceled)

76. (canceled)

77. (canceled)

78. (canceled)

79. The method of claim 51, wherein one or more of the plurality of features for each subject in the cohort are determined by assaying blood and/or sequencing tumor DNA; or
wherein the cancer-associated VTE is pulmonary embolism or lower extremity deep vein thrombosis (DVT), optionally wherein lower extremity DVT includes thrombi involving a common iliac vein, an external iliac vein, a common femoral vein, a superficial femoral vein, a deep femoral vein, a popliteal vein, a peroneal vein, an anterior tibial vein, a posterior tibial vein, or a deep calf vein.

80. The method of claim 66, wherein the cancer-associated VTE is pulmonary embolism or lower extremity deep vein thrombosis (DVT), optionally wherein lower extremity DVT includes thrombi involving a common iliac vein, an external iliac vein, a common femoral vein, a superficial femoral vein, a deep femoral vein, a popliteal vein, a peroneal vein, an anterior tibial vein, a posterior tibial vein, or a deep calf vein; or wherein one or more of the plurality of features for each subject in the cohort are determined by assaying blood and/or sequencing tumor DNA.

81.-136. (canceled)


SEQUENCE LISTING (23 sequences)

SEQ ID NO: 1 - DNA, 3008 bp, Homo sapiens
  source 1..3008: mol_type = genomic DNA
taattatgggtctgtaaccaccctggactgggtgctcctcactgacggacttgtctgaacctctctttgtctccagcgcccagcactgggcctggcaaaacctgagacgcccggtacatgttggccaaatgaatgaaccagattcagaccggcaggggcgctgtggtttaggaggggcctggggtttctcccaggaggtttttgggcttgcgctggagggctctggactcccgtttgcgccagtggcctgcatcctggtcctgtcttcctcatgtttgaatttctttgctttcctagtctggggagcagggaggagccctgtgccctgtcccaggatccatgggtaggaacaccatggacagggagagcaaacggggccatctgtcaccaggggcttagggaaggccgagccagcctgggtcaaagaagtcaaaggggctgcctggaggaggcagcctgtcagctggtgcatcagaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctgcagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccgggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattcttcaataaaagttgcttttcaaaaaaaaaaaaaaaaaaaaaa

SEQ ID NO: 2 - DNA, 6240 bp, Homo sapiens
  source 1..6240: mol_type = genomic DNA
agatgcgatccagcggctctgggggcggcagcggtggtagcagctggtacctcccgccgcctctgttcggagggtcgcggggcaccgaggtgctttccggccgccctctggtcggccacccaaagccgcgggcgctgatgatgggtgaggagggggcggcaagatttcgggcgcccctgccctgaacgccctcagctgctgccgccggggccgctccagtgcctgcgaactctgaggagccgaggcgccggtgagagcaaggacgctgcaaacttgcgcagcgcgggggctgggattcacgcccagaagttcagcaggcagacagtccgaagccttcccgcagcggagagatagcttgagggtgcgcaagacggcagcctccgccctcggttcccgcccagaccgggcagaagagcttggaggagccaaaaggaacgcaaaaggcggccaggacagcgtgcagcagctgggagccgccgttctcagccttaaaagttgcagagattggaggctgccccgagaggggacagaccccagctccgactgcggggggcaggagaggacggtacccaactgccacctcccttcaaccatagtagttcctctgtaccgagcgcagcgagctacagacgggggcgcggcactcggcgcggagagcgggaggctcaaggtcccagccagtgagcccagtgtgcttgagtgtctctggactcgcccctgagcttccaggtctgtttcatttagactcctgctcgcctccgtgcagttgggggaaagcaagagacttgcgcgcacgcacagtcctctggagatcaggtggaaggagccgctgggtaccaaggactgttcagagcctcttcccatctcggggagagcgaagggtgaggctgggcccggagagcagtgtaaacggcctcctccggcgggatgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccttctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtggaggagagagaggcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttctttcaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgactaa

SEQ ID NO: 3 - DNA, 977 bp, Homo sapiens
  source 1..977: mol_type = genomic DNA
attcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttcctt

SEQ ID NO: 4 - DNA, 9687 bp, Homo sapiens
  source 1..9687: mol_type = genomic DNA
cttcccccaatcccctcaggctcggctgcgcccggggccgcgggccggtacctgaggtggcccaggcgccctccgcccgcggcgccgcccgggccgctcctccccgcgccccccgcgccccccgctcctccgcctccgcctccgcctccgcctcccccagctctccgcctcccttccccctccccgcccgacagcggccgctcgggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggagaatttgcagccttcaagtagccaccatcatggcagcatctgctcttatttcttaagtcttgtgttcgtacaatttgttaacatcaaaacacagttctgttcctcaaatctttttttaaagatacaaaatttccaatgcataagctgatgtggaacagaatggaatttcccatccaacaaaagaggaaagaatgttttaggaaccagaattctctgctgccagtgtttcttcaacaaaaataccacgagcatacaagtctgcccagtcccaggaagaaagaggagagaccctgaattctgaccttttgatggtcaggcatgatggaaagaaactgctgctacagcttgggagatttgctatggaaagtctgccagtcaactttgcccttctaaccaccagatcaatttgtggctgatcatctgatggggcagtttcaatcaccaagcatcgttctctttcctgttctggaattttgttttggagctctttcccctagtgaccaccagttagtttctgagggatggaacaaaaatgcagcttgccctttctatgtggtgcgtgttcaggccttgacagattttatcaaaaggaaactattttatttaaatggaggctgagtggtgagtagatgtgtcttggtatggaggaaaagggcatgctgcatcttcttcctgacctccggggtctctggccttttgtttccttgctcactgaggggtctgtctaaccaagcaggctagatagtgctggcacacattgccttctttctcattgggtccagcaatgaagataagtgtttgggttttttttttttcctccacaatgtagcaaattctcaggaaatacagtttatatcttcctcctatgctcttccagtcaccaactacttatgcggctactttgtccagggcacaaaatgccgtggcagtatctaactaaacccccacaaaactgcttaataacagttttgaatgtgagaaatttagataatttaaatataaggtacaggttttaatttctgagtttcttcttttctatttttattaaaaagaaaataattttcagatttaattgaattggaaaaaaacaatacttcccaccagaattatatatcctgaaaattgtatttttgttatataaacaacttttaagaaagatcattatccttttctctacctaaatatgaggagtcttagcataatgacaaatatttataatttttcaattaatggtacttgctggatccacactaacatctttgctaataatctcattgtttcttccaactgattcctaacactatatcccacatcttctttctagtcttttatctagaatatgcaacctaaaataaaaatggtggcgtctccattcattctccttcttccttttttcccaagcctggtcttcaaaaggttgggcaatttggcagctgaattcccagacagagaatagagcaattttagggatattaggactgagggagggtgtgggaaagctgtcatcagttgtttttatagaaagaactggcattcattaagaacctaaatcttatctttgcacaaatggaaaatataacctagttatagcttcctttggcctttattaaagggtaatatcaatcacagtcatagcaaagaaagcggatgtattaatggcaaattaatggaaaacctcccttatcaggaatctagactcagaatttaggaacacaaatcaaatcagaccaaccaagctatagccaaggacttgaaagaaattaaacaagacccagaataaatcaaggaattagaaattgttatttaaaaatttcagattgtaactccaggccctgctgtctatattgcagccactaaaagctcactaccattagatttttgctaacatacatgtattcagaagaaagcctattgaaattttcattgtcttgtaaaaggttgtcctagtaaaatggaaaagatccttaagttattaatcagtttgaaaagcaaatttgtttttaagttttacatcagcagggcagtgtcttacaaaattcagaaattgcaaaggtggaaataattcacgctgatttgaagaacatcttctgtgcaataatactgcctctcttgaaaagcattggctgttttttctttttaaatatatctctagatgcttttaaatgtggctgtgttccctttaccaagattggcttcaagtttccgcaggtagagagacctgggcttgaacaagaggatgtgtttcatgtcctgctgaggaggtagaacatgtgcagcctgggtccgggactgcctccgtggggcaggggcaggggcggtaccattagggaggaagcttagcatttcagtttcttaaacaatattcagggtgatacactttttcttcccttgcattttagaataggctggtatctcatttgaacgggggagcagacttgatctcaaatgaagctgtgcccaggagccaggcttagcatattgagatttttatagataccttaaaaaataaaatatttaaacctctcttttcttcctttttctatgaaataggttttttctctagtttacaaatgacatgaaaataggttttatttgtgttttatctgctttattttttgatgcttagacaacagttagacttactgagctcctaaaaaaacgaggaagaagtccttatttgtgaaaagcactttatgagtaattgtatagacagtatgtggctgcgtcactgatcatcttgtaagggtgtaacagtcttgtctgtaaagtggctgcagtgccttctgtagtgtgttttatttttggtagggagaggtgaagccttctgaaaaatttgagagcaactacagaggattgtttgtaactgtgtagtattcctgatggacttttttcatcgttagagtcaaggacctagacttttgccactgaaataatattgaccaaaaaaatagtttataaaagggatttgtgaatagaaaattcagtgtgatcatttgttgttaatgtgcaccttaaaagaagattctgtctagctgtcaaattctggttcccgaatatctcacccctgattgtatttgagatctagtagggcatactggggcattttagaagataaaatcccatacaaatgatatatgctatatttatgttggtgttggagaagaaagagcagtatataaagaaataattcaagactgcagcactgtcaacctgaaactttgtaaatatttcctagcttctggtttggtgcggtgacagcactttcatcacaggatgttaccttgtattcaccaggcggagtgcgagctgctgcacatcctcctcagatctcacctgtccccactgtacatccacccgccagctgcttgcaaacctcatctctagctttagttcgaaaccacattgcagggttcaggtgacctctacaaaaaactacctcttcagaatgaggtaatgaatagttatttattttaaaatatgaaaagtcaggagctctagaacatgacgatgatttaagattttaacttttttgtgtacttgtatttgagcactctcattttgtcctaaagggcattatacatttaagcagtaatactgtaaaaaaatgtgttgctcggaatatctgaatgttgttgaaagtggtgccagaaccggtttaggggtacgtttcagaatcttaaccttgagtcaattgcatgaaattaaatagctgtggtatcacttcactaacagtgatgtaattttaattttcagtaggcttggcatgacagtacatcctcataatgagtttgctgcagctttgtcacatgcacaggcattcatagaaagaccacccagctaagagggtagaatgattactctttttgcaagattctcttctttgtccaagttggcattgttagtgctaggaataccagcaccttgagacgagcagattccaaccattaggctataaacaccatagccagagatggaaggtttactgtgagtatgaacagcaaatagcttacaggtcatgagttgaaatggtgtaggtgaggctctagaaaaataccttgacaatttgccaaatgatcttactgtgccttcatgatgcaataaaaaagctaacattttagcagaaatcagtgatttgtgaagagagcagccactctggtttaactcagctgtgttaataatttttagagtgcaatttagactgcataggtaaatgcactaaagagtttatagccaaaatcacatttaacaatgagaaaacacacaggtaaattttcagtgaacaaaattatttttttaaagcacataatccctagtatagtcagatatatttatcacatagagcaactaggttgcaaatatagttcagtgacatttctagagaaactttttctactcccataggctcttcaaagcatggaacttttatacaacagaaatgttgacagaaattgctgtagtttagggttgaagtactgtatgatgggcagcaatcatgtattaacttagaaggggaaattgaaatataggaccgaatttggttttatcagtttccagagtactgctgccaacctagacactgatttttcagagtttgaaatgtaaatttcttcccgggacttgattgcacatgaagctggactgcgttagtcatcctgtcccaaagcgctgtgggggccagggtggaggtctcaaggcatcctttatgacctggccattggatgtaaaagaaaacatattccatgctgtggttcttgtatcttgtttcattcctcaccattgaaagagaaagtccatgtattgtctccagcacatccttgaaatgttatactgggatggattactgatgcccatcggtagttgagccccagaagagggtagtagcatctctgcctcaggtgatgatttgtagcttggccagaggagagcggagtcaccagtatatctgtggtccatgttgctagctctggtaaaattaaaaatactggtaagatgtttgttttattagtacactagacagtaagctctgttttgttgttttcaaataacctattttcacttttgtttgggcaaagacatttaaattgaaattcaattctaatttttgttaattgtggaaagggtaattaacagttcctatcaggtatttttaatgtggaaaaggacagaaacccaactcctaaaatcttaaattaaggtaacagtgctttaaaaaaaaaaaatgcatggggcaattagtcggcaactcaatgagtgactaaagtacttttatttaacatccacaacttcaactgttaagttttattaattactaaatcagctttattaaaatgttgacatttatttagctattttgaataattatagtgacttgacgagtgtgtatgaggacacagccaatgtaagccagtgtatccattttttagaggtgcatttttttttaaagaattctgtagatagaagtgctctgaaaacaactaaaatatgtttattcatggtagtatcaaaaaatgtttgtacaaaccatctgcttctcccggccagccgagttcattctccagcaccgtgaccgctggttctcatgtacagcacatatgcgggagagttggcagaaaatttgtgaagagatgccgcaaaggaagggtctgttgacgggtgggattgggggttttgatgaagttgcttagtcctggttttgttttgaaaattactgcgttgcatttttgtgttaagtttttgaacccacgtgtgttttggtggagtatgagttggaagtcactgcaaactagcataaacaacaaagctcacagagtaggcacagatgtagagaacagagaccaaaatggggtgaggtggcagtaaatctaggatagggaaaaattaatgtgagggtgggaaataaactgtaattacctgaaatcaaatgtaagagtgcaataagtatgctttttattctaagctgtgaacggtttttttaagaatcattccttcctaatacatttgtgtatgttccatagctgattaaaaccagctatatcaacatataatgcctttttattcatgttaatgaccaacgtaagtggctagcctttatgtcttatttatcttcatgttatgttagtttacatacaggggtgtatgtctctgtgctgtccccttctcctgccttcattttaaaatgcatccatgggtcctccgtgtttcctttggccatgccacatatatagactcagtttggccttcatgatatcgcctgatttttgaggactgtatcacagtgatatgtatttgtggtaatctcatttgttggttgtacatctgatcctttcctcaacatggcaattgctgcctttcctaagataggatcatacaactgatcaggggattgaatttgatcattcatcaacatgtgtctctgaattttattcagtagttgtcattgctctttggtttagaccaagaaaaaggaaatccccccttttcatgtattccttggtttgaggacatgactcctgtaagggagaggaaagggagatgcttcctgtttgaactgcagtgaattcacggttcctgtttcaccactccaaaccttatggcgactcacacacacattcctcttttctgttactgccaaaggttcgggtttagtacacttcagttccactcaagcattgaaaaggttctcgtggagtctggggcgtgcccagtgaaaagatggggactttttaattgtccacagacctctctatacctgctttgcaaaaattacaatggagtaactatttttaaagcttatttttcaattcataaaaaagacatttattttcagtcaaatggatgatgtctccctcttttcccctattctcaatgtttgcttgaatcttttattattttttttaattctcccccatacccacttcctgatactttggttctctttcctgctcaggtcccttcatttgtactttggagtttttctcatgtaaatttgtataacagaaaatattgttcagtttggatagaaagcatggagaataaaaaaagatagctgaaattcagattgaagaaatttatttctgtgtaaagttatttaaaaactgtattatataaaaggcaaaaaaagttctatgtacttgatgtgaatatgcgaatactgctataataaagattgactgcatgga

SEQ ID NO: 5 - DNA, 10631 bp, Homo sapiens
  source 1..10631: mol_type = genomic DNA
actccttcatggaatctaaaaaattgtattcagagaagcagagagtggaatggtggttaccaggggctgggaaggtgtgagcttggggagatttggtgaaaggacatagaatctcagttagacaggaggaataagttaaagagatctattgcacatcatggtaactgtagttagtgacaatgtattgtatacatgaaaattgctaagagagtagattttaagtgttctcaccacaccaaaaaaaggtatgtgcagtaatacagtcattaattagcttgatgtagccattccacaatggatacatatatcaaaacatcatgttgtataccataaatatatactgtctctttatgtaaatttaaaaataagataaaataaatgttattcacttgtcgtggatgtggtggggacaggtgtgggatagccctccctgtacaactaggacccaggggtgatctagtgacactagccatttatcaggacgtatgggtgccagtcaggatgataaagcttccttttggccactatactacttagaaatgccctgcaaaaggtgcacatcaaagattgaaagctcaatcctggattttaagtgcttcaaaagtgcacttaattgccacatttttgtcaaacattttcccaggtagtatttttcctcatgtaaaacaacagcaatttaatttgaacagaaagcattttgaaacatacttttggcagggttccttgcagatcagaatggaaatgattaacagggcaattatcaatcatggacttttggcggcagaaggaactgtattgtttggtacagtctgggccagggccacacaccgtaacggagatactctattctgtggacggttggagggggctgtgctgagcagggtaactgcatcttttcctagactgttcacactgctgccacgaaggagtcttgtttagactggacctggctttcttcttcgcaatgagtgttgcagactcccgacaaaggccaggtggtaaagtgtggtgtctgtgagcgagagcctgagatgcctgagctgacctgtcctcagccacctgccatcgtgcagagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctattatgctctcaaatacccctaagcatctatactagcctggtatgggtatgaaagatacaaagataaataaaacatagtccctgattctaagaaattcacaatttagcaaaggaaatggactcatagatgctaaccttaaaacaacgtgacaaatgccagacaggacccatcagccaggcactgtgagagcacagagcagggaggttgggtcctgcctgaggagacctggaagggaggcctcacaggaggatgaccaggtctcagtcagcggggaggtggaaagtgcaggtgcatcaggggcaccctgaccgaggaaacagctgccagaggcctccactgctaaagtccacataaggctgaggtcagtcaccctaaacaacctgctccctctaagccaggggatgagcttggagcatcccacaagttccctaaaagttgcagcccccagggggattttgagctatcatctctgcacatgcttagtgagaagactacacaacatttctaagaatctgagattttatattgtcagttaaccactttcattattcattcacctcaggacatgcagaaatatttcagtcagaactgggaaacagaaggacctacattctgctgtcacttatgtgtcaagaagcagatgatcgatgaggcaggtcagttgtaagtgagtcacattgtagcattaaattctagtatttttgtagtttgaaacagtaacttaataaaagagcaaaagctattctagctttcttcttcatattttaattttccaccataaagtttagttgctaaattctattaattttaagattgtgcttcccaaaatagttctcacttcatctgtccagggaggcacagttctgtctggtagaagccgcaaagcccttagcctcttcacggatctggcgactgtgatgggcaggtcaggagaggagctgcccaaagtcccatgattttcacctaacagccctgatcagtcagtactcaaagcttggactccatccctgaaggtcttcctgattgatagcctggccttaataccctacagaaagcctgtccattggctgtttcttcctcagtcagttcctggaagaccttaccccatgaccccagcttcagatgtggtctttggaaacagaggtcgaaggaaagtaaggagctgagagctcacattcataggtgccgccagccttcgtgcatcttcttgcatcatctctaaggagctcctctaattacaccatgcccgtcaccccatgagggatcagagaagggatgagtcttctaaactctatattcgctgtgagtccaggttgtaagggggagcactgtggatgcatcctattgcactccagctgatgacaccaaagcttaggtgtttgctgaaagttcttgatgttgtgacttaccacccctgcctcacaactgcagacataaggggactatggattgcttagcaggaaaggcactggttctcaagggcggctgcccttgggaatcttctggtcccaaccagaaagactgtggcttgattttctcaggtgcagcccagccgtagggccttttcagagcaccccctggttattgcaacattcatcaaagtttctagaacctctggcctaaaggaagggcctggtgggatctacttggcactcgctggggggccaccccccagtgccactctcactaggcctctgattgcacttgtgtaggatgaagctggtgggtgatgggaactcagcacctcccctcaggcagaaaagaatcatctgtggagcttcaaaagaaggggcctggagtctctgcagaccaattcaacccaaatctcgggggctctttcatgattctaatgggcaaccagggttgaaacccttatttctagggtcttcagttgtacaagactgtgggtctgtaccagagcccccgtcagagtagaataaaaggctgggtagggtagagattcccatgtgcagtggagagaacaatctgcagtcactgataagcctgagacttggctcatttcaaaagcgttcaattcatcctcaccagcagttcagctggaaaggggcaaatacccccacctgagctttgaaaacgccctgggaccctctgcattctctaagtaagttatagaaaccagtctcttccctcctttgtgagtgagctgctattccacgtaggcaacacctgttgaaattgccctcaatgtctactctgcatttctttcttgtgataagcacacacttttattgcaacataatgatctgctcacatttccttgcctgggggctgtaaaaccttacagaacagaaatccttgcctctttcaccagccacacctgccataccaggggtacagctttgtactattgaagacacagacaggatttttaaatgtaaatctatttttgtaactttgttgcgggatatagttctctttatgtagcactgaactttgtacaatatatttttagaaactcatttttctactaaaacaaacacagtttactttagagagactgcaatagaatcaaaatttgaaactgaaatctttgtttaaaagggttaagttgaggcaagaggaaagccctttctctctcttataaaaaggcacaacctcattggggagctaagctaggtcattgtcatggtgaagaagagaagcatcgtttttatatttaggaaattttaaaagatgatggaaagcacatttagcttggtctgaggcaggttctgttggggcagtgttaatggaaagggctcactgttgttactactagaaaaatccagttgcatgccatactctcatcatctgccagtgtaaccctgtacatgtaagaaaagcaataacatagcactttgttggtttatatatataatgtgacttcaatgcaaattttatttttatatttacaattgatatgcatttaccagtataaactagacatgtctggagagcctaataatgttcagcacactttggttagttcaccaacagtcttaccaagcctgggcccagccaccctagagaagttattcagccctggctgcagtgacatcacctgaggagcttttaaaagcttgaagcccagctacacctcagaccgattaaacgcaaatctctggggctgaaacccaagcattcgtagtttttaaagctcctgaggtcattccaatgtgcggccaaagttgagaactactggcctagggattagccacaaggacatggacttggaggcaaattctgcaggtgtatgtgattctcaggcctagagagctaagacacaaagacctccacatctgtcgctgagagtcaagaacctgaacagagtttccatgaaggttctccaagcactagaagggagagtgtctaaacaatggttgaaaagcaaaggaaatataaaacagacacctctttccatttcctaaggtttctctctttattaagggtggactagtaataaaatataatattcttgctgcttatgcagctgacattgttgccctccctaaagcaaccaagtagcctttatttcccacagtgaaagaaaacgctggcctatcagttacattacaaaaggcagatttcaagaggattgagtaagtagttggatggctttcataaaaacaagaattcaagaagaggattcatgctttaagaaacatttgttatacattcctcacaaattatacctgggataaaaactatgtagcaggcagtgtgttttccttccatgtctctctgcactacctgcagtgtgtcctctgaggctgcaagtctgtcctatctgaattcccagcagaagcactaagaagctccaccctatcacctagcagataaaactatggggaaaacttaaatctgtgcatacatttctggatgcatttacttatctttaaaaaaaaaggaatcctatgacctgatttggccacaaaaataatcttgctgtacaatacaatctcttggaaattaagagatcctatggatttgatgactggtattagaggtgacaatgtaaccgattaacaacagacagcaataacttcgttttagaaacattcaagcaatagctttatagcttcaacatatggtacgttttaaccttgaaagttttgcaatgatgaaagcagtatttgtacaaatgaaaagcagaattctcttttatatggtttatactgttgatcagaaatgttgattgtgcattgagtattaaaaaattagatgtatattattcattgttctttactcctgagtaccttataataataataatgtattctttgttaacaa

SEQ ID NO: 6 - DNA, 4858 bp, Homo sapiens
  source 1..4858: mol_type = genomic DNA
gttctttattctactctccgctgaagtccacacagtttaaattaaagttcccggatttttgtgggcgcctgccccgcccctcgtccccctgctgtgtccatatatcgaggcgatagggttaagggaaggcggacgcctgatgggttaatgagcaaactgaagtgttttccatgatcttttttgagtcgcaattgaagtaccacctcccgagggtgattgcttccccatgcggggtagaacctttgctgtcctgttcaccactctacctccagcacagaatttggcttatgcctactcaatgtgaagatgatgaggatgaaaacctttgtgatgatccacttccacttaatgaatggtggcaaagcaaagctatattcaagaccacatgcaaagctactccctgagcaaagagtcacagataaaacgggggcaccagtagaatggccaggacaaacgcagtgcagcacagagactcagaccctggcagccatgcctgcgcaggcagtgatgagagtgacatgtactgttgtggacatgcacaaaagtgagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaaca

SEQ ID NO: 7 - DNA, 4624 bp, Homo sapiens
  source 1..4624: mol_type = genomic DNA
gagagcgcggtggagagccgagcgggcgggcggcgggtgcggagcgggcgagggagcgcgcgcggccgccacaaagctcgggcgccgcggggctgcatgcggcgtacctggcccggcgcggcgactgctctccgggctggcgggggccggccgcgagccccgggggccccgaggccgcagcttgcctgcgcgctctgagccttcgcaactcgcgagcaaagtttggtggaggcaacgccaagcctgagtcctttcttcctctcgttccccaaatccgagggcagcccgcgggcgtcatgcccgcgctcctccgcagcctggggtacgcgtgaagcccgggaggcttggcgccggcgaagacccaaggaccactcttctgcgtttggagttgctccccgcaaccccgggctcgtcgctttctccatcccgacccacgcggggcgcggggacaacacaggtcgcggaggagcgttgccattcaagtgactgcagcagcagcggcagcgcctcggttcctgagcccaccgcaggctgaaggcattgcgcgtagtccatgcccgtagaggaagtgtgcagatgggattaacgtccacatggagatatggaagaggaccggggattggtaccgtaaccatggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgaggaatacttggacctcagccaacctctcgaacagtattcacctagttaccctgacacaagaagttcttgttcttcaggagatgattctgttttttctccagaccccatgccttacgaaccatgccttcctcagtatccacacataaacggcagtgttaaaacatgaatgactgtgtctgcctgtccccaaacaggacagcactgggaacctagctacactgagcagggagaccatgcctcccagagcttgttgtctccacttgtatatatggatcagaggagtaaataattggaaaagtaatcagcatatgtgtaaagatttatacagttgaaaacttgtaatcttccccaggaggagaagaaggtttctggagcagtggactgccacaagccaccatgtaacccctctcacctgccgtgcgtactggctgtggaccagtaggactcaaggtggacgtgcgttctgccttccttgttaattttgtaataattggagaagatttatgtcagcacacacttacagagcacaaatgcagtatataggtgctggatgtatgtaaatatattcaaattatgtataaatatatattatatatttacaaggagttattttttgtattgattttaaatggatgtcccaatgcacctagaaaattggtctctctttttttaatagctatttgctaaatgctgttcttacacataatttcttaattttcaccgagcagaggtggaaaaatacttttgctttcagggaaaatggtataacgttaatttattaataaattggtaatatacaaaacaattaatcatttatagttttttttgtaatttaagtggcatttctatgcaggcagcacagcagactagttaatctattgcttggacttaactagttatcagatcctttgaaaagagaatatttacaatatatgactaatttggggaaaatgaagttttgatttatttgtgtttaaatgctgctgtcagacgattgttcttagacctcctaaatgccccatattaaaagaactcattcataggaaggtgtttcattttggtgtgcaaccctgtcattacgtcaacgcaacgtctaactggacttcccaagataaatggtaccagcgtcctcttaaaagatgccttaatccattccttgaggacagaccttagttgaaatgatagcagaatgtgcttctctctggcagctggccttctgcttctgagttgcacattaatcagattagcctgtattctcttcagtgaattttgataatggcttccagactctttggcgttggagacgcctgttaggatcttcaagtcccatcatagaaaattgaaacacagagttgttctgctgatagttttggggatacgtccatctttttaagggattgctttcatctaattctggcaggacctcaccaaaagatccagcctcatacctacatcagacaaaatatcgccgttgttccttctgtactaaagtattgtgttttgctttggaaacacccactcactttgcaatagccgtgcaagatgaatgcagattacactgatcttatgtgttacaaaattggagaaagtatttaataaaacctgttaatttttatactgacaataaaaatgtttctacagatattaatgttaacaagacaaaataaatgtcacgcaacttatttttttaa

SEQ ID NO: 8 - DNA, 4307 bp, Homo sapiens
  source 1..4307: mol_type = genomic DNA
agtgcgcggtggcggcggcgtcgcgggcagctggcgccgcgcggtcctgctctgccggtcgcacggacgcaccggcgggccgccggccggagggacggggcgggagctgggcccgcggacagcgagccggagcgggagccgcgcgtagcgagccgggctccggcgctcgccagtctcccgagcggcgcccgcctcccgccggtgcccgcgccgggccgtggggggcagcatgcccgcgcgcgctgcctgaggacgccgcggcccccgcccccgccatgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagtcctggatcagtgagagtgtggaggccgacgtgcgcctccgcctggccaatgtgtcggagcgggacgggggcgagtacctctgtcgagccaccaatttcataggcgtggccgagaaggccttttggctgagcgttcacgggccccgagcagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgagtacctggacctgtcggcgcctttcgagcagtactccccgggtggccaggacacccccagctccagctcctcaggggacgactccgtgtttgcccacgacctgctgcccccggccccacccagcagtgggggctcgcggacgtgaagggccactggtccccaacaatgtgaggggtccctagcagcccaccctgctgctggtgcacagccactccccggcatgagactcagtgcagatggagagacagctacacagagctttggtctgtgtgtgtgtgtgtgcgtgtgtgtgtgtgtgtgtgcacatccgcgtgtgcctgtgtgcgtgcgcatcttgcctccaggtgcagaggtaccctgggtgtccccgctgctgtgcaacggtctcctgactggtgctgcagcaccgaggggcctttgttctggggggacccagtgcagaatgtaagtgggcccacccggtgggacccccgtggggcagggagctgggcccgacatggctccggcctctgcctttgcaccacgggacatcacagggtgggcctcggcccctcccacacccaaagctgagcctgcagggaagccccacatgtccagcaccttgtgcctggggtgttagtggcaccgcctccccacctccaggctttcccacttcccaccctgcccctcagagactgaaattacgggtacctgaagatgggagcctttaccttttatgcaaaaggtttattccggaaactagtgtacatttctataaatagatgctgtgtatatggtatatatacatatatatatataacatatatggaagaggaaaaggctggtacaacggaggcctgcgaccctgggggcacaggaggcaggcatggccctgggcggggcgtgggggggcgtggagggaggccccagggggtctcacccatgcaagcagaggaccagggccttttctggcaccgcagttttgttttaaaactggacctgtatatttgtaaagctatttatgggcccctggcactcttgttcccacaccccaacacttccagcatttagctggccacatggcggagagttttaatttttaacttattgacaaccgagaaggtttatcccgccgatagagggacggccaagaatgtacgtccagcctgccccggagctggaggatcccctccaagcctaaaaggttgttaatagttggaggtgattccagtgaagatattttatttcctttgtcctttttcaggagaattagatttctataggatttttctttaggagatttattttttggacttcaaagcaagctggtattttcatacaaattcttctaattgctgtgtgtcccaggcagggagacggtttccagggaggggccggccctgtgtgcaggttccgatgttattagatgttacaagtttatatatatctatatatataatttattgagtttttacaagatgtatttgttgtagacttaacacttcttacgcaatgcttctagagttttatagcctggactgctacctttcaaagcttggagggaagccgtgaattcagttggttcgttctgtactgttactgggccctgagtctgggcagctgtcccttgcttgcctgcagggccatggctcagggtggtctcttcttggggcccagtgcatggtggccagaggtgtcacccaaaccggcaggtgcgattttgttaacccagcgacgaactttccgaaaaataaagacacctggttgctaa

SEQ ID NO: 9 - DNA, 2565 bp, Homo sapiens
  source 1..2565: mol_type = genomic DNA
cttttcgggcgtcccgaggccgctccccaaccgacaaccaagaccccgcaggccacgcagccctggagccgaggccccccgacggcggaggcgcccgcgggtcccctacagccaaggtccctgagtgccagaggtggtggtgttgcttatcttctggaaccccatgcagccagatcccaggcctagcggggctggggcctgctgccgattcctgcccctgcagtcacagtgccctgagggggcaggggacgcggtgatgtacgcctccactgagtgcaaggcggaggtgacgccctcccagcatggcaaccgcaccttcagctacaccctggaggatcataccaagcaggcctttggcatcatgaacgagctgcggctcagccagcagctgtgtgacgtcacactgcaggtcaagtaccaggatgcaccggccgcccagttcatggcccacaaggtggtgctggcctcatccagccctgtcttcaaggccatgttcaccaacgggctgcgggagcagggcatggaggtggtgtccattgagggtatccaccccaaggtcatggagcgcctcattgaattcgcctacacggcctccatctccatgggcgagaagtgtgtcctccacgtcatgaacggtgctgtcatgtaccagatcgacagcgttgtccgtgcctgcagtgacttcctggtgcagcagctggaccccagcaatgccatcggcatcgccaacttcgctgagcagattggctgtgtggagttgcaccagcgtgcccgggagtacatctacatgcattttggggaggtggccaagcaagaggagttcttcaacctgtcccactgccaactggtgaccctcatcagccgggacgacctgaacgtgcgctgcgagtccgaggtcttccacgcctgcatcaactgggtcaagtacgactgcgaacagcgacggttctacgtccaggcgctgctgcgggccgtgcgctgccactcgttgacgccgaacttcctgcagatgcagctgcagaagtgcgagatcctgcagtccgactcccgctgcaaggactacctggtcaagatcttcgaggagctcaccctgcacaagcccacgcaggtgatgccctgccgggcgcccaaggtgggccgcctgatctacaccgcgggcggctacttccgacagtcgctcagctacctggaggcttacaaccccagtgacggcacctggctccggttggcggacctgcaggtgccgcggagcggcctggccggctgcgtggtgggcgggctgttgtacgccgtgggcggcaggaacaactcgcccgacggcaacaccgactccagcgccctggactgttacaaccccatgaccaatcagtggtcgccctgcgcccccatgagcgtgccccgtaaccgcatcggggtgggggtcatcgatggccacatctatgccgtcggcggctcccacggctgcatccaccacaacagtgtggagaggtatgagccagagcgggatgagtggcacttggtggccccaatgctgacacgaaggatcggggtgggcgtggctgtcctcaatcgtctcctttatgccgtggggggctttgacgggacaaaccgccttaattcagctgagtgttactacccagagaggaacgagtggcgaatgatcacagcaatgaacaccatccgaagcggggcaggcgtctgcgtcctgcacaactgtatctatgctgctgggggctatgatggtcaggaccagctgaacagcgtggagcgctacgatgtggaaacagagacgtggactttcgtagcccccatgaagcaccggcgaagtgccctggggatcactgtccaccaggggagaatctacgtccttggaggctatgatggtcacacgttcctggacagtgtggagtgttacgacccagatacagacacctggagcgaggtgacccgaatgacatcgggccggagtggggtgggcgtggctgtcaccatggagccctgccggaagcagattgaccagcagaactgtacctgttgaggcacttttgtttcttgggcaaaaatacagtccaatggggagtatcattgtttttgtacaaaaaccgggactaaaagaaaagacagcactgcaaataacccatcttccgggaagggaggccaggatgcctcagtgttaaaatgacatctcaaaagaagtccaaagcgggaatcatgtgcccctcagcggagccccgggagtgtccaagacagcctggctgggaaagggggtgtggaaagagcaggcttccaggagagaggcccccaaaccctctggccgggtaataggcctgggtcccactcacccatgccggcagctgtcaccatgtgatttattcttggatacctgggagggggccaatgggggcctcagggggaggccccctctggaaatgtggttcccagggatgggcctgtacatagaagccaccggatggcacttccccaccggatggacagttattttgttgataagtaaccctgtaattttccaaggaaaataaagaacagactaactagtgtctttca

SEQ ID NO: 10 - DNA, 5430 bp, Homo sapiens
  source 1..5430: mol_type = genomic DNA
ctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagagagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggcttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccacacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgac

SEQ ID NO: 11 - DNA, 2022 bp, Homo sapiens
  source 1..2022: mol_type = genomic DNA
agagaagccagcaagtagttgagtgtgacgggtgcatcggttcgggtcgaaggaaatgaagctggagaggaccaacttggaggccttgcagaagaagctggaggagctagagcttgatgagcagcagcgaaagcgccttgaggcctttcttacccagaagcagaaggtgggagaactgaaggatgacgactttgagaagatcagtgagctgggggctggcaatggcggtgtggtgttcaaggtctcccacaagccttctggcctggtcatggccagaaagctaattcatctggagatcaaacccgcaatccggaaccagatcataagggagctgcaggttctgcatgagtgcaactctccgtacatcgtgggcttctatggtgcgttctacagcgatggcgagatcagtatctgcatggagcacatggtaataaaaggcctgacatatctgagggagaagcacaagatcatgcacagagatgtcaagccctccaacatcctagtcaactcccgtggggagatcaagctctgtgactttggggtcagcgggcagctcatcgactccatggccaactccttcgtgggcacaaggtcctacatgtcgccagaaagactccaggggactcattactctgtgcagtcagacatctggagcatgggactgtctctggtagagatggcggttgggaggtatcccatccctcctccagatgccaaggagctggagctgatgtttgggtgccaggtggaaggagatgcggctgagaccccacccaggccaaggacccccgggaggccccttagctcatacggaatggacagccgacctcccatggcaatttttgagttgttggattacatagtcaacgagcctcctccaaaactgcccagtggagtgttcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccagcccagcacaccaacccatgctgctggcgtctaagtgtttgggaagcaacaaagagcgagtcccctgcccggtggtttgccatgtcgcttttgggcctccttcccatgcctgtctctgttcagatgtgcatttcacctgtgacaaaggatgaagaacacagcatgtgccaagattctactcttgtcatttttaatattactgtctttattcttattactattattgttcccctaagtggattggctttgtgcttggggctatttgtgtgtatgctgatgatcaaaacctgtgccaggctgaattacagtgaaattttggtgaatgtgggtagtcattcttacaattgcactgctgttcctgctccatgactggctgtctgcctgtattttcgggattctttgacatttggtggtactttattcttgctgggcatactttctctctaggagggagccttgtgagatccttcacaggcagtgcatgtgaagcatgctttgctgctatgaaaatgagcatcagagagtgtacatcatgttattttattattattatttgcttttcatgtagaactcagcagttgacatccaaatctagccagagcccttcactgccatgatagctggggcttcaccagtctgtctactgtggtgatctgtagacttctggttgtatttctatatttattttcagtatactgtgtgggatacttagtggtatgtctctttaagttttgattaatgtttcttaaatggaattattttgaatgtcacaaattgatcaagatattaaaatgtcggatttatctttccccatatccaagtaccaatgctgttgtaaacaacgtgtatagtgcctaaaattgtatgaaaatccttttaaccattttaacctagatgtttaacaaatctaatctcttattctaataaatatactatgaaataaaaaaaaaaggatgaaagcta

SEQ ID NO: 12 - DNA, 6876 bp, Homo sapiens
  source 1..6876: mol_type = genomic DNA
agacacgtgctggggcgggcaggcgagcgcctcagtctggtcgcctggcggtgcctccggccccaacgcgcccgggccgccgcgggccgcgcgcgccgatgcccggctgagtcactggcagggcagcgcgcgtgtgggaaggggcggagggagtgcggccggcgggcgggcggggcgctgggctcagcccggccgcaggtgacccggaggccctcgccgcccgcggcgccccgagcgctttgtgagcagatgcggagccgagtggagggcgcgagccagatgcggggcgacagctgacttgctgagaggaggcggggaggcgcggagcgcgcgtgtggtccttgcgccgctgacttctccactggttcctgggcaccgaaagataaacctctcataatgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtacttggtggaaagaacctctcaacattgtcagttttctattttgctttgccagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatcatagtgctagtactatgtcaaagcaacagtccacactttgtccaatggttttttcactgcctgacctttaaaaggccatcgatattctttgctcttgccaaaattgcactattataggacttgtattgttatttaaattactggattctaaggaatttcttatctgacagagcatcagaaccagaggcttggtcccacaggccacggaccaatggcctgcagccgtgacaacactcctgtcatattggagtccaaaacttgaattctgggttgaattttttaaaaatcaggtaccacttgatttcatatgggaaattgaagcaggaaatattgagggcttcttgatcacagaaaactcagaagagatagtaatgctcaggacaggagcggcagccccagaacaggccactcatttagaattctagtgtttcaaaacacttttgtgtgttgtatggtcaataacatttttcattactgatggtgtcattcacccattaggtaaacattcccttttaaatgtttgtttgttttttgagacaggatctcactctgttgccagggctgtagtgcagtggtgtgatcatagctcactgcaacctccacctcccaggctcaagcctcccgaatagctgggactacaggcgcacaccaccatccccggctaatttttgtattttttgtagagacggggttttgccatgttgccaaggctggtttcaaactcctggactcaagaaatccacccacctcagcctcccaaagtgctaggattacaggcatgagccactgcgcccagcccttataaatttttgtatagacattcctttggttggaagaatatttataggcaatacagtcaaagtttcaaaatagcatcacacaaaacatgtttataaatgaacaggatgtaatgtacatagatgacattaagaaaatttgtatgaaataatttagtcatcatgaaatatttagttgtcatataaaaacccactgtttgagaatgatgctactctgatctaatgaatgtgaacatgtagatgttttgtgtgtatttttttaaatgaaaactcaaaataagacaagtaatttgttgataaatatttttaaagataactcagcatgtttgtaaagcaggatacattttactaaaaggttcattggttccaatcacagctcataggtagagcaaagaaagggtggatggattgaaaagattagcctctgtctcggtggcaggttcccacctcgcaagcaattggaaacaaaacttttggggagttttattttgcattagggtgtgttttatgttaagcaaaacatactttagaaacaaatgaaaaaggcaattgaaaatcccagctatttcacctagatggaatagccaccctgagcagaactttgtgatgcttcattctgtggaattttgtgcttgctactgtatagtgcatgtggtgtaggttactctaactggttttgtcgacgtaaacatttaaagtgttatattttttataaaaatgtttatttttaatgatatgagaaaaattttgttaggccacaaaaacactgcactgtgaacattttagaaaaggtatgtcagactgggattaatgacagcatgattttcaatgactgtaaattgcgataaggaaatgtactgattgccaatacaccccaccctcattacatcatcaggacttgaagccaagggttaacccagcaagctacaaagagggtgtgtcacactgaaactcaatagttgagtttggctgttgttgcaggaaaatgattataactaaaagctctctgatagtgcagagacttaccagaagacacaaggaattgtactgaagagctattacaatccaaatattgccgtttcataaatgtaataagtaatactaattcacagagtattgtaaatggtggatgacaaaagaaaatctgctctgtggaaagaaagaactgtctctaccagggtcaagagcatgaacgcatcaatagaaagaactcggggaaacatcccatcaacaggactacacacttgtatatacattcttgagaacactgcaatgtgaaaatcacgtttgctatttataaacttgtccttagattaatgtgtctggacagattgtgggagtaagtgattcttctaagaattagatacttgtcactgcctatacctgcagctgaactgaatggtacttcgtatgttaatagttgttctgataaatcatgcaattaaagtaaagtgatgcaa

SEQ ID NO: 13 - DNA, 4326 bp, Homo sapiens
  source 1..4326: mol_type = genomic DNA
ggggccggaagtgccgctccttggtgggggctgttcatggcggttccggggtctccaacatttttcccggctgtggtcctaaatctgtccaaagcagaggcagtggagcttgaggttcttgctggtgtgaaatgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaacaagatacttttaaagttttgtcagaaaagagccactttcaagctgcactgacaccctggtcctgacttccctggaggagaagtattcctgttgctgtcttcagtctcacagagaagctcctgctacttccccagctctcagtagtttagtacaataatctctatttgagaagttctcagaataactacctcctcacttggctgtctgaccagagaatgcacctcttgttactccctgttatttttctgccctgggttcttccacagcacaaacacacctctgccaccccaggtttttcatctgaaaagcagttcatgtctgaaacagagaaccaaaccgcaaacgtgaaattctattgaaaacagtgtcttgagctctaaagtagcaactgctggtgattttttttttctttttactgttgaacttagaactatgctaatttttggagaaatgtcataaattactgttttgccaagaatatagttattattgctgtttggtttgtttataatgttatcggctctattctctaaactggcatctgctctagattcataaatacaaaaatgaatactgaattttgagtctatcctagtcttcacaactttgacgtaattaaatccaactttcacagtgaagtgcctttttcctagaagtggtttgtagacttcctttataatatttcagtggaatagatgtctcaaaaatccttatgcatgaaatgaatgtctgagatacgtctgtgacttatctaccattgaaggaaagctatatctatttgagagcagatgccattttgtacatgtatgaaattggttttccagaggcctgttttggggctttcccaggagaaagatgaaactgaaagcacatgaataatttcacttaataatttttacctaatctccacttttttcataggttactacctatacaatgtatgtaatttgtttcccctagcttactgataaacctaatattcaatgaacttccatttgtattcaaatttgtgtcataccagaaagctctacatttgcagatgttcaaatattgtaaaactttggtgcattgttatttaatagctgtgatcagtgattttcaaacctcaaatatagtatattaacaaattacattttcactgtatatcatggtatcttaatgatgtatataattgccttcaatccccttctcaccccaccctctacagcttcccccacagcaataggggcttgattatttcagttgagtaaagcatggtgctaatggaccagggtcacagtttcaaaacttgaacaatccagttagcatcacagagaaagaaattcttctgcatttgctcattgcaccagtaactccagctagtaattttgctaggtagctgcagttagccctgcaaggaaagaagaggtcagttagcacaaaccctttaccatgactggaaaactcagtatcacgtatttaaacatttttttttcttttagccatgtagaaactctaaattaagccaatattctcatttgagaatgaggatgtctcagctgagaaacgttttaaattctctttattcataatgttctttgaagggtttaaaacaagatgttgataaatctaagctgatgagtttgctcaaaacaggaagttgaaattgttgagacaggaatggaaaatataattaattgatacctatgaggatttggaggcttggcattttaatttgcagataataccctggtaattctcatgaaaaatagacttggataacttttgataaaagactaattccaaaatggccactttgttcctgtctttaatatctaaatacttactgaggtcctccatcttctatattatgaattttcatttattaagcaaatgtcatattaccttgaaattcagaagagaagaaacatatactgtgtccagagtataatgaacctgcagagttgtgcttcttactgctaattctgggagctttcacagtactgtcatcatttgtaaatggaaattctgcttttctgtttctgctccttctggagcagtgctactctgtaattttcctgaggcttatcacctcagtcatttcttttttaaatgtctgtgactggcagtgattctttttcttaaaaatctattaaatttgatgtcaaattagggagaaagatagttactcatcttgggctcttgtgccaatagcccttgtatgtatgtacttagagttttccaagtatgttctaagcacagaagtttctaaatggggccaaaattcagacttgagtatgttctttgaataccttaagaagttacaattagccgggcatggtggcccgtgcctgtagtcccagctacttgagaggctgaggcaggagaatcacttcaacccaggaggtggaggttacagtgagcagagatcgtgccactgcactccagcctgggtgacaagagagacttgtctccaaaaaaaaagttacacctaggtgtgaattttggcacaaaggagtgacaaacttatagttaaaagctgaataacttcagtgtggtataaaacgtggtttttaggctatgtttgtgattgctgaaaagaattctagtttacctcaaaatccttctctttccccaaattaagtgcctggccagctgtcataaattacatattccttttggtttttttaaaggttacatgttcaagagtgaaaataagatgttctgtctgaaggctaccatgccggatctgtaaatgaacctgttaaatgctgtatttgctccaacggcttactatagaatgttacttaatacaatatcatacttattacaatttttactataggagtgtaataggtaaaattaatctctattttagtgggcccatgtttagtctttcaccatcctttaaactgctgtgaatttttttgtcatgacttgaaagcaaggatagagaaacactttagagatatgtggggtttttttaccattccagagcttgtgagcataatcatatttgctttatatttatagtcatgaactcctaagttggcagctacaaccaagaaccaaaaaatggtgcgttctgcttcttgtaattcatctctgctaataaattataagaagcaaggaaaattagggaaaatattttatttggatggtttctataaacaagggactataattcttgtacattatttttcatctttgctgtttctttgagcagtctaatgtgccacacaattatctaaggtatttgttttctataagaattgttttaaaagtattcttgttaccagagtagttgtattatatttcaaaacgtaagatgatttttaaaagcctgagtactgacctaagatggaattgtatgaactctgctctggagggaggggaggatgtccgtggaagttgtaagacttttatttttttgtgccatcaaatataggtaaaaataattgtgcaattctgctgtttaaacaggaactattggcctccttggccctaaatggaagggccgatattttaagttgattattttattgtaaattaatccaacctagttctttttaatttggttgaatgttttttcttgttaaatgatgtttaaaaaataaaaactggaagttcttggcttagtcataa

SEQ ID NO: 14 - DNA, 9259 bp, Homo sapiens
  source 1..9259: mol_type = genomic DNA
agttccggtgccgccgctgcggccgctgaggtgtcgggctgctgctgccgcggccgctgggactggggctggggccgccggcgaggcagggctcgggcccggccgggcagctccggagcggcgggggagaggggccgggaggcgggggccgtgccgcccgctctcctctccctcggcgccgccgccgccgcccgcggggctgggacccgatgcggttagagccgcggagcctggaagagccccgagcgtttctgctttgggacaaccatacatctaattccttaaagtagttttatatgtaaaacttgcaaagaatcagaacaatgcctccacgaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactgaaaagataactgagaaaatgaaagctcactctggattccacactgcactgttaataactctcagcaggcaaagaccgattgcataggaattgcacaatccatgaacagcattagaatttacagcaagaacagaaataaaatactatataatttaaataatgtaaacgcaaacagggtttgatagcacttaaactagttcatttcaaaattaagctttagaataatgcgcaatttcatgttatgccttaagtccaaaaaggtaaactttgaagattgtttgtatctttttttaaaaaacaaaacaaaacaaaaatccccaaaatatatagaaatgatggagaaggaaaaagtgatggttttttttgtcttgcaaatgttctatgttttgaaatgtggacacaacaaaggctgttattgcattaggtgtaagtaaactggagtttatgttaaattacattgattggaaaagaatgaaaatttcttatttttccattgctgttcaatttatagtttgaagtgggtttttgactgcttgtttaatgaagaaaaatgcttggggtggaagggactcttgagatttcaccagagactttttctttttaataaatcaaaccttttgatgatttgaggttttatctgcagttttggaagcagtcacaaatgagacctgttataaggtggtatttttttttttcttctggacagtatttaaaggatcttattcttatttcccagggaaattctgggctcccacaaagtaaaaaaaaaaaaaaatcatagaaaaagaatgagcaggaatagttcttattccagaattgtacagtattcaccttaagttgattttttttctccttctgcaattgaactgaatacatttttcatgcatgttttccagaaaatagaagtattaatgttattaaaaagattattttttttattaaaggctatttatattatagaaactatcattaatatatattctttatttacatgatctgtcccatagtcatgcattgttttgcaccccaaattttttattgttcatagcagcatggtcagctttcttcttgatctatagatgaggctcaggcactatcccatttataccaataaccagtgtataactacttaaggaaaacataaaaacttcatcttctttccttttatttcttatgtgaatctcccgtcttccattctcttttataattgagaatgtctcaatcatatgaaattagttaccagaattaacacaatttagactatcttcctgattccttaaacccctttactgaagtatactcatgaataatactttaaaatatgggggaatagaaaccatgaactttttacctttttaaactatttatccatatctccaaagtagaacattaaaccattttaagatatgtctcattcccaagtagtcagagctcactctccaactttattaaatactatttgagcacaggacacattcttaaacattttgaaaaacattaacccaagatgtagaggctactgctagtcgtcattctagaatctgatattttactctgtatttgaaatgaatgattaatgtcctaggaaattagctttagcagatgtccaggtgccacatcaaaaaagtgcaataattattgacagttttttagattaggcatattattggaaaacaactttataaagagtgaacattgtatactctagtaaaacagcatcactttaaaaatattcatttatgaaatctgttacctatagttgaagtcttgagtagtgaacaagggactctaataccaatactcttaatatctggctattttagatcccttaaagggcataattattggaaatttaggtatttcactaaagcatgtatataatattgccaacaagaaaagtaaatttgaagattaagggaacttacttctgcaaactgtcttgcgatagttaagcagaatttaaactctgttttaagcaggaaaccagaaagattattttgcagttgtagaagatttcataacttattaaaacttattaacattttgtgttgtttagatataggcagttgatacatactaacatcccagccttttcaatatcagggttaaattataggaaaactcagtaaaatggtacaaatctgaaagtttgatggtagaaactgaagatttaacagagaactgtgttttacccgagtgccaaaaatgctgtgagcctccttgcacaaaatttataccacttttgcatttttatctatcagtccagatagttgtctcccctccttctcccaggacctctccaccattaaaatgcacaaaccacatggccgatttcaccatttacatttattttcaaaagttactacaaccaaattaattctattagaagaaatgtagacaaattctataaagactatagattgtgacctaagaaagaaatgaggcaaagaaccaaacattgaattaaatgctacatgggtgactaagatctgtttcaagtcagtgataatatagccacttctgggtacttcagtatcagagatcagttctcgtggtttagacagttcctatctatagctgactatccttgtccttgaatatggtgtaactgactattggctctacagttttattgggccacttaagaaatatttccttgaataattattttgagaaaaagtctaaaagtaataaaaataattttaaacacactgtagtaagaaatgactgttggaaaattatgctttcactttctaccatattctcagctatacaaaaccatttattttgaagatttttagactactgttaatttgaaatctgttactcttattgtggaatttgtttttttaaaaaagatgtttctaattggatttttaaaagaagaatggaatttggttgctattttacaatagaacctaagctttttgtggttcttagtgtcctatgtaaaacttagtgtcaaagtaatcaactttgagattttcccttctattctgctttatattaaaagcccattagaaaatgggaacctggtgaatatataatgaattgtaaaatattttaatgtgtaactttttcaactgtgaaactgacttgattttttgatgaaaacagctgctgataaagtattttgtgtaaagtgtagttcttattaatcaggaaaatgatgacttgattagactgtatatgccctcttggattttattttaaatggattggtgactttcacataggtaaaacacagtccatctgtattcttttttccatcaaaaatcgagtgatttggaattataaaaaaattgtgagcagcctatttgaaaggcatcatggaaatttcacagcacaataacacggatttgttttttcttaatgatgtaaatccgtttaattcatactttgatcaatagcccatgcttgccaactctgaagaaatttaatttccagcagtattttaaagctagcctgttaactttttctgaatatttaaagttcctcttttttctatgtctgcacaaactgcagacctgggctggacccacatactcaagagtccaccttaagaaattattttgatgtccaagacatcactaaaatatttaagtttaaagataatatgtggtgttaatagattgtggtgcttttactatttaaagacaactttcatacttcagatgtttttgagaagaggggaatgtgaggggagggggcagaacagggaggagttgtttgaatgaattacattctttatatccatcctgctcatttggggcatgtctttaagagaaggctgaaagttgtgagagtatattgtataccgtaagagaatcaactcttcatcatggatgggattgtgaaggctgaactataaaattcagcattgacagcatcctcaattaataattcttggtgacagaataatacagctgggctgttttttaaaatataaacaataccatttttaattattacattaaaaattgtaaatatatctatgtgccatggcctgggaagcctgctttcttttttcataaaaattatttttactgtatgaaaagatcatggggtttagctcaaaatatctgtggtcctgataaaattggattggtaactctacctcagaaggaaaatgggaaaaaaaaatagatgagtcacaattcaatacttcaagctcagaaactgtgcagatcactgaattttagatttataaagtcagagttggcatgccttgtttttaatgatatggaagaccttaagaaaaaaacttggctgaagtttaatcgttggtccagccatttgaaaaaggcaatagtttgaggaggttcccgaattcggcatttgaaattcattttgttctctcttcttcattattagtgcatttggtgtgtgtatacttgcacacaattctgtttgtgtacacactgcttgcttagccctagtcaagaggcatcttttataaaaggtgtaaagaaatatcaaggttctaaaattcggaagagtttagaatttattaggagtttcccaagttgggatgttagtctttaaataaacttcatgcacctattccacttaaggttttgcacctcctttttattagtgcagtgccatttcttctgcttgattttaggtatgttaatattccagccttgctagttagcataaagtgacaggtgtgagccatgaggaaattttctgacttaatttgtacacaactacatataagagttttagtggaggaaaaaaattagtcccttgtgcgtatacagtagttaggtaaatgatttttctaccaacagtatactccattcctcatgtaggtaagtacagaaaaggtttttaaatgtatttttttagccagttaaagtctatgaatctatctgcaaccttatttaatctgtcactataataattttgtggttatgctaagaaccatgtatacttttaggtattcttatttttgtcaatttttctaggttggcaaggaggcagaaaaccttcattgtttcatattaaaatataattagactaaacttaattctagtatgaatttccaaaatcattatctatttatttcatttttatttaattttgtttttatttcatttttaaaagtcccttgttcaatttaacttatgttcctaagagaggttggagaacttggccttcatctgatttcaaaaatgttttgagtttcaaatgaagttaatggtttcagtgtgattcagtcctcagacctaattgggttgaataaaatctaaaagaatatacccttttggagcataacattttaataccttggggaatgtggcactaccaaaagaagactactaacacgtcagatgttcacctggaagctttatcaagaaattcgaaccacccttttggccccattaattgtagcaagtttatttctctatattttgtcattcagtgaattgaagtcctgtggtatactgcattcattagaagaaaaacgtttttaatgtccttttaatgatggcccagaaagcatttgacacagcaagatgcatgtgttactatattgagaatatagaataataacagtatcactaaatttaagacctcttcccagtcttgctgttcctagcaagaagtttggcctgtgactgcacttactgtttatgctcatcagaaactgtcaatgtctgcttttctttaactctgcagtctgtaacatcacgctgtttattaaaaaaaaaaagaaaaatta

SEQ ID NO: 15 - DNA, 4385 bp, Homo sapiens
  source 1..4385: mol_type = genomic DNA
agtcccgcgaccgaagcagggcgcgcagcagcgctgagtgccccggaacgtgcgtcgcgcccccagtgtccgtcgcgtccgccgcgccccgggcggggatggggcggccagactgagcgccgcacccgccatccagacccgccggccctagccgcagtccctccagccgtggccccagcgcgcacgggcgatggcgaaggcgacgtccggtgccgcggggctgcgtctgctgttgctgctgctgctgccgctgctaggcaaagtggcattgggcctctacttctcgagggatgcttactgggagaagctgtatgtggaccaggcagccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggcatgtcagacccgaactggcctggagagagtcctgtaccactcacgagagctgatggcactaacactgggtttccaagatatccaaatgatagtgtatatgctaactggatgctttcaccctcagcggcaaaattaatggacacgtttgatagttaacatttctttgtgaaagatgcacaacactcctccagtcttgtgggggcagcttttgggaagtctcagcagctcttctggctgtgttgtcagcactgtaacttcgcagaaaagagtcggattaccaaaacactgcctgctcttcagacttaaagcactgataggacttaaaatagtctcattcaaatactgtattttatataggcatttcacaaaaacagcaaaattgtggcattttgtgaggccaaggcttggatgcgtgtgtaatagagccttgtggtgtgtgcgcacacacccagagggagagtttgaaaaatgcttattggacacgtaacctggctctaatttgggctgtttttcagatacactgtgataagttcttttacaaatatctatagacatggtaaacttttggttttcagatatgcttaatgatagtcttactaaatgcagaaataagaataaactttctcaaattattaaaaatgcctacacagtaagtgtgaattgctgcaacaggtttgttctcaggagggtaagaactccaggtctaaacagctgacccagtgatggggaatttatccttgaccaatttatccttgaccaataacctaattgtctattcctgagttataaaagtccccatccttattagctctactggaattttcatacacgtaaatgcagaagttactaagtattaagtattactgagtattaagtagtaatctgtcagttattaaaatttgtaaaatctatttatgaaaggtcattaaaccagatcatgttcctttttttgtaatcaaggtgactaagaaaatcagttgtgtaaataaaatcatgtatcataaaa

SEQ ID NO: 16 - DNA, 8469 bp, Homo sapiens
  source 1..8469: mol_type = genomic DNA
gcacttctaagaactaacctttagtcactgggtgactttatgggagtaaaaggaagctgttatgaaatagctcttatggaactgttacaagctttcaagcattcaaaggtctaaatgaaaaaggctaagtattatttcaaaaggcaagtatatcctaatatagcaaaacaaacaaagcaaaatccatcagctactcctccaattgaagtgatgaagcccaaataattcatatagcaaaatggagaaaattagaccggccatctaaaaatctgccattggtgaagtgatgaagaacatttactgtcttattccgaagcttgtcaattttgcaactcttggctgcctatggatttctgtggtgcagtgtacagttttaaatagctgcctaaagtcgtgtgtaactaatctgggccagcagcttgaccttggcacaccacataatctgagtgaaccgtgtatccaaggatgtcacttttggaactctgtagatcagaaaaactgtgctttaaagtgtcgggagtcgtgtgaggttggctgtagcagcgcggaaggtgcatatgaagaggaagtactggaaaatgcagacctaccaactgctccctttgcttcttccattggaagccacaatatgacattacgatggaaatctgcaaacttctctggagtaaaatacatcattcagtggaaatatgcacaacttctgggaagctggacttatactaagactgtgtccagaccgtcctatgtggtcaagcccctgcaccccttcactgagtacattttccgagtggtttggatcttcacagcgcagctgcagctctactcccctccaagtcccagttacaggactcatcctcatggagttcctgaaactgcacctttgattaggaatattgagagctcaagtcccgacactgtggaagtcagctgggatccacctcaattcccaggtggacctattttgggttataacttaaggctgatcagcaaaaatcaaaaattagatgcagggacacagagaaccagtttccagttttactccactttaccaaatactatctacaggttttctattgcagcagtaaatgaagttggtgagggtccagaagcagaatctagtattaccacttcatcttcagcagttcaacaagaggaacagtggctctttttatccagaaaaacttctctaagaaagagatctttaaaacatttagtagatgaagcacattgccttcggttggatgctatataccataatattacaggaatatctgttgatgtccaccagcaaattgtttatttctctgaaggaactctcatatgggcgaagaaggctgccaacatgtctgatgtatctgacctgagaattttttacagaggttcaggattaatttcttctatctccatagattggctttatcaaagaatgtatttcatcatggatgaactggtatgtgtctgtgatttagagaactgctcaaacatcgaggaaattactccaccctctattagtgcacctcaaaaaattgtggctgattcatacaatgggtatgtcttttacctcctgagagatggcatttatagagcagaccttcctgtaccatctggccggtgtgcagaagctgtgcgtattgtggagagttgcacgttaaaggactttgcaatcaagccacaagccaagcgaatcatttacttcaatgacactgcccaagtcttcatgtcaacatttctggatggctctgcttcccatctcatcctacctcgcatcccctttgctgatgtgaaaagttttgcttgtgaaaacaatgactttcttgtcacagatggcaaggtcattttccaacaggatgctttgtcttttaatgaattcatcgtgggatgtgacctgagtcacatagaagaatttgggtttggtaacttggtcatctttggctcatcctcccagctgcaccctctgccaggccgcccgcaggagctttcggtgctgtttggctctcaccaggctcttgttcaatggaagcctcctgcccttgccataggagccaatgtcatcctgatcagtgatattattgaactctttgaattaggcccttctgcctggcagaactggacctatgaggtgaaagtatccacccaagaccctcctgaagtcactcatattttcttgaacataagtggaaccatgctgaatgtacctgagctgcagagtgctatgaaatacaaggtttctgtgagagcaagttctccaaagaggccaggcccctggtcagagccctcagtgggtactaccctggtgccagctagtgaaccaccatttatcatggctgtgaaagaagatgggctttggagtaaaccattaaatagctttggcccaggagagttcttatcctctgatataggaaatgtgtcagacatggattggtataacaacagcctctactacagtgacacgaaaggcgacgtttttgtgtggctgctgaatgggacggatatctcagagaattatcacctacccagcattgcaggagcaggggctttagcttttgagtggctgggtcactttctctactgggctggaaagacatatgtgatacaaaggcagtctgtgttgacgggacacacagacattgttacccacgtgaagctattggtgaatgacatggtggtggattcagttggtggatatctctactggaccacactctattcagtggaaagcaccagactaaatggggaaagttcccttgtactacagacacagccttggttttctgggaaaaaggtaattgctctaactttagacctcagtgatgggctcctgtattggttggttcaagacagtcaatgtattcacctgtacacagctgttcttcggggacagagcactggggataccaccatcacagaatttgcagcctggagtacttctgaaatttcccagaatgcactgatgtactatagtggtcggctgttctggatcaatggctttaggattatcacaactcaagaaataggtcagaaaaccagtgtctctgttttggaaccagccagatttaatcagttcacaattattcagacatcccttaagcccctgccagggaacttttcctttacccctaaggttattccagattctgttcaagagtcttcatttaggattgaaggaaatgcttcaagttttcaaatcctgtggaatggtccccctgcggtagactggggtgtagttttctacagtgtagaatttagtgctcattctaagttcttggctagtgaacaacactctttacctgtatttactgtggaaggactggaaccttatgccttatttaatctttctgtcactccttatacctactggggaaagggccccaaaacatctctgtcacttcgagcacctgaaacagttccatcagcaccagagaaccccagaatatttatattaccaagtggaaaatgctgcaacaagaatgaagttgtggtggaatttaggtggaacaaacctaagcatgaaaatggggtgttaacaaaatttgaaattttctacaatatatccaatcaaagtattacaaacaaaacatgtgaagactggattgctgtcaatgtcactccctcagtgatgtcttttcaacttgaaggcatgagtcccagatgctttattgccttccaggttagggcctttacatctaaggggccaggaccatatgctgacgttgtaaagtctacaacatcagaaatcaacccatttcctcacctcataactcttcttggtaacaagatagtttttttagatatggatcaaaatcaagttgtgtggacgttttcagcagaaagagttatcagtgccgtttgctacacagctgataatgagatgggatattatgctgaaggggactcactctttcttctgcacttgcacaatcgctctagctctgagcttttccaagattcactggtttttgatatcacagttattacaattgactggatttcaaggcacctctactttgcactgaaagaatcacaaaatggaatgcaagtatttgatgttgatcttgaacacaaggtgaaatatcccagagaggtgaagattcacaataggaattcaacaataatttctttttctgtatatcctcttttaagtcgcttgtattggacagaagtttccaattttggctaccagatgttctactacagtattatcagtcacaccttgcaccgaattctgcaacccacagctacaaaccaacaaaacaaaaggaatcaatgttcttgtaatgtgactgaatttgagttaagtggagcaatggctattgatacctctaacctagagaaaccattgatatactttgccaaagcacaagagatctgggcaatggatctggaaggctgtcagtgttggagagttatcacagtacctgctatgctcgcaggaaaaacccttgttagcttaactgtggatggagatcttatatactggatcatcacagcaaaggacagcacacagatttatcaggcaaagaaaggaaatggggccatcgtttcccaggtgaaggccctaaggagtaggcatatcttggcttacagttcagttatgcagccttttccagataaagcgtttctgtctctagcttcagacactgtggaaccaactatacttaatgccactaacactagcctcacaatcagattacctctggccaagacaaacctcacatggtatggcatcaccagccctactccaacatacctggtttattatgcagaagttaatgacaggaaaaacagctctgacttgaaatatagaattctggaatttcaggacagtatagctcttattgaagatttacaaccattttcaacatacatgatacagatagctgtaaaaaattattattcagatcctttggaacatttaccaccaggaaaagagatttggggaaaaactaaaaatggagtaccagaggcagtgcagctcattaatacaactgtgcggtcagacaccagcctcattatatcttggagagaatctcacaagccaaatggacctaaagaatcagtccgttatcagttggcaatctcacacctggccctaattcctgaaactcctctaagacaaagtgaatttccaaatggaaggctcactctccttgttactagactgtctggtggaaatatttatgtgttaaaggttcttgcctgccactctgaggaaatgtggtgtacagagagtcatcctgtcactgtggaaatgtttaacacaccagagaaaccttattccttggttccagagaacactagtttgcaatttaattggaaggctccattgaatgttaacctcatcagattttgggttgagctacagaagtggaaatacaatgagttttaccatgttaaaacttcatgcagccaaggtcctgcttatgtctgtaatatcacaaatctacaaccttatacttcatataatgtcagagtagtggtggtttataagacgggagaaaatagcacctcacttccagaaagctttaagacaaaagctggagtcccaaataaaccaggcattcccaaattactagaagggagtaaaaattcaatacagtgggagaaagctgaagataatggatgtagaattacatactatatccttgagataagaaagagcacttcaaataatttacagaaccagaatttaaggtggaagatgacatttaatggatcctgcagtagtgtttgcacatggaagtccaaaaacctgaaaggaatatttcagttcagagtagtagctgcaaataatctagggtttggtgaatatagtggaatcagtgagaatattatattagttggagatgatttttggataccagaaacaagtttcatacttactattatagttggaatatttctggttgttacaatcccactgacctttgtctggcatagaagattaaagaatcaaaaaagtgccaaggaaggggtgacagtgcttataaacgaagacaaagagttggctgagctgcgaggtctggcagccggagtaggcctggctaatgcctgctatgcaatacatactcttccaacccaagaggagattgaaaatcttcctgccttccctcgggaaaaactgactctgcgtctcttgctgggaagtggagcctttggagaagtgtatgaaggaacagcagtggacatcttaggagttggaagtggagaaatcaaagtagcagtgaagactttgaagaagggttccacagaccaggagaagattgaattcctgaaggaggcacatctgatgagcaaatttaatcatcccaacattctgaagcagcttggagtttgtctgctgaatgaaccccaatacattatcctggaactgatggagggaggagaccttcttacttatttgcgtaaagcccggatggcaacgttttatggtcctttactcaccttggttgaccttgtagacctgtgtgtagatatttcaaaaggctgtgtctacttggaacggatgcatttcattcacagggatctggcagctagaaattgccttgtttccgtgaaagactataccagtccacggatagtgaagattggagactttggactcgccagagacatctataaaaatgattactatagaaagagaggggaaggcctgctcccagttcggtggatggctccagaaagtttgatggatggaatcttcactactcaatctgatgtatggtcttttggaattctgatttgggagattttaactcttggtcatcagccttatccagctcattccaaccttgatgtgttaaactatgtgcaaacaggagggagactggagccaccaagaaattgtcctgatgatctgtggaatttaatgacccagtgctgggctcaagaacccgaccaaagacctacttttcatagaattcaggaccaacttcagttattcagaaattttttcttaaatagcatttataagtccagagatgaagcaaacaacagtggagtcataaatgaaagctttgaaggtgaagatggcgatgtgatttgtttgaattcagatgacattatgccagttgctttaatggaaacgaagaaccgagaagggttaaactatatggtacttgctacagaatgtggccaaggtgaagaaaagtctgagggtcctctaggctcccaggaatctgaatcttgtggtctgaggaaagaagagaaggaaccacatgcagacaaagatttctgccaagaaaaacaagtggcttactgcccttctggcaagcctgaaggcctgaactatgcctgtctcactcacagtggatatggagatgggtctgattaatagcgttgtttgggaaatagagagttgagataaacactctcattcagtagttactgaaagaaaactctgctagaatgataaatgtcatggtggtctataactccaaataaacaatgcaacgttcctgatttctaatcttggttctgagagccatttggtttcagttgtagcaatccccataccagctgcctgactttcagtagaattatgagatgaacactaagcatgtggaaagcttaggaagactcagaagtctggaagggaaacactgctctcccttctcccttgaggtgctttaggctcttacccacctttcagtttgggctgtaataaaaatatcttggccacatgtttagagacagaataggtgtgttcagcgatataaagaagaggctaaggagtaggctcaggggggtcaactgaactacagataatctcaaatgggaccaaggaaatgagaaataatttcacacatacagaagaaaccagcacctgtgacttgagaaatcacttggaaagctgttactgcaatgatatatatattatctttttttaattttttttttttttttttgagacgaagtcttgctctgttgcccaggctggagttcaatggcacgatctcggcactgcaaactccacctcctgggttcaagcaattctcgtgcctcagcctcctaagtagctgggattacaggcgtgtgccaccacgcccggctaatttttgtatttttagtagagatgaggtttcaccatattggccaggctggtctagaactcctgacctcgggatccacctgccttggcctcccaaagtgctggattacaggtgtgagccaccatgcctagccgatatatattgtctttaatcactactgtaaaatattttgtagttttgaggcttacaacagtagattcagtcatgttgaaaataagactgtgaagatcttttaagtcctgaagttttgcattctgtaatcttcagttgtataaaatcactctgacttgtgtgctattatggaaattaactagtataaagattgctatttgccatatctattttatgtataaaatacttaagattacattttgtatcaaattatgcttaaaattaaatataaatgattatacaatgttaa

SEQ ID NO: 17 - DNA, 3293 bp, Homo sapiens
  source 1..3293: mol_type = genomic DNA
gaggtaaacaagatggcggcggcgtgtcgggcgcggaagggggaggcggcccggggcgcccgcgagtgaggcgcggggcggcgaagggagcgcgggtggcggcacttgctgccgcggccttggatgggctgggcccccctcgccgctccgcctcctccacacgcgcggcggccgcggcgagggggacgcgccgcccggggcccggcaccttcgggaaccccccggcccggagcctgcggcctgcgccgcctcggccgccgggagccccgtggagcccccgccgccgcgccgccccgcggaccggacgctgagggcactcggggcggggcgcgcgctcgggcagacgtttgcggggaggggggcgcctgccgggccccggcgaccaccttgggggtcgcgggccggctcggggggcgcccagtgcgggccctcgcgggcgccgggcagcgaccagccctgagcggagctgttggccgcggcgggaggcctcccggacgcccccagccccccgaacgctcgcccgggccggcgggagtcggcgccccccgggaggtccgctcggtcgtccgcggcggagcgtttgctcctgggacaggcggtgggaccggggcgtcgccggagacgcccccagcgaagttgggctctccaggtgtgggggtcccggggggtagcgacgtcgcggacccggcctgtgggatgggcggcccggagaagactgcgctcggccgtgttcatacttgtccgtgggcctgaggtccccggaggatgacctagcactgaaaagccccggccggcctccccagggtccccgaggacgaagttgaccctgaccgggccgtctcccagttctgaggcccgggtcccactggaactcgcgtctgagccgccgtcccggacccccggtgcccgccggtccgcagaccctgcaccgggcttggactcgcagccgggactgacgtgtagaacaatcgtttctgttggaagaagggtttttcccttccttttggggtttttgttgccttttttttttcttttttctttgtaaaattttggagaagggaagtcggaacacaaggaaggaccgctcacccgcggactcagggctggcggcgggactccaggaccctgggtccagcatggaggtggtggacccgcagcagctgggcatgttcacggagggcgagctgatgtcggtgggtatggacacgttcatccaccgcatcgactccaccgaggtcatctaccagccgcgccgcaagcgggccaagctcatcggcaagtacctgatgggggacctgctgggggaaggctcttacggcaaggtgaaggaggtgctggactcggagacgctgtgcaggagggccgtcaagatcctcaagaagaagaagttgcgaaggatccccaacggggaggccaacgtgaagaaggaaattcaactactgaggaggttacggcacaaaaatgtcatccagctggtggatgtgttatacaacgaagagaagcagaaaatgtatatggtgatggagtactgcgtgtgtggcatgcaggaaatgctggacagcgtgccggagaagcgtttcccagtgtgccaggcccacgggtacttctgtcagctgattgacggcctggagtacctgcatagccagggcattgtgcacaaggacatcaagccggggaacctgctgctcaccaccggtggcaccctcaaaatctccgacctgggcgtggccgaggcactgcacccgttcgcggcggacgacacctgccggaccagccagggctccccggctttccagccgcccgagattgccaacggcctggacaccttctccggcttcaaggtggacatctggtcggctggggtcaccctctacaacatcaccacgggtctgtaccccttcgaaggggacaacatctacaagttgtttgagaacatcgggaaggggagctacgccatcccgggcgactgtggccccccgctctctgacctgctgaaagggatgcttgagtacgaaccggccaagaggttctccatccggcagatccggcagcacagctggttccggaagaaacatcctccggctgaagcaccagtgcccatcccaccgagcccagacaccaaggaccggtggcgcagcatgactgtggtgccgtacttggaggacctgcacggcgcggacgaggacgaggacctcttcgacatcgaggatgacatcatctacactcaggacttcacggtgcccggacaggtcccagaagaggaggccagtcacaatggacagcgccggggcctccccaaggccgtgtgtatgaacggcacagaggcggcgcagctgagcaccaaatccagggcggagggccgggcccccaaccctgcccgcaaggcctgctccgccagcagcaagatccgccggctgtcggcctgcaagcagcagtgaggctggccgcctgcagcccgtgtccaggagccccgccaggtgcccgcgccaggccctcagtcttcctgccggttccgcccgccctcccggagaggtggccgccatgcttctgtgccgaccacgccccaggacctccggagcgccctgcagggccgggcagggggacagcagggaccgggcgcagccctcccccctcggccgcccggcagtgcacgcggcttgttgacttcgcagccccgggcggagccttcccgggcgggcgtgggaggagggaggcggcctccatgcactttatgtggagactactggccccgcccgtggcctcgtgctccgcagggcgcccagcgccgtccggcggccccgccgcagaccagctggcgggtgtggagaccaggctcctgaccccgccatgcatgcagcgccacctggaagccgcgcggccgctttggttttttgtttggttggttccattttctttttttctttttttttttaagaaaaaataaaaggtggatttgagctgtggctgtgaggggtgtttgggagctgctgggtggcaggggggctgtggggtcgggctcacgtcgcggccgcctttgcgctctcgggtcaccctgctttggcggcccggccggagggcaggaccctcacctctcccccaaggccactgcgctcttgggaccccagagaaaacccggagcaagcaggagtgtgcggtcaatatttatatcatccagaaaagaaaaacacgagaaacgccatcgcgggatggtgcagacgcggcggggactcggagggtgccgtgcgggcgaggccgcccaaatttggcaataaataaagcttgggaagcttgga

SEQ ID NO: 18 - DNA, 2512 bp, Homo sapiens
  source 1..2512: mol_type = genomic DNA
ctcaaaagtctagagccaccgtccagggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgcca

SEQ ID NO: 19 - DNA, 2669 bp, Homo sapiens
  source 1..2669: mol_type = genomic DNA
ggaggcctggcagctgcagctgggagcgcacagacggctgccccgcctgagcgaggcgggcgccgccgcgatgctgcgaggcggacggcgcgggcagcttggctggcacagctgggctgcggggccgggcagcctgctggcttggctgatactggcatctgcgggcgccgcaccctgccccgatgcctgctgcccccacggctcctcgggactgcgatgcacccgggatggggccctggatagcctccaccacctgcccggcgcagagaacctgactgagctctacatcgagaaccagcagcatctgcagcatctggagctccgtgatctgaggggcctgggggagctgagaaacctcaccatcgtgaagagtggtctccgtttcgtggcgccagatgccttccatttcactcctcggctcagtcgcctgaatctctccttcaacgctctggagtctctctcctggaaaactgtgcagggcctctccttacaggaactggtcctgtcggggaaccctctgcactgttcttgtgccctgcgctggctacagcgctgggaggaggagggactgggcggagtgcctgaacagaagctgcagtgtcatgggcaagggcccctggcccacatgcccaatgccagctgtggtgtgcccacgctgaaggtccaggtgcccaatgcctcggtggatgtgggggacgacgtgctgctgcggtgccaggtggaggggcggggcctggagcaggccggctggatcctcacagagctggagcagtcagccacggtgatgaaatctgggggtctgccatccctggggctgaccctggccaatgtcaccagtgacctcaacaggaagaacgtgacgtgctgggcagagaacgatgtgggccgggcagaggtctctgttcaggtcaacgtctccttcccggccagtgtgcagctgcacacggcggtggagatgcaccactggtgcatccccttctctgtggatgggcagccggcaccgtctctgcgctggctcttcaatggctccgtgctcaatgagaccagcttcatcttcactgagttcctggagccggcagccaatgagaccgtgcggcacgggtgtctgcgcctcaaccagcccacccacgtcaacaacggcaactacacgctgctggctgccaaccccttcggccaggcctccgcctccatcatggctgccttcatggacaaccctttcgagttcaaccccgaggaccccatccctgtctccttctcgccggtggacactaacagcacatctggagacccggtggagaagaaggacgaaacaccttttggggtctcggtggctgtgggcctggccgtctttgcctgcctcttcctttctacgctgctccttgtgctcaacaaatgtggacggagaaacaagtttgggatcaaccgcccggctgtgctggctccagaggatgggctggccatgtccctgcatttcatgacattgggtggcagctccctgtcccccaccgagggcaaaggctctgggctccaaggccacatcatcgagaacccacaatacttcagtgatgcctgtgttcaccacatcaagcgccgggacatcgtgctcaagtgggagctgggggagggcgcctttgggaaggtcttccttgctgagtgccacaacctcctgcctgagcaggacaagatgctggtggctgtcaaggcactgaaggaggcgtccgagagtgctcggcaggacttccagcgtgaggctgagctgctcaccatgctgcagcaccagcacatcgtgcgcttcttcggcgtctgcaccgagggccgccccctgctcatggtctttgagtatatgcggcacggggacctcaaccgcttcctccgatcccatggacctgatgccaagctgctggctggtggggaggatgtggctccaggccccctgggtctggggcagctgctggccgtggctagccaggtcgctgcggggatggtgtacctggcgggtctgcattttgtgcaccgggacctggccacacgcaactgtctagtgggccagggactggtggtcaagattggtgattttggcatgagcagggatatctacagcaccgactattaccgtgtgggaggccgcaccatgctgcccattcgctggatgccgcccgagagcatcctgtaccgtaagttcaccaccgagagcgacgtgtggagcttcggcgtggtgctctgggagatcttcacctacggcaagcagccctggtaccagctctccaacacggaggcaatcgactgcatcacgcagggacgtgagttggagcggccacgtgcctgcccaccagaggtctacgccatcatgcggggctgctggcagcgggagccccagcaacgccacagcatcaaggatgtgcacgcccggctgcaagccctggcccaggcacctcctgtctacctggatgtcctgggctagggggccggcccaggggctgggagtggttagccggaatactggggcctgccctcagcatcccccatagctcccagcagccccagggtgatctcaaagtatctaattcaccctcagcatgtgggaagggacaggtgggggctgggagtagaggatgttcctgcttctctaggcaaggtcccgtcatagcaattatatttattatcccttg

SEQ ID NO: 20 - DNA, 5697 bp, Homo sapiens
  source 1..5697: mol_type = genomic DNA
gcatagcgctcggagcgctcttgcggccacaggcgcggcgtcctcggcggcgggcggcagctagcgggagccgggacgccggtgcagccgcagcgcgcggaggaacccgggtgtgccgggagctgggcggccacgtccggacgggaccgagacccctcgtagcgcattgcggcgacctcgccttccccggccgcgagcgcgccgctgcttgaaaagccgcggaacccaaggacttttctccggtccgagctcggggcgccccgcagggcgcacggtacccgtgctgcagtcgggcacgccgcggcgccggggcctccgcagggcgatggagcccggtctgcaaggaaagtgaggcgccgccgctgcgttctggaggaggggggcacaaggtctggagaccccgggtggcggacgggagccctccccccgccccgcctccggggcaccagctccggctccattgttcccgcccgggctggaggcgccgagcaccgagcgccgccgggagtcgagcgccggccgcggagctcttgcgaccccgccaggacccgaacagagcccgggggcggcgggccggagccggggacgcgggcacacgcccgctcgcacaagccacggcggactctcccgaggcggaacctccacgccgagcgagggtcagtttgaaaaggaggatcgagctcactgtggagtatccatggagatgtggagccttgtcaccaacctctaactgcagaactgggatgtggagctggaagtgcctcctcttctgggctgtgctggtcacagccacactctgcaccgctaggccgtccccgaccttgcctgaacaagcccagccctggggagcccctgtggaagtggagtccttcctggtccaccccggtgacctgctgcagcttcgctgtcggctgcgggacgatgtgcagagcatcaactggctgcgggacggggtgcagctggcggaaagcaaccgcacccgcatcacaggggaggaggtggaggtgcaggactccgtgcccgcagactccggcctctatgcttgcgtaaccagcagcccctcgggcagtgacaccacctacttctccgtcaatgtttcagatgctctcccctcctcggaggatgatgatgatgatgatgactcctcttcagaggagaaagaaacagataacaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtaacagtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctgactgccacccacacgccctccccagactccaccgtcagctgtaaccctcacccacagcccctgctgggcccaccacctgtccgtccctgtcccctttcctgctggcaggagccggctgcctaccaggggccttcctgtgtggcctgccttcaccccactcagctcacctctccctccacctcctctccacctgctggtgagaggtgcaaagaggcagatctttgctgccagccacttcatcccctcccagatgttggaccaacacccctccctgccaccaggcactgcctggagggcagggagtgggagccaatgaacaggcatgcaagtgagagcttcctgagctttctcctgtcggtttggtctgttttgccttcacccataagcccctcgcactctggtggcaggtgccttgtcctcagggctacagcagtagggaggtcagtgcttcgtgcctcgattgaaggtgacctctgccccagataggtggtgccagtggcttattaattccgatactagtttgctttgctgaccaaatgcctggtaccagaggatggtgaggcgaaggccaggttgggggcagtgttgtggccctggggcccagccccaaactgggggctctgtatatagctatgaagaaaacacaaagtgtataaatctgagtatatatttacatgtctttttaaaagggtcgttaccagagatttacccatcgggtaagatgctcctggtggctgggaggcatcagttgctatatattaaaaacaaaaaagaaaaaaaaggaaaatgtttttaaaaaggtcatatattttttgctacttttgctgttttatttttttaaattatgttctaaacctattttcagtttaggtccctcaataaaaattgctgctgcttcatttatctatgggctgtatgaaaagggtgggaatgtccactggaaagaagggacacccacgggccctggggctaggtctgtcccgagggcaccgcatgctcccggcgcaggttccttgtaacctcttcttcctaggtcctgcacccagacctcacgacgcacctcctgcctctccgctgcttttggaaagtcagaaaaagaagatgtctgcttcgagggcaggaaccccatccatgcagtagaggcgctgggcagagagtcaaggcccagcagccatcgaccatggatggtttcctccaaggaaaccggtggggttgggctggggagggggcacctacctaggaatagccacggggtagagctacagtgattaagaggaaagcaagggcgcggttgctcacgcctgtaatcccagcactttgggacaccgaggtgggcagatcacttcaggtcaggagtttgagaccagcctggccaacttagtgaaaccccatctctactaaaaatgcaaaaattatccaggcatggtggcacacgcctgtaatcccagctccacaggaggctgaggcagaatcccttgaagctgggaggcggaggttgcagtgagccgagattgcgccattgcactccagcctgggcaacagagaaaacaaaaaggaaaacaaatgatgaaggtctgcagaaactgaaacccagacatgtgtctgccccctctatgtgggcatggttttgccagtgcttctaagtgcaggagaacatgtcacctgaggctagttttgcattcaggtccctggcttcgtttcttgttggtatgcctccccagatcgtccttcctgtatccatgtgaccagactgtatttgttgggactgtcgcagatcttggcttcttacagttcttcctgtccaaactccatcctgtccctcaggaacggggggaaaattctccgaatgtttttggttttttggctgcttggaatttacttctgccacctgctggtcatcactgtcctcactaagtggattctggctcccccgtacctcatggctcaaactaccactcctcagtcgctatattaaagcttatattttgctggattactgctaaatacaaaagaaagttcaatatgttttcatttctgtagggaaaatgggattgctgctttaaatttctgagctagggattttttggcagctgcagtgttggcgactattgtaaaattctctttgtttctctctgtaaatagcacctgctaacattacaatttgtatttatgtttaaagaaggcatcatttggtgaacagaactaggaaatgaatttttagctcttaaaagcatttgctttgagaccgcacaggagtgtctttccttgtaaaacagtgatgataatttctgccttggccctaccttgaagcaatgttgtgtgaagggatgaagaatctaaaagtcttcataagtccttgggagaggtgctagaaaaatataaggcactatcataattacagtgatgtccttgctgttactactcaaatcacccacaaatttccccaaagactgcgctagctgtcaaataaaagacagtgaaattga

SEQ ID NO: 21 - DNA, 4515 bp, Homo sapiens
  source 1..4515: mol_type = genomic DNA
ggagtttattcataacgcgctctccaagtatacgtggcaatgcgttgctgggttattttaatcattctaggcatcgttttcctccttatgcctctatcattcctccctatctacactaacatcccacgctctgaacgcgcgcccattaatacccttctttcctccactctccctgggactcttgatcaaagcgcggccctttccccagccttagcgaggcgccctgcagcctggtacgcgcgtggcgtggcggtgggcgcgcagtgcgttctcggtgtggagggcagctgttccgcctgcgatgatttatactcacaggacaaggatgcggtttgtcaaacagtactgctacggaggagcagcagagaaagggagagggtttgagagggagcaaaagaaaatggtaggcgcgcgtagttaattcatgcggctctcttactctgtttacatcctagagctagagtgctcggctgcccggctgagtctcctccccaccttccccaccctccccaccctccccataagcgcccctcccgggttcccaaagcagagggcgtgggggaaaagaaaaaagatcctctctcgctaatctccgcccaccggccctttataatgcgagggtctggacggctgaggacccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaatcttaagttgtgaatgttttgtttcgtttcttccccctcccaaccaccaccatccctgtttgttttcatcaattgccccttcagagggtggtcttaagaaaggcaagagttttcctctgttgaaatgggtctgggggccttaaggtctttaagttcttggaggttctaagatgcttcctggagactatgataacagccagagttgacagttagaaggaatggcagaaggcaggtgagaaggtgagaggtaggcaaaggagatacaagaggtcaaaggtagcagttaagtacacaaagaggcataaggactggggagttgggaggaaggtgaggaagaaactcctgttactttagttaaccagtgccagtcccctgctcactccaaacccaggaattctgcccagttgatggggacacggtgggaaccagcttctgctgccttcacaaccaggcgccagtcctgtccatgggttatctcgcaaaccccagaggatctctgggaggaatgctactattaaccctatttcacaaacaaggaaatagaagagctcaaagaggttatgtaacttatctgtagccacgcagataatacaaagcagcaatctggacccattctgttcaaaacacttaacccttcgctatcatgccttggttcatctgggtctaatgtgctgagatcaagaaggtttaggacctaatggacagactcaagtcataacaatgctaagctctatttgtgtcccaagcactcctaagcattttatccctaactctacatcaaccccatgaaggagatactgttgatttccccatattagaagtagagagggaagctgaggcacacaaagactcatccacatgcccaagattcactgatagggaaaagtggaagcgagatttgaacccaggctgtttactcctaacctgtccaagccacctctcagacgacggtaggaatcagctggctgcttgtgagtacaggagttacagtccagtgggttatgttttttaagtctcaacatctaagcctggtcaggcatcagttcccctttttttgtgatttattttgtttttattttgttgttcattgtttaatttttccttttacaatgagaaggtcaccatcttgactcctaccttagccatttgttgaatcagactcatgacggctcctgggaagaagccagttcagatcataaaataaaacatatttattctttgtcatgggagtcattattttagaaactacaaactctccttgcttccatccttttttacatactcatgacacatgctcatcctgagtccttgaaaaggtatttttgaacatgtgtattaattataagcctctgaaaacctatggcccaaaccagaaatgatgttgattatataggtaaatgaaggatgctattgctgttctaattacctcattgtctcagtctcaaagtaggtcttcagctccctgtactttgggattttaatctaccaccacccataaatcaataaataattactttctttga

SEQ ID NO: 22 - DNA, 8515 bp, Homo sapiens
  source 1..8515: mol_type = genomic DNA
gttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggctggcacatccagggacccgggccggttttaaacctcccgtgcgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaagggttttgctacattctaatgcatgtattcgggttaggggaatggagggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaacgacttctccatctcctgtgtaatcaaggccagtgctaaaattcagatgctgttagtacctacatcagtcaacaacttacacttattttactagttttcaatcataatacctgctgtggatgcttcatgtgctgcctgcaagcttcttttttctcattaaatataaaatattttgtaatgctgcacagaaattttcaatttgagattctacagtaagcgttttttttctttgaagatttatgatgcacttattcaatagctgtcagccgttccacccttttgaccttacacattctattacaatgaattttgcagttttgcacattttttaaatgtcattaactgttagggaattttacttgaatactgaatacatataatgtttatattaaaaaggacatttgtgttaaaaaggaaattagagttgcagtaaactttcaatgctgcacacaaaaaaaagacatttgatttttcagtagaaattgtcctacatgtgctttattgatttgctattgaaagaatagggtttttttttttttttttttttttttttttaaatgtgcagtgttgaatcatttcttcatagtgctcccccgagttgggactagggcttcaatttcacttcttaaaaaaaatcatcatatatttgatatgcccagactgcatacgattttaagcggagtacaactactattgtaaagctaatgtgaagatattattaaaaaggtttttttttccagaaatttggtgtcttcaaattataccttcaccttgacatttgaatatccagccattttgtttcttaatggtataaaattccattttcaataacttattggtgctgaaattgttcactagctgtggtctgacctagttaatttacaaatacagattgaataggacctactagagcagcatttatagagtttgatggcaaatagattaggcagaacttcatctaaaatattcttagtaaataatgttgacacgttttccataccttgtcagtttcattcaacaatttttaaatttttaacaaagctcttaggatttacacatttatatttaaacattgatatatagagtattgattgattgctcataagttaaattggtaaagttagagacaactattctaacacctcaccattgaaatttatatgccaccttgtctttcataaaagctgaaaattgttacctaaaatgaaaatcaacttcatgttttgaagatagttataaatattgttctttgttacaatttcgggcaccgcatattaaaacgtaactttattgttccaatatgtaacatggagggccaggtcataaataatgacattataatgggcttttgcactgttattatttttcctttggaatgtgaaggtctgaatgagggttttgattttgaatgtttcaatgtttttgagaagccttgcttacattttatggtgtagtcattggaaatggaaaaatggcattatatatattatatatataaatatatattatacatactctccttactttatttcagttaccatccccatagaatttgacaagaattgctatgactgaaaggttttcgagtcctaattaaaactttatttatggcagtattcataattagcctgaaatgcattctgtaggtaatctctgagtttctggaatattttcttagactttttggatgtgcagcagcttacatgtctgaagttacttgaaggcatcacttttaagaaagcttacagttgggccctgtaccatcccaagtcctttgtagctcctcttgaacatgtttgccatacttttaaaagggtagttgaataaatagcatcaccattctttgctgtggcacaggttataaacttaagtggagtttaccggcagcatcaaatgtttcagctttaaaaaataaaagtagggtacaagtttaatgtttagttctagaaattttgtgcaatatgttcataacgatggctgtggttgccacaaagtgcctcgtttacctttaaatactgttaatgtgtcatgcatgcagatggaaggggtggaactgtgcactaaagtgggggctttaactgtagtatttggcagagttgccttctacctgccagttcaaaagttcaacctgttttcatatagaatatatatactaaaaaatttcagtctgttaaacagccttactctgattcagcctcttcagatactcttgtgctgtgcagcagtggctctgtgtgtaaatgctatgcactgaggatacacaaaaataccaatatgatgtgtacaggataatgcctcatcccaatcagatgtccatttgttattgtgtttgttaacaaccctttatctcttagtgttataaactccacttaaaactgattaaagtctcattcttgtcattgtgtgggtgttttattaaatgagagtttataattcaaattgcttaagtccattgaagttttaattaatgggcagccaaatgtgaatacaaagttttcagtttttttttttcctgctgtccttcaaagcctactgtttaaaaaaaaaaaaaaaaaaaaacatggcctgagagtagagtatctgtctactcatgtttaattaaggaaaaacacttatttttagggctttagtcatcacttcataaattgtataagcacattaaatagcgttctagtcctgaaaaagtccaagattcttagaaaattgtgcatatttttattatgacagatgtttgaagataattccccagaatggatttgatactttagatttcaattttgtggcttttgtctattattctgtactctgccatcagcatatggaaagcttcatttactcatcatgacttgtgccatataaaaattgatatttcggaatagtctaaaggactttttgtacttgaatttaatcatgttgtttctaatattcttaaaagcttgaagactaaagcatatcctttcaacaaagcatagtaaggtaataagaaagtgtagtttgtacaagtgttaaaaaaataaagtagacaatgttacagtgggacttattatttcaagtttacattttctccatgtaattttttaaaaagtaaatgaaaaaatgtgcaataatgtaaaatatgaagtgtatgtgtacacacattttatttttcggtatcttgggtatacgtatggttgaaaactatactggagtctaaaagtattctaatttataagaagacattttggtgatgtttgaaaaatagaaatgtgctagttttgtttttatatcatgtcctttgtacgttgtaatatgagctggcttggttcagtaaatgccatcaccatttccattgagaatttaaaactcaccagtgtttaatatgcaggcttccaaaggcttatgaaaaaaatcaagacccttaaatctagttaatttgctgctaacatgaaactctttggttcttttatttttgccagataattagacacacatctaaagcttagtcttaaatggcttaagtgtagctattgattagtgctgttgctagttcagaaagaaatgtttgtgaatggaaacaagaatattcagtccaaactgttgtaaggacagtacctgaaaaccaggaaacaggataatggaaaaagtcttttaaagatgaaatgttggagccaactttcttatagaattaattgtatgtggctatagaaagcctaatgattgttgcttatttttgagagcatattattcttttatgaccataatcttgctgtttttccatcttccaaaagatcttccttctaatatgtatatcagaatgtgggtagccagtcagacaaattcatattggttggtagctttaaaaagtttgtaatgtgaagacaggaaaggacaaaatagtttgctttggtggtagtactctggttgttaagctaggtattttgagactacttccccatcacaacaacaataaaataatcactcataatcctatcacctggagacatagccatcgttaatatgttagtgactatacaatcatgttttcttctgtatatccatgtatattctttaaaaatgaaatttatactgtacctgatctcaaagctttttagcttagtatatctgtcatgaatttgtaggatgttccattgcatcagaaaacggacagtgatttgattactttctaatgccacagatgcagattacatgtagttattgagaatcctttcgaattcagtggcttaatcatgaatgtctaaatattgttgacattaggatgatacatgtaaattaaagttacatttgtttagcatagacaagcttaacattgtagatgtttctcttcaaaaatcatcttaaacatttgcatttggaattgtgttaaatagaatgtgtgaaacactgtattagtaaacttcatcacctttctacttccttatagtttgaacttttcagtttttgtagttcccaaacagttgctcaatttagagcaaattaatttaacacctgccaaaaaaaggctgctgttggcttatcagttgtctttaaattcaaatgctcatgtgacttttatcacatcaaaaaatatttcattaatgattcacctttagctctgaaaattaccgcgtttagtaattatagtgggcttataaaaacatgcaactctttttgatagttatttgagaattttggtgaaaaatatttagctgagggcagtatagaacttataaaccaatatattgatatttttaaaacatttttacatataagtaaactgccatctttgagcataactacatttaaaaataaagctgcatatttttaaatcaagtgtttaacaagaatttatattttttattttttaaaattaaaaataatttatatttcctctgttgcatgaggattctcatctgtgcttataatggttagagattttatttgtgtggaatgaagtgaggcttgtagtcatggttctagtgtttcagtttgccaagtctgtttactgcagtgaaattcatcaaatgtttcagtgtggttttctgtagcctatcatttactggctatttttttatgtacacctttaggattttctgcctactctatccagttgtccaaatgatatcctacattttacaaatgccctttcagtttctattttctttttccattaaattgccctcatgtcctaatgtgcagtttgtaagtgtgtgtgtgtgtgtctgtgtgtgtgtgaatttgattttcaagagtgctagacttccaatttgagagattaaataatttaattcaggcaaacatttttcattggaatttcacagttcattgtaatgaaaatgttaatcctggatgacctttgacatacagtaatgaatcttggatattaatgaatttgttagtagcatcttgatgtgtgttttaatgagttattttcaaagttgtgcattaaaccaaagttggcatactggaagtgtttatatcaagttccatttggctactgatggacaaaaaatagaaatgccttcctatggagagtatttttcctttaaaaaattaaaaaggttaattattttgacta

SEQ ID NO: 23 - DNA, 9605 bp, Homo sapiens
  source 1..9605: mol_type = genomic DNA
gttgtgactgaaacccgtcaatatggcggcgatcggccgcggccgctctctgaagaacctccgagtacgagggcggaatgacagcggcgaggagaacgtcccgctggatctgacccgagaaccttctgataacttaagagagattctccaaaatgtggccagattgcagggagtatcaaatatgagaaagctaggccatctgaataactttactaagcttctttgtgatattggccacagtgaagaaaaactgggctttcactatgaggatatcataatttgtttgcggttagctttattaaatgaagcaaaagaagtgcgagcagcagggctacgagcgcttcgatatctcatccaagactccagtattctccagaaggtgctaaaattgaaagtggactatttaatagctaggtgcattgacatacaacagagcaacgaggtagagaggacacaagcacttcgattagtcagaaagatgattactgtgaatgcttccttgtttcctagttctgtgaccaactcattaattgcagttggaaatgatggacttcaagaaagagacagaatggtccgagcatgcattgccattatctgtgaactagcacttcagaatccagaggtggtggcccttcgaggtggactaaacaccatcttgaaaaatgtgatcgattgccaattaagtcgaataaatgaggccctaattactacaattttgcaccttcttaatcatccaaagactcgacagtatgtgcgagctgatgtagaattagagagaattttagcaccctatactgattttcactacagacatagtccagatacagctgaaggacagctcaaagaagacagagaagcacgatttctagccagtaaaatgggaatcatagcaacattccgatcatgggcaggtattattaatttatgtaaacctggaaattctgggatccagtctctaataggagtactttgcataccaaatatggaaataaggcgaggtctacttgaagtgctttatgatatatttcgtcttcctctacctgttgtgactgaggagttcatagaagcactactcagtgtagatccagggaggttccaagacagttggaggctttcagatggctttgtggcagctgaggcaaaaactattcttcctcatcgtgccagatccaggccagacctcatggataattatttggcactgatactctctgcatttattcgtaatggacttttagagggtctagttgaagtgataacaaacagtgatgatcatatctcagttagagctaccatccttttaggagagcttttacatatggcaaacacaattcttcctcattcacatagccatcatttacactgcttgccaaccctaatgaatatggctgcatcctttgatatccccaaggaaaagagactgcgagccagtgcagccttgaactgtttaaaacgcttccatgaaatgaagaaacgaggacctaagccttatagtcttcatttagaccacattattcagaaagcaattgcaacacaccagaaacgggatcagtatctccgagttcagaaagatatatttatccttaaggatacagaggaagctcttttaattaaccttagagatagccaagtccttcaacataaagagaatcttgaatggaattggaatcttatagggaccattcttaagtggccaaatgtaaatctaagaaactataaagatgaacagttacacaggtttgtacgaagactactttatttttacaagcccagcagtaaattatatgccaacctggatctggattttgccaaggccaaacagctcacggttgtaggttgccagtttacagaatttcttcttgaatctgaagaggatgggcaaggctacttagaagatctagtaaaggatattgttcagtggctcaatgcttcatctggaatgaaacccgaaagaagtcttcaaaataatggtttattgaccacccttagtcaacactactttttatttattggaacactttcttgccaccctcatggagttaaaatgctggaaaaatgcagtgtatttcagtgtctccttaatctttgctccttgaaaaaccaagatcacttgctaaaacttactgtttctagcttggactatagcagagatggattggctagagtcatcctttccaaaattttaactgcagctactgatgcctgcagactctatgcaacaaaacatttaagggtattattgagagctaatgttgaattctttaataattggggaattgagttgttagtgacccagctacatgataaaaacaaaacgatttcctctgaagctcttgatatcctcgatgaagcatgtgaagacaaggccaatcttcatgctctcattcagatgaaaccagcgttatcccaccttggagacaagggtttgcttctcctgctgagatttctctccattccaaaaggattttcctatctgaatgaaagaggttatgtagcaaaacaattggaaaagtggcacagggaatacaactccaaatatgttgacttgattgaggaacaactcaatgaagcacttactacttaccggaagcctgttgatggtgataactatgttcgtcggagtaaccaaagattacagcgtcctcacgtctacctgcctatacacctttatggacaactagtacaccataaaacaggctgccatttgttggaagtacagaatattattacagaactctgtcgtaatgttcgtacaccagatttggataagtgggaagaaattaaaaaactgaaagcatctctttgggccttgggaaatatcggctcatcaaattggggtctcaatttgctacaggaagaaaacgtgattccagatatactaaaacttgcaaaacagtgtgaagttctttccatcagagggacctgtgtatatgtacttgggctcatagctaaaaccaaacaaggctgtgatattctaaaatgtcacaactgggatgctgtgaggcatagtcgcaaacatctgtggccagtggttccagatgatgtggaacaactctgtaatgaactttcatctatcccaagcactctaagtttgaactcggagtcaaccagctctagacataatagtgaaagtgaatctgtgccatcgagtatgttcatattggaggatgaccggtttggcagcagctctactagcacatttttccttgatatcaatgaagatacagagccaacattttatgaccgatctggacccataaaggataaaaattcattccctttctttgcttctagtaaacttgtgaagaatcgtatcttaaattcgcttactttgcctaacaaaaaacatcgtagtagcagtgatccaaaaggagggaaattatcatctgaaagtaagacaagcaacaggcgaatcagaacacttacggagcccagtgttgattttaatcatagtgatgattttacacccatatccactgtacagaaaacattacaattagagacttcatttatggggaataagcacattgaagacactggtagtacaccaagcattggagaaaatgacttaaaattcaccaagaattttggtacagagaatcacagagaaaatacaagccgagagaggttagtagtagaaagttcaacgagctcacatatgaagatacgtagccaaagtttcaatacagacactacaacaagtggcataagttcaatgagctcaagtccttcacgagagacagtaggtgtagatgctacaactatggacacagactgtggaagcatgagtactgtggtaagtactaaaactattaagacaagccactatttgacgccacagtctaaccatctgtctctctccaaatcaaattcggtgtccctggtgcctccaggttcttctcatacgcttcctagaagagcacagtcccttaaagcaccctctattgctacaattaaaagtctagcagattgtaactttagttacacaagttctagagatgcttttggctatgctacactgaaaagactacagcaacaaagaatgcatccatccttatctcactctgaagctttggcatctccagcaaaagatgtgctatttactgataccatcaccatgaaggccaacagttttgagtccagattaacaccaagcaggatcgattttaaaaagaagcatgtcgggggaatcaggagcttaagacctacaataacaaacaaccttttcaggttcatgaaagccttaagttatgcatcattagataaagaagatttattgagtcctattaatcaaaataccctgcaacgatcttcctcagtgcggtccatggtgtccagtgccacatatgggggttcagatgattacattggtcttgctctcccggtggatataaatgatatattccaggtaaaggatattccctattttcagacaaaaaacataccaccacatgatgatcgaggtgcaagagcatttgcccatgatgcaggaggtcttccatctggaactggaggtcttgtaaaaaattcttttcacttgctacgacagcagatgagtcttacggaaataatgaattcaatccattcagatgcctctctgtttttagaaagtacagaagacactggactacaggaacatacagatgataactgcctttattgtgtctgtattgaaattctgggtttccagcccagcaaccaactgagtgcaatatgtagtcattcagactttcaagatattccatattctgattggtgtgagcagactatccataatcctttagaagtggttccctctaagttttcggggatttctggatgcagtgatggggtgtctcaagaaggctcagctagcagcaccaaaagcacagaattgttactaggtgttaaaacaattccagatgatacaccaatgtgccgtatactccttcgcaaagaagttctaagattagtcattaatttgagtagttcagtttcaactaaatgtcatgagactgggcttttaacaattaaggagaagtatcctcaaacatttgatgacatatgcctttactctgaggtttcccatttgctgtcacactgcacattcagacttccgtgtcggaggttcatacaagaattatttcaagatgtacagtttctacaaatgcatgaagaagcagaggctgtgttggcaacaccaccaaagcaacctatagttgatacatctgctgaatcctgacctcatatttatgatggatatagatacatactatatatattcatatttgtggatttcctaaaagcctcagaaaatacgactgactaggcagcaaagacaggagtatcttctgtacactgttccgcagttactggtacatgaacagttggaactgctgactttcctaaccaaaacaacttccttctctcctttgttgagccttttgaggggttcatgattcattaccacagttttaagagtttcagttaccattgtatgcaagagccaagcactgaatacctacataggttttctattttctttcattttaaaagcataatgacagtggaacaataatgggatatgcagaagcacccttcacaagttatttctgaatgatttttagggtaaataatacagatgccttgtttgttaactaacttgtggaaagcaggaatcagtgtctctaaggctgcatcctattaccacaatggggtgtgctataactgctggtattagagagggaactttggccctttcacgtttttcttaatgtttgtaacactacttcagaggtttataacctcaaagcagaagaagagcctcaacaacccgggacttataagttatttttatgttactagacttgcataaagattcttgttttccaactcttcattttgttgcaatgtgttattacaggatatatgaaccaattaaggtttttcactacagttcttgaataaaatttaaaaatcattttttattttaattaaaaatatttcccatttatagaatgcatatatttgcaatggacttccactttcatcaactttccatctcatcgctttaaacaggaacttgaacaagcactgttagtttagacctaaaggataggaaagcattaaataatactttggatctcctgaggaaaagataagtttgcttgcaatttacacattccatggggaaagaagagccatatttccttaaaaaaaacattaataaagcttgttattgagaaaaattgtagtgaaaagccttaagtaccaaattttaaagcagcagtaacttaatttttatatcagtgtttttgttttgcacaaactaaatgcagtggtaggtgggtttatgagtatattaattgcctttatccatttgtgaagttaagttgatgagggcaaggtttttgtttgtttaatttgtatatgtctaaaggtatttggaactttttacaggaattaaacatatatgcaaatttgtatataaaaatagcatggccatcatttgaatgcttgtaaatgaaaggattatcttttttgagatctatatataaatagaaatagaaaatccagctggactgattaggattcttttttaattcatttgtgtataacatttttattacaattacacatcagttttgacacagtcatagcaacattaatattttcccatgatgcagatcctttttgtaatgggcttgttctttgagatctctgtaaagaaccctgtgaactagaaaacataactcacagagatacttttttaaaaaatttatttactggaactgaaagttccagttgggatgaagcatttcatctcacttcataacacctctttgactgcacttcagtgaattgttcttatgtgcactgtgtagcaacttacattataacaaagcagataagggctgtaagctgctgcttatgttgaaaagtggttcttcagattttctctcataaaatccagttgaagataaataatttttttatactttatcactgaacccaagtgtttatttaaatgtcaacagtacttctaagaacgttgcctgtcatcgtggtctttggtcttggataactaaactgcctttccagagaaccaaatgtcagagttactagaccaaatagtggttaaaacctccaaaggaagtaatgtaatcttattcataatgggattaacatattttagacattcattttaaacactacctcagttaatatagagtataaaaatctgtggtttaatccctcaaaagttaacagtaattttttttttgtcttacacacacacacaccccctcccccaccatcactatccctgtaccctcaccttggtcatctatcctgaaataaggcttagttagtattggcctgaatgttttgtgtttttttttttgttttttttttttactgttactttgaaaaatatgtatgtataccttatcatatctgcctatatcacttactttggggagatactcagagctttgtggttatcagtatactaaaaaaaaaaaaaagtctacgcttaaatttatagtgctatttggtttctccatgatttcactgacaggtctaatacattttctttgagtacttgtttgtaaaaagtagactttatggtgaaaaatacatgcagtgccaagtgattaacttaagtgtttaaaaatattaaattatagcagaagaggttaggaatgatatcagcagtaatagaaataattgagaaaatcatctataaataatagatattacagactatagaataccaaaataatgtcaatactgtagtttttaaagattttaggattaatcttagtccatataaatttgtactattggtaattattgaataattgggaggaatctgggcagttgtgctggttgtaaactatgaatttctaatcgtaaagtgaattgttatttctaattgaactttttttcaagaacagatttcagcctcacatactaagtaaatactgataaataaggaaattagaaatttagtattcataattaaatatgctctaaaatttcctatacttttatttcctgtttattcttaggtagattggaagggggaaacagtctgttctccctaattaaattttttctaataacgattagtagaatatggacattctatatgacagtgacattaaaagaggctctttggaagtatatacattattaacataatgtgtacaagtccttttgaaatgacaactttaatgggtttcagctcttttatctagagcttgagataattcaagctgagtttttcagggcatatcacaacggccaagtgttcagcagtgggatatcaatgcttatttacattttcctactgctatttatataaaatgttattccattcagaggatgccttttatccccacattaaagcacagatcattaagcaataaaaaccaaattgtctgtcattcaaattataactgcagttatttttgcatggtaagagtgaggtgctaattttgtgtgagatgaactttgtaaactactttgggaaatgttctttggaagtaaggttttttctcctttagtcttatgcttccacttttgtctcagattcacaatccattaaaacatggggaaaaaagaaaaggtaaaattgagagacttttgttagaggagctatttggaatgaaccaacatttcagattttccaaaatgtaagttaggaagtctccattgtctctgcattaacaaaatacactgttactatcttaatctcaagagtgtcattacagtgagaatctcatttaaaagcataccagtgaaattaatagcagtgcttatcaaagaacactgaaatctgtgagaatctttctaggagcattcttttcttcttttagttccaagttccagggtatttttcattcctagtaggtttatatgactcacagaatgtggacttttttcctgtttggagtatttttgtaatgtaagtatcggatagctgcaccacagcatgcataaattgcacattttgttttactttctttatagaatatttaatttcaaaaatataatttatgccaaaaaaagcatacctttcaattttgctacttggttgatttagcacaaaatgcaaagtcttggggcagagagggggagtgaaaaaaattttataggtaattgttacaaaaatacctgtcagaaaccctaaagctgcattgtaaaacaaatggtgtaaactagttttgaaaagtggtaaggaattgtgaaaaaaatctcagacttaatgctctctaaccacatgagtttcttcttttttatttagtaatacgctgctacatatttggaggttctggtgtttgtaggtcactgaacagacattgaaatctgatttatattgtataactgtaacatagaaagaaaaagtatttatattttttctgtaagaatatttcattgagttgtgtataatttaaataagatttgtccccaaatggttttgctcaccttgattttttttgttgtgattttcttgtttttgtataatgtgtatagtttatgtcaagggcattaaaagcctcctgaagcataatcttatcaaagggatacattgttaataaaatgtacttaaaattcttaaa